(19) United States
(12) Patent Application Publication
(10) Pub. No.: US 2024/0139222 A1
(43) Pub. Date: May 2, 2024

(54) HUMAN MILK OLIGOSACCHARIDE COMPOSITIONS FOR USE WITH BACTERIOTHERAPIES

(71) Applicant: PROLACTA BIOSCIENCE, INC., Duarte, CA (US)

(72) Inventors: Julie E. BUTTON, Duarte, CA (US); Gregory MCKENZIE, Duarte, CA (US); Scott ELSTER, Duarte, CA (US); Abigail REENS, Duarte, CA (US); Jessica PIERCE, Duarte, CA (US)

(21) Appl. No.: 18/021,043

(22) PCT Filed: Aug. 13, 2021
(86) PCT No.: PCT/US2021/045951

§ 371 Date: Feb. 13, 2023

Related U.S. Application Data
(60) Provisional application No. 63/065,991, filed on Aug. 14, 2020.
(60) Provisional application No. 63/225,170, filed on Jul. 23, 2021.

(51) Int. Cl. A61K 31/702; A61K 35/745; A61P 43/00

(52) U.S. Cl. CPC A61K 31/702; A61K 35/745; A61P 43/00

(57) ABSTRACT

Provided herein are methods and compositions for increasing the safety and effectiveness of a bacteriotherapy. In some aspects, a mixture of prebiotics, e.g., human milk oligosaccharides, are administered to a subject who has or will undergo a bacteriotherapy. In certain aspects, the mixture of prebiotics is administered in combination with a probiotic capable of internalizing and/or consuming the prebiotics. Also provided are methods for preparing or identifying strains of bacteria suitable for a bacteriotherapy, such as by incubating a mixture of bacteria obtained from stool in the presence of one or more of agents, e.g., human milk oligosaccharides, capable of promoting the growth of beneficial bacteria.

### CROSS-REFERENCE TO RELATED APPLICATIONS

This application claims priority from U.S. provisional application No. 63/065,991, filed on Aug. 14, 2020, entitled “HUMAN MILK OLIGOSACCHARIDE COMPOSITIONS FOR USE WITH BACTERIOTHERAPIES” and U.S. provisional application No. 63/225,170, filed on Jul. 23, 2021, entitled “HUMAN MILK OLIGOSACCHARIDE COMPOSITIONS FOR USE WITH BACTERIOTHERAPIES”; the contents of each are incorporated by reference in their entirety.

### INCORPORATION BY REFERENCE OF SEQUENCE LISTING

The present application is being filed along with a Sequence Listing in electronic format. The Sequence Listing is provided as a file entitled PROLO4102WOSEQLIST_ST25, created on Aug. 12, 2021, which is 372 kilobytes in size. The information in the electronic format of the Sequence Listing is incorporated by reference in its entirety.

### FIELD OF THE INVENTION

Provided herein are methods and compositions for increasing the safety and effectiveness of a bacteriotherapy. In some aspects, a mixture of prebiotics, e.g., human milk oligosaccharides, are administered to a subject who has or will undergo a bacteriotherapy. In certain aspects, the mixture of prebiotics is administered in combination with a probiotic capable of internalizing and/or consuming the prebiotics. Also provided are methods for preparing or identifying strains suitable for a bacteriotherapy, such as by incubating a mixture of bacteria obtained from stool in the presence of one or more of agents, e.g., human milk oligosaccharides, capable of promoting the growth of beneficial bacteria.

### BACKGROUND OF THE INVENTION

The human gut microbiome is unique to each individual and can include more than a thousand different species. Disruptions to the microbiome are associated with numerous diseases and conditions. Treatments aimed at correcting dysbiosis of the intestinal microbiome have traditionally relied on prebiotics, probiotics, and antibiotics and have generally failed to produce stable, long-term improvements. Fecal microbiota transplantation (FMT) which involves the transfer of fecal microbiota from a healthy donor to an ill recipient is a more recent alternative. While this strategy is reported to have some success for specific indications such as C. difficile infection, its usefulness for other indications including inflammatory bowel disease, functional gastrointestinal disorders, obesity, and metabolic syndrome appears limited. Furthermore, there are safety concerns associated with FMT and similar bacteriotherapies, such as risk of severe or life-threatening bacterial infections caused by drug-resistant bacteria.

What is needed in the art are compositions and methods to improve the efficacy and safety of bacteriotherapies such as FMT.

### SUMMARY OF THE INVENTION

Provided herein is a method of treating or preventing a disease, disorder, or condition associated with one or more of inflammation, immune dysfunction, cancer, allergy, or dysbiosis of the intestinal microbiome in a subject in need thereof, the method comprising administering to the subject a prebiotic mixture comprising one or more human milk oligosaccharides; wherein the subject has undergone or will undergo a bacteriotherapy. Also provided herein is a method of treating or preventing a disease, disorder, or condition associated with one or more of inflammation, immune dysfunction, cancer, allergy, or dysbiosis of the intestinal microbiome in a subject in need thereof, the method comprising administering to the subject i) a prebiotic mixture comprising one or more human milk oligosaccharides and ii) at least one probiotic strain of bacterium capable of consuming the one or more human milk oligosaccharides; wherein the subject has undergone or will undergo a bacteriotherapy.

In some embodiments, the bacteriotherapy comprises one or more bacteria species, subspecies, or strains obtained from, isolated from, derived from, or present in a human intestinal microbiome. In some embodiments, the bacteriotherapy comprises one or more bacteria species, subspecies, or strains obtained from, isolated from, derived from, or present in a human stool. In some embodiments, the bacteriotherapy comprises all or a portion of the bacteria present a human stool or a human intestinal microbiome. In some embodiments, the bacteriotherapy comprises a fecal microbiota transfer composition. In some embodiments, the bacteriotherapy comprises one or more bacterial species, subspecies, or strains that have been enriched, purified, or isolated from a stool sample obtained from of one or more healthy donors.

In some embodiments, the one or more bacterial species, subspecies, or strains were isolated from the stool of one or more healthy donors by a method comprising fractionating, gradient purification, solvent treatment, heat treatment, acid treatment, immunoprecipitation. In some embodiments, the bacteriotherapy is incubated in culture prior to administration to the subject. In some embodiments, the bacteriotherapy is incubated in the presence of an enrichment agent prior to administration to the subject, wherein the enrichment agent comprises a human milk oligosaccharide, the prebiotic mixture, a short chain fatty acid, acetate, butyrate, or lactate. In some embodiments, the bacteriotherapy comprises at least one butyrate producing strain of bacterium.

Also provided herein is a method of treating or preventing a disease, disorder, or condition associated with one or more of inflammation, immune dysfunction, cancer, allergy, or dysbiosis of the intestinal microbiome in a subject in need thereof, the method comprising administering to the subject i) a prebiotic mixture comprising one or more human milk oligosaccharides, ii) at least one probiotic strain of bacterium capable of consuming the one or more human milk oligosaccharides; and iii) at least one butyrate producing strain of bacterium. In some embodiments, the at least one butyrate producing strain is administered orally.

In some embodiments, the at least one butyrate producing strain of bacteria comprises between one and five strains of butyrate producing bacteria. In some embodiments, the at least one butyrate producing strain comprises a strain of Clostridium Cluster IV or Clostridium Cluster XIVa bacteria. In certain embodiments, the at least one butyrate producing strain comprises a 16S nucleic sequence having at least 95%, 98%, 99%, or 100% identity to any sequence set forth in SEQ ID NOS: 77-167. In some embodiments, the at least one butyrate producing strain comprises a 16S nucleic sequence having at least 95%, 98%, 99%, or 100% identity to any sequence set forth in SEQ ID NOS: 77-162. In particular embodiments, the at least one butyrate producing strain comprises at least one of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis. In some embodiments, the at least one butyrate producing strain comprises at least one of Anaerostipes caccae, Clostridium innocuum, Roseburia hominis, or Roseburia intestinalis.

In some embodiments, prebiotic mixture comprises at least 2, at least 5, at least 10, at least 25, at least 50, at least 100, or at least 150 human milk oligosaccharides. In certain embodiments, the prebiotic mixture comprises 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In some embodiments, the prebiotic mixture comprises at least 25 human milk oligosaccharides. In some embodiments, the prebiotic mixture is, is derived from, or comprises a human milk permeate, wherein the human milk permeate is obtained from the ultra-filtration of pooled human skim milk. In some embodiments, the pooled human milk is pooled from the milk of at least 25, 50, or 100 individual donors.

In certain embodiments, the prebiotic mixture comprises one or more of 2′-fucosyllactose, 3-fucosyllactose, difucosyllactose, 3′-sialyllactose, 6′-sialyllactose, Lacto-N-tetraose, Lacto-N-neo-tetraose, Lacto-N-fucopentaose I, Lacto-N-fucopentaose II, Lacto-N-fucopentaose III, Sialyl-lacto-N-tetraose a, Sialyl-lacto-N-tetraose b, Sialyl-lacto-N-tetraose c, Lacto-N-difuco-hexaose I, Lacto-N-difuco-hexaose II, Lacto-N-hexaose, para-Lacto-N-hexaose, Disialyllacto-N-tetraose, Fucosyl-Lacto-N-hexaose, Difucosyl-Lacto-N-hexaose a, Difucosyl-Lacto-N-hexaose b, lactodifucotetraose, 6′galactosyllactose, 3′galactosyllactose, 3-Sialyl-3-fucosyllactose, Sialylfucosyllacto-N-tetraose, Sialyllacto-N-fucopentaose V, disialyl-lacto-n-fucopentaose II, disialyl-lacto-n-fucopentaose V, Lacto-N-neo-difucohexaose II, 3-Fucosyl-sialylacto-N-tetraose c, para-Lacto-N-neohexose, Lacto-N-octaose, Lacto-N-neooctaose, Lacto-N-neohexaose, Lacto-N-fucopentaose V, iso-Lacto-N-octaose, para-Lacto-N-octaose, Lacto-decaose, or Sialyl-lacto-N-fucopentaose I.

In some embodiments, the prebiotic mixture comprises one or more of 2′-fucosyl-lactose, 3′-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In some embodiments, the prebiotic mixture comprises 2′-fucosyl-lactose, 3′-fucosyl-lactose, 3′-sialyl-lactose, 6′-sialyl-lactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose. In particular embodiments, the prebiotic mixture comprises one or more of 2′-fucosyllactose, 3-fucosyllactose, Lacto-N-tetraose, or lacto-N-neotetraose. In some embodiments, the prebiotic mixture comprises one or both of 2′-fucosyllactose and lacto-N-neotetraose. In certain embodiments, the prebiotic mixture comprises 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose.

In particular embodiments, wherein the prebiotic mixture is, is derived from, or comprises a concentrated human milk permeate, wherein the concentrated human milk permeate is obtained by a process comprising the steps of ultra-filtering human skim milk to obtain human milk permeate and concentrating the human milk oligosaccharide content of the human milk permeate. In certain embodiments, the human skim milk is obtained from human milk pooled from at least 25, 50, or 100 individual donors.

In some embodiments, the method further comprises administering at least one probiotic strain of bacterium capable of consuming the human milk oligosaccharides of the mixture. In some embodiments, the at least one probiotic strain comprises a species, subspecies, or strain of the genus Bifidobacterium. In some embodiments, the at least one probiotic strain comprises B. breve, B. bifidum, B. longum subsp. infantis, or B. longum subsp. longum. In some embodiments, the at least one probiotic strain comprises B. longum subsp. infantis.

Also provided herein is a method of treating or preventing a disease, disorder, or condition associated with one or more of inflammation, immune dysfunction, cancer, allergy, or dysbiosis of the intestinal microbiome in a subject in need thereof, the method administering to the subject a prebiotic mixture comprising at least 10 human milk oligosaccharides and a probiotic strain of bacterium, wherein the probiotic strain is B. longum subsp. infantis; and wherein the subject has undergone or will undergo a bacteriotherapy.

Also provided herein is a method of treating or preventing a disease, disorder, or condition associated with one or more of inflammation, immune dysfunction, cancer, allergy, or dysbiosis of the intestinal microbiome in a subject in need thereof, the method comprising administering to the subject i) a prebiotic mixture comprising one or more human milk oligosaccharides, ii) at least one probiotic strain of bacterium, wherein the at least one probiotic strain comprises B. longum subsp. infantis; and iii) at least one butyrate producing strain of bacterium, wherein the at least one butyrate producing strain comprises one or more of Agathobacter rectalis, Anaerobutyricum hallii, Anaerostipes caccae, Blautia producta, Clostridium innocuum, Clostridium leptum, Faecalibacterium prausnitzii, Anaerobutyricum soehngenii, Roseburia hominis, or Roseburia intestinalis.

Also provided herein is a method of treating or preventing a disease, disorder, or condition associated with one or more of inflammation, immune dysfunction, cancer, allergy, or dysbiosis of the intestinal microbiome in a subject in need thereof, the method administering to the subject (i) a prebiotic mixture comprising at least 10, at least 25, or at least 50 human milk oligosaccharides, wherein the human milk oligosaccharides comprise 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, and disialyllacto-N-tetraose and (ii) a probiotic strain of bacterium, wherein the probiotic strain is B. longum subsp. infantis; wherein the subject has undergone or will undergo a bacteriotherapy. In some embodiments, the bacteriotherapy comprises oral administration of at least one butyrate producing strain of bacterium, wherein the butyrate comprises one or more of Agathobacter rectalis, Anaerobutyricum hallii, Anaerostipes caccae, Blautia producta, Clostridium innocuum, Clostridium leptum, Faecalibacterium prausnitzii, Anaerobutyricum soehngenii, Roseburia hominis, or Roseburia intestinalis.

In some embodiments, the subject has, is suspected of having, or is at risk of having one or more of obesity, type II diabetes, a chronic inflammatory disease, an autoimmune disease, an infection, an infectious disease domination, bowel resection, a condition associated with chronic diarrhea, irritable bowel syndrome (IBS), inflammatory bowel disease (IBD), short bowel syndrome (SBS), celiac disease, small intestinal bacterial overgrowth (SIBO), gastroenteritis, leaky gut syndrome, pouchitis, gastric lymphoma, bacterial, viral, or parasitic infection or overgrowth, infection by drug-resistant bacteria, cancer, or graft versus host disease.

In some embodiments, one or both of the prebiotic mixture or the probiotic strain are administered prior to administration of the bacteriotherapy. In some embodiments, one or both of the prebiotic mixture or the probiotic strain are administered within 28 days, 21 days, 14 days, 7 days, 5 days, 3 days, or 1 day prior to administration of the bacteriotherapy. In some embodiments, the prebiotic mixture and the probiotic strain are both administered on the same days for at least 3, 5, 7, 14, 21, or 28 consecutive days prior to administration of the bacteriotherapy. In some embodiments, one or both of the prebiotic mixture or the probiotic strain are administered after administration of the bacteriotherapy. In some embodiments, one or both of the prebiotic mixture or the probiotic strain are administered within 28 days, 21 days, 14 days, 7 days, 5 days, 3 days, or 1 day after administration of the bacteriotherapy. In some embodiments, the prebiotic mixture and the probiotic strain are both administered on the same days for at least 3, 5, 7, 14, 21, or 28 consecutive days after to administration of the bacteriotherapy. In some embodiments, one or both of the prebiotic mixture and the probiotic strain are administered orally.

In some embodiments, the probiotic strain is administered in an amount of at least 1×10², 1×10³, 1×10⁴, 1×10⁵, 1×10⁶, 5×10⁶, 1×10⁷, 5×10⁷, 1×10⁸, 5×10⁸, 1×10⁹, 1×10¹⁰, 5×10¹⁰, 5×10¹¹, or 1×10¹² colony forming units (CFU) per day. In some embodiments, the probiotic strain is administered in an amount of between 1×10⁷ and 1×10¹⁰ colony forming units (CFU) per day. In some embodiments, the prebiotic mixture and/or the probiotic strain is administered with an enteric coating. In some embodiments, the prebiotic mixture is administered in an amount of at least 100 mg, 500 mg, 1 g, 2 g, 3 g, 4 g, 5 g, or 10 g total human milk oligosaccharides per day. In some embodiments, the prebiotic mixture is administered in an amount of between 100 mg and 50 g, 500 mg and 25 g, 1 g and 5 g, 2 g and 3 g, 3 g and 6 g, 5 g and 10 g, 8 g and 10 g, 10 g and 20 g, 15 g and 20 g, or 17 g and 19 g of total human milk oligosaccharides per day.

Additionally provided herein is a method of preparing a bacteriotherapy to treat or prevent a condition associated with dysbiosis of the intestinal microbiome; the method comprising incubating a mixture of bacteria obtained from stool with one or more enrichment agents in a culture, thereby obtaining a cultured mixture of bacteria, wherein the one or more enrichment agents comprise one or more of an oligosaccharide, a short chain fatty acid, or lactate; and collecting, harvesting, or isolating at least one cultured bacterium from the cultured mixture of bacteria, thereby obtaining a bacteriotherapy.

Also provided herein is a method for identifying one or more bacteria suitable for a bacteriotherapy; the method comprising incubating a mixture of bacteria obtained from stool with one or more enrichment agents in a culture, thereby obtaining a cultured mixture of bacteria, wherein the one or more enrichment agents comprise one or more of an oligosaccharide, a short chain fatty acid, or lactate; and collecting, harvesting, or isolating at least one cultured bacterium from the cultured mixture of bacteria, thereby obtaining a bacteriotherapy.

In some embodiments, the mixture of bacteria and the one or more enrichment agents are incubated in the presence a probiotic strain. In some embodiments, the probiotic strain is not present in the mixture of bacteria prior to the incubation. In some embodiments, the probiotic strain is a Bifidobacterium. In some embodiments, the probiotic strain is B. longum subsp. infantis. In some embodiments, the stool is human stool. In some embodiments, the stool is obtained from a healthy adult human. In some embodiments, the stool is pooled from the stool of two or more adult humans. In some embodiments, the stool is obtained from a healthy adult with a low risk or probability for a condition associated with dysbiosis of the intestinal microbiome.

In some embodiments, the enrichment agent comprises one or more oligosaccharides. In some embodiments, the one or more oligosaccharides comprise one or more human milk oligosaccharides. In some embodiments, the enrichment agent is a mixture of human milk oligosaccharides. In some embodiments, the prebiotic mixture comprises at least 2, at least 5, at least 10, at least 25, at least 30, at least 50, at least 100, or at least 150 human milk oligosaccharides. In some embodiments, the enrichment agent is a prebiotic mixture that is, is derived from, or comprises a concentrated human milk permeate from obtained from the ultra-filtration of pooled human skim milk. In some embodiments, the enrichment agent comprises a short chain fatty acid, optionally acetate. In some embodiments, the enrichment agent comprises lactate.

In some embodiments, the at least one cultured bacterium comprises a plurality of cultured bacteria. In some embodiments, the at least one cultured bacterium comprises all or essentially all of the cultured bacteria present in the cultured mixture of bacteria. In some embodiments, the at least one bacterium is collected, harvested, or isolated from the cultured mixture of bacteria by a method comprising one or more of gradient purification, differential centrifugation, solvent treatment, heat treatment, acid treatment, immunoprecipitation, or antibiotic selection

In some embodiments, a control mixture of bacteria is incubated in a culture in the absence of the enrichment agent, thereby obtaining a control cultured mixture of bacteria; and wherein the least one cultured bacterium from the cultured mixture of bacteria has a greater enrichment, level, presence, or abundance the cultured mixture of bacteria than in the control cultured mixture of bacteria and/or has faster growth or expansion in the presence of the enrichment agent than in the absence of the enrichment agent. In some embodiments, the control mixture of bacteria is obtained from the same stool as the mixture of bacteria. In some embodiments, the control mixture in incubated in the culture with one or more control agents. In some embodiments, wherein the mixture of bacteria is obtained from a stool sample collected from a healthy donor, wherein the healthy donor was administered a prebiotic mixture and a probiotic strain within less than four weeks prior to collection of the stool sample.

Also provided herein is a method of preparing a bacteriotherapy, the method comprising collecting, harvesting, or isolating at least one bacterium from a mixture of mixture of bacteria, wherein the mixture of bacteria is, originates from, or is obtained from a stool sample from a healthy donor, wherein the healthy donor was administered a prebiotic mixture and a probiotic strain capable of consuming the prebiotic mixture within less than four weeks prior to collection of the stool sample.

In some embodiments, the at least one bacterium comprises a plurality of bacteria that comprises two or more bacterial species, subspecies, or strains. In some embodiments, the at least one bacterium comprises all or essentially all of the cultured bacteria present in the mixture of bacteria. In some embodiments, the at least one bacterium is collected, harvested, or isolated from the cultured mixture of bacteria by a method comprising one or more of gradient purification, differential centrifugation, solvent treatment, heat treatment, acid treatment, immunoprecipitation, or antibiotic selection.

In some embodiments, the prebiotic mixture comprises at least 2, at least 5, at least 10, at least 25, at least 50, at least 100, or at least 150 human milk oligosaccharides, optionally at least 25 human milk oligosaccharides. In some embodiments, the prebiotic mixture is, is derived from, or comprises a human milk permeate, wherein the human milk permeate is obtained from the ultra-filtration of pooled human skim milk, optionally wherein the pooled human milk is pooled from the milk of at least 25, 50, or 100 individual donors. In some embodiments, the at least one probiotic strain of bacterium comprises B. breve, B. bifidum, B. longum subsp. infantis, or B. longum subsp. longum, optionally B. longum subsp. infantis.

In some embodiments, one or both of the prebiotic mixture and the probiotic strain are administered to the donor on the same days for at least 3, 5, 7, 14, 21, or 28 consecutive days prior to the collection of the stool sample. In some embodiments, the probiotic strain is administered in an amount at least 1×10⁷ colony forming units (CFU) per day, optionally between 1×10⁷ and 1×10¹⁰ colony forming units; and wherein the prebiotic mixture is administered in an amount of at least 1 g human milk oligosaccharides per day, optionally between 1 g and 20 g human milk oligosaccharides per day.

### BRIEF DESCRIPTION OF THE DRAWINGS

FIGS. 1A-1F display growth of pathogenic bacteria and of B. longum subsp. infantis. FIGS. 1A-1C show the growth of Klebsiella pneumoniae ATCC 43816 and B. longum subsp. infantis co-cultured at starting ratios of 1:1 (FIG. 1A), 10:1 (FIG. 1B), and 100:1 (FIG. 1C) K. pneumoniae cells to B. longum subsp. infantis cells. FIGS. 1D-1F display the growth of Klebsiella pneumoniae MH258 (FIG. 1D), E. clocacae (FIG. 1E), and E. coli (FIG. 1F) in the presence or absence of B. longum subsp. infantis. Error bars show standard deviation.

FIGS. 2A-2C display growth (FIGS. 2A and 2B) and short chain fatty acid production (FIG. 2C) in cultures of Anaerostipes caccae (A. caccae), B. longum subsp. infantis (B. infantis), or co-cultures of both (B. infantis+A. caccae). FIGS. 2A and 2B display cell culture growth as assessed by qPCR targeting Anaerostipes caccae (FIG. 2A) and B. longum sub. sp. infantis (FIG. 2B) of samples collected at various time points. FIG. 2C shows levels of butyrate, lactate, and acetate measured in cultures of Anaerostipes caccae, B. longum subsp. infantis, or both.

FIGS. 3A-3C display growth (FIGS. 3A and 3B) and butyrate production (FIG. 3C) in cultures of Anaerostipes caccae (AC), B. longum subsp. infantis (BI), or co-cultures of both. FIGS. 3A and 3B display cell culture growth as assessed by qPCR targeting Anaerostipes caccae (FIG. 3A) and B. longum sub. sp. infantis (FIG. 3B) of samples collected at various time points. FIG. 3C shows levels of butyrate measured in cultures of Anaerostipes caccae in the presence of HMO cultured with or without B. longum subsp. infantis.

FIGS. 4A-4I display growth (FIGS. 4A-4F) and butyrate production (FIGS. 4G-4I) in cultures of bacteria strains capable of producing butyrate (Anaerostipes caccae (AC; FIGS. 4A and 4B), Roseburia intestinalis (RI; FIGS. 4C and 4D), or Roseburia hominis (RH; FIGS. 4E and 4F)), B. longum subsp. infantis (BI), or co-cultures of both. FIGS. 4A-4F display cell culture growth as assessed by qPCR targeting Anaerostipes caccae (FIG. 4A), Roseburia intestinalis (FIG. 4C), Roseburia hominis (FIG. 4E), and B. longum sub. sp. infantis (FIGS. 4B, 4D, and 4F) of samples collected at various time points. FIGS. 4G-4I show levels of butyrate measured in cultures of Anaerostipes caccae (FIG. 4G), Roseburia intestinalis (FIG. 411), and Roseburia hominis (FIG. 4I). Conditions included the bacterial strains capable of producing butyrate: cultured alone with a carbon source lacking HMO (vehicle), cultured alone and in the presence of HMO (+HMO), cultured with B. longum subsp. infantis in the absence of HMO (+BI), or cocultured with B. longum subsp. infantis in the presence of HMO (+BI+HMO).

FIGS. 5A-5C show levels of butyrate measured in cultures of isolated strains of Anaerostipes caccae (FIGS. 5A and 5B) or Clostridium innocuum (FIG. 5C). Conditions included the bacterial strains capable of producing butyrate: cultured alone with a carbon source lacking HMO (vehicle), cultured alone and in the presence of HMO (+HMO), cultured with B. longum subsp. infantis in the absence of HMO (+BI), or cocultured with B. longum subsp. infantis in the presence of HMO (+BI+HMO).

FIG. 6 shows a graph displaying results of qPCR targeting B. longum subsp. infantis from fecal pellets of individual germ-free mice at various timepoints. Experimental groups of germ free mice included germ free mice that received FMT from the stool of a healthy human adult donor and treatment with HMO (open circles); germ free mice that received FMT from the stool of a healthy human adult donor and treatment with B. longum subsp. infantis and HMO (solid triangles); germ free mice that received FMT from the stool of a healthy human infant donor and treatment with B. longum subsp. infantis and HMO (open diamonds); and germ free mice received treatment with B. longum subsp. infantis and HMO but not FMT (solid squares).

### DETAILED DESCRIPTION

Provided herein are compositions and methods for improving, enhancing, augmenting, or increasing the one or more of the safety, efficiency, or efficacy of a bacteriotherapy, e.g., a fecal microbiota transfer (FMT). In some aspects, a prebiotic mixture, e.g., containing human milk oligosaccharides, is administered to the subject prior to, during, and/or after the administration of the bacteriotherapy. In certain aspects, a probiotic strain of bacterium, such as one capable of consuming or even internalizing and consuming the prebiotic mixture is also administered.

Bacteriotherapies including fecal microbiota transplants (FMT) have shown promise for the treatment of C. difficile infections, and some researchers believe in the potential of FMT to resolve other diseases or conditions that have been associated with dysbiosis of the intestinal microbiome. However, there is a risk that some bacteriotherapies, even those derived from stool of healthy individuals, may contain pathogenic bacteria that may go undetected and potentially lead to infection and serious complications, including invasive infections caused by extended-spectrum beta-lactamase (ESBL)-producing Escherichia coli (E. coli). At present, FMT is still an investigative procedure, and there are no “gold standard” safety precautions or standard regulations for clinicians to follow. Furthermore, while success of some FMT treatments in treating C. difficile infections have been reported, success for other indications such as inflammatory bowel disease, functional gastrointestinal disorders, obesity, and metabolic syndrome has been more limited and remains controversial. Given the large variation of microbiome compositions among healthy individuals, the microbial features of a bacteriotherapy required for successfully treating different indications remain uncertain. What is needed in the art are compositions and techniques to improve the safety and efficacy of bacteriotherapies such as FMT.

The provided compositions and methods address these needs. In particular, the present invention includes specific mixtures of prebiotics, such as human milk oligosaccharides, optionally in combination with probiotics, such as probiotics that consume human milk oligosaccharides, e.g., B. longum subspecies (subsp.) infantis (also referred to herein as B. infantis), that promote or facilitate conditions suitable for engraftment, colonization, or expansion of the beneficial bacteria present in a bacteriotherapy, e.g., an FMT, while reducing, minimizing, or preventing the engraftment, colonization, or expansion of pathogenic or potentially pathogenic microorganisms present in the subject's microbiome or within the bacteriotherapy.

In some aspects, the provided compositions and methods interact, e.g., synergistically, in vivo to influence the intestinal microbiome such as to correct or prevent dysbiosis. For example, in some aspects, the prebiotic mixture interacts with the probiotic strain and/or particular species, subspecies, or strains of the bacteriotherapy to synergistically reduce intestinal pH, increase microbial production of acetate, lactate, and/or butyrate, inhibit pathogenic taxa such as Enterobacteriaceae. In some instances, the prebiotic mixture is selectively consumed by particular bacteria capable of producing acetate and lactate, which in some instances may be the administered probiotic, e.g., B. longum subsp. infantis, and/or a bacterium present in the bacteriotherapy. In some instances, the synergistic production of acetate and lactate would efficiently lower pH, which inhibits pathogenic taxa, and/or stimulate, e.g., synergistically, microbial production of butyrate, which would promote health and repair of the host's gut epithelium. Particular embodiments contemplate that some or all of the resulting microbial production of acetate, lactate, and/or butyrate; the regulation of intestinal pH, the inhibition pathogenic taxa; and repair of the host's intestinal epithelium occurs to a much greater degree than would be expected from administering the prebiotic mixture, the probiotic strain, or the bacteriotherapy alone. Thus, in certain aspects, the provided compositions and methods interact synergistically to treat, correct, or prevent dysbiosis.

By improving the safety of bacteriotherapies such as FMT, the provided compositions and methods improve clinical outcomes for subjects who receive FMT for indications beyond C. difficile infection, and do so while reducing the safety risks and concerns associated with various bacteriotherapies. Thus, the provided compositions and methods may be used in conjunction with bacteriotherapies to treat and/or improve clinical outcomes in subjects suffering from various conditions or diseases, including but not limited to autoimmune diseases, immune dysfunction, inflammatory diseases, allergies, and cancers.

In some aspects, administering the prebiotic mixture, e.g., mixture of human milk oligosaccharides, to a subject who has undergone or will undergo a bacteriotherapy, e.g., an FMT, reduces colonization resistance of the subject's microbiome thereby allowing for more efficient engraftment or colonization of beneficial bacteria strains present in the bacteriotherapy. This promotion of the beneficial bacteria strains may be accomplished by directly promoting the growth of the beneficial strains within the bacteriotherapy; and/or by facilitating the growth of beneficial bacteria already present in the subject's microbiome that in turn promotes growth of the beneficial bacteria of the bacteriotherapy. In certain aspects, these effects on beneficial bacteria may be further improved by administering a probiotic capable of consuming the prebiotic mixture, e.g., B. longum subsp. infantis.

In certain aspects, administration of the prebiotic mixture promotes engraftment, colonization, or expansion of beneficial strains while reducing, impairing, or preventing the presence, abundance, expansion, or colonization of potentially pathogenic strains such as by influencing the environment of the intestinal microflora. In an exemplary model, administering the prebiotic mixture may result in or be associated with a reduction in intestinal pH, which may promote or be concurrent with expansion of strains that produce short chain fatty acids (SCFAs), e.g., acetate. In some aspects, the environmental influence may also include promoting butyrate and lactate production, such as by bacteria capable of consuming short chain fatty acids or prebiotic oligosaccharides. In certain aspects, these or similar influences on the environment may also promote or be concurrent with production of lactate and butyrate. In some aspects, the influence on the environment is augmented or increased by the additional administration of a probiotic, e.g., B. longum subsp. infantis, such as by influencing pH or SCFA, acetate, butyrate, or lactate production synergistically with the prebiotic mixture. In some embodiments, the environmental influence of the provided treatments (e.g., prebiotic mixture and probiotic strain) promote the engraftment, colonization, expansion, or growth of the beneficial bacteria and prevents or impairs the engraftment, colonization, expansion, or growth of potentially pathogenic bacteria, such as Enterobacteriaceae, Enterococcus, or Staphylococcus, in subjects administered a bacteriotherapy, e.g., an FMT.

In order to facilitate colonization of beneficial strains present within a bacteriotherapy, e.g., an FMT, subjects often receive antibiotic treatment prior to administration of a bacteriotherapy. In such instances, antibiotic treatments are thought to reduce the presence or abundance of pathogenic bacteria and facilitate colonization of strains present in the bacteriotherapy. However, antibiotic treatments do not successfully promote engraftment or colonization of the bacteriotherapy for all indications or all for varieties of bacteriotherapies. Furthermore, antibiotic treatments are often accompanied by unwanted side effects, and may not be safe for certain patient populations, such as young children with severe allergies. The provided compositions and methods provide a means to facilitate engraftment and colonization of a bacteriotherapy and reduce gut domination of pathogenic taxa, e.g., Enterobacteriaceae, Enterococcus, and Staphylococcus, without any reliance on antibiotic pretreatments.

In some embodiments, both the prebiotic mixture and the probiotic strain, e.g., B. longum subsp. infantis, are administered to a subject that has undergone or that will undergo a bacteriotherapy, e.g., an FMT. In some embodiments, the prebiotic mixture is selectively consumed by the probiotic, and the presence, abundance, growth, or expansion of the probiotic within the subject's microbiome may be tailored by altering dosage and timing for prebiotic administration. Thus, the effects of the probiotic may be upregulated or downregulated prior to, during, or after administration of the bacteriotherapy by adjusting the prebiotic regimen. Furthermore, it is contemplated that in some instances, the prebiotic mixture may also directly or indirectly promote engraftment or expansion of strains within the bacteriotherapy. Thus, in some instances, the timing and dosing of the prebiotic mixture may be adjusted by clinicians to promote or extend engraftment, colonization, or expansion of strains within the bacteriotherapy itself.

The present invention is based, at least in part, on the surprising realization that administration of the prebiotic mixture, e.g., human milk oligosaccharides, extends duration of colonization of the probiotic strain, e.g., B. longum subsp. infantis, within in the subject's microbiome. Surprisingly, administration of the prebiotic compositions provided herein result in engraftment and/or an expansion of the probiotic that may be detected days after the probiotic has been administered. Likewise, in certain embodiments, the provided prebiotic compositions prolong the colonization of different strains of beneficial microflora and/or increase the number of individual strains of the bacteriotherapy that successfully colonize, such as compared to administration of the bacteriotherapy in the absence of the prebiotic mixture.

Also provided herein are compositions and methods for screening bacteria suitable for incorporation into a bacteriotherapy. In some aspects, a mixture or collection of bacteria, such as from stool or an intestinal microbiome, are incubated in the presence of one or more enrichment agents that promote growth or expansion of beneficial bacteria and/or impair or prevent growth and expansion of pathogenic bacteria. In some aspects, such agents may include oligosaccharides such as human milk oligosaccharides, short chain fatty acids (SCFAs) such as acetate or butyrate, or lactate. In particular aspects, one or more bacterial strains are isolated or harvested from the mixture or collection following the incubation, such as for use as or in a bacteriotherapy or to be evaluated further as a candidate bacteriotherapy strain.

Among the potential drawbacks of bacteriotherapies such as FMT are the variations among the compositions of intestinal microbiomes across healthy individuals. It is not always certain that bacteriotherapies prepared from microbiota of different healthy individuals will have the same efficacy for treating subjects with dysbiosis or related disorders, nor is it presently understood which features of a successful bacteriotherapy actually contributed to the successful outcome. It is therefore difficult, at present, to predict which bacteriotherapies may be successful in subjects with different disorders or conditions, as well as to purposefully design a bacteriotherapy with specific features to improve the likelihood or degree of its success.

Furthermore, there are safety issues associated with bacteriotherapies, e.g., FMT, such as the risk of serious adverse reactions due to transmission of multi-drug resistant organisms. For example, the FDA issued a safety alert in 2019 informing health care providers and patients of the potential risk of serious or life-threatening infections with the use of FMT. Two immunocompromised adults who received investigational FMT developed invasive infections caused by extended-spectrum beta-lactamase (ESBL) producing Escherichia co/i (E. coli), and one of the individuals died. This event highlighted that risk that some bacteriotherapies, even those derived from stool of healthy individuals, may contain pathogenic bacteria that may go undetected and potentially lead to infection and serious complications.

The present compositions and methods for screening or preparing a bacteriotherapy address these issues. The provided methods for screening and preparing a bacteriotherapy are based, at least in part, on the realization that the capability for short chain fatty acid production, e.g., butyrate production, is a desirable feature that can be enriched, selected, or isolated from starting material with one or more of agents provided herein. The provided methods for screening and preparing allow for the design and production of more consistent and reliable bacteriotherapy treatments.

For example, in some aspects, incubation of bacteria, such as those obtained from stool or an intestinal microbiome, with the provided enrichment agents, e.g., human milk oligosaccharides, selectively promotes growth or expansion of strains possessing qualities that are desirable in a bacteriotherapy, e.g., growth in the presence of lactate or butyrate, while selectively slowing or impairing growth or expansion of potentially pathogenic taxa, e.g., Enterobacteriaceae, Enterococcus, and Staphylococcus. Following the incubation, some or all of the cultured bacteria may be harvested for use in a bacteriotherapy, or alternatively, strains may be isolated from the cultured bacteria for further evaluation prior to incorporation as a bacteriotherapy.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. COMPOSITIONS, KITS, AND ARTICLES OF MANUFACTURE

Provided herein are compositions, kits, and articles of manufacture that are or include one or both of at least one strain of probiotic bacterium (also referred to herein as a probiotic strain of bacterium or a probiotic strain) and a mixture of oligosaccharides. In some embodiments, the compositions are useful, inter alia, for administering to a subject who has received or will receive a bacteriotherapy, e.g., an FMT, such as to increase or improve the safety of the bacteriotherapy and/or to improve the effectiveness or efficiency of the bacteriotherapy. In particular embodiments, the prebiotic mixture and/or the probiotic strain are useful, inter alia, for use with a bacteriotherapy to improve one or more clinical outcomes in a disease, condition, or disorder, e.g., that are associated or accompanied with dysbiosis of the intestinal microbiome. In some embodiments, the compositions, kits, and articles of manufacture include a bacteriotherapy and/or an apparatus, reagent, or equipment suitable for preparing a bacteriotherapy.

In certain embodiments, the provided compositions, kits, and articles of manufacture are or include a prebiotic mixture, e.g., of oligosaccharides such as human milk oligosaccharides. In certain embodiments, the provided compositions, kits, and articles of manufacture are or include a probiotic strain. In certain embodiments, the provided compositions, kits, and articles of manufacture are or include the prebiotic mixture and the probiotic strain. In certain embodiments, the probiotic strain(s) and the mixture are included in separate compositions, e.g., are administered separately to a subject. Thus, in certain embodiments, provided herein are kits and articles of manufacture that include both of a composition that is or includes a probiotic strain and a composition that is or includes a mixture of oligosaccharides.

Also provided are kits and articles of manufacture that are or include one or more compositions that each contain both the probiotic strain and the mixture of oligosaccharides. In some embodiments, the kits and articles of manufacture also include a bacteriotherapy or an apparatus, reagent, or equipment suitable for use in preparing a bacteriotherapy. In certain embodiments, the provided compositions, kits, and articles of manufacture contain or include any of the mixtures of oligosaccharides, e.g., HMOs, that are described herein, such as in Section I-A. In particular embodiments, the provided compositions, kits, and articles of manufacture contain or include any of the probiotic strains, e.g., Bifidobacterium, described herein, such as those described in Section I-B. In particular embodiments, the compositions, kits, or articles of manufacture are useful, inter alia, for improving the safety or the efficacy of one or more bacteriotherapies, e.g., bacteriotherapies described herein such as in Section I-C. In certain embodiments, the compositions, kits, or articles of manufacture are one or more bacteriotherapies, e.g., bacteriotherapies described herein such as in Section I-C, or an apparatus, reagent, or equipment for preparing or administering bacteriotherapies. In some embodiments, the bacteriotherapy is or includes one or more strains of beneficial bacteria, e.g., in a form suitable for oral administration. In some embodiments, the bacteriotherapy is or includes at least one strain of bacterium capable of producing butyrate, e.g., any of the butyrate producing strains described herein such as in Section I-C-(i). The provided kits and articles of manufacture may also include labels or instructions for use. In some embodiments, such labels or instructions for use may describe any of the uses or methods provided herein, such as those described in Section II or Section III.

A. Prebiotic Mixtures

In some embodiments, the prebiotic mixture is a mixture of non-digestible carbohydrates, e.g., oligosaccharides such as human milk oligosaccharides (HMOs), that promotes the growth or expansion of the probiotic strain, e.g., in vivo such as in the human gut and/or within the human gut microbiome. In certain embodiments, the prebiotic mixture, e.g., of non-digestible carbohydrates such as HMOs, promotes, e.g., selectively or exclusively, the colonization, expansion, extension, or increased presence of the probiotic strain within the microbiome. In particular embodiments, the mixture of non-digestible carbohydrates, e.g., HMOs, promotes the growth or expansion of a probiotic strain of Bifidobacterium such as B. longum subsp. infantis, e.g., in vivo such as in the human gut. In certain embodiments, the prebiotic mixture is a mixture of oligosaccharides, e.g., HMOs, that promote, e.g., selectively or exclusively, the colonization, expansion, extension, or increased presence of one or more strains of Bifidobacterium, e.g., B. longum subsp. infantis, within the microbiome.

In some embodiments, the prebiotic mixture is or includes a mixture of non-digestible carbohydrates. In various embodiments, the prebiotic mixture is or includes a mixture of oligosaccharides. In particular embodiments, the prebiotic mixture is a mixture of one or more human milk oligosaccharides.

In some embodiments, the non-digestible carbohydrates are or include oligosaccharides. In particular embodiments, the non-digestible carbohydrates are or include milk oligosaccharides. In certain embodiments, the non-digestible carbohydrates are or include human milk oligosaccharides (HMOs). In some embodiments, the prebiotic mixture is a mixture of non-digestible carbohydrates that are or include human milk oligosaccharides. In particular embodiments, the prebiotic mixture is a mixture of human milk oligosaccharides, such as those that are obtained or derived from permeate, e.g., permeate derived or obtained from pooled human milk.

In some embodiments, the provided mixture may contain any oligosaccharide that may be internalized by one or more strains of Bifidobacterium such as a strain of B. longum subsp. infantis. In some embodiments, the oligosaccharides of the mixture may include one or more of a fructo-oligosacharide (FOS), galactooligosaccharide (GOS), transgalactooligosaccharide (TOS), gluco-oligosaccharide, xylo-oligosaccharide (XOS), chitosan oligosaccharide (COS), soy oligosaccharide (SOS), isomalto-oligosaccharide (IMOS), or derivatives thereof. In certain embodiments, such derivatives include those with modifications that may increase the likelihood or probability of consumption, metabolism, and/or internalization (such as by transport or import) of the oligosaccharide by the probiotic strain, e.g., B. infantis. Such modifications may include but are not limited to fucosylation or sialylation. In some embodiments, the oligosaccharides of the mixture may include one or more of a FOS, GOS, TOS, gluco-oligosaccharide, XOS, COS, SOS, IMOS, or derivatives or any or all of the foregoing, that are capable of being metabolized, consumed, and/or internalized by one or more strains, species, or subspecies of Bifidobacterium, e.g., B. longum subsp. infantis. In certain embodiments, the oligosaccharides of the mixture include one or more oligosaccharides that are obtained or derived from a resistant starch, pectin, psyllium, arabinogalactan, glucomannan, galactomannan, xylan, lactosucrose, lactulose, lactitol and various other types of gums such as tara gum, acacia, carob, oat, bamboo, citrus fibers, such as by treatment with enzymes that hydrolyze fiber or polysaccharides. In some embodiments, the one or more oligosaccharides of the mixture that are obtained by these means are capable of being consumed, metabolized, and/or internalized by at least one strain of Bifidobacterium such as B. longum subsp. infantis.

In certain embodiments, the prebiotic mixture is a mixture that is or includes at least one HMO. In some embodiments, the prebiotic mixture is a mixture of HMOs that is or includes a plurality of HMOs. In some embodiments, the prebiotic mixture is or includes a plurality of, of about, or at least 2, 3, 5, 10, 25, 50, 75, 100, 125, 150 different individual HMOs, e.g., HMOs with different individual chemical formulas or chemical structures. In certain embodiments, the prebiotic mixture is or includes a plurality of, of about, or at least 10, 25, 50, 75, 100, 125, 150 different individual HMOs. In some embodiments, the prebiotic mixture is or includes a plurality of, of about, or at least 25 different individual HMOs. In some embodiments, the prebiotic mixture is or includes a plurality of, of about, or of at least 80 different individual HMOs. Particular embodiments contemplate that one of skill may determine if an oligosaccharide is an HMO, such as if the oligosaccharide has a chemical formula and structure that is identical to an oligosaccharide that is found in human milk, as a matter of routine.

In certain embodiments, the prebiotic mixture contains one or more synthetic HMOs, e.g., HMOs that are obtained, purified, or synthesized from a source other than human milk. In some aspects, synthetic HMOs, as well as methods for synthesizing oligosaccharides and HMOs, are known, and include but are not limited to those described in PCT Publication Nos.: WO2017101958, WO2015197082, WO2015032413, WO2014167538, WO2014167537, WO2014135167, WO2013190531, WO2013190530, WO2013139344, WO2013182206, WO2013044928, WO2019043029, WO2019008133, WO2018077892, WO2017042382, WO2015150328, WO2015106943, WO2015049331, WO2015036138, and WO2012097950, each of which is incorporated by reference herein in its entirety.

In some embodiments, the prebiotic mixture includes some or all of 2′-fucosyllactose, 3′-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose. In particular embodiments, the mixture includes all of 2′-fucosyllactose, 3′-fucosyllactose, 3′-sialyllactose, 6′-sialyllactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose.

In certain embodiments, the prebiotic mixture includes some or all of 2-fucosyllactose, lacto-N-tetraorose, 3-sialyllactose, 3-fucosyllactose, lacto-N-fucopentaose I, lacto-N-fucopentaose II, and 6′sialyllactose. In particular embodiments, the prebiotic mixture includes some or all of 2′-fucosyl-lactose, 3′-fucosyl-lactose, 3′-sialyl-lactose, 6′-sialyl-lactose, Lacto-N-tetraose, Lacto-N-neo-tetraose, Lacto-N-fucopentaose I, Lacto-N-fucopentaose II, Lacto-N-fucopentaose III, Sialyl-lacto-N-tetraose b, Sialyl-lacto-N-tetraose c, Lacto-N-difuco-hexaose I, Lacto-N-difuco-hexaose II, Lacto-N-hexaose, para-Lacto-N-hexaose, Disialyllacto-N-tetraose, Fucosyl-Lacto-N-hexaose, Difucosyl-Lacto-N-hexaose a, and Difucosyl-Lacto-N-hexaose b.

In certain embodiments, the prebiotic mixture contains at least 25, 50, 100, 125, or 150 HMOs which include all of 2-fucosyllactose, lacto-N-tetraorose, 3-sialyllactose, 3-fucosyllactose, lacto-N-fucopentaose I, lacto-N-fucopentaose II, and 6′sialyllactose. In particular embodiments, the prebiotic mixture contains at least 10, 25, 50, 100, 125, or 150 HMOs which include all of 2′-fucosyl-lactose, 3′-fucosyl-lactose, 3′-sialyl-lactose, 6′-sialyl-lactose, Lacto-N-tetraose, Lacto-N-neo-tetraose, Lacto-N-fucopentaose I, Lacto-N-fucopentaose II, Lacto-N-fucopentaose III, Sialyl-lacto-N-tetraose b, Sialyl-lacto-N-tetraose c, Lacto-N-difuco-hexaose I, Lacto-N-difuco-hexaose II, Lacto-N-hexaose, para-Lacto-N-hexaose, Disialyllacto-N-tetraose, Fucosyl-Lacto-N-hexaose, Difucosyl-Lacto-N-hexaose a, and Difucosyl-Lacto-N-hexaose b. In some embodiments, the prebiotic mixture is a mixture of human milk oligosaccharides that is, includes, or is obtained or derived from human milk or a fraction thereof. In certain embodiments, the prebiotic mixture is or includes a mixture of HMOs that is or is obtained from an ultra-filtered permeate from human skim milk. In various embodiments, the prebiotic mixture is, includes, or is derived or produced from a concentrated human milk permeate. In some embodiments, the mixture of HMOs or the concentrated human milk permeate is or is obtained from a process described herein, e.g., in Section I-A-i. In certain embodiments, the prebiotic mixture is a concentrated human milk permeate, such as those described in U.S. Pat. No. 8,927,027 or in PCT Application No. WO 2018053535, incorporated herein by reference. In some embodiment, the prebiotic mixture is a prebiotic mixture produced or resulting from any of the methods described herein, e.g., in Section I-A-(i).

In some embodiments, the prebiotic mixture is or includes a concentrated human milk permeate that contains a plurality of human milk oligosaccharides. In particular aspects, the human milk permeate is obtained by filtering human skim milk that was obtained by separating cream from whole human milk. In some embodiments, the permeate was ultra-filtered from the human skim milk. In particular embodiments, the permeate is further concentrated, e.g., by reverse osmosis or nanofiltration, to increase the HMO content within the permeate. In some embodiments, the human milk permeate, e.g., the concentrated human milk permeate, has a concentration of at least 0.5%, 1%, 2.5%, 5%, or 10% w/v HMO. The human milk permeate may undergo additional processing steps, such as to digest or remove sugars, e.g., lactose, prior to its formulation or incorporate as a prebiotic mixture.

In certain embodiments, the concentrated human milk permeate includes a plurality of, of about, or of at least 1, 2, 3, 5, 10, 25, 50, 75, 100, 125, 150 different individual HMOs, e.g., HMOs with different individual chemical formulas or chemical structures. In certain embodiments, the prebiotic mixture is or includes a plurality of, of about, or of at least 10, 25, 50, 75, 100, 125, 150 different individual HMOs. In some embodiments, the prebiotic mixture is or includes a plurality of, of about, or of at least 25 different individual HMOs. In some embodiments, the prebiotic mixture is or includes a plurality of, of about, or of at least 80 different individual HMOs. Thus, in some embodiments, the prebiotic mixture is or includes a concentrated human milk permeate and contains at least 10, 25, 50, 75, 100, 125, 150 different individual HMOs. In particular embodiments, the concentrated human milk permeate contains at least 10, 25, 50, 100, 125, or 150 HMOs which include all of 2-fucosyllactose, lacto-N-tetraorose, 3-sialyllactose, 3-fucosyllactose, lacto-N-fucopentaose I, lacto-N-fucopentaose II, and 6′sialyllactose.

In certain embodiments, the concentrated human milk permeate and/or the prebiotic mixture has an increased amount, level, or concentration of one or more HMOs as compared to what is typically found human milk. In particular embodiments, the prebiotic mixture has an increased amount, level, or concentration of one or more HMOs as compared to what is typically found in untreated human milk permeate, e.g., permeate resulting from ultrafiltration of pooled human skim milk. In particular embodiments, the prebiotic mixture is or includes at least 25, 50, 75, 100, 125, 150, of the different HMOs found, present, or detected in pooled human milk or in permeate (e.g., permeate resulting from ultra-filtering skim) obtained from pooled human milk. In some embodiments, the prebiotic mixture is or includes at least 50%, 60%, 70%, 80%, 85%, 90%, 95%, 97%, 99%, or 99.9% of the different HMOs found, present, or detected in pooled human milk or in permeate (e.g., permeate resulting from ultra-filtering skim) obtained from pooled human milk. In certain embodiments, the prebiotic mixture is or includes at least 50%, 60%, 70%, 80%, 85%, 90%, 95%, 97%, 99%, or 99.9% of the individual HMOs that may be found, present, or detected across samples of human milk, e.g., samples of milk obtained from different individuals. In some embodiments, the prebiotic mixture of HMOs is or includes the same or substantially the same HMOs found, present, or detected in pooled human milk or in permeate (e.g., permeate resulting from ultra-filtering skim) obtained from pooled human milk. In certain embodiments the prebiotic mixture is or includes a human milk permeate resulting from the ultrafiltration of human whole or skim milk pooled from at milk collected from at least 10, 25, 50, or 100 individual human milk donors that is further concentrated, e.g., by nanofiltration or reverse osmosis, to increase the concentration of total HMO (e.g., by w/w).

In certain embodiments, the prebiotic mixture is free or essentially free of oligosaccharides that are not HMOs. In certain embodiments, the prebiotic mixture contains human milk permeate, e.g., concentrated human milk permeate, and one or more synthetic HMOs, e.g., one or more of synthetically derived 2′-fucosyl-lactose, 3′-fucosyl-lactose, 3′-sialyl-lactose, 6′-sialyl-lactose, Lacto-N-tetraose, Lacto-N-neo-tetraose, Lacto-N-fucopentaose I, Lacto-N-fucopentaose II, Lacto-N-fucopentaose III, Sialyl-lacto-N-tetraose b, Sialyl-lacto-N-tetraose c, Lacto-N-difuco-hexaose I, Lacto-N-difuco-hexaose II, Lacto-N-hexaose, para-Lacto-N-hexaose, Disialyllacto-N-tetraose, Fucosyl-Lacto-N-hexaose, Difucosyl-Lacto-N-hexaose a, and Difucosyl-Lacto-N-hexaose b.

Prebiotic mixtures containing human milk oligosaccharides for use in the compositions and methods disclosed herein may be obtained according to methods known in the art, including, but not limited to, chemical synthesis and purification from human milk. For example, processes to obtain HMO compositions from human milk are described below and are detailed in PCT Pub. Nos. WO/2010/065652 and WO/2018/053535, the contents of which are hereby incorporated in their entirety.

In some embodiments, the prebiotic mixture is a mixture of HMOs that are or are derived from a concentrated ultra-filtered human milk permeate, e.g., any ultra-filtered human milk permeate described herein such as in Section I-A-(i).

In some embodiments, the prebiotic mixtures are mixtures of HMOs having an HMO profile that is substantially similar both structurally and functionally to the profile of HMOs observed across the population of whole human milk. That is to say, in some aspects, since the prebiotic mixtures may be obtained from a source of human milk derived from a pool of donors rather than an individual donor, the array of HMOs will be more diverse than in any one typical individual, and will represent or more closely represent the spectrum of HMOs that are found among human milk across a population as opposed to the spectrum of HMOs that are found or typically found in the human milk produced by any particular individual. Thus, in some embodiments, the prebiotic mixture and the concentrated human milk permeate have more individual HMO species than what can be found in human milk obtained from an individual donor.

In some instances, the ratio of the amount or concentration of individual human milk oligosaccharide species to total human milk oligosaccharides of the prebiotic mixture or the concentrated human milk permeate may be different from what would be observed in whole human milk or pooled human milk.

In some embodiments, the prebiotic mixture is or includes a greater amount of different individual HMOs than the number of different individual HMOs found in human milk from an individual donor. In certain embodiments, the prebiotic mixture includes at least 1, 2, 3, 4, 5, 10, 15, 20, 25, 30, 35, 40, 45, or 50 more individual HMOs than the number of different individual HMOs found in human milk from an individual donor. In particular embodiments, a prebiotic mixture is or includes a greater amount of different individual HMOs than the mean or median number of different individual HMOs found in a plurality of human milk samples from individual donors. In certain embodiments, the prebiotic mixture includes at least 1, 2, 3, 4, 5, 10, 15, 20, 25, 30, 35, 40, 45, or 50 more individual HMOs than the number of different individual HMOs found in human milk from an individual donor.

In some aspects, one of the biggest variables in HMO diversity derives from the mother's Lewis blood group and specifically whether or not she has an active fucosyltrasferase 2 (FUT2) and/or fucosyltrasferase 3 (FUT3) gene. When there is an active FUT2 gene, an al-2 linked fucose is produced, whereas fucose residues are al-4 linked when the FUT3 gene is active. The result of this “secretor status” is, generally, that “secretors” (i.e. those with an active FUT2 gene) produce a much more diverse profile of HMOs dominated by al-2 linked oligosaccharides, whereas “non-secretors” (i.e. those without an active FUT2 gene) may comprise a more varied array of, for example α1,-4 linked oligosaccharides (as compared to secretors), but comprise an overall decrease in diversity since they are unable to synthesize a major component of the secretor's HMO repertoire. In some embodiments, the prebiotic mixture includes human milk oligosaccharides that include al-2 linked fucose and human milk oligosaccharides that include al-4 linked fucose.

In some embodiments, the prebiotic mixture is or includes at least 5% total HMO (w/w). In particular embodiments, the prebiotic mixture is or includes least 5%, 6%, 7%, 8%, 9%, 10%, 11%, 12%, 15%, 20%, 25%, or 50% total HMO (w/w). In certain embodiments, the prebiotic mixture is or includes between 5% and 15%, 7.5% and 12.5%, 8% and 12%, 8.5% and 11%, or 8.4% and 10.6% total HMO (w/w). In certain embodiments, the prebiotic mixture is or includes between 8.5% and 11% total HMO (w/w). In some embodiments, the prebiotic mixture is or includes between 8.4% and 10.6% total HMO (w/w).

In some embodiments, the prebiotic mixture has a pH of between 4.0 and 5.5. In certain embodiments, the prebiotic mixture has less than 10%, 5%, 1%, or 0.1% lactose (w/w). In some embodiments, the prebiotic mixture has less than 10%, 5%, 1%, or 0.1% glucose (w/w). In particular embodiments, the prebiotic mixture has less than 10%, 5%, 1%, or 0.1% galactose (w/w). In certain embodiments, the prebiotic mixture has less than 10% galactose, less than 10% glucose, and less than 0.1% lactose.

In some embodiments, the prebiotic mixture is a liquid formulation. In some embodiments, the prebiotic mixture is in powdered form, e.g., a lyophilized or spray dried composition. In certain embodiments, the prebiotic mixture is incorporated into a dosage form that is a separate composition from the probiotic strain. In some embodiments, the prebiotic mixture is incorporated into a dosage form that is a separate composition from the bacteriotherapy

i) Exemplary Methods for Manufacturing Prebiotic Mixtures

In some embodiments, the prebiotic mixture is or includes human milk oligosaccharides (HMOs) obtained or purified from ultra-filtered permeate from donated human milk. In certain embodiments, the donated human milk is pooled to provide a pool of human milk. In some embodiments, a pool of human milk comprises milk from two or more (e.g., ten or more) donors. In certain embodiments, the pooled human milk contains milk from at least 50, 75, 100, 150, or 200 individual donors. In certain embodiments, the pooled human milk contains human milk from at least 100 individual donors or between 100 and 300 individual donors. In some embodiments, the pooled human milk contains milk from at least ten, at least twenty-five, at least fifty, at least seventy-five, at least one hundred, or at least one hundred fifty individual human milk donors.

In some embodiments, the human milk oligosaccharides that are contained or included in the prebiotic mixture are synthetic human milk oligosaccharides, such as those derived from non-human milk sources, e.g., derived or obtained as oligosaccharides or precursors from transgenic microorganisms and/or chemically synthesized. In some aspects, synthetic oligosaccharides and HMOs, as well as methods and techniques for synthesizing oligosaccharides and HMOs, are known, and include but are not limited to those described in PCT Publication Nos.: WO2017101958, WO2015197082, WO2015032413, WO2014167538, WO2014167537, WO2014135167, WO2013190531, WO2013190530, WO2013139344, WO2013182206, WO2013044928, WO2019043029, WO2019008133, WO2018077892, WO2017042382, WO2015150328, WO2015106943, WO2015049331, WO2015036138, and WO2012097950, each of which is incorporated by reference herein in its entirety.

In particular embodiments, one or more synthetically derived human milk oligosaccharides are added to a concentrated human milk permeate to arrive at a prebiotic mixture.

In certain embodiments, the mixture of oligosaccharides described herein are produced from human milk permeate, e.g., concentrated ultra-filtered permeate from pooled human milk. In some embodiments, the mixture of oligosaccharides described herein contain or are formulated with human milk permeate, e.g., concentrated ultra-filtered permeate from pooled human milk. In some embodiments, the concentrated ultra-filtered permeate may be made according to any suitable method or technique known in the art. In some aspects, suitable methods and techniques include those described in U.S. Pat. No. 8,927,027 and PCT Pub. No. WO2018053535, hereby incorporated by reference in their entirety. Exemplary methods and techniques for producing the human milk compositions are briefly summarized herein.

In some embodiments, the prebiotic mixture is or include human milk permeate that has been generated or produced by a method described herein. In some embodiments, whole human milk is pooled from multiple donors, and then cream and skim are separated by any suitable technique known in the art, e.g., centrifugation; and then the skim milk is filtered, e.g., ultra-filtered, to obtain human milk permeate and retentate. The human milk permeate may be further processed, such as to remove or digest one or more components, e.g., lactose, or to increase the concentration of human milk oligosaccharides, such as by nanofiltration or reverse osmosis.

In some embodiments, the donor milk is received frozen, and when desired, is thawed and pooled. In some embodiments, donor milk is then screened, e.g., to identify contaminants, by one or more of the methods discussed herein.

In some embodiments, the pooled milk is filtered, e.g., through about a 200-micron filter. In some embodiments, the pooled milk is heated, e.g., at about 63° C. or greater for about 30 minutes or more. In some embodiments, the milk is transferred to a separator, e.g., a centrifuge, to separate the cream from the skim. In some embodiments, the cream may go separation once again to yield more skim. In some embodiments, a desired amount of cream is added to the skim prior to ultra-filtration. In certain embodiments, material that that did not pass through the filter is collected as the retentate fraction, and material that passes through the filter is collected as the permeate fraction.

In some embodiments, the skim fraction undergoes ultra-filtration. In some embodiments, the ultrafiltration is performed with a filter between 1 kDa and 1000 kDa to obtain a protein rich retentate and the HMO-containing permeate. Details of this process can be found, for example, in U.S. Pat. Nos. 8,545,920; 7,914,822; 7,943,315; 8,278,046; 8,628,921; and 9,149,052, each of which is hereby incorporated by reference in its entirety. In some embodiments, the ultra-filtration is performed with a filter that is between 1 kDa and 100 kDa, 5 kDa and 50 kDa, or 10 kDa and 25 kDa. In certain embodiments, the filter is about or at least 5 kDa, 10 kDa, 20 kDa, 25 kDa, 50 kDa, or 100 kDa. In some embodiments, the skim fraction undergoes ultrafiltration with a filter that is about 10 kDa. In certain embodiments, the skim fraction undergoes ultrafiltration with a filter that is about 25 kDa. In particular embodiments, the skim fraction undergoes ultrafiltration with a filter that is about 50 kDa.

In some embodiments, the ultra-filtered permeate undergoes a process for reducing lactose. In certain embodiments, a process for producing a concentrated human milk permeate composition with substantially reduced levels of lactose is provided. In certain embodiments, the substantial reduction includes or requires the biochemical and/or enzymatic removal of lactose from the lactose-rich human milk permeate fraction, without loss of yield or change in molecular profile of the HMO content of human milk permeate. And, in particular embodiments, without leaving residual inactivated foreign protein, if enzymatic digestion is used to reduce lactose. In certain embodiments, about or at least 50%, 60%, 70%, 80%, 90%, 95%, 99%, 99.9%, or 99.99% of the lactose present in the permeate after ultrafiltration is removed, e.g., enzymatically digested. In certain embodiments, the permeate is free or essentially free of lactose following the enzymatic digestion.

In certain embodiments, the process for reducing lactose from human milk permeate includes one or more of the steps of a) adjusting the pH of the permeate mixture; b) heating the pH adjusted mixture; c) adding lactase enzyme to the heated permeate mixture to create a permeate/lactase mixture and incubating a period of time; d) removing the lactase from the mixture and filtering the mixture to remove lactase; and e) concentrating human milk oligosaccharides. In some embodiments, the order of when steps (a)-(c) are performed may be varied. Thus, in some aspects, the steps may be performed in the order of (a)-(b)-(c); (a)-(c)-(b); (c)-(b)-(a); (c)-(a)-(b); (b)-(a)-(c); or (b)-(c)-(a), such that, for example, the lactase enzyme may be added prior to heating the mixture, or, alternatively at any point during the heating process. Similarly, and also by way of example only, the mixture may be heated prior to adjustment of the pH. Furthermore, several steps may be grouped into a single step, for example “enzymatically digesting lactose” or “lactases digestion of lactose” involves steps (a)-(c) as described. These steps may be performed concurrently or consecutive in any order. Therefore, as used herein “lactose digestion” refers to the performance of at least these three steps, in any order, consecutively or concurrently.

In certain embodiments, the pH of the permeate is adjusted to a pH of about 3 to about 7.5. In some embodiments, the pH is adjusted to a pH of about 3.5 to about 7.0. In particular embodiments, the pH is adjusted to a pH of about 3.0 to about 6.0. In certain embodiments, the pH is adjusted to a pH of about 4 to about 6.5. In some embodiments, pH is adjusted to a pH of about 4.5 to about 6.0. In particular embodiments, the pH is adjusted to a pH of about 5.0 to about 5.5. In certain embodiments, the pH is adjusted to a pH of about 4.3 to about 4.7, preferably 4.5. The pH may be adjusted by adding acid or base. In some embodiments, pH is adjusted by adding acid, for example HCl. In particular embodiments, pH is adjusted by adding 1N HCl and mixing for a period of time e.g. about 15 minutes.

In some embodiments, the pH-adjusted permeate is heated to a temperature of about of about 25° C. to about 60° C. In certain embodiments, the permeate is heated to a temperature of about 30° C. to about 55° C. In some embodiments, the permeate is heated to a temperature of about 40° C. to about 50° C. In certain embodiments, the permeate is heated to a temperature of about 48° C. to about 50° C. In some embodiments, the permeate is heated to a temperature about 50° C. In some embodiments, the permeate is heated to a temperature less than or equal to about 40° C.

In particular embodiments, lactase enzyme is added to the pH-adjusted, heated permeate to create a permeate/lactase mixture. In certain embodiments, lactose within the permeate/lactase mixture is broken down into monosaccharides. In certain embodiments, lactase enzyme is added at about 0.1% w/w to about 0.5% w/w concentration. In certain embodiments, lactase enzyme is added at about 0.1% w/w, or 0.2% or 0.3% or 0.4% or 0.5% w/w. There are many commercially available lactase enzymes that may be used. As such, the lactase enzyme may be derived from any origin (e.g. fungal or bacterial in origin).

In some embodiments, the pH-adjusted, heated permeate is incubated with the lactase enzyme for about 5 to about 225 minutes. In certain embodiments, the incubation time is about 15 min to about 90 min. In some embodiments, the incubation time is about 30 minutes to about 90 minutes. In particular embodiments, the incubation time is about 60 minutes. Some aspects contemplate that that incubation time is dependent upon myriad of factors including, but not limited to, the source of the enzyme used, the temperature and pH of the mixture and the concentration of enzyme used. Thus, in some embodiments, incubation time with the lactase enzyme may be adjusted to factor in such variables as a matter of routine. While the pH, temperature, and enzyme incubation conditions provided here are what work optimally for the process described herein, one of skill in the art would understand that modifications may be made to one or more of these variables to achieve similar results. For example, if less enzyme is used than the about 0.1% w/w to about 0.5% w/w described herein, the incubation time may need to be extended to achieve the same level of lactose digestion. Similar adjustments may be made to both the temperature and pH variables as well.

In certain embodiments, after incubation the permeate/lactase mixture is cooled to a temperature of about 20° C. to about 30° C. In a particular embodiment, the permeate/lactase mixture is cooled to a temperature of about 25° C.

In some embodiments, the permeate/lactase mixture is clarified to remove insoluble constituents. In certain embodiments, insoluble material may form throughout the change in pH and temperature. Thus, in some embodiments, it may be necessary or beneficial to clarify the mixture to remove these insoluble constituents, for example, through a depth filter. The filters may be 0.1 to 10 micron filters. In some embodiments, the filters are about 1 to about 5 micron filters. Alternatively, removal of insoluble constituents can be achieved through a centrifugation process or a combination of centrifugation and membrane filtration. The clarification step is not essential for the preparation of a diverse HMO composition, as described herein, rather, this optional step aids in obtaining a more purified permeate composition. Furthermore, the clarification step is important in the reusability of the filtration membranes and thus to the scalability of the process. Some aspects contemplate that, without adequate clarification, substantially more filter material is required, increasing the difficulty and expense to produce permeate compositions at clinical scale. However, one will understand that more or less stringent clarification may be formed at this stage in order to produce more or less purified permeate compositions, depending on formulation and application. For example, precipitated minerals may be less of a problem for a formulation destined for lyophilization.

In certain embodiments, to remove the spent and excess lactase enzyme from the clarified permeate/lactase mixture. There may, however, be some instances where the inactivated foreign protein will carry no biological risk and therefore the added steps of lactase removal or even inactivation may not be necessary. In some embodiments, the spent and excess lactase is inactivated, for example by high temperature, pressure, or both. In some embodiments, the inactivated lactase is not removed from the composition.

In other embodiments, however, a further purification to remove foreign proteins will be called for. In such embodiments lactase enzyme removal may be accomplished by ultrafiltration. In some embodiments, ultrafiltration is accomplished using an ultrafiltration membrane, for example using a membrane with molecular weight cut-off of ≤50,000 Dalton, e.g. a BIOMAX-50K. In some embodiments, the molecular weight cut-off less than or equal to about 10 kDa. In certain embodiments, the molecular weight cut-off less than or equal to about 25 kDa. In particular embodiments, the molecular weight cut-off less than or equal to about 50 kDa.

In certain embodiments, an additional ultrafiltration is performed through a smaller membrane than the initial a membrane with molecular weight cut-off of ≤50,000 Dalton. In some embodiments, the additional ultrafiltration is performed with a membrane with a molecular weight cut off of between 10 kDa and 50 kDa, 1 kDa and 10 kDa, 1 kDa and 5 kDa, or 2 kDa and 3 kDa. In certain embodiments, the additional ultrafiltration is performed with a membrane with a molecular weight cut off of between 2 kDa and 3 kDa. In certain embodiments, an additional ultrafiltration is not performed. In some embodiments, the additional filtration step is performed, such as to aid in the overall purity of the permeate product, such as by assisting in the removal of smaller potentially bioactive and/or immunogenic factors such as microRNAs and exosomes.

In some embodiments, the clarified mixture that has undergone at least one, and in some cases two or more rounds of ultrafiltration (or alternative lactase removal means) is further filtered to purify and concentrate human milk oligosaccharides and to reduce the mineral and monosaccharides content.

In some embodiments, filtration can be accomplished using a nanofiltration membrane. In some embodiments, the membrane has a molecular weight cut-off of ≤1,000 Dalton. In certain embodiments, the membrane has a molecular weight cut-off of between 1 kDa and 1,000 kDa. In certain embodiments, the membrane has a molecular weight cut-off of less than 600 Da. In certain embodiments, the membrane has a molecular weight cut-off between 400 Da and 500 Da. In some aspects, the additional nanofiltration removes monosaccharides, minerals, particularly calcium, and smaller molecules to produce the final purified HMO composition.

In some embodiments, additional or alternative steps may be taken for the removal of minerals. Such an additional step may include, for example, centrifugation, membrane clarification (≤0.6 micron), or combination of centrifugation and membrane filtration of heated (≥40° C.) or refrigerated/frozen and thawing of HMO Concentrate. The collected supernatant or filtrate of these additional or alternative steps, in some embodiments, is concentrated further using a nanofiltration membrane. In some embodiments, the nanofiltration comprises filtration through a membrane with a molecular cut off of ≤600 Dalton. In some embodiments, these additional steps may be performed at any stage of the process, including but not limited to prior to or after pasteurization.

In some embodiments, the physical property of nanofiltration membranes can be modified, such as chemical modification, to selectively concentrate sialylated HMOs, for example, allowing greater efficiency of neutral HMOs removal from HMO concentrate, in instances where concentrated sialylated HMOs are preferred.

In certain embodiments, the permeate may be further processed, e.g., concentrated or diluted. In some embodiments, the permeate may be concentrated by a suitable process such as nanofiltration, reverse osmosis, or dried, e.g., lyophilized.

In some embodiments, the permeate is treated to reduce bioburden, such as by any means known in the art. In some embodiments, the purified HMO composition is pasteurized. In some aspects, pasteurization is accomplished at ≥63° C. for a minimum of 30 minutes. Following pasteurization, the composition is cooled to about 25° C. to about 30° C. and clarified through a 0.2 micron filter to remove any residual precipitated material.

In certain embodiments, the mixture of oligosaccharides is formulated with an ultra-filtered permeate obtained from human milk. In some embodiments, the mixture of oligosaccharides is formulated with permeate that has been ultra filtered from the skim fraction of pooled human milk. In certain embodiments, lactose is removed, e.g., enzymatically degraded, prior to formulation into the mixture of oligosaccharides.

B. Probiotic Strains

In particular embodiments, provided herein are compositions that are or include a probiotic strain of bacteria, e.g., a strain of Bifidobacteria such as B. longum subsp. infantis. In some embodiments, the probiotic strain, e.g., B. longum subsp. infantis, is contained or included in the same composition as the mixture of oligosaccharides, e.g., the mixture described herein such as in Section I-A. In certain embodiments, the probiotic strain, e.g., the probiotic strain, e.g., B. longum subsp. infantis, is contained or included in a separate composition from the mixture. In certain embodiments, the probiotic strain is or includes two or more strains of bacterium, such as any two or more of those described herein.

In particular embodiments, the probiotic strain is capable of consuming or metabolizing non-digestible carbohydrates, e.g., oligosaccharides such as HMOs. In particular embodiments, the probiotic strain is capable of consuming or metabolizing oligosaccharides such as HMOs. In some embodiments, the probiotic strain is capable of utilizing the prebiotics of the prebiotic mixture, e.g., HMOs, as a carbon source. In particular embodiments, HMOs, are preferentially consumed or metabolized by the probiotic strain, e.g., as compared to other microbes or bacteria present in the gut or microbiome. In certain embodiments, the probiotic strain is capable of consuming or metabolizing one or more prebiotics of the mixture, including those of any of the mixtures described herein, e.g., in Section I-A. In certain embodiments, the probiotic strain is capable of consuming or metabolizing all or essentially all of the oligosaccharides of the mixture. In certain embodiments, the probiotic strain is capable of consuming or metabolizing HMOs. Particular embodiments contemplate that probiotic strains that consume or metabolize HMOs are known, and may be identified by routine techniques such as those described in Gotoh et al. Sci Rep. 2018 Sep. 18; 8(1):13958, incorporated by reference herein in its entirety.

In some embodiments, the probiotic strain contains one or more enzymes capable of hydrolyzing the prebiotics of the mixture. In certain embodiments, the probiotic strain contains one or more enzymes capable of hydrolyzing the human milk oligosaccharides. In particular embodiments, the one or more enzymes hydrolyze external oligosaccharides, e.g., oligosaccharides such as HMOs that are outside of the probiotic cell. In some embodiments, the one or more enzymes hydrolyze oligosaccharides such as human milk oligosaccharides internally or within the probiotic cell. In certain embodiments, the one or more enzymes hydrolyze internalized human milk oligosaccharides.

In particular embodiments, the probiotic strain contains one or more enzymes capable of hydrolyzing one or more HMOs. In particular embodiments, the one or more enzymes hydrolyze external HMOs. In some embodiments, the one or more enzymes hydrolyze HMOs that are outside of the probiotic cell. In some embodiments, the one or more enzymes hydrolyze HMOs internally. In particular embodiments, the one or more enzymes hydrolyze HMOs within the probiotic cell. In certain embodiments, the one or more enzymes hydrolyze internalized HMOs.

In some embodiments, the probiotic strain is capable of internalizing human milk oligosaccharides. In certain embodiments, the probiotic strain internalizes human milk oligosaccharides prior to hydrolyzing the human milk oligosaccharides. In various embodiments, the at least one probiotic selectively or exclusively utilizes human milk oligosaccharides as a carbon source. Thus, in certain embodiments, if the at least one probiotic is administered to the subject and/or has engrafted, e.g., within the subject's microbiome (such as the intestinal microbiome), the at least one probiotic is present, expands, or increases in amount within the subject's microbiome when human milk oligosaccharides are administered to and/or ingested by the subject, and, in certain embodiments, the at least one probiotic is no longer present and/or decreases in amount within the subject's microbiome when the human milk oligosaccharides are no longer ingested or administered.

In some embodiments, the probiotic strain is capable of internalizing oligosaccharides, such as to consume or metabolize the oligosaccharides. In certain embodiments, the probiotic strain is capable of internalizing one or more oligosaccharides of the mixture, including those of any of the oligosaccharides or mixtures described herein, e.g., in Section I-A. In certain embodiments, the probiotic strain is capable of internalizing HMOs.

In certain embodiments, the probiotic strain is one or more of a Bifidobacterium, Lactobacillis, Clostridium, Eubacterium, or Stretococcus strain, e.g., capable of consuming or metabolizing HMOs. In certain embodiments, the probiotic strain is or includes at least one strain of Bifidobacterium such as, but not limited to, B. adolescentis, B. animalis (e.g., B. animalis sub sp. animalis or B. animalis sub sp. lactis), B. bijidum, B. breve, B. catenulatum, B. longum (e.g., B. longum subsp. infantis or B. longum subsp. longum), B. pseudocatanulatum, B. pseudolongum; and/or at least one strain of Lactobacillus, such as L. acidophilus, L. antri, L. brevis, L. casei, L. coleohominis, L. crispatus, L. curvatus, L. delbrueckii, L. fermentum, L. gasseri, L. johnsonii, L. mucosae, L. pentosus, L. plantarum, L. reuteri, L rhamnosus, L. sakei, L. salivarius, L. paracasei, L. kisonensis, L. paralimentarius, L. perolens, L. apis, L. ghanensis, L. dextrinicus, L. harbinensis; and/or at least one strain of Bacteroides such as Bacteroides vulgatus or non-toxigenic Bacteroides fragilis; and/or at least one strain of Clostridium such as C. difficile or C. perfringens; and/or at least one species of Streptococcus such as S. thermophilus; and/or at least one strain of Faecalibacterium; and/or at least one strain of Pediococcus, such as P. parvulus, P. lohi, P. acidilactici, P. argentinicus, P. claussenii, P. pentosaceus, or P. stilesii; and/or at least one strain of Lactococcus lactis. In some embodiments, the one or more probiotic may contain more than one strain, such as two or more of any of the species listed herein. As used herein, the terms “B. longum subsp. infantis” and “B. infantis” are be used interchangeably unless otherwise indicated. The terms “B. longum subsp. longum” and “B. longum” are also used interchangeably herein, unless indicated otherwise.

In particular embodiments, the probiotic strain is or more of a strain of B. longum subsp. infantis, B. bifidum, Bacteroides fragilis, Bacteroides vulgatus, Faecalibacterium prausnitzii, Eubacterium rectale, Lactobacillus acidophilus, Lactobacillus delbrueckii, Lactococcus lactis, or Streptococcus thermophilus, e.g., that is capable of consuming or metabolizing HMOs. In some embodiments, the probiotic strain is one or more strains of B. longum subsp. infantis, B. bifidum, Bacteroides fragilis, or Bacteroides vulgatus, e.g., that is capable of consuming or metabolizing HMOs.

In particular embodiments, the species or subspecies of a given probiotic strain may be identified by routine techniques. For example, in some embodiments, the species or subspecies is identified by assessing the sequence similarity of one or more genes to corresponding sequences of known members of bacterial species or subspecies. In certain embodiments, a probiotic strain falls within a species or subspecies if all or a portion of its 16 S gene has at least 97% sequence identity to all or a portion of a known 16S sequence of a known strain falling within the species. In particular embodiments, a probiotic strain falls within a species or subspecies if all or a portion of its 16S gene has at least 97% sequence identity to all or a portion of a known 16S sequence of a known strain falling within the species. Exemplary full or partial 16S sequences are summarized in Table 1.

TABLE 1
Exemplary 16S sequences

| Bacterial species or subspecies | Exemplary 16S Sequence |
|---|---|
| B. longum subsp. infantis | SEQ ID NOS: 1-7 |
| B. adolescentis | SEQ ID NO: 8 |
| B. animalis subsp. animalis | SEQ ID NO: 9 |
| B. animalis subsp. lactis | SEQ ID NO: 10 |
| B. bifidum | SEQ ID NO: 11 |
| B. breve | SEQ ID NO: 12 |
| B. catenulatum | SEQ ID NO: 13 |
| B. longum subsp. longum | SEQ ID NO: 14 |
| B. pseudocatanulatum | SEQ ID NO: 15 |
| B. pseudolongum | SEQ ID NO: 16 |
| L. acidophilus | SEQ ID NO: 17 |
| L. antri | SEQ ID NO: 18 |
| L. brevis | SEQ ID NO: 19 |
| L. casei | SEQ ID NO: 20 |
| L. coleohominis | SEQ ID NO: 21 |
| L. crispatus | SEQ ID NO: 22 |
| L. curvatus | SEQ ID NO: 23 |
| L. delbrueckii | SEQ ID NO: 24 |
| L. fermentum | SEQ ID NO: 25 |
| L. gasseri | SEQ ID NO: 26 |
| L. johnsonii | SEQ ID NO: 27 |
| L. harbinensis | SEQ ID NO: 28 |
| L. mucosae | SEQ ID NO: 29 |
| L. pentosus | SEQ ID NO: 30 |
| L. plantarum | SEQ ID NO: 31 |
| L. reuteri | SEQ ID NO: 32 |
| L rhamnosus | SEQ ID NO: 33 |
| L. sakei | SEQ ID NO: 34 |
| L. salivarius | SEQ ID NO: 35 |
| L. paracasei | SEQ ID NO: 36 |
| L. kisonensis | SEQ ID NO: 37 |
| L. paralimentarius | SEQ ID NO: 38 |
| L. perolens | SEQ ID NO: 39 |
| L. apis | SEQ ID NO: 40 |
| L. ghanensis | SEQ ID NO: 41 |
| L. dextrinicus | SEQ ID NO: 42 |
| Lactococcus lactis | SEQ ID NO: 43 |
| Bacteroides vulgatus | SEQ ID NO: 44 |
| Bacteroides fragilis | SEQ ID NO: 45 |
| S. thermophilus | SEQ ID NO: 46 |
| P. parvulus | SEQ ID NO: 47 |
| P. lolii or P. acidilactci | SEQ ID NO: 48 or 49 |
| P. argentinicus | SEQ ID NO: 50 |
| P. claussenii | SEQ ID NO: 51 |
| P. pentosaceus | SEQ ID NO: 52 |
| P. stilesii | SEQ ID NO: 53 |

In certain embodiments, the at least one probiotic strain has or includes a nucleic acid sequence with at least 97%, at least 98%, at least 99%, or at least 99.5% identity to a nucleic acid sequence set forth in any of SEQ ID NOS: 1-53 or 57-76. In particular embodiments, the at least one probiotic strain has or includes a nucleic acid sequence with at least 97%, at least 98%, at least 99%, or at least 99.5% identity to a nucleic acid sequence set forth in any of SEQ ID NOS: 1-16 or 43-45. In certain embodiments, the at least one probiotic strain has or includes a nucleic acid sequence with at least 97%, at least 98%, at least 99%, or at least 99.5% identity to a nucleic acid sequence set forth in any of SEQ ID NOS: 1-7, 11, 12, 17, 24, or 43-46. In some embodiments, the at least one probiotic strain has or includes a nucleic acid sequence with at least 97%, at least 98%, at least 99%, or at least 99.5% identity to a nucleic acid sequence set forth in any of SEQ ID NOS: 1-7, 11, 44, 45 or 57-76. In certain embodiments, the at least one probiotic strain has or includes a nucleic acid sequence with at least 97%, at least 98%, at least 99%, or at least 99.5% identity to a nucleic acid sequence set forth in any of SEQ ID NOS: 1-16 or 57-76. In particular embodiments, the at least one probiotic strain has or includes a nucleic acid sequence with at least 97%, at least 98%, at least 99%, or at least 99.5% identity to a nucleic acid sequence set forth in any of SEQ ID NOS: 1-7 or 57-76.

In particular embodiments, the at least one probiotic strain is or includes a strain of B. longum subsp. infantis. In particular embodiments, the strain of B. longum subsp. infantis has or includes a nucleic acid sequence with at least 97%, at least 98%, at least 99%, or at least 99.5% identity to a nucleic acid sequence set forth in any of SEQ ID NOS: 1-7. In particular embodiments, the strain of B. longum subsp. infantis has or includes a nucleic acid sequence of at least 200, 300, 400, 500, 600, 700, 800, 900, 1,000, 1,200, or 1,500 nucleotides in length with at least 60%, 70%, 80%, 90%, 95%, 99%, or 99.9% sequence identity to a nucleic acid sequence set forth in SEQ ID NOS: 57-76. In some embodiments, the strain of B. longum subsp. infantis has or includes a nucleic acid sequence having at least 60%, 70%, 80%, 90%, 95%, 99%, or 99.9% sequence identity to a nucleic acid sequence set forth in SEQ ID NOS: 57-76. In certain embodiments, the strain of B. longum subsp. infantis has or includes a nucleic acid sequence having at least 70%, 80%, or 90%, sequence identity to a nucleic acid sequence set forth in SEQ ID NOS: 57-67. In some embodiments, the strain of B. longum subsp. infantis has or includes a nucleic acid sequence having at least 80%, 90%, or 95% sequence identity to a nucleic acid sequence set forth in SEQ ID NOS: 68-72. In particular embodiments, the strain of B. longum subsp. infantis has or includes a nucleic acid sequence having at least 90%, 95%, or 99% sequence identity to a nucleic acid sequence set forth in SEQ ID NOS: 73-76. In some embodiments, the strain of B. longum subsp. infantis has or includes nucleic acid sequences having at least 90%, 95%, or 99% sequence identity to one or more of the nucleic acid sequences set forth in SEQ ID NOS: 57-76. In some embodiments, the strain of B. longum subsp. infantis has or includes the nucleic acid sequences set forth in one or more of SEQ ID NOS: 57-76. In particular embodiments, the strain of B. longum subsp. infantis has or includes nucleic acid sequences having at least 90%, 95%, or 99% sequence identity to all of the nucleic acid sequences set forth in SEQ ID NOS: 57-76. In various embodiments, the strain of B. longum subsp. infantis has or includes the nucleic acid sequences set forth in SEQ ID NOS: 57-76.

In some embodiments, the probiotic strain is at least one strain of Bifidobacterium or a Bacteroides capable of consuming, metabolizing, and/or internalizing HMOs. In some aspects, HMO cannot be metabolized by the host, e.g., mammals such as humans, or most bacteria, including most bacteria commonly found in the microbiome of adult humans. In particular aspects, some strains, species, or subspecies of Bifidobacterium, such as B. longum subsp. infantis, or Bacteroides have enzymatic activity able to degrade specific alpha and beta bonds of HMOs. Five monosaccharides can be found in different HMO structures, glucose, galactose, N-acetyl glucosamine, fucose, and sialic acid (also referred to herein as N-acetyl neuraminic acid). Some strains, species, or subspecies of Bifidobacterium are able to fully degrade HMO intracellularly. Such Bifidobacterium possess genes encoding specific transporters (e.g., ABC transporters such as those described in Sela et al. PNAS (2008) 105 (48) 18964-18969; Schell, et al. PNAS. (2002) 99(22):14422-14427 and LoCascio et al. Appl Environ Microbiol. (2010) 76(22):7373-81), incorporated by reference herein, that selectively transport or import HMO and enzymes necessary for HMO degradation (alpha-fucosidase, alpha-sialidase, beta-galactosidase, and beta-N-hexosaminidase). Other Bifidobacterium strains, such as for example B. bifidum degrades HMO externally or extracellularly, such as for example by lacto-N-biosidase, which cleaves LNB from HMO. The LNB is then internalized by a transporter and degraded by LNB-phosphorylase. In some embodiments, the probiotic strain is a at least one strain of bacterium having one or more genes encoding all or a portion of a transporter, e.g., an ABC transporter, capable of internalizing an oligosaccharide such as an HMO. In particular embodiments, the probiotic strain is a bacterium having one or more genes encoding one or more enzymes, e.g., alpha-fucosidase, alpha-sialidase, beta-galactosidase, and beta-N-hexosaminidase, capable degrading an oligosaccharide such as an HMO. In certain embodiments, the probiotic strain is at least one strain of Bifidobacterium or Bacteroides having one or more genes encoding all or a portion of a transporter, e.g., an ABC transporter, capable of internalizing an oligosaccharide, e.g., an HMO.

In some embodiments, the probiotic strain is B. longum subsp. infantis. Particular embodiments contemplate that B. longum subsp. infantis is known and readily identifiable by those of skill in the art using routine techniques. In some embodiments, B. longum subsp. infantis, including its genome and biology, are known and for example have been described, including in Sela et al. PNAS (2008) 105 (48) 18964-18969; Underwood et al., Pediatr Res. (2015) 77(0): 229-235, incorporated by reference herein. In certain embodiments, Bifidobacterium, e.g., B. longum subsp. infantis, may be isolated using known selective microbiological media, e.g., De Man, Rogosa and Sharpe agar (MRS), optionally in combination with mupirocin, or those described in O'Sullivan et al., J Appl Microbiol. 2011 August; 111(2):467-73, incorporated by reference herein. In some embodiments, suitable sources for isolating Bifidobacterium, e.g., B infantis, are known, and include stool samples obtained from breast fed infants. In certain embodiments, bacterial colonies may be identified or characterized by routine biochemical techniques, such as PCR. In some embodiments, B. longum subsp. infantis is identified by tagman qPCR, such as described in Lawley et al., Peer J. 2017 May 25; 5:e3375. e.g., as performed with forward primer sequence ATACAGCAGAACCTTGGCCT (SEQ ID NO: 54), reverse primer sequence GCGATCACATGGACGAGAAC (SEQ ID NO: 55) and probe sequence [FAM dye]-TTTCACGGA-[ZEN quencher]-TCACCGGACCATACG-[3IABkFQ quencher] (SEQ ID NO: 56). In some aspects, a strain may be confirmed as B. longum subsp. infantis by observing growth when HMOs are provided as the sole carbon source, such as with an assay described in Gotoh et al. Sci Rep. 2018 Sep. 18; 8(1):13958, incorporated by reference herein.

C. Bacteriotherapies

In some embodiments, a prebiotic mixture, e.g., as described herein such as in Section I-A, is administered to a subject who has received or who will receive a bacteriotherapy. In certain embodiments, a prebiotic mixture and a probiotic strain, e.g., as described herein such as in Section I-B, is administered to a subject who has received or who will receive a bacteriotherapy. In some embodiments, the provided methods include administering to a subject in need thereof a prebiotic mixture, a probiotic strain, and a bacteriotherapy, such as to treat or prevent a disease or condition, e.g., associated with or accompanied by dysbiosis of the intestinal microbiome.

In some embodiments, the bacteriotherapy is a therapy that delivers bacteria to a subject. Unless otherwise specified, the term “bacteriotherapy” as referred to herein is a separate medicament, therapy, or composition from the probiotic strain, e.g., the probiotic strain described in Section I-B. Thus, unless otherwise specified, the probiotic strain and the bacteriotherapy may be administered to the subject separately in different dosage forms, at different times, and on different days. If, in some instances, the same species, subspecies, or strain of the probiotic strain were also present in the bacteriotherapy, in such instances the bacteriotherapy would still be distinguishable from the probiotic strain at least by way of a different formulation, preparation, dosage form, or composition, or by way of the fact that that the bacteriotherapy further contains additional bacteria species, subspecies, and strains that are different from probiotic strain.

In certain embodiments, the bacteriotherapy includes one or more species, subspecies, or strains that are found within a human microbiome, e.g., an intestinal microbiome of a healthy human adult. In certain embodiments, the bacteriotherapy is or includes one or more bacteria species, subspecies, or strains obtained from, isolated from, derived from, or present in a human intestinal microbiome, e.g., of a healthy adult human.

In particular embodiments, the bacteriotherapy is or includes a plurality or mixture of different species, subspecies, or strains of bacteria. In some embodiments, the different species, subspecies, or strains of bacteria are present or found in, or are obtained or derived from, the microbiome of a healthy human adult. In certain embodiments, the different species, subspecies, or strains of bacteria are present or found in, or are obtained or derived from, the stool of a healthy human adult. In particular embodiments, the different species, subspecies, or strains of bacteria are present or found in, or are obtained or derived from, a stool sample of a healthy human adult.

In some embodiments, the bacteriotherapy is or includes a live biotherapeutic product. In certain embodiments, the bacteriotherapy is or includes a single species, subspecies, strain, or clone of bacterium that is different from the probiotic strain. In some embodiments, the bacteriotherapy is or includes defined bacterial consortia.

In some embodiments, the bacteriotherapy is a fecal microbiota transplant (FMT; also referred to in the art as a fecal transplant, a fecal bacteriotherapy, or a fecal microbiota transfer). In certain embodiments, the FMT is or includes include introducing a fecal sample of a healthy donor, or a donor having one or more desired characteristics, into a gastrointestinal tract of a patient to repopulate a healthy or desirable gut microbiota. In certain embodiments, the bacteriotherapy is an FMT that is or includes human fecal material containing viable gut flora from a patient or donor.

In particular embodiments, the bacteriotherapies contain or are derived from human fecal material, e.g., a stool sample. In certain embodiments, fecal material, e.g., a donated stool sample, is screened for the presence of pathogenic microorganisms prior to its use in the microbiota restoration therapy, such as to reduce the risk or probability of infection in the recipient. Screening may be performed to detect for the presence of Clostridium species including C. difficile, Norovirus, Adenovirus, enteric pathogens, antigens to Giardia species, Cryptosporidia species and other pathogens, including acid-fast bacteria, enterococci, including but not limited to vancomycin-resistant enterococci (VRE), methicillin-resistant Staphylococcus aureus (MSRA), as well as any ova or parasitic bodies, or spore-forming parasites, including but not limited to Isospora, Clyslospora, and Cryptospora.

In certain embodiments, bacteriotherapy has been cultured prior to administration to a subject. In certain embodiments, the bacteriotherapy is or is obtained, derived, purified, or isolated fecal material that is cultured under conditions that favor growth expansion, or enrichment of beneficial or otherwise desirable microflora. In some embodiments, one or more species, subspecies, or strains of bacteria, e.g., beneficial or otherwise desirable bacteria, are isolated, purified, or removed from the culture by any suitable known technique, including but not limited to gradient purification, differential centrifugation, solvent treatment, heat treatment, acid treatment, immunoprecipitation, or antibiotic selection. Exemplary techniques have been described, inter alia, in Palm et al, 2014 Cell 158(5): P1000-1010; Browne et al 2016 Nature 533: 543-546; and Greub 2012 Clinical Microbiology and Infection 18(12): 1157-1159.

In some embodiments, the bacteriotherapy includes one or more species, subspecies, or strains of bacteria that are obtained from and/or present in the microbiome or intestinal microflora of a healthy human donor. In certain embodiments, the bacteriotherapy includes one or more species, subspecies, or strains of bacteria that are derived, obtained, or purified from fecal material from a healthy human donor. In some embodiments, the donor is an adult. In certain embodiments, the donor has a decreased risk, probability, or likelihood of having or experience dysbiosis of the intestinal microbiome.

In some embodiments, the bacteriotherapy is monoclonal, e.g., is or includes a single species, subspecies or strain of bacterium. In particular embodiments, the bacteriotherapy is or includes at least 1, 2, 5, 10, 20, 30, 40, 50, 75, 100, 150, 200, 250, 300, 350, 400, or 500 species, subspecies, or strains of bacteria. In some embodiments, the bacteriotherapy is or includes at least 1, 2, 5, 10, 20, 30, 40, 50, 75, 100, 150, 200, 250, 300, 350, 400, or 500 species, subspecies, or strains of bacteria that are obtained from and/or present in the microbiome or intestinal microflora of a healthy human donor. In certain embodiments, the bacteriotherapy is or includes at least 1, 2, 5, 10, 20, 30, 40, 50, 75, 100, 150, 200, 250, 300, 350, 400, or 500 species, subspecies, or strains of bacteria that are derived, obtained, or purified from fecal material from a healthy human donor.

Bacteriotherapies such as FMT or modifications thereof containing species, subspecies, and strains present in stool and in the microbiomes of healthy human adults are known and include those described in U.S. Pat. Nos. 9,956,282, 10,064,900, 10,076,546, 10,258,655, 10,391,064, 10,226,431, 9,433,651, and US App Pub No.: 20200093870, 20120149584 and PCT Pub. No.: WO2019227085, WO2019191694, WO2019191390, WO2017091783, WO2017008026, which are incorporated herein by reference.

In certain embodiments, the bacteriotherapy is or includes one or more species, subspecies, or strains of bacteria capable of producing a short chain fatty acid. In some embodiments, the bacteriotherapy is or includes one or more species, subspecies, or strains of bacteria capable of producing acetate. In some embodiments, the bacteriotherapy is or includes one or more species, subspecies, or strains of bacteria capable of producing lactate. In particular embodiments, the bacteriotherapy is or includes one or more species, subspecies, or strains of bacteria capable of producing butyrate.

In various embodiments, the bacteriotherapy includes one or more non-pathogenic bacterial species, subspecies, or strains that are capable of engraftment in a subject's GI tract. In some embodiments, the bacteriotherapy is or includes one or more non-pathogenic bacterial species, subspecies, or strains that are able to colonize a patient's mucosal barrier. In some embodiments, the bacteriotherapy is or includes one or more bacterial species, subspecies, or strains capable of preserving and/or enhancing mucosal barrier integrity and function in a patient. In some embodiments, bacteriotherapy is or includes one or more bacterial species, subspecies, or strains that are capable of decolonizing pathogenic infectious agents. In certain embodiments, the bacteriotherapy is or includes one or more bacterial species, subspecies, or strains capable of competing with pathogenic infectious agents for resources (e.g., niche and/or nutrients). In certain embodiments, the bacteriotherapy is or includes one or more bacterial species, subspecies, or strains capable of enhancing production of one or more of butyrate, acetate, propionate, or lactate, in the gut. In various embodiments, the bacteriotherapy is or includes one or more bacterial species, subspecies, or strains that supplement short chain fatty acid production in the gut. In various embodiments, the bacteriotherapy is capable of inducing proliferation and/or accumulation of Foxp3⁺ cells, e.g., regulatory T cells (Tregs) in the mucosal lining of the gut. In various embodiments, the bacteriotherapy is capable of inducing proliferation and/or accumulation of interleukin-10 (IL-10) in the gut. In various embodiments, the bacteriotherapy reduces proliferation and/or accumulation of interleukin-12 (IL-12), interleukin-4 (IL-4), and/or and gamma interferon (IFN-gamma) in the gut.

Particular embodiments contemplate that the capabilities of the bacteriotherapy (and/or the species, subspecies, or strains of bacteria of the bacteriotherapy) to engraft in a subject's GI tract; colonize a patient's mucosal barrier; preserve and/or enhance mucosal barrier integrity and function; decolonize pathogenic infectious agents; compete with pathogenic infectious agents for resources enhance production of one or more of butyrate, acetate, propionate, or lactate; supplement short chain fatty acid production; induce proliferation and/or accumulation of Foxp3⁺ cells in the mucosal lining; induce proliferation and/or accumulation of IL-10; and/or reduce proliferation and/or accumulation of IL-12, IL-4, and/or IFN-gamma are increased or enhanced, e.g., synergistically increased or enhanced, when the prebiotic mixture and the probiotic strain are administered to the same subject.

In particular embodiments, the bacteriotherapy is administered to a patient using an enema or other suitable technique. In certain embodiments, however, it may be desirable to orally administer the bacteriotherapy composition. In particular embodiments, bacteriotherapies may be prepared or formulated to a form suitable for oral administration, such as by processing, lyophilizing, or freeze-drying (or otherwise converting from a liquid to a solid), adding one or more additives and/or excipients, and/or formulating the bacteriotherapy into a tablet, capsule, or the like.

i) Butyrate Producing Strains of Bacterium

In some embodiments, the bacteriotherapy is or includes at least one species, subspecies, or strain of bacterium capable of producing butyrate (also referred to herein as butyrate producing species, subspecies, or strains of bacteria). In certain embodiments, the bacteriotherapy is or includes one or more species, subspecies, or strains of bacteria capable of producing butyrate that may be administered orally to the subject, e.g., as a probiotic. In particular embodiments, the bacteriotherapy is or includes a butyrate producing strain (i.e., a strain of bacterium capable of producing butyrate).

In certain embodiments, the at least one butyrate producing strain is capable of consuming short chain fatty acids. In some embodiments, the at least one butyrate producing strain is capable of consuming lactate and/or acetate. In particular embodiments, the at least one butyrate producing strain is capable of producing, generating, and/or creating butyrate in the presence of one or more short chain fatty acids. In certain embodiments, the at least one butyrate producing strain is capable of producing, generating, and/or creating butyrate in the presence of lactate and/or acetate. In certain embodiments, the at least one butyrate producing strain is capable of producing, generating, and/or creating butyrate in the presence of the at least one probiotic strain, e.g., a probiotic strain described herein such as in Section I-B, and the prebiotic mixture, e.g., a prebiotic mixture described herein such as in Section I-A.

In some embodiments, the at least one butyrate producing strain is or includes at least one, two, three, four, five, six, seven, eight, nine, ten, or more species, subspecies, or strains of bacteria capable of producing butyrate. In particular embodiments, the butyrate producing strain is or includes at least three strains of bacteria capable of producing butyrate. In some embodiments, the butyrate producing strain is or includes between one and five species, subspecies, or strains of bacteria capable of producing butyrate.

In certain embodiments, the at least one butyrate producing strain has one or more genes that contribute to the production, generation, or making of butyrate. In particular embodiments, the at least one butyrate producing strain has a functional butyryl-CoA:acetate CoA-transferase (but) gene. In certain embodiments, the at least one butyrate producing strain has a functional butyrate kinase (buk) gene. In some embodiments, the at least one butyrate producing strain has a functional butyryl-CoA:4-hydroxybutyrate CoA transferase (4Hbt) gene. In particular embodiments, the at least one butyrate producing strain has a functional butyryl-CoA:acetoacetate CoA transferase (Ato) gene.

In some embodiments, the at least one butyrate producing strain is or includes one or more species, subspecies, or strains of Clostridium Cluster IV bacteria. In certain embodiments, the at least one butyrate producing strain is or includes one or more species, subspecies, or strains of Clostridium Cluster XIVa bacteria. In certain embodiments, the at least one butyrate producing strain is or includes one or more species, subspecies, or strains of bacteria belonging to the Clostridium, Eubacterium, Ruminococcus, Coprococcus, Dorea, Lachnospira, Roseburia, Butyrivibrio, or Anaerofilum genera. In some embodiments, the at least one butyrate producing strain is or includes one or more species, subspecies, or strains of bacteria belonging to the Clostridium, Eubacterium, Ruminococcus, Coprococcus, Dorea, Lachnospira, Roseburia or Butyrivibrio genera. In particular embodiments, the at least one butyrate producing strain is or includes one or more species, subspecies, or strains of bacteria belonging to the Clostridium, Eubacterium, Ruminococcus or Anaerofilum genera.

In some embodiments, the at least one butyrate producing strain is or includes one or more species, subspecies, or strains of Agathobacter rectalis (also referred to as Eubacterium rectale), Anaerobutyricum hallii (also referred to as Eubacterium halihi), Anaerobutyricum soehngenii, Anaerocolumna aminovalerica (also referred to as Clostridium aminovalericum), Anaerostipes butyraticus, Anaerostipes caccae, Anaerostipes hadrus (also referred to as Eubacterium hadrum), Anaerostipes rhamnosivorans, Anaerotruncus colihominis, Blautia argi, Blautia caecimuris, Blautia coccoides (also referred to as Clostridium coccoides), Blautia faecicola, Blautia faecis, Blautia glucerasea, Blautia hansenii (also referred to as Streptococcus hansenii), Blautia schinkii, Blautia stercoris, Blautia wexlerae, Blautia hominis, Blautia hydrogenotrophica (also referred to as Ruminococcus hydrogenotrophicus), Blautia luti (also referred to as Ruminococcus luti), Blautia obeum (also referred to as Ruminococcus obeum), Blautia producta (also referred to as Ruminococcus productus; Streptococcus productus; Peptostreptococcus productus), Blautia schinkii (also referred to as Ruminococcus schinkii), Blautia stercoris, Blautia wexlerae, Butyrivibrio crossotus, Clostridium cellulosi, Clostridium hylemonae, Clostridium innocuum, Clostridium leptum, Clostridium nexile, Clostridium orbiscidens, Clostridium scindens, Clostridium sporosphaeroides, Clostridium symbiosum, Clostridium viride, Coprococcus catus, Coprococcus comes, Coprococcus eutactus, Coprococcus phoceensis, Eisenbergiella tayi, Enterocloster aldenensis (also referred to as Clostridium aldenense), Enterocloster asparagiformis (also referred to as Clostridium asparagiforme), Enterocloster bolteae (also referred to as Clostridium bolteae), Enterocloster citroniae (Clostridium citroniae), Enterocloster clostridioformis (also referred to as Clostridium clostridioforme), Enterocloster lavalensis (Clostridium lavalense), Erysipelatoclostridium ramosum, Eubacterium siraeum, Eubacterium ventriosum, Dorea formicigenerans (also referred to as Eubacterium formicigenerans), Dorea longicatena, Faecalibacterium prausnitzii (also referred to as Fusobacterium prausnitzii), Faecalicatena contorta (also referred to as Eubacterium contortum), Faecalica tenafissicatena (also referred to as Eubacterium fissicatena), Faecalicatena orotica (also referred to as Clostridium oroticum), Faecalimonas umbilicate, Flavonifractor plautii (also referred to as Clostridium orbiscindens), Hungatella effluvii, Hungatella hathewayi (also referred to as Clostridium hathewayi), Hungatella xylanolytica, Lachnospira eligens (also referred to as Eubacterium eligens), Lachnospira multipara, Lachnospira pectinoschiza, Lacrimispora aerotolerans (also referred to as Clostridium aerotolerans), Lacrimispora algidixylanolytica (also referred to as Clostridium algidixylanolyticum), Lacrimispora amygdalina (also referred to as Clostridium amygdalinum), Lacrimispora celerecrescens (also referred to as Clostridium celerecrescens), Lacrimispora indolis (also referred to as Clostridium indolis), Lacrimispora saccharolytica (also referred to as Clostridium saccharolyticum), Lacrimispora sphenoides (also referred to as Bacillus sphenoides), Lacrimispora xylanolytica (also referred to as Clostridium xylanolyticum), Lactonifactor longoviformis, Marvinbryantia formatexigens (also referred to as Bryantella formatexigens), Mediterraneibacter butyricigenes, Mediterraneibacter faecis (also referred to as Ruminococcus faecis), Mediterraneibacter glycyrrhizinilyticus (also referred to as Clostridium glycyrrhizinilyticum), Mediterraneibacter gnavus (also referred to as Ruminococcus gnavus), Mediterraneibacter lactaris (also referred to as Ruminococcus lactaris), Mediterraneibacter massiliensis, Mediterraneibacter torques (also referred to as Ruminococcus torques), Merdimonas faecis, Murimonas intestini, Oscillibacter massiliensis, Papillibacter cinnamivorans, Pseudoflavonfractor capillosus, Robinsoniella peoriensis, Roseburia faecis, Roseburia hominis, Roseburia intestinalis, Roseburia inulinivorans, Ruminococcus albus, Ruminococcus bromii, Subdoligranulum variabile, or Syntrophococcus sucromutans.

In certain embodiments, the at least one butyrate producing strain is or includes one or more species, subspecies, or strains of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis. In some embodiments, the at least one butyrate producing strain is or includes one or more species, subspecies, or strains of Anaerostipes caccae, Clostridium innocuum, Roseburia hominis, or Roseburia intestinalis.

In particular embodiments, the species or subspecies of a given butyrate producing strain may be identified by routine techniques. For example, in some embodiments, the species or subspecies is identified by assessing the sequence similarity of one or more genes to corresponding sequences of known members of bacterial species or subspecies. In certain embodiments, a probiotic strain falls within a species or subspecies if all or a portion of its 165 gene has at least 97% sequence identity to all or a portion of a known 165 sequence of a known strain falling within the species. Exemplary full or partial 165 sequences are summarized in Table 2.

TABLE 2
Exemplary 16S Sequences

| Bacterial species or subspecies | Exemplary 16S Sequence |
|---|---|
| Faecalibacterium prausnitzii | SEQ ID NO: 77 |
| Agathobacter rectalis | SEQ ID NO: 78 |
| Anaerobutyricum hallii | SEQ ID NO: 79 |
| Anaerobutyricum soehngenii | SEQ ID NO: 80 |
| Anaerostipes caccae | SEQ ID NO: 81 |
| Blautia producta | SEQ ID NO: 82 |
| Clostridium leptum | SEQ ID NO: 83 |
| Roseburia hominis | SEQ ID NO: 84 |
| Roseburia intestinalis | SEQ ID NO: 85 |
| Anaerocolumna aminovalerica | SEQ ID NO: 86 |
| Anaerostipes butyraticus | SEQ ID NO: 87 |
| Anaerostipes hadrus | SEQ ID NO: 88 |
| Anaerostipes rhamnosivorans | SEQ ID NO: 89 |
| Anaerotruncus colihominis | SEQ ID NO: 90 |
| Blautia argi | SEQ ID NO: 91 |
| Blautia caecimuris | SEQ ID NO: 92 |
| Blautia coccoides | SEQ ID NO: 93 |
| Blautia faecicola | SEQ ID NO: 94 |
| Blautia faecis | SEQ ID NO: 95 |
| Blautia glucerasea | SEQ ID NO: 96 |
| Blautia hansenii | SEQ ID NO: 97 |
| Blautia hominis | SEQ ID NO: 98 |
| Blautia hydrogenotrophica | SEQ ID NO: 99 |
| Blautia luti | SEQ ID NO: 100 |
| Blautia obeum | SEQ ID NO: 101 |
| Blautia schinkii | SEQ ID NO: 102 |
| Blautia stercoris | SEQ ID NO: 103 |
| Blautia wexlerae | SEQ ID NO: 104 |
| Butyrivibrio crossotus | SEQ ID NO: 105 |
| Clostridium hylemonae | SEQ ID NO: 106 |
| Clostridium nexile | SEQ ID NO: 107 |
| Clostridium scindens | SEQ ID NO: 108 |
| Clostridium sporosphaeroides | SEQ ID NO: 109 |
| Clostridium symbiosum | SEQ ID NO: 110 |
| Clostridium viride | SEQ ID NO: 111 |
| Coprococcus catus | SEQ ID NO: 112 |
| Coprococcus comes | SEQ ID NO: 113 |
| Coprococcus eutactus | SEQ ID NO: 114 |
| Coprococcus phoceensis | SEQ ID NO: 115 |
| Dorea formicigenerans | SEQ ID NO: 116 |
| Dorea longicatena | SEQ ID NO: 117 |
| Eisenbergiella tayi | SEQ ID NO: 118 |
| Enterocloster aldenensis | SEQ ID NO: 119 |
| Enterocloster asparagiformis | SEQ ID NO: 120 |
| Enterocloster bolteae | SEQ ID NO: 121 |
| Enterocloster citroniae | SEQ ID NO: 122 |
| Enterocloster clostridioformis | SEQ ID NO: 123 |
| Enterocloster lavalensis | SEQ ID NO: 124 |
| Eubacterium siraeum | SEQ ID NO: 125 |
| Eubacterium ventriosum | SEQ ID NO: 126 |
| Faecalicatena contorta | SEQ ID NO: 127 |
| Faecalicatena fissicatena | SEQ ID NO: 128 |
| Faecalicatena orotica | SEQ ID NO: 129 |
| Flavonifractor plautii | SEQ ID NO: 130 |
| Hungatella effluvia | SEQ ID NO: 131 |
| Hungatella hathewayi | SEQ ID NO: 132 |
| Hungatella xylanolytica | SEQ ID NO: 133 |
| Lachnospira eligens | SEQ ID NO: 134 |
| Lachnospira multipara | SEQ ID NO: 135 |
| Lachnospira pectinoschiza | SEQ ID NO: 136 |
| Lacrimispora aerotolerans | SEQ ID NO: 137 |
| Lacrimispora algidixylanolytica | SEQ ID NO: 138 |
| Lacrimispora amygdalina | SEQ ID NO: 139 |
| Lacrimispora celerecrescens | SEQ ID NO: 140 |
| Lacrimispora indolis | SEQ ID NO: 141 |
| Lacrimispora saccharolytica | SEQ ID NO: 142 |
| Lacrimispora sphenoides | SEQ ID NO: 143 |
| Lacrimispora xylanolytica | SEQ ID NO: 144 |
| Lactonifactor longoviformis | SEQ ID NO: 145 |
| Marvinbryantia formatexigens | SEQ ID NO: 146 |
| Mediterraneibacter butyricigenes | SEQ ID NO: 147 |
| Mediterraneibacter faecis | SEQ ID NO: 148 |
| Mediterraneibacter glycyrrhizinilyticus | SEQ ID NO: 149 |
| Mediterraneibacter gnavus | SEQ ID NO: 150 |
| Mediterraneibacter lactaris | SEQ ID NO: 151 |
| Mediterraneibacter massiliensis | SEQ ID NO: 152 |
| Mediterraneibacter torques | SEQ ID NO: 153 |
| Murimonas intestine | SEQ ID NO: 154 |
| Oscillibacter massiliensis | SEQ ID NO: 155 |
| Robinsoniella peoriensis | SEQ ID NO: 156 |
| Roseburia faecis | SEQ ID NO: 157 |
| Roseburia inulinivorans | SEQ ID NO: 158 |
| Ruminococcus albus | SEQ ID NO: 159 |
| Ruminococcus bromii | SEQ ID NO: 160 |
| Subdoligranulum variabile | SEQ ID NO: 161 |
| Syntrophococcus sucromutans | SEQ ID NO: 162 |
| Faecalimonas umbilicate | SEQ ID NO: 163 |
| Merdimonas faecis | SEQ ID NO: 164 |
| Clostridium innocuum | SEQ ID NO: 165 |
| Erysipelatoclostridium ramosum | SEQ ID NO: 166 |
| Ruminococcaceae sp. | SEQ ID NO: 167 |

In certain embodiments, the butyrate producing strain has or includes a 16S nucleic acid sequence with at least 97%, at least 98%, at least 99%, or at least 99.5% identity to a nucleic acid sequence set forth in any of SEQ ID NOS: 77-162 or SEQ ID NOS: 77-167. In certain embodiments, the butyrate producing strain has or includes a nucleic acid sequence of at least 200, 300, 400, 500, 1,000, 1,500, or 2,000 nucleotides in length with at least 60%, 70%, 80%, 90%, 95%, 99%, or 99.9% sequence identity to a nucleic acid sequence set forth in any of SEQ ID NOS: 77-167. In particular embodiments, the butyrate producing strain has or includes a nucleic acid sequence encoding 16S that is identical to a nucleic acid sequence set forth in any of SEQ ID NOS: 77-162 or SEQ ID NOS: 77-167. In some embodiments, the butyrate producing strain has or includes a 16S nucleic acid sequence with at least 97%, at least 98%, at least 99%, or at least 99.5% identity to a nucleic acid sequence set forth in any of SEQ ID NOS: 77-85. In particular embodiments, the butyrate producing strain has or includes a nucleic acid sequence encoding 16S that is identical to a nucleic acid sequence set forth in any of SEQ ID NOS: 77-85. In some embodiments, the butyrate producing strain has or includes a 16S nucleic acid sequence with at least 97%, at least 98%, at least 99%, or at least 99.5% identity to a nucleic acid sequence set forth in any of ID NOS: 81, 84, 85, or 165. In particular embodiments, the butyrate producing strain has or includes a nucleic acid sequence encoding 16S that is identical to a nucleic acid sequence set forth in any of SEQ ID NOS: 81, 84, 85, or 165.

In particular embodiments, the at least one butyrate producing strain is or includes at least one, two, three, four, five, six, seven, eight, nine, ten, fifteen, twenty, twenty-five or more species, subspecies, or strains of bacteria listed in Table 2. In certain embodiments, the at least one butyrate producing strain is or includes at least three species, subspecies, or strains of bacteria listed in Table 2. In some embodiments, the at least one butyrate producing strain is or includes from one to five species, subspecies, or strains of bacteria listed in Table 2. In certain embodiments, the at least one butyrate producing strain is or includes at least five species, subspecies, or strains of bacteria listed in Table 2.

In some embodiments, the at least one butyrate producing strain is or includes some or all of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis. In particular embodiments, the at least one butyrate producing strain is or includes from one to five of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis. In particular embodiments, the at least one butyrate producing strain is or includes three of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis. In certain embodiments, the at least one butyrate producing strain is or includes five of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis.

In certain embodiments, the at least one butyrate producing strain is includes at least one, two, three, or all four of Anaerostipes caccae, Clostridium innocuum, Roseburia hominis, and Roseburia intestinalis.

In certain embodiments, a butyrate producing strain falls within a species or subspecies if it contains all or a portion of a gene encoding a butyryl-CoA:acetate CoA-transferase gene has at least 85%, at least 90%, at least 95%, at least 97%, or at least 99% sequence identity to all or a portion of a known gene sequence encoding a butyryl-CoA:acetate CoA-transferase of a known strain falling within the species. Exemplary full or partial butyryl-CoA:acetate CoA-transferase sequences are summarized in Table 3.

TABLE 3
Exemplary butyryl-CoA: acetate CoA-transferase sequences

| Bacterial species or subspecies | Exemplary Sequence |
|---|---|
| Agathobacter rectalis | SEQ ID NO: 168 |
| Anaerobutyricum hallii | SEQ ID NO: 169 |
| Anaerobutyricum soehngenii | SEQ ID NO: 170 |
| Anaerostipes caccae | SEQ ID NO: 171 |
| Faecalibacterium prausnitzii | SEQ ID NO: 172 |
| Roseburia hominis | SEQ ID NO: 173 |
| Roseburia intestinalis | SEQ ID NO: 174 |

In some embodiments, the butyrate producing strain has or includes a nucleic acid sequence encoding all or a portion of butyryl-CoA:acetate CoA-transferase with at least 97%, at least 98%, at least 99%, or at least 99.5% identity to all or a portion, e.g., spanning at least 100, 200, 300, 400, or 500 nucleotides, of a nucleic acid sequence set forth in any of SEQ ID NOS: 168-174. In certain embodiments, the butyrate producing strain has or includes a nucleic acid sequence encoding all or a portion of butyryl-CoA:acetate CoA-transferase that is identical to all or a portion of a nucleic acid sequence set forth in any of SEQ ID NOS: 168-174.

In particular embodiments, the at least one butyrate producing strain contains a butyryl-CoA:acetate CoA-transferase (but) gene, e.g., having at least 70%, 75%, 80%, 85%, 90%, 95%, 97%, or 99% or having 100% sequence identity to a but gene such as any of those set forth in SEQ ID NOS: 168-174. In certain embodiments, the at least one butyrate producing strain contains a butyrate kinase (Buk) gene, e.g., having at least 70%, 75%, 80%, 85%, 90%, 95%, 97%, or 99% or having 100% sequence identity to a known Buk gene, such as the sequence set forth in SEQ ID NO: 175. In particular embodiments, the at least one butyrate producing strain contains a butyryl-CoA:4-hydroxybutyrate CoA transferase (4Hbt) gene, e.g., having at least 70%, 75%, 80%, 85%, 90%, 95%, 97%, or 99% or having 100% sequence identity to a known 4Hbt gene such the sequence set forth in SEQ ID NO: 176. In certain embodiments, the at least one butyrate producing strain contains a butyryl-CoA:acetoacetate CoA transferase (Ato) gene, e.g., having at least 70%, 75%, 80%, 85%, 90%, 95%, 97%, or 99% or having 100% sequence identity to a known Ato gene, such as any set forth in SEQ ID NOS: 177 or 178.

D. Exemplary Compositions, Kits, and Articles of Manufacture

In certain embodiments, provided herein are compositions, kits, or articles of manufacture that are or include one or both of prebiotics, e.g., prebiotic mixtures of non-digestible carbohydrates such as human milk oligosaccharides, and at least one probiotic, e.g., a strain of Bifidobacteria such as B. longum subsp. infantis. In some embodiments, the compositions, kits, or articles of manufacture that are or include a combination of prebiotics, e.g., prebiotic mixtures of non-digestible carbohydrates such as human milk oligosaccharides, and at least one probiotic, e.g., a strain of Bifidobacteria such as B. infantis. In some embodiments, provided herein is a composition that includes the prebiotic mixture and the probiotic strain. In certain embodiments, the prebiotic mixture and the at least one probiotic are contained within separate compositions. In some embodiments, provided herein are kits or articles of manufacture that are or include separate prebiotic and probiotic compositions.

In some embodiments, provided herein are kits or articles of manufacture that are or include a composition that is a prebiotic mixture, e.g., of human milk oligosaccharides, and a composition that is or includes at least one strain of probiotic bacteria. In certain embodiments, the probiotic strain is capable of consuming (e.g., hydrolyzing) the prebiotics contained in the prebiotic mixture. In particular embodiments, the probiotic strain is capable of internalizing and consuming (e.g., hydrolyzing) the prebiotics of the probiotic mixture. In various embodiments, the probiotic strain is capable of internalizing and consuming (e.g., hydrolyzing) human milk oligosaccharides, e.g., the human milk oligosaccharides of the prebiotic mixture. In particular embodiments, a probiotic strain that is capable of consuming, internalizing, and/or hydrolyzing a prebiotic is capable of consuming, internalizing, and/or hydrolyzing the prebiotic in vivo such within the human gut.

In certain embodiments, the prebiotic mixture is or includes one or more human milk oligosaccharides and the probiotic strain is or includes any of the probiotic strains listed in Table 1. In various embodiments, the at least one probiotic is or includes a strain of Bifidobacteria and the prebiotic is or includes a mixture of human milk oligosaccharides. In certain embodiments, the strain of Bifidobacteria is or includes a strain of B. breve, B. Bifidum, B. infantis, or B. longum subsp. longum. In some embodiments, the prebiotic mixture includes one or more HMOs, e.g., one or more synthetic HMOs. In particular embodiments, the prebiotic mixture is or includes a mixture of at least 5, 10, 25, 50, 100, 125, or 150 human milk oligosaccharides. In some embodiments, the prebiotic mixture is or includes synthetic human milk oligosaccharides. In particular embodiments, the prebiotic mixture is or includes a concentrated human milk permeate, e.g., a concentrated human milk permeate described herein such as in Section I-A-(i).

In particular embodiments, the compositions, kits, or articles of manufacture also include a bacteriotherapy or an apparatus, reagent, or equipment for preparing a bacteriotherapy. In certain embodiments, the bacteriotherapy is or includes a plurality or mixture of different species, subspecies, or strains of bacteria that are present or found in, or are obtained or derived from, the microbiome of a healthy human adult. In some embodiments, the bacteriotherapy is a bacteriotherapy described herein, e.g., in Section I-C. In certain embodiments, the bacteriotherapy is an FMT.

In some embodiments, the compositions, kits, or articles of manufacture include a bacteriotherapy that is or includes at least one strain of bacterium capable of producing butyrate, e.g., that may be administered as a probiotic. In certain embodiments, the at least one butyrate producing strain can be administered orally to a subject, e.g., in conjunction with oral administration of the probiotic strain of bacteria.

In particular embodiments, provided herein is an article of manufacture containing one or more of the kits or compositions described herein. In certain embodiments, the article of manufacture contains the prebiotic mixture, e.g., of human milk oligosaccharides, and the probiotic strain, e.g., B. longum subsp. infantis, and instructions for use. In some embodiments, the instructions for use described methods, e.g., those described in Section II, for administering prebiotic mixture and the probiotic strain to a subject who has or will receive a bacteriotherapy, e.g., an FMT. In some embodiments, the article of manufacture includes a bacteriotherapy, such as described herein e.g., in Section I-C. In particular embodiments, the article of manufacture includes an apparatus, equipment, or reagent useful for preparing or administering a bacteriotherapy such as described herein e.g., in Section I-C. In certain embodiments, the article of manufacture includes at least one butyrate producing strain of bacteria, e.g., a butyrate producing strain described herein such as in Section I-C-(i).

### II. METHODS OF TREATMENT

Provided herein are methods for treating, preventing, or ameliorating one or more diseases, disorders, or conditions in a subject who has received or will receive a bacteriotherapy, e.g., an FMT, such as by administering a prebiotic mixture, optionally with a probiotic strain. In certain embodiments, the methods improve the effectiveness or efficiency of the bacteriotherapy for treating, alleviating, or ameliorating one or more symptoms of the disease, disorder or condition e.g., as compared to when the bacteriotherapy is provided in the absence of the prebiotic mixture and/or probiotic strain.

Provided herein are methods for treating, preventing, or ameliorating one or more diseases, disorders, or conditions in a subject who has received or will receive a bacteriotherapy, e.g., an FMT, such as by administering a prebiotic mixture, e.g., of human milk oligosaccharides, and optionally administering a probiotic strain of bacterium, e.g., B. longum subsp. infantis. In certain embodiments, the methods improve the effectiveness or efficiency of the bacteriotherapy for treating, alleviating, or ameliorating one or more symptoms of the disease, disorder or condition e.g., as compared to when the bacteriotherapy is provided in the absence of the prebiotic mixture and/or probiotic strain.

Also provided herein are methods for treating, prevention, or ameliorating a disease, disorder, or condition that is or includes administering a prebiotic mixture, e.g., of human milk oligosaccharides, and a probiotic strain of bacterium, e.g., B. longum subsp. infantis, and a bacteriotherapy, e.g., an FMT. In particular embodiments, the methods are more effective for treating, alleviating, or ameliorating one or more symptoms of the disease, disorder or condition e.g., as compared to an alternative treatment or as compared to administration of the bacteriotherapy, the prebiotic mixture, and/or probiotic strain alone.

In certain embodiments, the prebiotic mixture, e.g., a prebiotic mixture described herein such as in Section I-A, is administered to the subject who has undergone or will undergo a bacteriotherapy, e.g., a bacteriotherapy described herein such as in Section I-C. In some embodiments the prebiotic mixture is administered to the subject prior to the bacteriotherapy. In some embodiments, the prebiotic mixture is administered concurrent with the bacteriotherapy. In some embodiments, the prebiotic mixture is administered after the bacteriotherapy. In certain embodiments, the prebiotic mixture is administered before and after the bacteriotherapy.

In some embodiments, the probiotic strain, e.g., a probiotic strain described herein such as in Section I-B, is administered to the subject who has undergone or will undergo a bacteriotherapy, e.g., a bacteriotherapy described herein such as in Section I-C. In some embodiments the prebiotic mixture is administered to the subject prior to the bacteriotherapy. In certain embodiments, the prebiotic mixture is administered concurrent with the bacteriotherapy. In some embodiments, the prebiotic mixture is administered after the bacteriotherapy. In certain embodiments, the prebiotic mixture is administered before and after the bacteriotherapy.

In particular embodiments, both a prebiotic mixture, e.g., a prebiotic mixture described herein such as in Section I-A, and a probiotic strain, e.g., a probiotic strain described herein such as in Section I-B, is administered to a subject who has undergone or will undergo a bacteriotherapy, e.g., a bacteriotherapy described herein such as in Section I-C. In some embodiments, both the prebiotic mixture and the probiotic strain are administered to the subject prior to the bacteriotherapy. In various embodiments, both the prebiotic mixture and the probiotic strain are administered concurrent with the bacteriotherapy. In some embodiments, both the prebiotic mixture and the probiotic strain are administered after the bacteriotherapy. In certain embodiments, both the prebiotic mixture and the probiotic strain are administered before and after the bacteriotherapy.

A. Administering Provided Compositions to a Subject

In certain embodiments, the method includes one or more treatment phases where both the prebiotic mixture and the probiotic strain are administered. In some embodiments, the treatment phases where both the prebiotic mixture and the probiotic strain are administered may occur prior to, after, and/or concurrent with administration of the bacteriotherapy. In some embodiments, the method includes one or more treatment phases where prebiotic mixture, but not the probiotic strain is administered. In certain embodiments, the method is or includes a treatment phase where both the prebiotic mixture and the probiotic strain are administered, followed by a subsequent treatment phase where the prebiotic mixture and not the probiotic strain is administered. Such treatment phases may occur prior to or after administration of the bacteriotherapy. In certain embodiments, the bacteriotherapy is administered during a treatment phase where both the prebiotic mixture and the probiotic strain are administered. In some embodiments, the bacteriotherapy is administered during a treatment phase where only the prebiotic mixture is administered. In some embodiments, the prebiotic mixture and/or the probiotic strain are administered at least once daily during a treatment phase. In some embodiments, the treatment phases may cycle or repeat, such that after the subsequent treatment phase is complete, a new treatment phase similar to the first occurs where both the prebiotic mixture and the probiotic strain are administered.

In certain embodiments, the prebiotic mixture is administered daily for at least 2, 3, 4, 5, 7, 10, 14, 21, or 28 days, e.g., consecutive days. In certain embodiments, the prebiotic mixture is administered in an amount of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g per day, e.g., total weight of the probiotics such as non-digestible carbohydrates such as human milk oligosaccharides. In particular embodiments, the prebiotic mixture in an amount of at least 0.001 g, 0.01 g, 0.1 g, 1 g, 2 g, 3 g, 4 g, 5 g, 6 g, 7.5 g, 8 g, 9 g, 10 g, 12 g, 16 g, 18 g, 20 g, 25 g, or 50 g total human milk oligosaccharides per day. In some embodiments, the prebiotic mixture is administered in an amount of between 0.1 g and 50 g; 0.5 g and 25 g, 1 g and 20 g, 2 g and 18 g, 1 g and 5 g, 2 g and 3 g, 3 g and 6 g, 4 g and 5 g, 5 g and 10 g, 8 g and 10 g, 10 g and 20 g, 15 g and 20 g, or 17 g and 19 g total human milk oligosaccharides per day. In some embodiments, the prebiotic mixture is administered in an amount of, of about, or of at least 2 g, 4.5 g, 6 g, 9 g, 12 g, 16 g, or 18 g total human milk oligosaccharides per day.

In certain embodiments, the probiotic strain is administered daily for at least 2, 3, 4, 5, 7, 10, 14, 21, or 28 days, e.g., consecutive days. In some embodiments, the probiotic strain is administered in an amount of at least 1×10¹, 5×10¹,1×10², 1×10³, 1×10⁴, 1×10⁵, 1×10⁶, 5×10⁶, 1×10⁷, 1×10⁷, 5×10⁷, 1×10⁸, or 5×10⁸ colony forming units (CFU) per day. In various embodiments, the probiotic strain is administered in an amount of at least 1×10¹, 1×10², 1×10³, 1×10⁴, 1×10⁵,1×10⁶, 5×10⁶, 1×10⁷, 1×10⁷, 5×10⁷, 1×10⁸, or 5×10⁸ colony forming units (CFU) per dose. In certain embodiments, the probiotic strain is administered in an amount of between 1×10⁶ and 1×10¹², 5×10⁶ and 1×10¹⁰, 1×10⁷ and 1×10⁹, or 1×10⁷ and 1×10⁸ CFU per day. In some embodiments, the probiotic strain is administered in an amount of, of about, or at least 5×10⁶ colony forming units (CFU) per dose or per day. In some embodiments, the probiotic strain is administered in an amount of, of about, or at least 8×10⁷ colony forming units (CFU) per dose or per day.

In some embodiments, the prebiotic mixture is administered at least once within 3 months, 2 months, 1 months, 60 days, 45 days, 30 days, 6 weeks, 5 weeks, 4 weeks, 3 weeks, 28 days, 21 days, 14 days, 10 days, 7 days, 5 days, 3 days, or 1 day prior to administration of the bacteriotherapy. In certain embodiments, the prebiotic mixture is administered at least once daily for at least 2, 3, 4, 5, 6, 7, 8, 9, 10, 11, 12, 13, 14, 21, or 28 consecutive days prior to the bacteriotherapy. In certain embodiments, the prebiotic mixture is administered at least once within 3 months, 2 months, 1 months, 60 days, 45 days, 30 days, 6 weeks, 5 weeks, 4 weeks, 3 weeks, 28 days, 21 days, 14 days, 10 days, 7 days, 5 days, 3 days, or 1 day after administration of the bacteriotherapy. In certain embodiments, the prebiotic mixture is administered at least once daily for at least 2, 3, 4, 5, 6, 7, 8, 9, 10, 11, 12, 13, 14, 21, or 28 consecutive days after the bacteriotherapy. In various embodiments, the prebiotic mixture is administered prior to and after the bacteriotherapy.

In certain embodiments, the probiotic strain is administered at least once within 3 months, 2 months, 1 months, 60 days, 45 days, 30 days, 6 weeks, 5 weeks, 4 weeks, 3 weeks, 28 days, 21 days, 14 days, 10 days, 7 days, 5 days, 3 days, or 1 day prior to administration of the bacteriotherapy. In certain embodiments, the probiotic strain is administered at least once daily for at least 2, 3, 4, 5, 6, 7, 8, 9, 10, 11, 12, 13, 14, 21, or 28 consecutive days prior to the bacteriotherapy. In particular embodiments, the probiotic strain is administered at least once within 3 months, 2 months, 1 months, 60 days, 45 days, 30 days, 6 weeks, 5 weeks, 4 weeks, 3 weeks, 28 days, 21 days, 14 days, 10 days, 7 days, 5 days, 3 days, or 1 day after administration of the bacteriotherapy. In certain embodiments, the probiotic strain is administered at least once daily for at least 2, 3, 4, 5, 6, 7, 8, 9, 10, 11, 12, 13, 14, 21, or 28 consecutive days after the bacteriotherapy. In various embodiments, the probiotic strain is administered prior to and after the bacteriotherapy.

In certain embodiments, the prebiotic mixture and the probiotic strain are both administered at least once within 3 months, 2 months, 1 months, 60 days, 45 days, 30 days, 6 weeks, 5 weeks, 4 weeks, 3 weeks, 28 days, 21 days, 14 days, 10 days, 7 days, 5 days, 3 days, or 1 day prior to administration of the bacteriotherapy. In certain embodiments, the prebiotic mixture and the probiotic strain are both administered at least once daily for at least 2, 3, 4, 5, 6, 7, 8, 9, 10, 11, 12, 13, 14, 21, or 28 consecutive days prior to the bacteriotherapy. In particular embodiments, the prebiotic mixture and the probiotic strain are both administered at least once within 3 months, 2 months, 1 months, 60 days, 45 days, 30 days, 6 weeks, 5 weeks, 4 weeks, 3 weeks, 28 days, 21 days, 14 days, 10 days, 7 days, 5 days, 3 days, or 1 day after administration of the bacteriotherapy. In certain embodiments, the prebiotic mixture and the probiotic strain are both administered at least once daily for at least 2, 3, 4, 5, 6, 7, 8, 9, 10, 11, 12, 13, 14, 21, or 28 consecutive days after the bacteriotherapy. In various embodiments, the prebiotic mixture and the probiotic strain are both administered prior to and after the bacteriotherapy.

In some embodiments, the bacteriotherapy is administered by a suitable means known in the art, including but not limited to, anally such as by enema. In certain embodiments, the bacteriotherapy is formulated for oral consumption, such as in capsule or tablet form.

In some embodiments, the bacteriotherapy is or includes at least one butyrate producing strain of bacterium. In particular embodiments, the at least one butyrate producing strain may be administered orally. In certain embodiments, the at least one butyrate producing strain is or includes one or more strains described herein, e.g., in Section I-C-(i).

In some embodiments, the butyrate producing strain is administered daily for at least 2, 3, 4, 5, 7, 10, 14, 21, or 28 days, e.g., consecutive days. In some embodiments, the butyrate producing strain is administered in an amount of at least 1×10¹, 5×10¹,1×10², 1×10³, 1×10⁴, 1×10⁵, 1×10⁶, 5×10⁶, 1×10⁷, 1×10⁷, 5×10⁷, 1×10⁸, or 5×10⁸ colony forming units (CFU) per day. In some embodiments, the butyrate producing strain is administered in an amount of at least 1×10¹, 1×10², 1×10³, 1×10⁴, 1×10⁵, 1×10⁶, 5×10⁶, 1×10⁷, 1×10⁷, 5×10⁷, 1×10⁸, or 5×10⁸ colony forming units (CFU) per dose. In certain embodiments, the butyrate producing strain is administered in an amount of between 1×10⁶ and 1×10¹², 5×10⁶ and 1×10¹⁰, 1×10⁷ and 1×10⁹, or 1×10⁷ and 1×10⁸ CFU per day. In some embodiments, the butyrate producing strain is administered in an amount of, of about, or at least 5×10⁶ colony forming units (CFU) per dose or per day. In some embodiments, the butyrate producing strain is administered in an amount of, of about, or at least 8×10⁷ colony forming units (CFU) per dose or per day.

In particular embodiments, the at least one butyrate producing strain of bacteria and the probiotic strain are administered orally to the subject at least once on the same day. In some embodiments, the at least one butyrate producing strain of bacteria and the probiotic strain are administered on the same day on at least one, two, three, four, five, six, seven, eight, nine, ten, twelve, fourteen, twenty-one, or twenty-eight consecutive days or every day for at least one, two, three, four, five, or six weeks. In some embodiments, the prebiotic mixture is administered on all or a portion of the days where both the probiotic strain and the at least one butyrate producing strain are administered.

In certain embodiments, the prebiotic mixture, e.g., of HMOs, the at least one probiotic strain, e.g., B. longum subsp. infantis, and the at least one butyrate producing strain are administered as or within the same composition to the subject. In some embodiments, the prebiotic mixture, the at least one probiotic strain, and the at least one butyrate producing strain are administered to the subject in separate compositions.

In certain embodiments, the prebiotic mixture, the at least one probiotic strain, and the at least one butyrate producing strain are administered together or separately during the same treatment regimen. In certain embodiments, the treatment regimen has separate treatment phases. The treatment regimen may include separate treatment phases where different combinations, doses, or timing of doses for some or all of the prebiotic mixture, the at least one probiotic strain, and the at least one butyrate producing strain are administered to the subject. In some embodiments, all of the prebiotic mixture, the at least one probiotic strain, and the at least one butyrate producing strain are administered during a treatment phase that occurs during the treatment regimen, and, in a different treatment phase, only one or two of the prebiotic mixture, the at least one probiotic strain, and the at least one butyrate producing strain are administered in a different treatment phase that occurs during the same treatment regimen.

In certain embodiments, a treatment phase has a duration of at least one, two, three, four, five, six, seven, eight, nine, or ten days, or for at least one, two, three, four, five, or six weeks, or for at least one, two, three, four, five, or six months. In some embodiments, a treatment phase is or is at least seven days. In particular embodiments, a treatment phase is or is at least fourteen days.

In some embodiments, the treatment regimen includes more than one treatment phase. In particular embodiments, in one treatment phase, e.g., a first treatment phase, the prebiotic mixture and the at least one probiotic strain are administered. In some embodiments, the prebiotic mixture and the at least one probiotic strain are administered within the same composition during the treatment phase, e.g., the first treatment phase. In some embodiments, the prebiotic mixture and the at least one probiotic strain are administered as separate compositions during the treatment phase, e.g., the first treatment phase. In certain embodiments, one or both of the at least one probiotic mixture and the prebiotic mixture is administered at least once, at least twice, at least three times, at least once per week, at least twice per week, at least three time per week, every other day, and/or every day during the treatment phase, e.g., the first treatment phase. In some embodiments, the prebiotic mixture and the at least one probiotic strain are administered on the same day for at least one, some, or all of the days of the treatment phase, e.g., the first treatment phase. In particular embodiments, the at least one butyrate producing strain is also administered during the treatment phase, e.g., the first treatment phase. In some embodiments, the at least one butyrate producing strain is administered at least once, at least twice, at least three times, at least once per week, at least twice per week, at least three time per week, every other day, and/or every day during the treatment phase, e.g., the first treatment phase. In particular embodiments, the at least one butyrate producing strain is administered on the same days as the at least one probiotic strain during the treatment phase, e.g., the first treatment phase. In some embodiments, the treatment phase, e.g., the first treatment phase, has a duration of at least one, two, three, four, five, six, seven, ten, fourteen days, or at least one, two, three, four, five, or six weeks, or from one day to fourteen days or from three days to seven days.

In some embodiments, the treatment regimen includes a treatment phase, e.g., a second treatment phase, where the prebiotic mixture and not the at least one probiotic strain is administered. In certain embodiments, the prebiotic mixture is administered at least once, at least twice, at least three times, at least once per week, at least twice per week, at least three time per week, every other day, and/or every day during the treatment phase, e.g., the second treatment phase. In particular embodiments, the at least one butyrate producing strain is also administered during the second treatment phase. In some embodiments, the at least one butyrate producing strain is administered at least once, at least twice, at least three times, at least once per week, at least twice per week, at least three time per week, every other day, and/or every day during the second treatment phase. In certain embodiments, the at least one butyrate producing strain is not administered during the second treatment phase. In particular embodiments, the second treatment phase has a duration of at least one, two, three, four, five, six, seven, ten, fourteen days, or at least one, two, three, four, five, or six weeks, or from one day to fourteen days or from three days to seven days.

In some embodiments, the first and second treatment phases occur once during a treatment regimen. In particular embodiments, the first and second treatment phases occur more than once during a treatment regiment, such as cycling or repeating throughout the duration of the treatment regimen. In some embodiments, there may be a gap or duration, e.g., for at least one, two, three, five, seven, ten, or fourteen, days, with no treatments between cycles, e.g. after the end of the second treatment phase and before the beginning of a subsequent first treatment phase.

In some embodiments, the prebiotic mixture and the at least one probiotic strain are administered together or separately for a period of time, such as in a treatment regimen. In some embodiments, the administration of the prebiotic mixture, e.g., of HMOs, allows for the engraftment and expansion of the probiotic strain, e.g., B. longum subsp. infantis. In certain embodiments, the probiotic strain is exogenous to the subject's microbiome, e.g., intestinal microbiome. In particular embodiments, the probiotic strain is not present within the subject's microbiome prior to administration. In certain embodiments, the prebiotic mixture is administered concurrently with and/or subsequently to administration of the at least one probiotic strain. In some embodiments, the at least one probiotic strain is present and/or expands within the subject's microbiome during a time period in which the prebiotic mixture is administered. In certain embodiments, the present or amount of the at least one probiotic strain within the microbiome is reduced when administration of the prebiotic mixture ends, is ceased or is terminated. In particular embodiments, the probiotic strain is absent and/or undetectable following the termination or end of administration of the prebiotic mixture. In certain embodiments, the presence of the probiotic strain, e.g., B. infants, is transient and is regulated by administration of the prebiotic mixture.

In certain embodiments, at least one probiotic strain is capable of consuming or metabolizing some or all of the oligosaccharides, e.g., HMOs, of the prebiotic mixture. In some embodiments, the timing or dosing for administering the at least one probiotic strain and the prebiotic mixture achieves a growth or expansion of the probiotic strain in vivo, e.g., within the microbiome of the subject. In certain embodiments, the administered oligosaccharides, e.g., HMOs, selectively or exclusively serve as a carbon source for the at least one probiotic strain, e.g., as opposed to other bacterial strains present in the gut or microbiome. In some embodiments, the oligosaccharides of the mixture selectively or exclusively serve as an energy source for the at least one probiotic strain e.g., as opposed to other bacterial strains present in the gut or microbiome.

In certain embodiments, subjects are administered (e.g., at least once daily) all of the at least one probiotic strain, e.g., B. longum subsp. infantis, the at least one butyrate producer, and the prebiotic mixture, e.g., of human milk oligosaccharides, during a first or initial treatment phase, e.g., for at least 1, 3, 7, or 14 days, and then are administered the prebiotic mixture alone during a subsequent treatment phase, e.g., such that occurs immediately the first or initial treatment phase. In some embodiments, administration of the prebiotic mixture extends the duration of the colonization of the probiotic strain within the subject's gut and/or microbiome.

In certain embodiments, administering the prebiotic mixture regulates the expansion, level, or amount of the probiotic strain, e.g., B. longum subsp. infantis, and/or the production or generation of metabolites, e.g., lactate and/or acetate, by the probiotic strain. In some aspects, expansion of the probiotic strain and/or generation or production of metabolites by the probiotic strain promotes the engraftment and/or the expansion of the butyrate producing strain and/or promotes the production or generation of butyrate by the butyrate producing strain. Thus, in some aspects, the probiotic and butyrate producing strains are administered to the subject, and the concurrent or subsequent administration of the prebiotic mixture may be adjusted to provide a therapeutic response, e.g., to promote growth or expansion of beneficial microbiota and/or to promote the generation or production of butyrate within the subject's gut or microbime. In some embodiments, the dosage and/or duration of treatment with the prebiotic mixture, e.g., of HMOs, can depend on several factors, including severity and responsiveness of the disease, route of administration, time course of treatment (days to months to years), and time to amelioration of the disease.

As used herein, “subject” and “subject in need thereof” are used interchangeably. In some embodiments, the subject is an infant, a child, a juvenile, or an adult. In certain embodiments, the subject is at least 1 month, 3 months, 6 months, 12 months, 18 months, or 24 months of age. In certain embodiments, the subject is at least 1 year, 2 years, 5 years, 10 years, 12 years, 16 years, or at least 18 years of age. In some embodiments, the subject is at least 12 years old. In certain embodiments, the subject is at least 18 years old. In some embodiments, the subject is an adult.

In some embodiments, provided herein is a method for treating, reducing, ameliorating, or preventing dysbiosis. In particular embodiments method is or includes steps for administering to the subject a prebiotic mixture, such as any described herein e.g., in Section I-A, probiotic strain, such as a probiotic strain described herein, e.g., in Section I-B or listed in Table 1, and a bacteriotherapy, e.g., as described in such as in Section I-C. In some embodiments, the method treats or prevents one or more diseases or conditions that include or are associated with dysbiosis, e.g., of the intestinal microbiome. In certain embodiments, the microbiome is an intestinal microbiome of a human.

In certain embodiments, the probiotic strain may be administered by adding the probiotic strain directly to the bacteriotherapy prior to its administration. In some such embodiments, the probiotic strain is not present in and/or is exogenous to the bacteriotherapy prior to adding or contacting the probiotic strain. In particular embodiments, the probiotic strain is B. longum subsp. infantis, which in some aspects is not detectable in adult human microbiomes, and the bacteriotherapy is derived from adult human stool.

B. Conditions, Diseases, and Disorders Treated by the Provided Methods

In certain embodiments, the prebiotic mixture, e.g., of HMOs, the probiotic strain, e.g., B. longum subsp. infantis, and the bacteriotherapy are administered to a subject having, suspected of having, or at risk of having dysbiosis, e.g., of the intestinal microbiome. In certain embodiments, the transient presence, engraftment, or expansion of the probiotic strain, e.g., B. longum subsp. infantis, reduces, decreases, or ameliorates the dysbiosis. Particular embodiments contemplate that the presence, engraftment, or expansion of the probiotic strain, e.g., B. longum subsp. infantis, and the bacteriotherapy creates, promotes, or generates an environment and/or one or more conditions that (i) promotes the presence, growth, or expansion of beneficial microbiota; (ii) decreases the presence, growth, or expansion of pathogenic microbiota; (iii) promotes diversity of microbiota present within the microbiome; or (iv) any or all of (i) through (iii).

In certain embodiments, administration of the prebiotic mixture, e.g., of HMOs, the probiotic strain, e.g., B. longum subsp. infantis, and the bacteriotherapy reduces the presence or abundance of pathogenic bacteria in the subject's gut. In certain embodiments, administration of the mixture of oligosaccharides, e.g., HMOs, and probiotic strain of bacterium, e.g., B. longum subsp. infantis, reduces gut domination by pathogenic taxa (e.g., Enterobacteriaceae, Enterococcus, and Staphylococcus). In particular embodiments, growth or engraftment of the probiotic, e.g., B. longum subsp. infantis, and the bacteriotherapy within the gut or microbiome reduces the abundance, level, activity, or presence of pathogenic taxa. In certain embodiments, administration of the mixture of oligosaccharides, e.g., HMOs, and the probiotic strain, e.g., B. longum subsp. infantis, reduces the abundance, level, activity, or presence of pathogenic taxa by, by about, or by at least 10%, 20%, 30%, 40%, 50%, 60%, 70%, 75%, 80%, 90%, 95%, or 100%, e.g., as compared to prior to the treatment, as compared to administration of the bacteriotherapy alone, or as compared to an alternative treatment. In particular embodiments, the growth or engraftment of the probiotic strain, e.g., B. longum subsp. infantis, and the bacteriotherapy within the gut or microbiome increases the amount, production, presence, or concentration of acetate, lactate, or butyrate.

It is contemplated that correcting or restoring a healthy microbiome of a subject with a condition, disorder, or disease related to, associated with, or accompanied by dysbiosis will improve one or more symptoms of the condition, disease, or disorder and/or cure or trigger the remission of the condition, disease, or disorder.

In some embodiments, administration of the prebiotic mixture, the probiotic strain, and the bacteriotherapy impairs the growth of one or more pathogens. Such pathogens treated by the provided methods include, but are not limited to, Aeromonas hydrophila, Bacillus, e.g., Bacillus cereus, Bifidobacterium, Bordetella, Borrelia, Brucella, Burkholderia, C. difficile, Campylobacter, e.g., Campylobacter fetus and Campylobacter jejuni, Chlamydia, Chlamydophila, Clostridium, e.g., Clostridium botulinum, Clostridium difficile, and Clostridium perfringens, Corynebacterium, Coxiella, Ehrlichia, Enterobacteriaceae, e.g., Carbapenem-resistent Enterobacteriaceae (CRE) and Extended Spectrum Beta-Lactamase producing Enterobacteriaceae (ESBL-E), fluoroquinolone-resistant Enterobacteriaceae, Enterococcus, e.g., vancomycin-resistant enterococcus spp., extended spectrum beta-lactam resistant Enterococci (ESBL), and vancomycin-resistant Enterococci (VRE), Escherichia, e.g., enteroaggregative Escherichia coli, enterohemorrhagic Escherichia coli, enteroinvasive Escherichia coli, enteropathogenic E. coli, enterotoxigenic Escherichia coli (such as but not limited to LT and/or ST), Escherichia coli O157:H7, and multi-drug resistant bacteria E. coli, Francisella, Haemophilus, Helicobacter, e.g., Helicobacter pylori, Klebsiella, e.g., Klebsiellia pneumonia and multi-drug resistant bacteria Klebsiella, Legionella, Leptospira, Listeria, e.g., Lysteria monocytogenes, Morganella, Mycobacterium, Mycoplasma, Neisseria, Orientia, Plesiomonas shigelloides, Antibiotic-resistant Proteobacteria, Proteus, Pseudomonas, Rickettsia, Salmonella, e.g., Salmonella paratyphi, Salmonella spp., and Salmonella typhi, Shigella, e.g., Shigella spp., Staphylococcus, e.g., Staphylococcus aureus and Staphylococcus spp., Streptococcus, Treponema, Vibrio, e.g., Vibrio cholerae, Vibrio parahaemolyticus, Vibrio spp., and Vibrio vulnificus, and Yersinia, e.g., Yersinia enterocolitica. At least one of the one or more pathogens can be an antibiotic-resistant bacterium (ARB), e.g., Antibiotic-resistant Proteobacteria, Vancomycin Resistant Enterococcus (VRE), Carbapenem Resistant Enterobacteriaceae (CRE), fluoroquinolone-resistant Enterobacteriaceae, or Extended Spectrum Beta-Lactamase producing Enterobacteriaceae (ESBL-E).

In some embodiments, the condition, disease, or disorder is an autoimmune disorder including, but not limited to, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & Ill autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

In some embodiments, the condition, disease, or disorder is a diarrheal disease including, but not limited to, acute bloody diarrhea (e.g., dysentery), acute watery diarrhea (e.g., cholera), checkpoint inhibitor-associated colitis, diarrhea due to food poisoning, persistent diarrhea, and traveler's diarrhea.

In some embodiments, the condition, disease, or disorder is an inflammatory bowel disease (IBD) or related disease including, but not limited to, Behcet's disease, collagenous colitis, Crohn's disease, diversion colitis, fulminant colitis, intermediate colitis, left-sided colitis, lymphocytic colitis, pancolitis, pouchitis, proctosigmoiditis, short bowel syndrome, ulcerative colitis, and ulcerative proctitis.

In some embodiments, administration of the prebiotic strain, the probiotic mixture, and the bacteriotherapy treats or prevents various GI disorders known to result from or be associated or accompanied with dysbiosis of the intestinal microbiome. In certain embodiments, administration of the prebiotic strain, the probiotic mixture, and the bacteriotherapy reduces GI immunoactivation and inflammation.

In various embodiments, administration of the prebiotic strain, the probiotic mixture, and the bacteriotherapy treats or prevents various bloodstream infections (BSI). In certain embodiments, administration of the prebiotic strain, the probiotic mixture, and the bacteriotherapy treats or prevents catheter or intravascular-line infections (e.g., central-line infections). In some embodiments, administration of the prebiotic strain, the probiotic mixture, and the bacteriotherapy treats or prevents chronic inflammatory diseases.

In particular embodiments, administration of the prebiotic strain, the probiotic mixture, and the bacteriotherapy treats or prevents meningitis; pneumonia, e.g., ventilator-associated pneumonia; skin and soft tissue infections; surgical-site infections; urinary tract infections (e.g., antibiotic-resistant urinary tract infections and catheter-associated urinary tract infections); wound infections; and/or antibiotic-resistant infections and antibiotic-sensitive infections.

In certain embodiments, administration of the prebiotic strain, the probiotic mixture, and the bacteriotherapy treats or prevents diseases or disorders relating to the “gut-brain axis”, including neurodegenerative, neurodevelopmental, and neurocognitive disorders, such as anorexia, anxiety, autism-spectrum disorder, depression, Parkinson's, and Schizophrenia.

In some embodiments, administration of the prebiotic strain, the probiotic mixture, and the bacteriotherapy treats or prevents a side effect of an anti-cancer therapy and/or increases efficacy of an anti-cancer therapeutic agent and/or anti-cancer therapy. In embodiments, the anti-cancer therapy is surgery, radiation therapy, chemotherapy (including hormonal therapy) and/or targeted therapy (including an immunotherapy). Illustrative chemotherapeutics agents are provided elsewhere herein. In embodiments, the immunotherapy binds to and/or recognizes a tumor-cell antigen and/or a cancer-cell antigen, e.g., CTLA-4, PD-1, PD-L1, or PD-L2. In embodiments, the immunotherapy comprises administration of Keytruda (Pembrolizumab), Opdivo (Nivolumab), Yervoy (Ipilimumab), Tecentriq (atezolizumab), Bavencio (avelumab), and Imfinzi (durvalumab).

In some embodiments, the subject is refractory and/or non-responsive to an anti-cancer therapy (as described herein). In embodiments, the pharmaceutical composition treats a subject that presents a non-curative response, a limited response, or no response to the anti-cancer therapy, or even progress, after 12 weeks or so of receiving the anti-cancer therapy. Thus, a pharmaceutical composition of the present invention can rescue subjects that are refractory and/or non-responsive to the anti-cancer therapy. In embodiments, the subject is refractory and/or non-responsive to a treatment directed to a checkpoint molecule, e.g., CTLA-4, PD-1, PD-L1, and/or PD-L2. In embodiments, the treatment directed to a checkpoint molecule comprises administration of Keytruda (Pembrolizumab), Opdivo (Nivolumab), Yervoy (Ipilimumab), Tecentriq (atezolizumab), Bavencio (avelumab), or Imfinzi (durvalumab).

In certain embodiments, the prebiotic mixture, e.g., of human milk oligosaccharides, the probiotic strain, e.g., B. longum subsp. infantis, and a bacteriotherapy, e.g., an FMT, are administered to a subject to treat, ameliorate, remedy, or prevent a gastrointestinal condition, disease, or disorder associated with, related to, or caused by dysbiosis. In certain embodiments, the gastrointestinal condition, disease, or disorder is or includes one or more of a chronic inflammatory disease, an autoimmune disease, an infection, bowel resection, and/or a condition associated with chronic diarrhea. In certain embodiments, the gastrointestinal condition, disease, or disorder is or includes one or more of irritable bowel syndrome (IBS), inflammatory bowel disease (IBD, including Crohn's Disease and colitis), short bowel syndrome (SBS), celiac disease, small intestinal bacterial overgrowth (SIBO), gastroenteritis, leaky gut syndrome, and gastric lymphoma. In certain embodiments, the gastrointestinal condition, disease, or disorder is associated with a bacterial, viral, or parasitic infection or overgrowth. In a particular embodiment, the disease or disorder is associated with infection by drug-resistant bacteria, e.g., vancomycin-resistant enterococcus (VRE).

In some embodiments, the prebiotic mixture, e.g., of human milk oligosaccharides, the probiotic strain, e.g., B. longum subsp. infantis, and a bacteriotherapy, e.g., an FMT, are administered to an immunocompromised subject. In certain embodiments, the administration prevents, reduces, treats, or ameliorates an infection in the immunocompromised subject. In some embodiments, the administration prevents, reduces, treats, or ameliorates overgrowth or domination of pathogenic bacteria. In some embodiments, the immunocompromised subject has undergone one or more treatments for cancer. In some embodiments, the treatments are or include chemotherapy. In certain embodiments, the treatment is or includes an allogenic transplant, e.g., a hematopoietic stem cell transplant or bone marrow transplant. In certain embodiments, the administration prevents or reduces the probability or likelihood of a systemic infection by, by about, or by at least 10%, 20%, 30%, 40%, 50%, 60%, 70%, 75%, 80%, 90%, or 95%, e.g., as compared to an alternative treatment or treatment with the bacteriotherapy alone.

In certain embodiments, the prebiotic mixture, e.g., of human milk oligosaccharides, the probiotic strain, e.g., B. longum subsp. infantis, and a bacteriotherapy, e.g., an FMT, are administered to a subject who has or is at risk of sepsis. In some embodiments, the probability or likelihood of sepsis is reduced or decreased by at least 10%, 20%, 30%, 40%, 50%, 60%, 70%, 80%, 90%, 95%, or 99 e.g., as compared to an alternative treatment or the treatment with the bacteriotherapy alone. In certain embodiments, the administration of the prebiotic mixture and the at least one probiotic improves or increases the survival of the subject over 6 months, 12 months, 18 months, 1 year, 2 years, 5 years, 10 years, and/or 20 years or more by, by about, or by at least 5%, 10%, 20%, 25%, 30%, 40%, 50%, 60%, 70%, 75%, 80%, 90%, 95%, 100%, or 1-fold, 2-fold, 3-fold, 4-fold, or 5-fold greater, e.g., as compared to an alternative treatment or the treatment with the bacteriotherapy alone.

In particular embodiments, administration the prebiotic mixture, e.g., of human milk oligosaccharides, the probiotic strain, e.g., B. longum subsp. infantis, and a bacteriotherapy, e.g., an FMT, prevents, reduces, decreases, remedies, or ameliorates one or more symptoms associated with a gastrointestinal condition, disease, or disorder. In certain embodiments, the one or more symptoms associated with gastrointestinal condition, disease, or disorder may include, but are not limited to, diarrhea, fever, fatigue, abdominal pain and cramping, blood in stool, mouth sores, weight loss, fistula, inflammation (of skin, eyes, or joints), inflamed liver or bile ducts, delayed growth (in children). In particular embodiments, administration of the prebiotic mixture, the probiotic strain, and the bacteriotherapy reduces the risk or probability for the subject of experiencing one or more symptoms associated with the gastrointestinal condition, disease, or disorder by, by about, or by at least 10%, 20%, 30%, 40%, 50%, 60%, 70%, 75%, 80%, 90%, or 95%, e.g., as compared to an administered the bacteriotherapy alone. In certain embodiments administration of the prebiotic mixture, the probiotic strain, and the bacteriotherapy increases probability or likelihood for remission by, by about, or by at least 10%, 20%, 30%, 40%, 50%, 60%, 70%, 75%, 80%, 90%, 100%, or 1-fold, 2-fold, 5-fold, 10-fold, 20-fold, 50-fold, or 100-fold e.g., as compared to administration of the bacteriotherapy alone. In some embodiments, administration of the administration of the prebiotic mixture, the probiotic strain, and the bacteriotherapy increases probability or likelihood for remission within 12 weeks, 10 weeks, 8 weeks, 6 weeks, 4 weeks, or less than 4 weeks, e.g., from the initiation or termination of the administration.

In various embodiments, the prebiotic mixture, e.g., of human milk oligosaccharides, the probiotic strain, e.g., B. longum subsp. infantis, and a bacteriotherapy, e.g., an FMT, are administered to a subject to treat, ameliorate, remedy, or prevent a chronic inflammatory disease, an autoimmune disease, an infection, bowel resection, and/or a condition associated with chronic diarrhea. Such pathology includes, but is not limited to: irritable bowel syndrome (IBS), inflammatory bowel disease (IBD), short bowel syndrome (SBS), celiac disease, small intestinal bacterial overgrowth (SIBO), gastroenteritis, leaky gut syndrome, and gastric lymphoma. In some embodiments the disease or disorder is associated with a bacterial, viral, or parasitic infection or overgrowth, e.g. by drug-resistant bacteria. In some embodiments, administration of the prebiotic mixture, the probiotic strain, and the bacteriotherapy increases probability or likelihood for cure or remission of the chronic inflammatory disease, autoimmune disease, infection, bowel resection, and/or chronic diarrhea for by, by about, or by at least 10%, 20%, 30%, 40%, 50%, 60%, 70%, 75%, 80%, 90%, 100%, or 1-fold, 2-fold, 5-fold, 10-fold, 20-fold, 50-fold, or 100-fold e.g., as compared to a subject administered the bacteriotherapy alone. In some embodiments, administration of the prebiotic mixture, the probiotic strain, and the bacteriotherapy increases probability or likelihood for the cure or remission within 12 weeks, 10 weeks, 8 weeks, 6 weeks, 4 weeks, or less than 4 weeks, e.g., from the initiation or termination of the administration.

In some embodiments, the prebiotic mixture, e.g., of human milk oligosaccharides, the probiotic strain, e.g., B. longum subsp. infantis, and a bacteriotherapy, e.g., an FMT, are administered to a subject to treat, ameliorate, remedy, or prevent pouchitis. In certain aspects, pouchitis is inflammation that occurs in the lining of a pouch created during surgery to treat ulcerative colitis or certain other diseases. In some embodiments, the surgery is or includes removal of a diseased colon or portion thereof. In certain embodiments, the surgery is a J pouch surgery (ileoanal anastomosis IPAA).

In some embodiments, the prebiotic mixture, e.g., of human milk oligosaccharides, the probiotic strain, e.g., B. longum subsp. infantis, and a bacteriotherapy, e.g., an FMT, are administered to a subject to treat, ameliorate, remedy, or prevent pouchitis in a subject in need thereof, e.g., a subject who has undergone an IPAA surgery. In particular embodiments, administration of the prebiotic mixture, the probiotic strain, and the probiotic strain prevents, reduces, decreases, remedies, or ameliorates one or more symptoms associated with pouchitis. In certain embodiments, the one or more symptoms associated with pouchitis may include, but are not limited to, increased stool frequency, tenesmus, straining during defecation, blood in the stool, incontinence, seepage of waste matter during sleep, abdominal cramps, pelvic or abdominal discomfort, or tail bone pain. In certain embodiments, symptoms associated with more severe pouchitis include, but are not limited to, fever, dehydration, malnutrition, fatigue, iron-deficiency anemia, or joint pain. In particular embodiments, administration of the prebiotic mixture, the probiotic strain, and the bacteriotherapy reduces the risk or probability for the subject of experiencing pouchitis by, by about, or by at least 10%, 20%, 30%, 40%, 50%, 60%, 70%, 75%, 80%, 90%, or 95%, e.g., as compared to a subject not administered the probiotic strain and/or the mixture of oligosaccharides.

In some embodiments, the prebiotic mixture, e.g., of human milk oligosaccharides, the probiotic strain, e.g., B. longum subsp. infantis, and a bacteriotherapy, e.g., an FMT, are administered to a subject has undergone or will undergo an allogenic stem cell transplant. In certain embodiments, the allogenic transplant is a bone marrow transplant (BMT). In particular embodiments, the allogenic transplant is a hematopoietic stem cell transplantation (HSCT). In particular embodiments, the subject has undergone the allogenic stem cell transplant within 12 weeks, 8 weeks, 6 weeks, 4 weeks, 3 weeks, 2 weeks, 14 days, 12 days 10 days, 7 days, 5 days, 4 days, 3 days, 2 days, or 1 day prior to administration of a first dose of the prebiotic mixture, the probiotic strain, or the bacteriotherapy. In certain embodiments, the first dose of the prebiotic mixture or the probiotic strain is administered within 12 weeks, 8 weeks, 6 weeks, 4 weeks, 3 weeks, 2 weeks, 14 days, 12 days 10 days, 7 days, 5 days, 4 days, 3 days, 2 days, or 1 day prior to receiving the allogenic stem cell transplant.

In particular embodiments, administration of the prebiotic mixture, e.g., of human milk oligosaccharides, the probiotic strain, e.g., B. longum subsp. infantis, and a bacteriotherapy, e.g., an FMT, reduces or decreases the probability or likelihood of experiencing GVHD. In certain embodiments, the probability or likelihood is reduced or decreased by at least 10%, 20%, 30%, 40%, 50%, 60%, 70%, 80%, 90%, 95%, or 99%, e.g., as compared to a subject not administered the prebiotic mixture or the at least one probiotic. In certain embodiments, the probability or likelihood of experiencing GVHD within 20 years, 10 years, 7 years, 5 years, 2 years or 1 year, or within the subject's lifetime, is reduced or decreased, e.g., as compared untreated subject or a subject treated with the bacteriotherapy alone.

In certain embodiments, the prebiotic mixture, e.g., of human milk oligosaccharides, the probiotic strain, e.g., B. longum subsp. infantis, and a bacteriotherapy, e.g., an FMT, are administered to decrease or reduce mortality associated with an allogenic transplant, e.g., BMT or HSCT, or with GVHD. In some embodiments, the prebiotic mixture, e.g., of human milk oligosaccharides, the probiotic strain, e.g., B. longum subsp. infantis, and a bacteriotherapy, e.g., an FMT, are administered to increase survival of subjects who undergo an allogenic transplant, e.g., BMT or HSCT. In particular embodiments, administration of the prebiotic mixture, e.g., of human milk oligosaccharides, the probiotic strain, e.g., B. longum subsp. infantis, and a bacteriotherapy, e.g., an FMT, improves or increases the survival of the subject over 6 months, 12 months, 18 months, 1 year, 2 years, 5 years, 10 years, and/or 20 years or more by, by about, or by at least 5%, 10%, 20%, 25%, 30%, 40%, 50%, 60%, 70%, 75%, 80%, 90%, 95%, 100%, or 1-fold, 2-fold, 3-fold, 4-fold, or 5-fold greater, e.g., as compared to untreated subjects or subjects treated with the bacteriotherapy alone.

C. Exemplary Features of the Methods

Particular embodiments contemplate that the effectiveness of the provided methods relate, at least in part, to interactions, e.g., synergistic interactions, among the different compositions. For example, in some aspects, the provided methods more successfully correct or ameliorate dysbiosis and/or related disorders to a much greater extent than what would be expected from treatment with the prebiotic mixture, the probiotic strain, or the bacteriotherapy alone. Among the benefits of the provided methods are that the efficacy and safety of bacteriotherapies are improved, allowing for successful implementation of bacteriotherapies for treatments of indications or with vulnerable populations that would not otherwise be successfully treated by the bacteriotherapy.

Particular embodiments contemplate that the prebiotic mixture, e.g., of human milk oligosaccharides, is selectively consumed in vivo by the probiotic, e.g., B. longum subsp. infantis. In some embodiments, the prebiotic mixture directly promotes the growth and expansion of the probiotic within the host microbiome, so that adjusting the timing or dosage of the prebiotic mixture will in turn effect the abundance of the probiotic strain in vivo. Furthermore, in some embodiments, the probiotic strain is capable of producing factors such as acetate and lactate which influence the intestinal environment, e.g. such as by regulating intestinal pH. In some embodiments, the production of these factors by the probiotic strain allows for or improves engraftment by species, subspecies, and strains of the bacteriotherapy. Thus, in some aspects, administration of the prebiotic mixture also controls the engraftment and expansion of the bacteriotherapy when the probiotic strain is present within the microbiome. In some aspects, the bacteriotherapy is or includes bacteria capable of consuming the factors, e.g., lactate and acetate, produced by the probiotic strain. In some aspects, the consumption of these factors by the bacteriotherapy results in the bacteriotherapy's production of its own factors, e.g., butyrate, that will further influence the environment surrounding the microbiome, such as by promoting the health of the gut epithelium, improving host immune function, and inhibiting pathogenic taxa. Thus, in some aspects, the interplay between the prebiotic mixture, the probiotic strain, and the bacteriotherapy maximize the potential for the bacteriotherapy to engraft, expand, and correct dysbiosis within the host's microbiome. Furthermore, in certain aspects these effects may be controlled by adjusting the administration of the prebiotic mixture.

In some embodiments, the probiotic strain is capable of consuming or metabolizing some or all of the prebiotic mixture, e.g., of HMOs, and furthermore, the prebiotic mixture is selectively utilized by the probiotic strain in vivo, e.g., as opposed to other bacteria present within the microbiome. Thus, in some aspects, an advantage of the provided methods are that the probiotic mixture selectively feeds the probiotic strain, and thus minimizes the utilization of the prebiotics by other bacteria, allowing for a controlled interaction between the prebiotics and the probiotic strain. For example, in certain embodiments, the prebiotic mixture contains human milk oligosaccharides, which are not typically present in an adult diet and therefore not typically supplied to an adult human microbiome, and, in particular embodiments, the probiotic strain is B. longum subsp. infantis, which is capable of consuming HMO and is not typically detectable in an adult human microbiome. Thus, in particular aspects, the combination of the prebiotic mixture and probiotic strain greatly reduce the risk of unwanted or adverse effects by unintentionally promoting the growth of pathogenic bacteria.

In some aspects, the combination of the prebiotic mixture and the probiotic strain, e.g., the combination of human milk oligosaccharides and B. longum subsp. infantis, indirectly results in butyrate production through cross-feeding (e.g., by acetate and lactate production) to other members of the gut microbiota. In certain aspects, butyrate production within the microbiome benefits the subject. However, to maximize the benefits of this treatment, then butyrate producers would have to be present in the subject's microbiome. In certain aspects, this may not be the case with a dysbiotic microbiome. Thus, in some aspects, the bacteriotherapy serves, at least in part, to provide butyrate producing bacteria from a healthy microbiome, thus maximizing the benefits of the prebiotic/probiotic combination. In certain embodiments, the bacteriotherapy is or includes at least one butyrate producing strain. Therefore, in certain aspects, the combination of all three of the prebiotic mixture, the probiotic strain, and the bacteriotherapy improve the clinical outcomes, consistency, and safety of the methods.

In certain embodiments, a bacteriotherapy may be selected, that contains a sufficient number of species or strains of bacteria capable (i) of selectively consuming the prebiotic mixture, (ii) producing short chain fatty acids such as some or all of acetate, butyrate, and propionate, and/or (iii) directly or indirectly inhibiting pathogenic bacterial growth. In some such embodiments, the bacteriotherapy is or includes B. longum. subsp. infantis and one or more species, subspecies, or strains of bacteria capable of consuming acetate and lactate and producing butyrate. In some such embodiments, methods of administering the prebiotic mixture, the probiotic strain, and the bacteriotherapy may be substituted with administering only the prebiotic mixture and the bacteriotherapy to achieve similar results.

### III. METHODS OF SCREENING OR PREPARING A BACTERIOTHERAPY

In some embodiments, provided herein are methods of preparing a bacteriotherapy, e.g., for the treatment or prevention of a condition, disease or disorder described herein, e.g., in Section II. In particular embodiments, the provided methods for screening or preparing a bacteriotherapy is or includes a step of incubating a population, mixture, or culture of bacteria with an agent that promotes the growth or expansion or beneficial bacteria and/or impairs or prevents the growth or expansion of pathogenic bacteria. The provided methods for screening and preparing a bacteriotherapy therefor improve the consistency, efficacy, and/or the safety of a bacteriotherapy.

In some embodiments, the population or mixture of bacteria is obtained from fecal material such as a human stool sample. In some embodiments, the fecal material or stool sample is provided from a healthy human donor. In certain embodiments, the fecal material or stool sample is pooled from fecal material obtained from multiple healthy human donors. In some embodiments, the donor is an infant, a child, and adolescent, a teenager, or an adult. In certain embodiments, the donor is an adult. In certain embodiments, the donor is considered as having a low risk or probability for experiencing intestinal dysbiosis during his or her lifetime, e.g., as compared to the general population.

In some embodiments, the culture, population, or mixture of bacteria is incubated in the presence of one or more agents, e.g., enrichment agents. In certain embodiments, the enrichment agents may be incubated with mixtures, populations, or cultures of bacteria, such as to promote growth or expansion of beneficial or otherwise desirable bacteria and/or to reduce the presence or amount of potentially pathogenic taxa.

In particular embodiments, the enrichment agent is or includes one or more prebiotic molecules, e.g., oligosaccharides. In certain embodiments, the enrichment agent is or includes an oligosaccharide that may be internalized by one or more strains of lactic acid producing bacteria such as a strain of Bifidobacteria. In some embodiments, the enrichment agent may include one or more of a fructo-oligosacharide (FOS), galactooligosaccharide (GOS), transgalactooligosaccharide (TOS), gluco-oligosaccharide, xylo-oligosaccharide (XOS), chitosan oligosaccharide (COS), soy oligosaccharide (SOS), isomalto-oligosaccharide (IMOS), or derivatives thereof. In certain embodiments, such derivatives include those with modifications that may increase the likelihood or probability of consumption, metabolism, and/or internalization (such as by transport or import) of the oligosaccharide by a potentially beneficial species, subspecies, or strain of bacterium, e.g., a Bifidobacterium. Such modifications may include but are not limited to fucosylation or sialylation. In some embodiments, the oligosaccharides of the mixture may include one or more of a FOS, GOS, TOS, gluco-oligosaccharide, XOS, COS, SOS, IMOS, or derivatives or any or all of the foregoing, that are capable of being metabolized, consumed, and/or internalized by one or more strains, species, or subspecies of Bifidobacterium, e.g., B. longum subsp. infantis. In certain embodiments, the enrichment agent is or includes one or more oligosaccharides that are obtained or derived from a resistant starch, pectin, psyllium, arabinogalactan, glucomannan, galactomannan, xylan, lactosucrose, lactulose, lactitol and various other types of gums such as tara gum, acacia, carob, oat, bamboo, citrus fibers, such as by treatment with enzymes that hydrolyze fiber or polysaccharides.

In some embodiments, the enrichment agent is or includes one or more human milk oligosaccharides. In particular embodiments, the enrichment agent is or includes at least one human milk oligosaccharide. In some embodiments, the enrichment agent is a mixture or plurality of human milk oligosaccharides. In some embodiments, the enrichment agent is or includes a plurality of, of about, or of at least 2, 3, 5, 10, 25, 50, 75, 100, 125, 150 different individual HMOs, e.g., HMOs with different individual chemical formulas or chemical structures. In certain embodiments, the prebiotic mixture is or includes a plurality of, of about, or of at least 10, 25, 50, 75, 100, 125, 150 different individual HMOs. In some embodiments, the prebiotic mixture includes some or all of 2′-fucosyl-lactose, 3′-fucosyl-lactose, 3′-sialyl-lactose, 6′-sialyl-lactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose. In particular embodiments, the mixture includes all of 2′-fucosyl-lactose, 3′-fucosyl-lactose, 3′-sialyl-lactose, 6′-sialyl-lactose, Lacto-N-tetraose, lacto-N-difucohexaose I, lactodifucotetraose, Lacto-N-fucopentaose I, sialylacto-N-tetraose c, sialylacto-N-tetraose b, and disialyllacto-N-tetraose.

In some embodiments, the enrichment agent is or includes a prebiotic mixture described herein, e.g., in Section I-A. In particular embodiments, the enrichment agent is or includes a concentrated human milk permeate. In some aspects, the human milk permeate is obtained by the ultrafiltration of human skim milk. In some embodiments, the HMO content of the human milk permeate is concentrated, such as by nanofiltration or reverse osmosis.

In certain embodiments, the enrichment agents are contacted to or are incubated with mixtures, populations, or cultures of bacteria, such as to promote growth or expansion of beneficial or otherwise desirable bacteria and/or to reduce the presence or amount of potentially pathogenic taxa. In some embodiments, the enrichment agent promotes or expands beneficial bacteria that are or include bacteria capable of producing SCFA, e.g., butyrate. In certain embodiments, the beneficial bacteria are or include species or strains of the genus Roseburia. In some embodiments, the enrichment agent is or includes human milk oligosaccharides. In certain embodiments, human milk oligosaccharides are sufficient to increase or expand some beneficial bacteria, e.g., even without the addition or inclusion of a probiotic strain such as a Bifidobacterium or B. longum subsp. infantis. In particular embodiments, the mixtures, populations, or cultures of bacteria are contacted with or incubated with one or more human milk oligosaccharides to increase or expand one or more bacterium capable of producing SCFA, e.g., one or more species or strains of the genus Roseburia.

In some embodiments, the enrichment agent is lactic acid or lactate. Under normal conditions the concentration of lactic acid (lactate) in the mammalian gut is very low despite the fact that many bacterial species, such as Lactobacilli, Streptococci, Enterococci and Bifidobacteria that reside in the intestine produce this acid in large quantities as a fermentation end product. It has been hypothesized that the accumulation of lactic acid is normally prevented by the ability of certain other bacteria that inhabit the gut to consume lactic acid and to use it as a source of energy. At least some such lactic acid utilizing bacteria are thought to produce high levels of butyrate, which is considered to be beneficial for the host e.g., at least in part for anti-inflammatory effects. Thus, in certain embodiments, a populate, culture, or mixture of bacteria is incubated with lactate to enrich or promote butyrate producing bacteria.

In some embodiments, the enrichment agent is a short chain fatty acid. Short-chain fatty acids (SCFAs), the main metabolites produced by bacterial fermentation of dietary fiber in the gastrointestinal tract, are speculated to have a key role in cross talk between microbiota and host. In particular embodiments, production of SCFA is inhibitory to opportunistic, pathogenic taxa, e.g., Enterobacteriaceae, Enterococcus, or Staphylococcus. In certain embodiments, the enrichment agent is acetate, propionate, butyrate, valerate, isobutyrate, or isovalerate. In particular embodiments, the enrichment agent is acetate. In particular embodiments, the enrichment agent is butyrate. In some embodiments, the enrichment agent is propionate.

In certain embodiments, bacteria is also incubated in the presence of a probiotic strain, e.g., a probiotic strain described herein, e.g., in Section I-B. In certain embodiments, the probiotic strain is added to the culture, mixture, or population of bacteria prior to the incubation in the presence of the enrichment agent. In particular embodiments, the probiotic strain is not present within the culture, mixture, or population of bacteria prior to the incubation.

In some embodiments, the probiotic strain is capable consuming the enrichment agent. In particular embodiments, the probiotic strain is capable of consuming human milk oligosaccharides. In certain embodiments, the probiotic strain is or includes Bifidobacterium, Lactobacillis, Clostridium, Eubacterium, or Stretococcus. In some embodiments, the probiotic strain is a bacterium listed in Table 1. In certain embodiments, the probiotic strain is a Bifidobacterium. In particular embodiments, the probiotic strain is or includes B. breve, B. bifidum, B. longum subsp. infantis, or B. longum subsp. longum. In particular embodiments, the probiotic strain is or includes B. longum subsp. infantis.

In certain embodiments, the method is or includes a step for collecting, harvesting, or isolating one or more species, subspecies, or strains from the population, mixture, or culture of bacteria after the incubation. In some embodiments, at least 1, 2, 5, 10, 15, 20, 25, 30, 35, 40, 45, 50, 60, 70, 80, 90, 100, 125, 150, 200, 250, 300, 350, 400, or 500 species, subspecies, or strains of bacteria are collected, isolated, or purified from the mixture, culture, or population of bacteria following the incubation. In some embodiments, at least 0.01%, 0.1%, 1%, 5%, 10%, 20%, 30%, 40%, or 50% of the bacteria are collected, isolated, or purified from the mixture, culture, or population of bacteria following the incubation. In some embodiments, all or essentially all of the bacteria are collected following the incubation.

In some embodiments the provided methods also include a control incubation. In some embodiments, the control condition is or includes an incubation of the mixture, population, or culture of bacteria in the absence of an enrichment agent. In some embodiments, the control incubation is performed with a control agent. Thus, in some instances, populations, mixtures, or cultures of bacteria incubated with the enrichment agent are compared to the populations, mixtures, or cultures of bacteria from the control incubation, and species, subspecies, or strains of bacteria found to be enriched following incubation with the enrichment agent as compared to controls are collected, isolated, or purified.

In certain embodiments, bacteria may be collected, isolated, or purified by any suitable techniques that are known. Such techniques include, but are not limited to, one or more of fractionation, gradient purification, solvent treatment, heat treatment, acid treatment, immunoprecipitation.

In some embodiments, a bacteriotherapy is obtained or derived from fecal material, e.g., a stool sample, collected from a donor who was administered a prebiotic mixture, e.g., as described herein such as in Section I-A, and a probiotic strain, e.g., as described herein such as in Section I-B, prior to providing the stool sample. In certain embodiments, the prebiotic mixture is a mixture of human milk oligosaccharides. In some embodiments, the probiotic strain is B. longum subsp. infantis. In some embodiments, the stool sample is produced during or within a treatment regimen described in Example 2.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the described or claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” a value means within a range of 25%, ±10%, ±5%, ±1%, ±0.1%, or 0.01% of the value.

As used herein the term “pharmaceutical composition” means, for example, a mixture or formulation containing a specified amount, e.g. a therapeutically effective amount, of an active ingredient such as a human milk fraction, in a pharmaceutically acceptable carrier to be administered to a mammal, e.g., a human.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response, and other problem complications commensurate with a reasonable benefit/risk ratio. Such reasonable benefit/risk ratios may be determined by of skill as a matter of routine.

By “human milk oligosaccharide(s)” (also referred to herein as “HMO(s)”) is meant a family of structurally diverse unconjugated glycans that are found in human breast milk. As used herein human milk oligosaccharides include oligosaccharides found in human milk that contain lactose at the reducing end and, typically, fucose, sialic acid or N-acetylglucosamine at the non-reducing end (Morrow et al., J. Nutri. 2005 135:1304-1307). Unless otherwise indicated, human milk oligosaccharides also encompass 3′-sialyllactose (3′-SL) and 6′-sialyllactose (6′-SL) oligosaccharides that are found in human milk.

Glycans in milk are found as oligosaccharides or conjugated to milk proteins as glycoproteins, or lipid as glycolipids etc. HMO are free glycans that constitute the third most abundant component of human milk, after lactose and lipid (Morrow, 2005). The majority of HMO, however, are not metabolized by the infant and can be found in infant feces largely intact.

By “consisting essentially” of, as used herein refers to compositions containing particular recited components while excluding other major bioactive factors.

“Probiotic” as used herein, refers to any live, non-pathogenic microorganisms, e.g., bacteria, which can confer health benefits to a host organism, e.g., a mammal such as a human, that contains an appropriate amount of the microorganism. In some aspects, those of skill in the art may readily identify species, strains, and/or subtypes of non-pathogenic bacteria that are recognized as probiotic bacteria. Examples of probiotic bacteria strains include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium.

“Bifidobacterium” as used herein, refers to a genus of gram-positive, nonmotile, anaerobic bacteria. In some aspects, Bifidobacterium are ubiquitous inhabitants of the gastrointestinal tract, vagina, and mouth of mammals, including humans. In certain aspects, Bifidobacteria are one of the major genera of bacteria that make up the gastrointestinal tract microbiota in mammals. In certain aspects, some or all species, subspecies, or strains of Bifidobacterium are probiotics.

The term “dysbiosis” as used herein refers to a state of the microbiota of the gut or other body area in a subject, in which the normal diversity and/or function of the microbial populations is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health. According to non-limitative examples, essential functions may include enhancement of the gut mucosal barrier, direct or indirect reduction and elimination of invading pathogens, enhancement of the absorption of specific substances, and suppression of GI inflammation.

As used herein, the terms “gut microbiome” and “intestinal microbiome” are used interchangeably unless otherwise noted.

The term “essentially” such as when used in the phrase “essentially all” of a given substance may be used to infer that the substance, e.g., oligosaccharides, includes unavoidable impurities, e.g., no more impurities than one it. Likewise, when used in the phrase “essentially free” of a given substance (or “essentially no” or “essentially none of” a given substance) may mean no more of the given substance than is unavoidable, e.g., as an impurity.

The term “internalization” such as in reference to an internalization of an oligosaccharide by a bacterial cell refers to the transfer of the oligosaccharide from the outside of the bacterial cell to the inside of the bacterial cell. Unless otherwise indicated, “internalization of an oligosaccharide” refers to the internalization of the intact oligosaccharide.

### EXAMPLES

The following examples are included for illustrative purposes only and are not intended to limit the scope of the invention.

### Example 1: Manufacture of a Concentrated HMO Mixture

Human milk from previously screened and approved donors was tested to verify donor identity and then mixed together to generate a pool of donor milk. In a clean room environment, the pool of donor milk was further tested, including for specific pathogens and bovine proteins. After testing, the pooled donor milk was filtered through a 200 m filter, heated to a temperature of approximately 63° C. for 30 minutes, and then cooled to between 2° C. and 8° C. The pooled human milk was then transferred to a centrifuge to separate the cream from the skim. The resulting skim milk was ultra-filtered with a 10 kDa membrane, and the material that passed through the filter was collected as the permeate fraction. The permeate was frozen and stored at approximately −20° C.

Multiple samples of the permeate fractions were thawed and pooled. The pH of the pooled permeate was adjusted to a target pH of 4.5±0.2. The permeate was then heated to approximately 50° C. Lactase enzyme was added to the permeate at a 0.1% w/w concentration and incubated at approximately 50° C. for 60 minutes. The permeate and lactase enzyme mixture then was cooled to between 20° C. and 30° C. and clarified by depth filtration (Filtrox CH113P). The resulting depth filter filtrate was then ultra-filtered (Biomax-10K membrane) to remove the lactase. The ultra-filtered permeate was then concentrated by nanofiltration using membranes with estimated 400 to 500 Dalton molecular weight cut-off (GE G-5 UF). The concentrated HMO composition was then pasteurized and clarified though 0.2 μm sterile filters. This final HMO composition was then filled into containers and stored at ≤−20° C. The final concentrations of HMO were quantified using high performance anion exchange chromatography with pulsed amperometry detection (HPAEC-PAD) with commercially available standards.

### Example 2: Administration of B. longum Subsp. infantis and a Concentrated HMO Mixture to Healthy Adult Subjects

Healthy adult men and women between the ages of 18 and 44 are enrolled as subjects in a study to evaluate administration of a B. longum subsp. infantis probiotic and the concentrated HMO mixture prepared as described in Example 1. Subjects receiving the B. longum subsp. infantis probiotic consume a dose of at least 8×10⁹ CFU of a B. longum subsp. infantis probiotic daily for the first seven days (days 1-7) of the clinical study. Some of the subjects are assigned to take a single 20 mg dose of an over-the-counter proton pump inhibitor containing omeprazole with sodium bicarbonate (ZEGERID™) 1-2 hours prior to consuming the probiotic. Subjects assigned to receive the concentrated HMO mixture consume two doses daily for the first fourteen days of the study (days 1-14) for a total daily dose of 4.5 g/day, 9 g/day, or 18 g/day HMO. Subjects of an additional cohort consume the PPI and B. longum subsp. in/an/is probiotic on days 1-7 and 18 g/day HMO on days 1-14, and then this treatment regimen is repeated beginning on day 29 of the study. The experimental cohorts are summarized in Table E1.

TABLE E1
Experimental Cohorts

| | Target HMO Amount | | B. infantis | Acid Reducer |
|---|---|---|---|---|
| Cohort | (Days 1-14) | | (Day 1-7) | (Day 1-7) |
| 1 | 0 | g/day | Yes | Yes |
| 2 | 18 | g/day | No | Yes |
| 3 | 4.5 | g/day | Yes | Yes |
| 4 | 9 | g/day | Yes | Yes |
| 5* | 18 | g/day | Yes | Yes |
| 6 | 18 | g/day | Yes | No |

*Cohort 5 undergoes the same dosing regimen of HMO, B infantis, and PPI twice. A first dosing regimen from days 1-14 and a second dosing regimen from days 29-43.

Biological samples (e.g., blood, urine, and stool samples) are collected from the subjects at various timepoints throughout the study. In particular, stool samples and blood samples are collected at screening (day 0) and various timepoints throughout the study.

Stool samples for microbiome analysis are prepared for DNA extraction. After DNA extraction, a sample will be analyzed using species- and strain-specific quantitative PCR analysis, such as described in Lawley et al., Peer J. 2017 May 25; 5:e3375. e.g., with forward and reverse primers identical to SEQ ID NOS: 54 and 55 and a probe sequence identical to SEQ ID NO: 56, to evaluate B. longum subsp. infantis colonization both during and after HMO ingestion. Selected samples may also be analyzed by next generation DNA sequencing to determine the taxonomic composition, alpha- and beta-diversity and change of each subject's microbiome during the study. Methods may include 16S and/or whole metagenomic shotgun sequencing and culture-specific methods.

Aliquots of stool are refrigerated immediately after collection and then frozen at about −70° C. or colder within 24 hours. Some samples are sent to a facility with metabolomic capabilities, such as to measure production of short chain fatty acids, levels of HMO, and/or other microbial metabolites.

Blood samples collected at screening (day 0) are to be used to determine subject eligibility and baseline results. Blood samples will be collected and tested for the purpose of safety monitoring at various timepoints throughout the study. The following tests may be performed: CBC with differential and platelets; alkaline phosphatase, ALT, AST, LDH, total and conjugated bilirubin, albumin, and total protein for liver function; electrolytes Na, K, Cl, HCO3, and glucose; total calcium, magnesium, and phosphate; creatinine and BUN for renal function. Blood will also be tested for markers of immunological activity, including but not limited to, TGF beta.

The levels of B. longum subsp. infantis within the gut microbiome present at baseline and at subsequent study points are evaluated by quantitative PCR using primers for species and strain. Quantitation limits are demonstrated by qualification assays demonstrating the lower limit of detection in order to establish minimum log-fold change in B. longum subsp. infantis. Results from baseline samples may indicate that adults do not harbor detectable levels of B. longum subsp. infantis. Results may indicate that B. longum subsp. infantis is not detectable in samples from subjects administered B. longum subsp. infantis without a dose of the concentrated HMO mixture at study points after the B. longum subsp. infantis has been administered. Results may also indicate that B. longum subsp. infantis is not detectable in samples from subjects administered B. longum subsp. infantis without a PPI. Alternatively, results may indicate that administration of a PPI reduces the levels of B. longum subsp. infantis. Likewise, results may indicate that B. infants is not detectable in samples from subjects administered the concentrated HMO mixture but not the B. longum subsp. infantis probiotic.

Results will indicate that B. longum subsp. infantis is present in at least some of the samples from subjects administered the B. longum subsp. infantis probiotic and the concentrated HMO mixture and may indicate a synergy between probiotic and mixture with respect to the abundance of B. longum subsp. infantis in the microbiome (e.g., the abundance of B. longum subsp. infantis at one or more timepoints will be greater than what would have been expected based on the administration of the probiotic or the mixture alone). Results may also indicate that the abundance of B. longum subsp. infantis increases during the HMO dosing regimen, and then decreases after day 14 once HMOs are no longer administered.

Changes in stool microbiota will be measured as well as dynamic changes in the gut community structure. These changes will be evaluated by next generation sequencing using proportions of key bacterial operational taxonomy units (OTUs), relative abundance of various taxa, diversity (alpha and beta) and stability of communities and functional metabolomic changes. Results may indicate that administration of the concentrated HMO mixture or both the concentrated HMO mixture and the B. longum subsp. infantis probiotic measurably changes the gut community structure. The results may indicate that the changes persist after the dosing regimens are completed. Results may also indicate that administration of the concentrated HMO mixture increases microbiome diversity, which may further be increased by administration of the B. longum subsp. infantis probiotic. Observations of increased microbiome diversity may persist at time points from after the dosing regimens are completed.

Changes in viability of proteobacteria and Enterococcus will be determined by plating on selective media. These measurements will predictive for success in suppressing pathogenic bacterial relatives in future trials. Results may indicate greater suppression of proteobacteria and Enterococcus in samples from subjects administered both the concentrated HMO mixture and the B. longum subsp. infantis probiotic as compared to samples from subjects administered either one alone and/or samples taken at baseline.

If higher levels of B. longum subsp. infantis is observed in stool from subjects administered a PPI than those from subjects not administered a PPI, such results may indicate a need for protection of B. longum subsp. infantis from stomach acid in order for engraftment of the organism into the gut microbiome to occur. Results may indicate that protection from exposure to stomach acid at a physiological pH, e.g., stomach acid at a pH of 1.5 to 3.5, is required for B. longum subsp. infantis engraftment. Alternatively, results may indicate that administration of a PPI does not influence the levels of B. longum subsp. infantis detected in stool samples. Results may even indicate that administration of a PPI reduces the levels of B. longum subsp. infantis detected in stool samples, which would be consistent with an inhibition, reduction, or prevention of B. longum subsp. infantis engraftment associated with the administration of the PPI.

Any adverse events are reported. Results may indicate that no major adverse events are reported, as B. longum subsp. infantis, HMOs, and PPIs are generally considered safe.

### Example 3: Administration of B. longum Subsp. infantis and a Concentrated HMO Mixture to Healthy Adult Subjects

A clinical study was performed similar to as described in Example 2. The study was performed with 12 total subjects, with two subjects each assigned to cohorts 1-6 shown in Table E1. Subjects were administered B. longum subsp. infantis from days 1-8 and/or one or both of a concentrated HMO mixture and a PPI from days 1-15. Stool samples were collected from the subjects as described in Example 2 at day 1 prior to administration of the B. longum subsp. infantis and/or the concentrated HMO mixture, and at days 5, 8, and 15. The levels of B. longum subsp. infantis within the gut microbiome present at the study points were evaluated by quantitative PCR of DNA isolated from stool samples using primers for B. longum subsp. infantis similar to as described in Example 2. Results are summarized in Table E2.

TABLE E2
Summary of B. infantis qPCR results in samples by subject

| | Treatment | | | | Copy Number/Reaction | | | |
|---|---|---|---|---|---|---|---|---|
| Subject | B. inf | HMO | | PPI | Day 1 | Day 5 | Day 8 | Day 15 |
| 1 | Yes | 0 | g/day | Yes | − | + | + | − |
| 2 | Yes | 0 | g/day | Yes | − | + | + | − |
| 3 | No | 18 | g/day | Yes | − | − | − | − |
| 4 | No | 18 | g/day | Yes | − | − | − | − |
| 5 | Yes | 4.5 | g/day | Yes | − | + | + | + |
| 6 | Yes | 4.5 | g/day | Yes | − | − | + | + |
| 7 | Yes | 9 | g/day | Yes | − | + | + | − |
| 8 | Yes | 9 | g/day | Yes | − | + | + | − |
| 9 | Yes | 18 | g/day | Yes | − | + | − | − |
| 10 | Yes | 18 | g/day | Yes | − | + | + | N/A |
| 11 | Yes | 18 | g/day | No | − | ++ | ++ | ++ |
| 12 | Yes | 18 | g/day | No | − | ++ | ++ | ++ |

Limit of detection = about 10 copies per reaction.
“−” indicates at or below limit of detection; “+” indicates between 10 and 10,000 copies per reaction; and “++” indicates above 10,000 copies per reaction.
“N/A” indicates unavailability due to subject non-compliance.

As shown in Table E2, B. longum subsp. infantis was not detectable by qPCR in any of the baseline samples collected at day 1, consistent with B. longum subsp. infantis not being present in a majority of adult intestinal microbiomes. Subjects administered B. longum subsp. infantis were administered the probiotic daily during days 1-8, and B. longum subsp. infantis was detectable in one or both of the stool samples collected at days 5 and 8 in all these subjects. B. longum subsp. infantis was not detected in any of the samples collected from subjects who were not administered the probiotic.

Day 15 was 7 days after the last administration of B. longum subsp. infantis and the last day that the concentrated HMO mixtures were administered. B. longum subsp. infantis was detected in stool samples collected at day 15 from four subjects, with the highest levels observed in the two subjects that received 18 g/day HMO and B. longum subsp. infantis without the PPI. These results are consistent with administration of the concentrated HMO mixture supporting engraftment and expansion of B. longum subsp. infantis that persists in the subject's intestinal microbiome beyond the daily administration of the probiotic.

### Example 4: Engraftment of B. longum Subsp. infantis in Healthy Adult Subjects

Additional subjects were enrolled in the clinical study described in Example 3. Subjects were assigned to the same cohorts summarized in Table E1, with the following changes: subjects assigned to receive doses of B. longum subsp. infantis and the concentrated HMO mixture at doses of 4.5 and 9 g/day HMO were not administered a PPI, and subjects assigned to Cohort 5 were further split into two sub-cohorts, Cohort 5A, which received the H2-receptor antagonist famotidine on days 29-36, and Cohort 5B, which received no acid reducing drugs on days 29-36. One subject from Cohort 5 who had received Omeprazole on days 29-36 was included in Cohort 5A. A summary of the experimental cohorts including the subjects described in Example 3 and the additional subjects are summarized in Table E3.

TABLE E3
Summary of Experimental Cohorts

| Cohort | Subjects | B. infantis | HMO | | PPI |
|---|---|---|---|---|---|
| 1 | 10 | Yes | 0 | g | 2/10 Omeprazole, 8/10 none |
| 2 | 10 | No | 18 | g | 2/10 Omeprazole, 8/10 none |
| 3 | 10 | Yes | 4.5 | g | 2/10 Omeprazole, 8/10 none |
| 4 | 10 | Yes | 9 | g | 2/10 Omeprazole, 8/10 none |
| 5A | 1 | Yes | 18 | g | Omeprazole/ Omeprazole |
| 5A | 4 | Yes | 18 | g | Omeprazole/Famotidine |
| 5B | 5 | Yes | 18 | g | Omeprazole/None |
| 6 | 9 | Yes | 18 | g | None |

Subjects were administered B. longum subsp. infantis from days 1-8 and/or a concentrated HMO mixture from days 1-15. In addition, subjects assigned to Cohorts 5a and 5b were administered B. longum subsp. infantis and the concentrated HMO mixture from days 29-36 and the concentrated HMO mixture alone on days 37-43. Stool samples were collected from the subjects as described in Example 2 at day 1 prior to administration of the B. longum subsp. infantis and/or the concentrated HMO mixture, and at days 5, 8,15, 22, and 29 of the study. Stool samples from subjects in Cohorts 5a and 5b were also collected on days 33, 36, 43, 50, and 57. Levels of B. longum subsp. infantis within the gut microbiome present at the study points were evaluated by quantitative PCR of DNA isolated from stool samples using primers for B. longum subsp. infantis described in Example 2.

For Cohort 1 (administered B. longum subsp. infantis but not the concentrated HMO mixture), B. longum subsp. infantis was detected in the stool from all ten subjects on days 5 and 8. B. longum subsp. infantis was not detected in stool collected from these subjects on day 1 (prior to the administration of B. longum subsp. infantis) or on days 15, 22, and 29 (after the administration of B. longum subsp. infantis), with the exception of detectable levels of B. longum subsp. infantis in stool collected from a single Cohort 1 subject on day 15. In some aspects, B. longum subsp. infantis would not be expected to engraft in these subjects due to the absence of HMO. Thus, in certain aspects, the levels of B. longum subsp. infantis measured from samples collected on days 5 and 8 may be considered to be pass-through levels of B. longum subsp. infantis that could be expected during dosing.

For Cohort 2 (administered the concentrated HMO mixture but not B. longum subsp. infantis), B. longum subsp. infantis was not detected in stools from any of the ten subjects collected at days 1, 8, 15, 22 and 29. Among the stools collected from Cohort 2 at day 5, B. longum subsp. infantis was only detected in the stool from a single subject. A follow-up analysis suggested that this B. longum subsp. infantis detection may have been a false positive due to a technical error. As only one stool sample collected from only one individual at a single time point had detectable levels of B. longum subsp. infantis, these data are consistent with reported absence of B. infantis in the adult gastrointestinal tract (Underwood et al., Pediatr Res. 2015; 77(1-2):229-235).

The qPCR results from samples collected from subjects in Cohorts 3, 4, and 6 (administered B. longum subsp. infantis and the concentrated HMO mixture at 4.5 g, 9 g, and 18 g of HMO per day, respectively) were assessed to identify subjects with successful B. longum subsp. infantis colonization or engraftment. Positive qPCR results on days 5, 8, and 15 were required for a subject to be considered successfully engrafted or colonized with B. longum subsp. infantis. Results are summarized in Table E3. Data from only 9 subjects were evaluated from Cohort 6 as one subject in the cohort withdrew consent after the baseline timepoint. B. longum subsp. infantis was observed in some subjects from all cohorts.

TABLE E4
Subjects colonized with B. infantis

| | Treatment | | | Engraftment |
|---|---|---|---|---|
| Cohort | B. inf | HMO | | (Colonized subjects/total) |
| 3 | Yes | 4.5 | g/day | 3/10 |
| 4 | Yes | 9 | g/day | 2/10 |
| 5a and 5b | Yes | 18 | g/day | 5/10 |
| 6 | Yes | 18 | g/day | 6/9 |

As discussed above, subjects of Cohorts 5a and 5b received treatment with acid reducing drugs (Omeprazole on days 1-8) in addition to B. longum subsp. infantis and 18 g per day of HMO. As shown in Table E4, combined results from Cohorts 5a and 5b on days 5, 8, and 15 were similar to the results observed from Cohort 6 (not administered acid reducing drugs), consistent with no discernable effect of administration of acid reducing drugs on B. longum subsp. infantis engraftment.

As discussed above, subjects of Cohorts 5a and 5b also received a second round of treatments with the concentrated HMO mixture and B. longum subsp. infantis after a two-week washout period. In addition, subjects were administered either Omeprazole or Famotidine (Cohort 5a) or no acid reducing drug (Cohort 5b). Of the five subjects in Cohorts 5a and 5b deemed to have successful engraftment of B. longum subsp. infantis at days 5, 8, and 15, two subjects sustained successful engraftment again in the second round of dosing (detectable B. longum subsp. infantis in samples collected at days 33, 36, 43). These data are consistent with an ability for subsequent colonization of B. longum subsp. infantis to occur after prior treatments with B. longum subsp. infantis and HMO.

### Example 5: Lactate Production in Subjects with Engraftment of B. longum Subsp. infantis

Stool samples collected at days 1, 5, 8, and 15 from the subjects described in Example 4 were assessed for the various short chain fatty acids (SCFAs). Briefly, portions of the stool samples were collected, weighed, and suspended in a normalized volume of deionized water. The suspensions were centrifuged and the resulting supernatants were filtered through a membrane with a pore size of 200 μm. The resulting filtered fecal water extracts were assessed by gas chromatography-mass spectrometry (GC-MS). For analysis of lactate content, fecal water extracts were derivatized with methyl chloroformate (Smart et al., Nat Protoc 5(10): 1709-29 (2010)). Analysis was performed using gas chromatography (7890B, Agilent) coupled to a quadropole mass spectrometry detector (5977B, Agilent). Raw data was converted to netCDF format using ChemStation (Agilent) before data was imported and processed in Matlab R2018b (Mathworks, Inc) using the PARADISe software (Johnsen et al., J Chromatogr A 1503: 57-64 (2017)).

Samples collected from subjects described in Example 4 that sustained successful engraftment of B. longum subsp. infantis (detectable qPCR B. longum subsp. infantis signal at days 5, 8, and 15) were compared to the subjects described in Example 4 that did not sustain successful engraftment (see, Table E4). Significantly greater levels of lactate were detected in the samples collected at day 15 from subjects that sustained B. longum subsp. infantis as compared to the subjects that did not sustain engraftment. These data are consistent with B. longum subsp. infantis engraftment resulting from treatments of B. longum subsp. infantis and concentrated HMO mixtures increasing lactate production within the subject's microbiomes.

### Example 6: Manufacture of a Concentrated HMO Composition with Reduced Levels of Monosaccharides

A composition of donor milk derived HMOs was manufactured similar to as described in Example 1, with an additional step for removal of monosaccharides. After the ultra-filtered human milk permeate was concentrated by nanofiltration and prior to the pasteurization step, the concentrated HMO composition was subjected to diafiltration using a 400-500 Dalton nominal pore size filter element. Flow-through removed from the retentate side was continuously replaced with six diavolumes of deionized water to remove monosaccharides and other low weight molecules.

### Example 7: Co-Cultures of B. longum Subsp. infantis and Pathogenic Bacteria

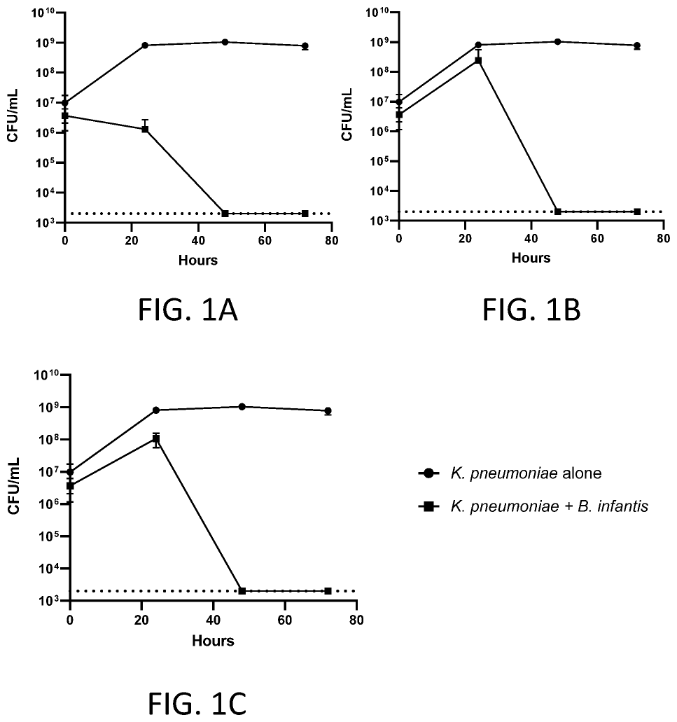

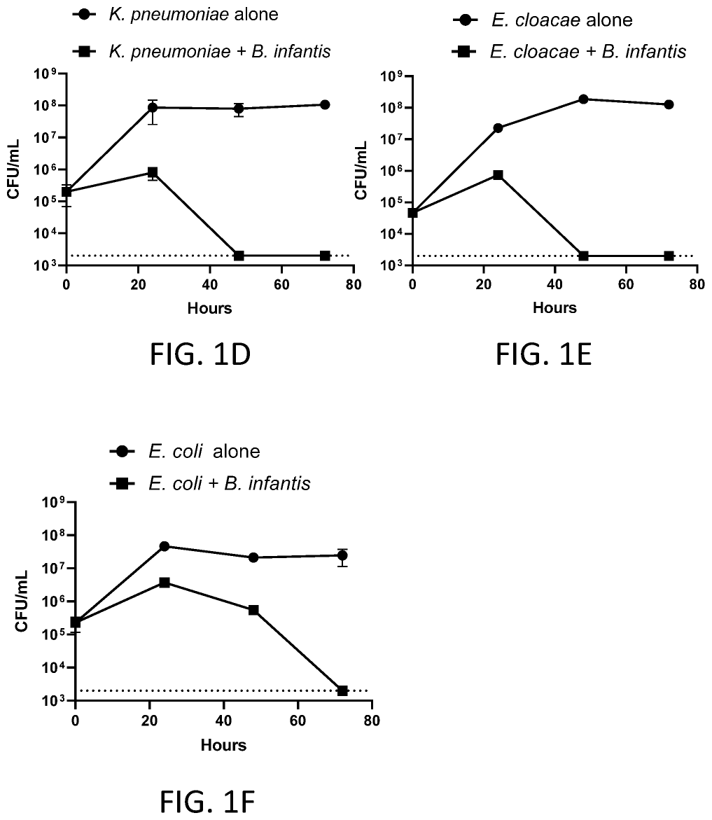

A strain of pathogenic bacteria, Klebsiella pneumoniae ATCC 43816, was co-cultured with B. longum subsp. infantis and incubated anaerobically in a carbon depleted modified Reinforced Clostridial Media (RCM) base media at starting ratios of 1:1 (FIG. 1A), 10:1 (FIG. 1B), and 100:1 (FIG. 1C) K. pneumoniae cells to B. longum subsp. infantis cells. For carbon sources, glucose and the concentrated HMO composition manufactured as described in Example 6 were utilized. Samples were collected following 24, 48, and 72 hours of incubation and plated aerobically to enumerate pathogen counts. As shown in FIGS. 1A-1C, co-culture with B. longum subsp. infantis inhibited the growth of K. pneumoniae at all ratios tested, with no viable K. pneumoniae detected at 48 and 72 hour time points.

A second strain of K. pneumoniae, Klebsiella pneumoniae MH258, was cultured as described above in the presence or absence of B. longum subsp. infantis. Strain MH258 was originally sourced from a clinical and is carbapenem-resistent (Xiong et at., 2015, Infect Immun 83(9):3418-27). Similar inhibition of Klebsiella pneumoniae MH258 was observed in co-cultures with B. longum subsp. infantis (FIG. 1D).

Two additional pathogenic bacteria strains, Enterobacteriaceae cloacae (E. cloacae) and Enterobacteriaceae coli (E. co/i), were cultured as described above in the presence or absence of B. longum subsp. infantis. As shown in FIGS. 1E and 1F, co-culture with B. longum subsp. infantis inhibited the growth of E. clocacae (FIG. 1E) and E. coli (FIG. 1F). No viable E. clocacae was detected at 48 and 72 hour time points (FIG. 1E) and no viable E. coli was detected at the 72 hour time point (FIG. 1F) following incubation with B. longum subsp. infantis.

Taken together, these data are consistent with an inhibition of the growth of pathogenic bacteria by B. longum subsp. infantis cultured in the presence of human milk oligosaccharides.

### Example 8: Co-Cultures of B. longum Subsp. infantis and Anaerostipes caccae

B. longum subsp. infantis and a strain of a bacteria species capable of producing butyrate, Anaerostipes caccae, were co-cultured or cultured alone and assessed for growth and short chain fatty acid (SCFA) production. A concentrated HMO mixture produced as described in Example 6 was used as a carbon source. Growth was assessed by qPCR with DNA isolated from cell culture samples collected following 0, 5, and 11 hours of incubation. Cell culture media was collected following 11 hours of incubation and levels of acetate, lactate, and butyrate were assessed by liquid chromatography with tandem mass spectrometry (LC-MS/MS).

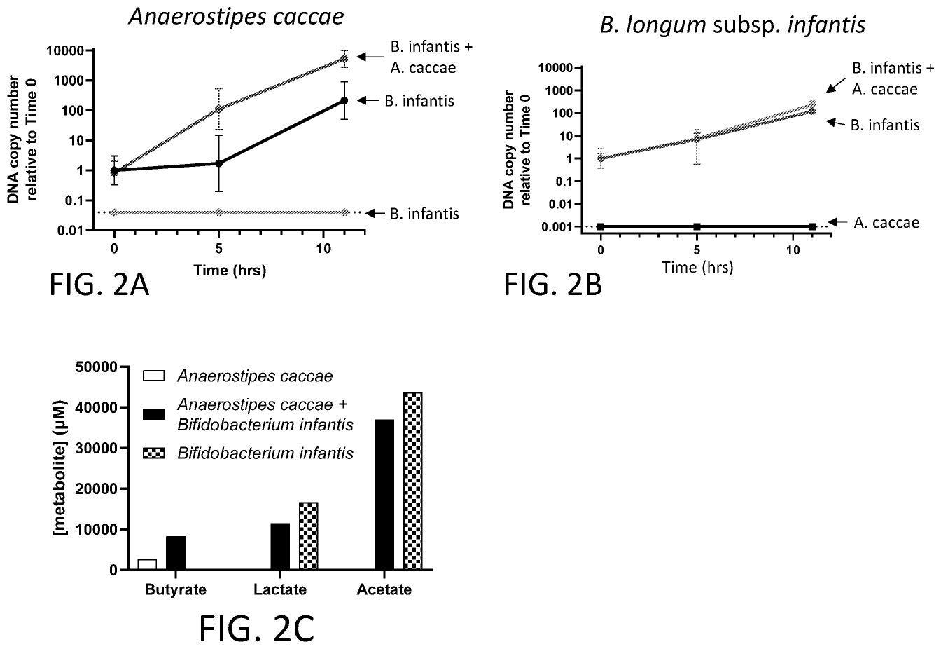

Co-culture of B. longum subsp. infantis and Anaerostipes caccae resulted in increased growth of Anaerostipes caccae as compared to the growth of Anaerostipes caccae when cultured alone (FIG. 2A). No effect of co-culture on the growth B. longum subsp. infantis was observed (FIG. 2B). Differences in levels of SCFAs were observed among the cell cultures. Lower levels of lactate and acetate were observed in co-cultures as compared to cultures of B. longum subsp. infantis alone, and higher levels of butyrate were observed in co-cultures as compared to cultures of Anaerostipes caccae alone (FIG. 2C). Lactate and acetate were not detected in media collected from cultures of Anaerostipes caccae alone, and butyrate was not detected in cultures of B. longum subsp. infantis alone. Taken together, these data are consistent with a cross feeding between B. longum subsp. infantis and Anaerostipes caccae in the presence of HMO, whereby Anaerostipes caccae consumes acetate and lactate produced by B. longum subsp. infantis resulting in growth and butyrate production.

### Example 9: Co-Cultures of B. longum Subsp. infantis and Butyrate Producing Bacteria

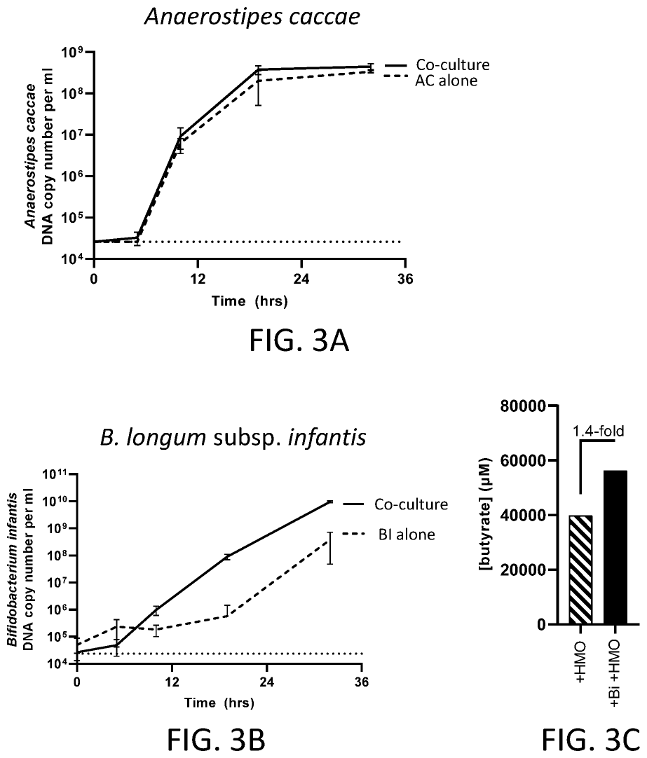

Strains of bacteria capable of producing butyrate were cultured in the presence or absence of B. longum subsp. infantis and assessed for growth and butyrate production. In one experiment, Anaerostipes caccae and B. longum subsp. infantis were cultured alone or together with the concentrated HMO mixture described in Example 6 as the sole carbon source. Growth and butyrate production were assessed as described in Example 8. Growth of Anaerostipes caccae and B. longum subsp. infantis alone and in co-culture are shown in FIGS. 3A and 3B. In media collected from cultures of Anaerostipes caccae grown for 11 hours, higher concentrations of butyrate were observed in co-cultures with B. longum subsp. infantis than in media from cultures of Anaerostipes caccae alone (FIG. 3C).

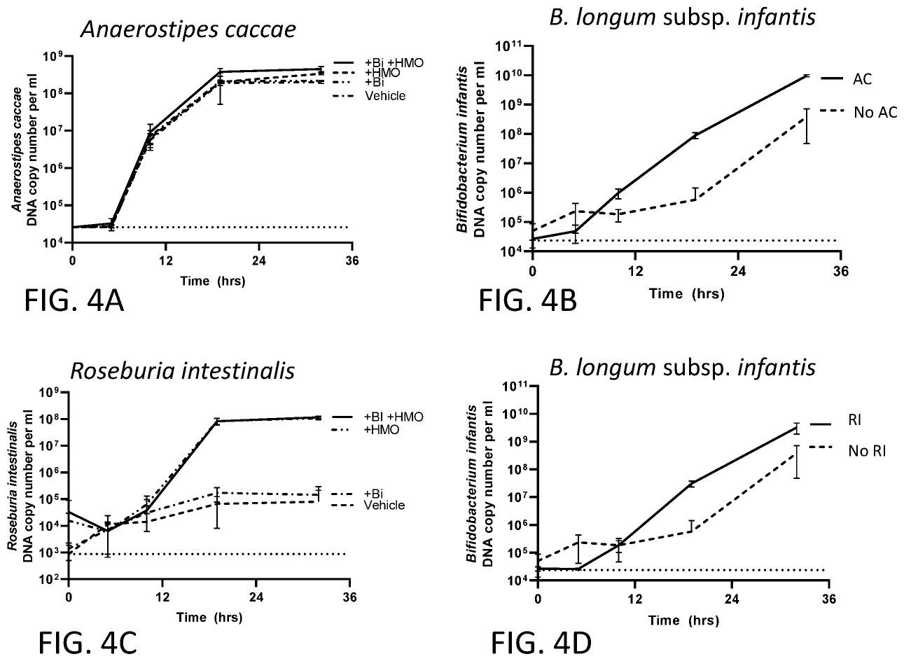

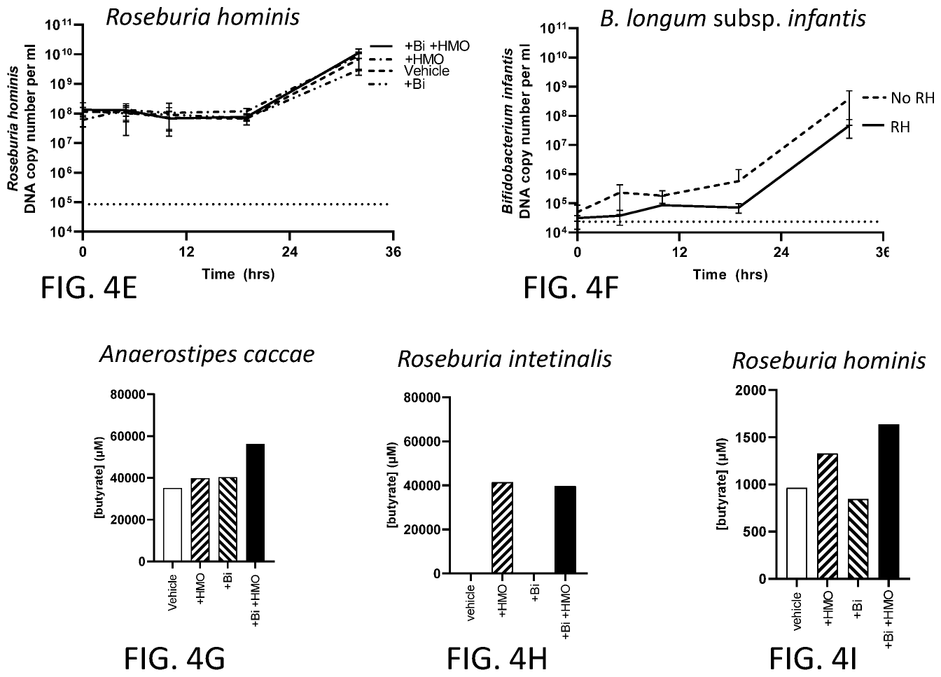

In an additional experiment, strains of bacteria capable of producing butyrate that included Anaerostipes caccae, Roseburia intestinalis, and Roseburia hominis were co-cultured with B. longum subsp. infantis in the presence of cellobiose, n-acetylglucosamine, and the concentrated HMO mixture as carbon sources. Controls included cultures of the strains capable of producing butyrate in the absence of B. longum subsp. infantis, in the absence of HMO, or the absence of both. Controls also included cultures of B. longum subsp. infantis alone. Growth and butyrate production were assessed similar to as described in Example 8. Butyrate production was assessed following 32 hours of incubation. Growth of strains capable of producing butyrate and B. longum subsp. infantis cultured alone and in co-culture are shown in FIGS. 4A-4F. In media collected from cultures of Anaerostipes caccae, higher concentrations of butyrate were observed in co-cultures with B. longum subsp. infantis in the presence of HMO than in media collected from controls (FIG. 4G). For Roseburia intestinalis and Roseburia hominis, greater concentrations of butyrate were observed in cultures of Roseburia alone with HMO and in cultures with HMO and B. longum subsp. infantis than in cultures lacking HMO (FIGS. 4H and 4I).

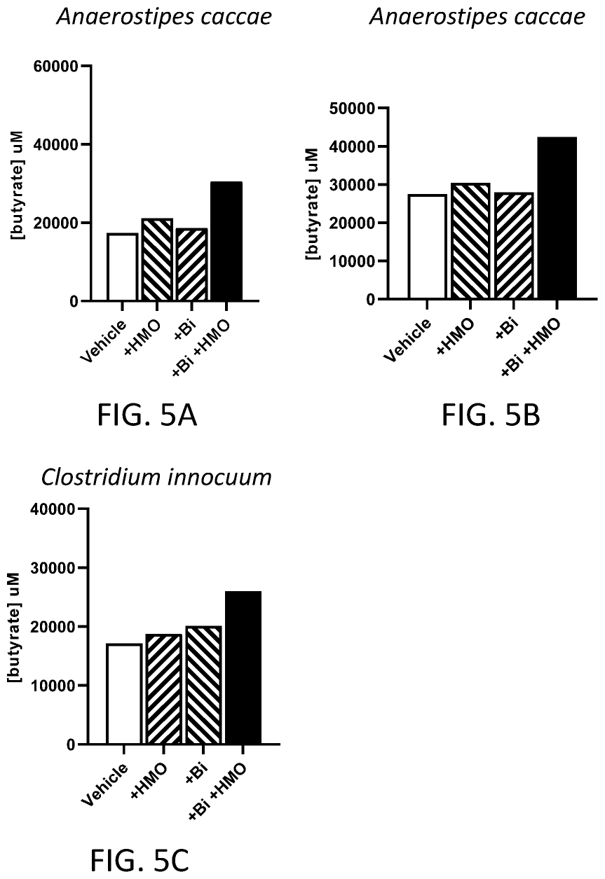

In a further experiment, strains of bacterium capable of producing butyrate, including two isolated strains of Anaerostipes caccae and a single strain of Clostridium innocuum were cultured in the presence or absence of B. longum subsp. infantis and with or without the concentrated HMO mixture described in Example 6. All experimental conditions included glucose, cellobiose, and n-acetylglucosamine for carbon sources. Butyrate production was assessed as described in Example 8 following 18 hours of incubation. Higher concentrations of butyrate were observed in media collected from cultures of Anaerostipes caccae (FIGS. 5A and 5B), and Clostridium innocuum (FIG. 5C) that were cultured in the presence of B. longum subsp. infantis and HMO as compared to controls.

Taken together, these results are consistent with a cross-feeding between B. longum subsp. infantis and some strains capable of producing butyrate in the presence of HMO. These results are also consistent with a capability of some strains for producing butyrate in the presence of HMO when B. longum subsp. infantis is not present.

### Example 10: B. longum Subsp. infantis Colonizes Mice Inoculated with a Humanized Intestinal Microbiome

A mouse model of a humanized intestinal microbiome was generated to assess B. longum subsp. infantis engraftment under different conditions. Germ free mice each received a single fecal microbiota transplant (FMT) from a healthy human adult or infant donor (day −3). FMTs were prepared from human stool samples that were processed into a 10% slurry in a saline buffer with glucose added for stability and stored at −80° C. Prior to inoculation, the stool samples were thawed under anaerobic conditions, centrifuged to remove buffer and glycerol, and resuspended in saline buffer alone. FMTs were administered in a 100 μl volume to germ free mice by oral gavage.

Beginning three days after the FMT (day 0), mice were administered doses of 1×10⁸ colony forming units (CFU) of B. longum subsp. infantis and 20 mg of total HMO from an HMO mixture (prepared as described in Example 1) twice daily for three days (day 0 to day 2). A group of germ free mice that did not receive an FMT also received treatment with B. longum subsp. infantis and HMO. A negative control group consisted of mice that received FMT from a healthy human adult donor but received a PBS vehicle in place of B. longum subsp. infantis and HMO. Fecal pellets were collected from mice in all experimental groups the day of the FMT (day −3) and then daily beginning on day 0 through day 5. DNA was isolated from the fecal pellets, and levels of B. longum subsp. infantis were assessed by qPCR performed similar to as described in Example 6.

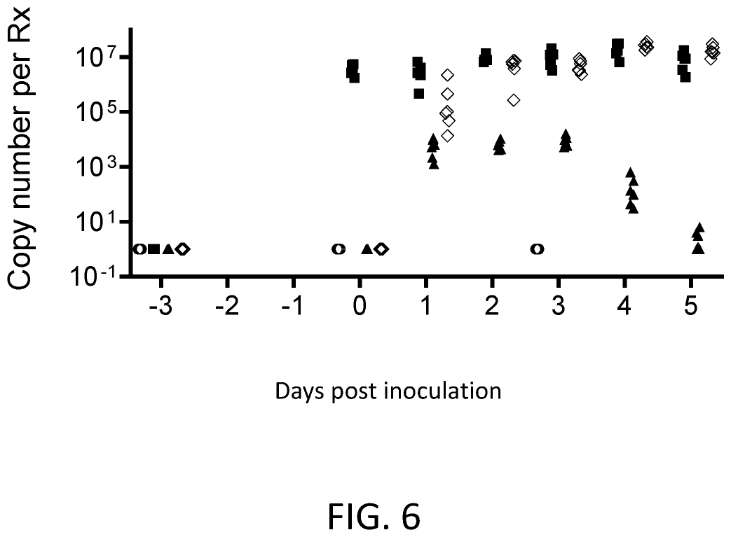

As shown in FIG. 6, B. longum subsp. infantis was detected in fecal pellets from mice inoculated with B. longum subsp. infantis but not in stool collected from mice in the negative control group. Consistent with colonization of B. longum subsp. infantis, this detection persisted to day 5, three days after the final B. longum subsp. infantis and HMO treatment was administered. Higher levels of B. longum subsp. infantis were detected in stool from the monocolonized mice (germ free mice that received B. longum subsp. infantis but not an FMT) and mice that received FMT from healthy infant donors than in mice that received an FMT from healthy adult donors. This trend is consistent with general observations that probiotic strains typically have greater expansion within microbiomes having lower microbial diversity (such as infant or dysbiotic microbiomes) than in healthy adult microbiomes which typically have high microbial diversity.

### Example 11: Butyrate Production in a Mice with a Humanized Intestinal Microbiome Following Administration of B. longum Subsp. infantis and Human Milk Oligosaccharides

A mouse model of a humanized intestinal microbiome similar as described in Example 10 was used to assess butyrate production following administration of B. longum subsp. infantis and human milk oligosaccharides. Germ free mice that received a fecal microbiota transplant (FMT) from a healthy human adult were administered B. longum subsp. infantis and either an HMO mixture prepared as described in Example 1 or a PBS vehicle control. On day 10 of the experiment, mice were sacrificed and cecal contents were collected for metabolite analysis by tandem liquid chromatography-mass spectrometry (LC-MS/MS). Greater levels of butyrate were detected in cecal material from mice treated with B. longum subsp. infantis and HMO as compared to control mice treated with B. longum subsp. infantis and PBS vehicle. This result is consistent with cross feeding between B. longum subsp. infantis and butyrate producing bacteria present among healthy human intestinal microbiota when HMOs are administered.

1-78. (canceled)

79. A method of treating or preventing a disease, disorder, or condition associated with one or more of inflammation, immune dysfunction, cancer, allergy, or dysbiosis of the intestinal microbiome in a subject in need thereof, the method comprising administering to the subject i) a prebiotic mixture comprising one or more human milk oligosaccharides, ii) at least one probiotic strain of bacterium comprises a species, subspecies, or strain of the genus Bifidobacterium capable of consuming the one or more human milk oligosaccharides; and iii) at least one butyrate producing strain of bacterium.

80. The method of claim 79, wherein the at least one butyrate producing strain comprises a strain of Clostridium Cluster IV or Clostridium Cluster XIVa bacteria.

81. The method of claim 79, wherein the at least one butyrate producing strain comprises a 16S nucleic sequence having at least 95%, 98%, 99%, or 100% identity to any sequence set forth in SEQ ID NOS: 77-167.

82. The method of claim 79, wherein the at least one butyrate producing strain comprises one or more of Agathobacter rectalis, Anaerobutyricum hallii, Anaerostipes caccae, Blautia producta, Clostridium leptum, Faecalibacterium prausnitzii, Anaerobutyricum soehngenii, Roseburia hominis, or Roseburia intestinalis.

83. The method of claim 79, wherein the at least one probiotic strain comprises B. breve, B. bifidum, B. longum subsp. infantis, or B. longum subsp. longum.

84. The method of claim 79, wherein the at least one probiotic strain comprises B. longum subsp. infantis.

85. The method of claim 79, wherein the prebiotic mixture comprises one or more of 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, or disialyllacto-N-tetraose.

86. The method of claim 79, wherein the prebiotic mixture comprises at least 10, at least 25, at least 50, at least 100, or at least 150 human milk oligosaccharides.

87. The method of claim 79, wherein the subject has, is suspected of having, or is at risk of having one or more of obesity, type II diabetes, a chronic inflammatory disease, an autoimmune disease, an infection, an infectious disease domination, bowel resection, a condition associated with chronic diarrhea, irritable bowel syndrome (IBS), inflammatory bowel disease (IBD), short bowel syndrome (SBS), celiac disease, small intestinal bacterial overgrowth (SIBO), gastroenteritis, leaky gut syndrome, pouchitis, gastric lymphoma, bacterial, viral, or parasitic infection or overgrowth, infection by drug-resistant bacteria, cancer, or graft versus host disease.

88. A method of treating or preventing a disease, disorder, or condition associated with one or more of inflammation, immune dysfunction, cancer, allergy, or dysbiosis of the intestinal microbiome in a subject in need thereof, the method comprising administering to the subject i) a prebiotic mixture comprising one or more human milk oligosaccharides and ii) at least one probiotic strain of bacterium capable of consuming the one or more human milk oligosaccharides comprising the at least one probiotic strain comprises a species, subspecies, or strain of the genus Bifidobacterium; wherein the subject has undergone or will undergo a bacteriotherapy.

89. The method of claim 88, wherein the bacteriotherapy comprises one or more bacteria species, subspecies, or strains obtained from, isolated from, derived from, or present in a human intestinal microbiome and/or a human stool.

90. The method of claim 88, wherein the bacteriotherapy comprises all or a portion of the bacteria present in a human stool and/or in a human intestinal microbiome.

91. The method of claim 88, wherein the bacteriotherapy comprises a fecal microbiota transfer composition.

92. The method of claim 88, wherein the at least one probiotic strain comprises B. longum subsp. infantis.

93. The method of claim 88, wherein the prebiotic mixture comprises one or more of 2′-fucosyllactose, 3-fucosyllactose, 3′-sialyllactose, 6′-sialyllactose, lacto-N-tetraose, lacto-N-difucohexaose I, lactodifucotetraose, lacto-N-fucopentaose I, sialylacto-N-tetraose c, sialylacto-N-tetraose b, or disialyllacto-N-tetraose.

94. The method of claim 88, wherein the prebiotic mixture comprises at least 10, at least 25, at least 50, at least 100, or at least 150 human milk oligosaccharides.

95. The method of claim 88, wherein the subject has, is suspected of having, or is at risk of having one or more of obesity, type II diabetes, a chronic inflammatory disease, an autoimmune disease, an infection, an infectious disease domination, bowel resection, a condition associated with chronic diarrhea, irritable bowel syndrome (IBS), inflammatory bowel disease (IBD), short bowel syndrome (SBS), celiac disease, small intestinal bacterial overgrowth (SIBO), gastroenteritis, leaky gut syndrome, pouchitis, gastric lymphoma, bacterial, viral, or parasitic infection or overgrowth, infection by drug-resistant bacteria, cancer, or graft versus host disease.

96. A method of preparing a bacteriotherapy to treat or prevent a condition associated with dysbiosis of the intestinal microbiome; the method comprising incubating a mixture of bacteria obtained from stool with one or more enrichment agents in a culture, thereby obtaining a cultured mixture of bacteria, wherein the one or more enrichment agents comprise one or more of a human milk oligosaccharide, a short chain fatty acid, or lactate; and
collecting, harvesting, or isolating at least one cultured bacterium from the cultured mixture of bacteria, thereby obtaining a bacteriotherapy.

97. A method for identifying one or more bacteria suitable for a bacteriotherapy; the method comprising incubating a mixture of bacteria obtained from stool with one or more enrichment agents in a culture, thereby obtaining a cultured mixture of bacteria, wherein the one or more enrichment agents comprise one or more of a human milk oligosaccharide, a short chain fatty acid, or lactate; and
collecting, harvesting, or isolating at least one cultured bacterium from the cultured mixture of bacteria, thereby obtaining a bacteriotherapy.

98. A method of preparing a bacteriotherapy, the method comprising collecting, harvesting, or isolating at least one bacterium from a mixture of bacteria, wherein the mixture of bacteria is, originates from, or is obtained from a stool sample from a healthy donor,
wherein the healthy donor was administered a prebiotic mixture and a probiotic strain comprising B. breve, B. bifidum, B. longum subsp. infantis, or B. longum subsp. longum comprising one or more human milk oligosaccharides, within less than four weeks prior to collection of the stool sample.


SEQUENCE LISTING (178 sequences)

SEQ ID NO: 1 - DNA, 1452 bp, Bifidobacterium infantis
  misc_feature: B. longum subsp. infantis partial 16S sequence GenBank LC071820.1
tggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgg

SEQ ID NO: 2 - DNA, 1537 bp, Bifidobacterium infantis
  misc_feature: B. longum subsp. infantis partial 16S sequence GenBank KP326372.1
tttttgtggagggttcgattctggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctttctacggaga

SEQ ID NO: 3 - DNA, 472 bp, Bifidobacterium infantis
  misc_feature: B. longum subsp. infantis partial 16S sequence AB116305.1
cgctggcggcgtgcttacacatgcaagtcgaacgggatccatcgggctttgcttggtggtgagagtggcgaacgggtgaataatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtagtgtacccgttgaataagcaccggttaactcgtgccagcagccgcgg

SEQ ID NO: 4 - DNA, 1458 bp, Bifidobacterium infantis
  misc_feature: B. longum subsp. infantis partial 16S sequence GenBank X70974.1
  misc_feature: n = a, t, c, or g
  misc_feature (50)..(50): n is a, c, g, or t
  misc_feature (176)..(176): n is a, c, g, or t
  misc_feature (234)..(234): n is a, c, g, or t
  misc_feature (266)..(266): n is a, c, g, or t
  misc_feature (353)..(353): n is a, c, g, or t
  misc_feature (363)..(363): n is a, c, g, or t
  misc_feature (370)..(370): n is a, c, g, or t
  misc_feature (398)..(398): n is a, c, g, or t
  misc_feature (437)..(437): n is a, c, g, or t
  misc_feature (451)..(451): n is a, c, g, or t
  misc_feature (453)..(453): n is a, c, g, or t
  misc_feature (462)..(462): n is a, c, g, or t
  misc_feature (467)..(467): n is a, c, g, or t
  misc_feature (474)..(474): n is a, c, g, or t
  misc_feature (522)..(522): n is a, c, g, or t
  misc_feature (528)..(528): n is a, c, g, or t
  misc_feature (548)..(548): n is a, c, g, or t
  misc_feature (721)..(721): n is a, c, g, or t
  misc_feature (807)..(807): n is a, c, g, or t
  misc_feature (809)..(809): n is a, c, g, or t
  misc_feature (813)..(817): n is a, c, g, or t
  misc_feature (827)..(828): n is a, c, g, or t
  misc_feature (857)..(857): n is a, c, g, or t
  misc_feature (862)..(862): n is a, c, g, or t
  misc_feature (868)..(950): n is a, c, g, or t
  misc_feature (997)..(1009): n is a, c, g, or t
  misc_feature (1013)..(1014): n is a, c, g, or t
  misc_feature (1018)..(1019): n is a, c, g, or t
  misc_feature (1028)..(1028): n is a, c, g, or t
  misc_feature (1032)..(1032): n is a, c, g, or t
  misc_feature (1046)..(1047): n is a, c, g, or t
  misc_feature (1051)..(1051): n is a, c, g, or t
  misc_feature (1054)..(1054): n is a, c, g, or t
  misc_feature (1056)..(1068): n is a, c, g, or t
  misc_feature (1089)..(1089): n is a, c, g, or t
  misc_feature (1097)..(1097): n is a, c, g, or t
  misc_feature (1111)..(1111): n is a, c, g, or t
  misc_feature (1113)..(1114): n is a, c, g, or t
  misc_feature (1121)..(1122): n is a, c, g, or t
  misc_feature (1132)..(1133): n is a, c, g, or t
  misc_feature (1146)..(1146): n is a, c, g, or t
  misc_feature (1187)..(1187): n is a, c, g, or t
  misc_feature (1270)..(1270): n is a, c, g, or t
  misc_feature (1272)..(1272): n is a, c, g, or t
  misc_feature (1276)..(1276): n is a, c, g, or t
  misc_feature (1295)..(1295): n is a, c, g, or t
  misc_feature (1337)..(1337): n is a, c, g, or t
  misc_feature (1340)..(1340): n is a, c, g, or t
  misc_feature (1382)..(1383): n is a, c, g, or t
  misc_feature (1393)..(1393): n is a, c, g, or t
  misc_feature (1406)..(1406): n is a, c, g, or t
  misc_feature (1423)..(1423): n is a, c, g, or t
  misc_feature (1431)..(1431): n is a, c, g, or t
  misc_feature (1439)..(1439): n is a, c, g, or t
tttgtggagggttcgattctggctcaggatgaacgctggcggcgtgcttnacacatgcaagtcgaacgggatccatcgggctttgcttggtggtgagagtggcgaacgggtgagtaatgcgtgaccgacctgccccatacaccggaatagctcctggaaacgggtggtaatgccgnatgttccagttgatcgcatggtcttctgggaaactttcgcggtatgggatggggtcgngtcctatcagcttgacggcggggtaacggccnaccgtggcttcgacgggtagccggcctgagagggcgaccggccacattgggactgagatacggcccagactcctacgggaggcagcngtggggaatnttgcacnatgggcgcaagcctgatgcagcgacgcngcgtgagggatggaggcttcgggttgtaaacctcttttntcggggagcaagcntnagtgagttnacccnttgaatnagcaccggctaactacgtgccagcagccgcggtaatacgtagggtgcnagcgtnatccggaattattgggcgtnaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggcngttactgacgcttaggagctaaagcgttgggagcgaacaggattagataccctggtagtccacgccgtaaacggtggatgctggangngggnnnnnttccacgggnnccgtgtcggacgaacgcgttaagcatccngcctngggagnnnnnnnnnnnnnnnnnnnnnnnnnnnnnnnnnnnnnnnnnnnnnnnnnnnnnnnnnnnnnnnnnnnnnnnnnnnnnnnnnnnaagaaccttacctgggcttgacatgttcccgacgatcccagaggggnnnnnnnnnnnnngggnnggtnnacaggtggngcanggtcgtcgtcagcnngtgncgngnnnnnnnnnnnnnagtcccgcaacgagcgcaacnctcgcccngtgttgccagcggntnntgccggnnactcacgggnnaccgccggggttnactcggaggaaggtggggatgacgtcagatcatcatgcccnttacgtccagggcttcacgcatgctacaatggccggtacaacgggatgcgacgcggcgacgcggagcggatccctgaaaaccngnctcngttcggatcgcagtctgcnactcgactgcgtgaaggcggagtcgctagtaatcgcgaatcngcngcgtcgcggtgaatgcgttcccgggccttgtacacaccgccnntcaagtcatnaaagtgggcagcncccgaagccggtggccnaacccctngtgggatngagccgtctaaggtgaggc

SEQ ID NO: 5 - DNA, 1515 bp, Bifidobacterium infantis
  misc_feature: B. longum subsp. infantis partial 16S sequence GenBank D86184.1
tttgatcatggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctta

SEQ ID NO: 6 - DNA, 658 bp, Bifidobacterium infantis
  misc_feature: B. longum subsp. infantis partial 16S sequence GenBank AB507102.1
ccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactg

SEQ ID NO: 7 - DNA, 1447 bp, Bifidobacterium infantis
  misc_feature: B. longum subsp. infantis strain FC13644 16S, partial sequence MK962475.1
gatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggact

SEQ ID NO: 8 - DNA, 1530 bp, Bifidobacterium adolescentis
  misc_feature: B. adolescentis 16S sequence NCBI Reference Sequence NR_074802.2
tttgtggagggttcgattctggctcaggatgaacgctggcggcgtgcttaacacatgcaagtcgaacgggatcggctggagcttgctccggccgtgagagtggcgaacgggtgagtaatgcgtgaccgacctgccccatacaccggaatagctcctggaaacgggtggtaatgccggatgctccagttggatgcatgtccttctgggaaagattctatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctccttt

SEQ ID NO: 9 - DNA, 1459 bp, Bifidobacterium animalis
  misc_feature: B. animalis subsp. animalis 16S sequence NCBI Reference Sequence NR_119007.1
  misc_feature (29)..(29): n is a, c, g, or t
  misc_feature (61)..(61): n is a, c, g, or t
  misc_feature (64)..(64): n is a, c, g, or t
  misc_feature (69)..(70): n is a, c, g, or t
  misc_feature (75)..(76): n is a, c, g, or t
  misc_feature (82)..(82): n is a, c, g, or t
  misc_feature (85)..(86): n is a, c, g, or t
  misc_feature (108)..(108): n is a, c, g, or t
  misc_feature (155)..(155): n is a, c, g, or t
  misc_feature (167)..(168): n is a, c, g, or t
  misc_feature (175)..(175): n is a, c, g, or t
  misc_feature (178)..(178): n is a, c, g, or t
  misc_feature (191)..(192): n is a, c, g, or t
  misc_feature (222)..(222): n is a, c, g, or t
  misc_feature (277)..(277): n is a, c, g, or t
  misc_feature (305)..(305): n is a, c, g, or t
  misc_feature (325)..(328): n is a, c, g, or t
  misc_feature (346)..(347): n is a, c, g, or t
  misc_feature (419)..(419): n is a, c, g, or t
  misc_feature (426)..(426): n is a, c, g, or t
  misc_feature (434)..(434): n is a, c, g, or t
  misc_feature (440)..(440): n is a, c, g, or t
  misc_feature (459)..(459): n is a, c, g, or t
  misc_feature (514)..(514): n is a, c, g, or t
  misc_feature (528)..(528): n is a, c, g, or t
  misc_feature (545)..(545): n is a, c, g, or t
  misc_feature (554)..(554): n is a, c, g, or t
  misc_feature (726)..(726): n is a, c, g, or t
  misc_feature (728)..(728): n is a, c, g, or t
  misc_feature (746)..(746): n is a, c, g, or t
  misc_feature (771)..(771): n is a, c, g, or t
  misc_feature (775)..(775): n is a, c, g, or t
  misc_feature (783)..(783): n is a, c, g, or t
  misc_feature (821)..(821): n is a, c, g, or t
  misc_feature (863)..(863): n is a, c, g, or t
  misc_feature (889)..(889): n is a, c, g, or t
  misc_feature (895)..(897): n is a, c, g, or t
  misc_feature (900)..(901): n is a, c, g, or t
  misc_feature (911)..(911): n is a, c, g, or t
  misc_feature (919)..(919): n is a, c, g, or t
  misc_feature (925)..(926): n is a, c, g, or t
  misc_feature (940)..(940): n is a, c, g, or t
  misc_feature (949)..(951): n is a, c, g, or t
  misc_feature (959)..(959): n is a, c, g, or t
  misc_feature (1004)..(1005): n is a, c, g, or t
  misc_feature (1009)..(1009): n is a, c, g, or t
  misc_feature (1033)..(1033): n is a, c, g, or t
  misc_feature (1073)..(1073): n is a, c, g, or t
  misc_feature (1086)..(1086): n is a, c, g, or t
  misc_feature (1091)..(1091): n is a, c, g, or t
  misc_feature (1118)..(1119): n is a, c, g, or t
  misc_feature (1126)..(1127): n is a, c, g, or t
  misc_feature (1151)..(1151): n is a, c, g, or t
  misc_feature (1195)..(1195): n is a, c, g, or t
  misc_feature (1209)..(1209): n is a, c, g, or t
  misc_feature (1218)..(1218): n is a, c, g, or t
  misc_feature (1220)..(1220): n is a, c, g, or t
  misc_feature (1275)..(1275): n is a, c, g, or t
  misc_feature (1277)..(1277): n is a, c, g, or t
  misc_feature (1281)..(1281): n is a, c, g, or t
  misc_feature (1300)..(1300): n is a, c, g, or t
  misc_feature (1328)..(1328): n is a, c, g, or t
  misc_feature (1331)..(1331): n is a, c, g, or t
  misc_feature (1379)..(1379): n is a, c, g, or t
  misc_feature (1386)..(1386): n is a, c, g, or t
  misc_feature (1388)..(1388): n is a, c, g, or t
  misc_feature (1390)..(1391): n is a, c, g, or t
  misc_feature (1394)..(1396): n is a, c, g, or t
  misc_feature (1398)..(1398): n is a, c, g, or t
  misc_feature (1411)..(1411): n is a, c, g, or t
  misc_feature (1416)..(1416): n is a, c, g, or t
  misc_feature (1429)..(1429): n is a, c, g, or t
  misc_feature (1452)..(1452): n is a, c, g, or t
ggttcgattctggctcaggatgaacgctngcggcgtgcttaacacatgcaagtcgaacggnatnctggnngcctnnctgccnggnngagagtggcgaacgggtgagtnatgcgtgaccaacctgccctgtgcaccggaatagctcctggaaacgngtggtaataccnnatctccnccnaccgcatggtggnntgggaaatgctttttgcggcatgggatggngtcgcgtcctatcagcttgttggcggggtgatggcccaccaaggcgttgacgggnagccggcctgagagggtgaccggccacnttgggactgagatacggccnnnnctcctacgggaggcagcnntggggaatattgcacaatgggcgcaagcctgatgcagcgacgccgcgtgcgggatggaggcttcgggttgtnaaccgcntttgttcnagggcnaggcacggcttcgggccgngttgagtggattgttcgaataagcaccggctaactacgtgccagcagccgcggtnatacgtagggtgcnagcgttatccggatttnttgggcgtnaagggctcgtaggcggttcgtcgcgtccggtgtgaaagtccatcgctaacggtggatctgcgccgggtacgggcgggctggagtgcggtaggggagactggaattcccggtgtaacggtggaatgtgtagatatcgggaagaacaccaatggcgaaggcaggtctctgggcngntactgacgctgaggagcnaaagcgtggggagcgaacaggattngatnccctggtngtccacgccgtaaacggtggatgctggatgtggggccntttccacgggtcctgtgtcggagccaacgcgttaagcatccngcctggggagtacggccgcaaggctnaaactnnnagnnattgacgggngccgcacnagcggnngagcatgcggattnattcgatgnnncgcgaagnaccttacctgggcttgacatgtgccggatcgccccggaaacgggnngtgncttcggggccggttcacaggtggngcatggtcgtcgtcagctcgtgtcgtgagatgttgggttnagtcccgcaacgngcgcnaccctcgccgcatgttgccagcgggtnntgccggnnactcatgtgggaccgccggggtcnactcggaggaaggtggggatgacgtcagatcatcatgccccttncgtccagggcttcncgcatgctncnatggccggtacaacgcgatgcgacacggtgacgtggggcggatcgctgaaaaccngnctcngttcggatcgcagtctgcnactcgactgcgtgaaggcggagtcgctngtnatcgcggatcagcaacgccgcggtgaatgcgttcccgggccttgtacncaccgcncntnnagnnntnaaagtgggtagcncccgnagccggtggcccnaccctcgtggggggagccgtctnaggtgag

SEQ ID NO: 10 - DNA, 1420 bp, Bifidobacterium animalis
  misc_feature: B. animalis subsp. lactis partial 16S sequence GenBank KU821112
caggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacgcggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacc

SEQ ID NO: 11 - DNA, 1488 bp, Bifidobacterium bifidum
  misc_feature: B. bifidum partial 16S sequence
gatgaacgctggcggcgtgcttaacacatgcaagtcgaacgggatccatcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctg

SEQ ID NO: 12 - DNA, 1520 bp, Bifidobacterium breve
  misc_feature: B. breve partial 16S sequence NCBI Reference Sequence NR_040783.1
ttcgattctggctcaggatgaacgctggcggcgtgcttaacacatgcaagtcgaacgggatccatcgggctttgcttggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgcgggagggagccgtctaaggtgaggctcgtgattgggactaagtcgtaacaaggtagccgtaccggaaggtgcggctggatcacctcctta

SEQ ID NO: 13 - DNA, 1445 bp, Bifidobacterium catenulatum
  misc_feature: B. catenulatum partial 16S sequence
tgaacgctggcggcgtgcttaacacatgcaagtcgaacgggatccaggcagcttgctgcctggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccccttgtgggatggagccgtctaaggtgagactcgtgattggg

SEQ ID NO: 14 - DNA, 1464 bp, Bifidobacterium longum
  misc_feature: B. longum partial 16S sequence GenBank LC306854.1
tggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgta

SEQ ID NO: 15 - DNA, 1513 bp, Bifidobacterium pseudocatenulatum
  misc_feature: B. pseudocatanulatum partial 16S sequence
gtttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgtggatggagccgtctaaggtgagactcgtgattgggactaagtcgtaacaaggtagccgtaccggaaggtgcggctggatcacc

SEQ ID NO: 16 - DNA, 1475 bp, Bifidobacterium pseudolongum
  misc_feature: B. pseudolongum partial 16S sequence GenBank M58742.1
  misc_feature: n = a,t,c, or g
  misc_feature (1)..(2): n is a, c, g, or t
  misc_feature (50)..(50): n is a, c, g, or t
  misc_feature (55)..(55): n is a, c, g, or t
  misc_feature (57)..(57): n is a, c, g, or t
  misc_feature (61)..(61): n is a, c, g, or t
  misc_feature (95)..(95): n is a, c, g, or t
  misc_feature (137)..(137): n is a, c, g, or t
  misc_feature (207)..(207): n is a, c, g, or t
  misc_feature (236)..(236): n is a, c, g, or t
  misc_feature (267)..(268): n is a, c, g, or t
  misc_feature (303)..(303): n is a, c, g, or t
  misc_feature (334)..(335): n is a, c, g, or t
  misc_feature (400)..(400): n is a, c, g, or t
  misc_feature (470)..(470): n is a, c, g, or t
  misc_feature (506)..(506): n is a, c, g, or t
  misc_feature (525)..(525): n is a, c, g, or t
  misc_feature (724)..(724): n is a, c, g, or t
  misc_feature (744)..(744): n is a, c, g, or t
  misc_feature (819)..(819): n is a, c, g, or t
  misc_feature (865)..(865): n is a, c, g, or t
  misc_feature (877)..(877): n is a, c, g, or t
  misc_feature (905)..(905): n is a, c, g, or t
  misc_feature (911)..(913): n is a, c, g, or t
  misc_feature (948)..(948): n is a, c, g, or t
  misc_feature (1005)..(1005): n is a, c, g, or t
  misc_feature (1118)..(1118): n is a, c, g, or t
  misc_feature (1133)..(1133): n is a, c, g, or t
  misc_feature (1136)..(1136): n is a, c, g, or t
  misc_feature (1150)..(1150): n is a, c, g, or t
  misc_feature (1169)..(1169): n is a, c, g, or t
  misc_feature (1276)..(1276): n is a, c, g, or t
  misc_feature (1367)..(1367): n is a, c, g, or t
  misc_feature (1386)..(1387): n is a, c, g, or t
  misc_feature (1393)..(1393): n is a, c, g, or t
  misc_feature (1428)..(1428): n is a, c, g, or t
  misc_feature (1453)..(1453): n is a, c, g, or t
nntttgtggagggttcgattctggctcaggatgaacgctggcggcgtgcntaacncntgcnagtcgaacgggatccatcaagcttgcttggtggngagagtggcgaacgggtgagtaatgcgtgaccgacctgcccnatacaccggaatagctcctggaaacgggtggtaatgccggatgttccagttgatcgcatggtcttctggnaaagctttcgcggtatgggatggggtcgngtcctatcagcttgacggcggggtaacggcnnaccgtggcttcgacgggtagccggcctgagagggngaccggccacattgggactgagatacggccnngactcctacgggaggcagcagtggggaatattgcacaatgggcgcaagcctgatgcagcgacgcngcgtgagggatggaggccttcgggttgtaaacctcttttatcggggagcaagcgagagtgagtttacccnttgaataagcaccggctaactacgtgccagcagccncggtaatacgtagggtgcnagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggcngttactgacgctgaggagcnaaagcgtggggagcgaacaggattagataccctggtagtccacgccgtaaacggtggatgctggatgtggggccngttccacgggttccgtgtcggagctaacgcgttaagcatcccgccnggggagtacggncgcaaggctaaaactcaaagaaattgangggggnnngcacaagcggcggagcatgcggattaattcgatgnaacgcgaagaaccttacctgggcttgacatgttcccgacggtcgtagagatacggcntcccttcggggcgggttcacaggtggtgcatggtcgtcgtcagctcgtgtcgtgagatgttgggttaagtcccgcaacgagcgcaaccctcgccccgtgttgccagcggattntgccgggaactcacnggncaccgccggggttnactcggaggaaggtggggntgacgtcagatcatcatgccccttacgtccagggcttcacgcatgctacaatggccggtacaacgggatgcgacgcggcgacgcggagcggatccctgaaaaccggnctcagttcggatcgcagtctgcaactcgactgcgtgaaggcggagtcgctagtaatcgcgaatcagcaacgtcgcggtgaatgcgttcccnggccttgtacacaccgccnntcaagncatgaaagtgggcagcacccgaagccggtggcctnaccccttgtgggatggagccgtctnaggtgaggctcgtgattgggtc

SEQ ID NO: 17 - DNA, 1478 bp, Lactobacillus acidophilus
  misc_feature: L. acidophilus partial 16S sequence NCBI Reference Sequence NR_043182.1
tcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggc

SEQ ID NO: 18 - DNA, 1520 bp, L. antri
  misc_feature: L. antri partial 16S sequence NCBI Reference Sequence NR_027206.1
ttgatcctggctcaggatgaacgccggcggtgtgcctaatacatgcaagtcgagcgcactggcccaactgaaatgacgtgcttgcacagaatggacgttggattcccagtgagcggcggacgggtgagtaacacgtgggcaacctgccccaaagcgggggataacatttggaaacagatgctaataccgcataagttggaaaaccacatggttttcccatcaaagatggtttcggctatcgctttgggatgggcccgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtaagagtaactgtttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtwaacgatgagtgctaggtgttggagggtttccgcccttcagtgccgaagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaacctcagagatgaggcgttcccttcggggacgcaaagacaggtggtgcatggtcgtcgtcagctcgtgtcgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatggccggtacaacgagcagctaacccgcgagggtgtgcaaatctcttaaagccggtctcagttcggactgcagtctgcaactcgactgcacgaagtcggaatcgctagtaatcgcggatcagcatgccgcggtgaatacgttcccgggccttgtacacaccgcccgtcacaccatggaaktttgcaatgcccaaagtcagtggcctaaccattatggagggasctgcctaaggcagggcagatgactggggtgaagtcgt

SEQ ID NO: 19 - DNA, 1513 bp, Lactobacillus brevis
  misc_feature: L. brevis partial 16S sequence NCBI Reference Sequence NR_044704.2
  misc_feature (570)..(570): n is a, c, g, or t
  misc_feature (714)..(714): n is a, c, g, or t
  misc_feature (789)..(789): n is a, c, g, or t
  misc_feature (957)..(957): n is a, c, g, or t
taagatgagagtttgatcctggctcaggacgaacgctggcggcatgcctaatacatgcaagtcgaacgagcttccgttgaatgacgtgcttgcactgatttcaacaatgaagctagtggcgaactggtgagtaacacgtgggaaatctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcnagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcngtggaatgcgtagatatatggaagaacaccagtggcgaaggcggctgtctagtctgtaactgacgctgaggctcnaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggccngcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagtcgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattaggg

SEQ ID NO: 20 - DNA, 1517 bp, Lactobacillus casei
  misc_feature: L. casei partial 16S sequence NR_041893.1
tcctggctcaggatgaacgctggcggcgtgcctaatacatgcaagtcgaacgagttttggtcgatgaacggtgcttgcactgagattcgacttaaaacgagtggcggacgggtgagtaacacgtgggtaacctgcccttaagtgggggataacatttggaaacagatgctaataccgcataaatccaagaaccgcatggttcttggctgaaagatggcgtcaagctatcgcttttggatggacccgcggcgtattagctagttggtgaggtaacggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcattgagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaag

SEQ ID NO: 21 - DNA, 1564 bp, L. coleohominis
  misc_feature: L. coleohominis partial 16S sequence NCBI Reference Sequence NR_042436.1
agtttgattctggctcaggatgaacgccggcggtgtgcctaatacatgcaagtcgaacgcgttggcccgactgattgatgatgcttgcatcagattgacgacggtttactaacgagtggcggacgggtgagtaacacgtaggcaacctgcccagaagcgggggacaacatttggaaacaagtgctaataccgcataacaacgaaaaccgcctggttttcgtttaaaagatggtttcggctatcacttctggatgggcctgcggcgcattagctagttggtaaggtaacggcttaccaaggcagtgatgcgtagccgagttgagagactgatcggccacaatggaactgagacacgggccatactcctacgggaggcagcagtagggaatcttccacaatgggcgcaagcctgatggagcaacaccgcgtgagtgaagaagggtttcggctcgtaaaactctgttgttgaagaagaacgtgcgtgagagtaactgttcacgcagtacggtattcaaccagaaagtcacggctaactacgtgccagcagccgcggtaatacgtaggtggcaagcgttatccggatttattgggcgtaaagcgagcgcaggcggttttctaagtctgatgtgaaagccttcggcttaaccggagaagggcatcggaaactggataacttgagtgcaagagaggacagtggaactccatgtgtagcggtggaatgcgtagatatatggaagaacaccagtggcgaaggcggctgtctagcttgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaacctcagagatgaggcgttcccttcggggacgcaatgacaggtggtgcatggtcgtcgtcagctcgtgttgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatgggcggtacaacgagcagcgaactcgcgagggtaagctaatctcttaaaaccgttctcagttcggactgcagtctgcaactcgactgcacgaagtcggaatcgctagtaatcgcggatcagcatgccgcggtgaatacgttcccgggccttgtacacaccgcccgtcacaccatgggagtttgcaatgcccaaagccggtggcctaaccttcgggaaggagccgtctaaggcagggcagatgactggggtgaagtcgtaacaaggtagccgtaggagaacctgcggcttgatcaccttcat

SEQ ID NO: 22 - DNA, 1518 bp, Lactobacillus crispatus
  misc_feature: L. crispatus partial 16S sequence NCBI Reference Sequence NR_041800.1
gacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcgtaagctgtcgctatgggatggccccgcggtgcattagctagttggtaaggtaaaggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgccatttgtagagatacaaagttcccttcggggacgctaagacaggtggtgcatggctgtcgtcagctcgtgtcgtgagatgttgggttaagtcccgcaacgagcgcaacccttgttattagttgccagcattaagttgggcactctaatgagactgccggtgacaaaccggaggaaggtggggatgacgtcaagtcatcatgccccttatgacctgggctacacacgtgctacaatgggcagtacaacgagaagcgagcctgcgaaggcaagcgaatctctgaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggcagggcagatgactggggtgaagtcgtaacaaggtagccgtaggagaactgc

SEQ ID NO: 23 - DNA, 1518 bp, Lactobacillus curvatus; identical to SEQ ID NO: 22
  misc_feature: L. curvatus partial 16S sequence NCBI Reference Sequence NR_041800.1

SEQ ID NO: 24 - DNA, 1515 bp, Lactobacillus delbrueckii
  misc_feature: L. delbrueckii partial 16S sequence NCBI Reference Sequence NR_029106.1
gaacgctggcggcgtgcctaatacatgcaagtcgagcgagctgaattcaaagatcccttcggggtgatttgttggacgctagcggcggatgggtgagtaacacgtgggcaatctgccctaaagactgggataccacttggaaacaggtgctaataccggataacaacatgaatcgcatgattcaagtttgaaaggcggcgcaagctgtcactttaggatgagcccgcggcgcattagctagttggtggggtaaaggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgcgctacacctagagataggtggttcccttcggggacgcagagacaggtggtgcatggctgtcgtcagctcgtgtcgtgagatgttgggttaagtcccgcaacgagcgcaacccttgtctttagttgccatcattaagttgggcactctagagagactgccggtgacaaaccggaggaaggtggggatgacgtcaagtcatcatgccccttatgacctgggctacacacgtgctacaatgggcagtacaacgagaagcgaacccgcgagggtaagcggatctcttaaagctgttctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgggataaccttttaggagtcagccgcctaaggcagggcagatgactggggtgaagtcgtaacaaggtagccgtaggagaactgcg

SEQ ID NO: 25 - DNA, 1502 bp, Lactobacillus fermentum
  misc_feature: L. fermentum partial 16S sequence NCBI Reference Sequence NR_104927.1
cggcggtgtgcctaatacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcgaactcgcgagggcaagcaaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgtaacacccaaagtcggtggggtaaccttttaggagccagccgcctaaggtgggacagatgattagggtgaagtcgtaacaag

SEQ ID NO: 26 - DNA, 1573 bp, Lactobacillus gasseri
  misc_feature: L. gasseri 16S sequence NCBI Reference Sequence NR_075051.2
gaaaatgagagtttgatcctggctcaggacgaacgctggcggcgtgcctaatacatgcaagtcgagcgagcttgcctagatgaatttggtgcttgcaccagatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctctgaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaagtcgtaacaaggtagccgtaggagaacctgcggctggatcacctccttt

SEQ ID NO: 27 - DNA, 1487 bp, Lactobacillus johnsonii
  misc_feature: L. johnsonii partial 16S sequence NCBI Reference Sequence NR_025273.1
ggcggcgtgcctaatacatgcaagtcgagcgagcttgcctagatgattttagtgcttgcactaaatgaaactagatacaagcgagcggcggacgggtgagtaacacgtgggtaacctgcccaagagactgggataacacctggaaacagatgctaataccggataacaacactagacgcatgtctagagtttgaaagatggttctgctatcactcttggatggacctgcggtgcattagctagttggtaaggtaatggcttaccaaggcgatgatgcatagccgagttgagagactgatcggccacattgggactgagacacggcccaaactcctacgggaggcagcagtagggaatcttccacaatggacgaaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaacgcttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcatgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctcttaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaa

SEQ ID NO: 28 - DNA, 1507 bp, L. harbinensis
  misc_feature: L. harbinensis partial 16S sequence NCBI Reference Sequence NR_041263.1
gctggcggcgtgcctaatacatgcaagtcgaacgaggtttggtcagtttgcggtggtgcttgcatcaccaattaccgatcaaaccgagtggcggacgggtgagtaacacgtgggtaacctgcccttcagcaggggataacatttggaaacagatgctaataccgtataaccacggagaccgcatggtctccgggtaaaagatggcgcaagctatcactgaaggatggacccgcggcgtattagccagttggtggggtaacggcctaccaaagcgatgatacgtagccgacctgagagggtaatcggccacattgggactgagacacggcccaaactcctacgggaggcagcagtagggaatcttccacaatggacgcaagtctgatggagcaacgccgcgtgagtgaagaaggctttcgggtcgtaaaactctgttattgaagaagaacgtgtgtgacagtaactggtcatgcagtgacggtattcaatcagaaagtcacggctaactacgtgccagcagccgcggtaatacgtaggtggcaagcgttgtccggatttattgggcgtaaagcgagtgcaggcggtcttttaagtctgatgtgaaagccttcggcttaaccgaagaagggcatcggaaactgggagacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacaggattagataccctggtagtccacgccgtaaacgatgaatactaagtgttggagggtttccgcccttcagtgctgcagctaacgcattaagtattccgcctggggagtacgaccgcaaggttgaaactcaaaggaattgacgggggcccgcacaagcggtggagcatgtggtttaattcgaagcaacgcgaagaaccttaccaggtcttgacatcttctgccaggctgagagatcagctgttcccttcggggacagaatgacaggtggtgcatggttgtcgtcagctcgtgtcgtgagatgttgggttaagtcccgcaacgagcgcaacccttatgatcagttgccagcattcagttgggcactctggtcagactgccggtgacaaaccggaggaaggcggggatgacgtcaaatcatcatgccccttatgacctgggctacacacgtgctacaatgggtggtacaacgagcagcgagaccgcgaggtcaagcgaatctctaaaaaccatcctcagttcggattgcaggctgcaactcgcctgcatgaagctggaatcgctagtaatcgcggatcagcacgccgcggtgaatacgttcccgggccttgtacacaccgcccgtcacaccatgagagtttgtaacacccaaagccggtgagacaaccgcaaggagtcagccgtctaaggtgggacaaatgattagggtgaagtcgtaaacaaggtaaca

SEQ ID NO: 29 - DNA, 1568 bp, L. mucosae
  misc_feature: L. mucosae partial 16S sequence NCBI Reference Sequence NR_024994.1
agagtttgatcctggctcaggatgaacgccggcggtgtgcctaatacatgcaagtcgaacgcgttggcccaactgattgaacgtgcttgcacggacttgacgttggtttaccagcgagtggcggacgggtgagtaacacgtaggtaacctgccccaaagcgggggataacatttggaaacagatgctaataccgcataacaatttgaatcgcatgattcaaatttaaaagatggcttcggctatcactttgggatggacctgcggcgcattagcttgttggtagggtaacggcctaccaaggctgtgatgcgtagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttagagaagaacgtgcgtgagagcaactgttcacgcagtgacggtatctaaccagaaagtcacggctaactacgtgccagcagccgcggtaatacgtaggtggcaagcgttatccggatttattgggcgtaaagcgagcgcaggcggtttgataagtctgatgtgaaagcctttggcttaaccaaagaagtgcatcggaaactgtcagacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatggacggtacaacgagtcgcgaactcgcgagggcaagctaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgcaacacccaaagtcggtggggtaacccttcggggagctagccgcctaaggtggggcagatgattagggtgaagtcgtaacaaggtagccgtaggagaacctgcggctggatcacctcct

SEQ ID NO: 30 - DNA, 1519 bp, Lactobacillus pentosus
  misc_feature: L. pentosus partial 16S sequence
gacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtagggagaac

SEQ ID NO: 31 - DNA, 1517 bp, Lactobacillus plantarum
  misc_feature: L. plantarum partial 16S sequence NCBI Reference Sequence NR_042254.1
ckaacgcyggcggcgtgcctaatacatgcaagtcgaacgaactctggtattgattggtgcttgcatcatgatttacatttgagtgagtggcgaactggtgagtaacacgtgggaaacctgcccaggaagcgggggataacacctggaaacakatgctaataccgcataacaacttggaccgcatggtccgagtttgaaagatggcttcggctatcacttttggatggycccgcggcgtattakctakatggtggggtaacggctcaccatggcaatgatacgtacccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtatatatatggaasaacaccagtggcgaaggcggctgtctggttctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaa

SEQ ID NO: 32 - DNA, 1535 bp, Lactobacillus reuteri
  misc_feature: L. reuteri partial 16S sequence NCBI Reference Sequence NR_025911.1
agagtttgatcctggctcaggatgaacgccggcagtgtgcctaatacatgcaagtcgtacgcactggcccaactaattgatggtgcttgctgaattgacgatggatcaccagtgagtggcggacgggtgagtaacacgtaggtaacctgccccggagcggggaataacatttggaaacagatgctaataccgcataacaacaaaagccgcatggtttttctggaaagatggctttggctatcactctgggatggacctgcggtgcatttagctagttggtaaggtaacggcttacccaaggcgatgatgcatagccgagttgagagactgatcggccacaatgggaactgagacacggtccataacttctacgggaggcagcagtagggaatcttccacaatgggcgcaagctgatggagcaacaccgcgttattaagaaagggtttcggccgcttaaactctgttgttggagaagaacgtgcgttagagtaactgttacgcagtgacggtatccaaccagaaagtcacggctaactacgtgccagcagccgcggtaatacgtaggtggcaagcgttatccggatttattgggcgtaaagcgagcgcaggcggttgcttaggtctgatgtggaaactcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgcctgttctaacgcattaatgcactccgcctggggagtacgaccgcaaggttgaaactcaaaggaattgacgggggcccgcacaagcggtgaagcatgtggtttaattcgaagctacgcgaagaaccttaccaggtcttgacatcttgcgctaaccttagagataaggcgttcccttcggggacgttaatgacaggtggtgcatggtcgtcgtcagctcgtgtcgtgagatgttgggttaagtcccgcaacgagcgcaacccttgttactagttgccagcattaagttggggactctagtgagactgccggtgacaaaccggaggaaggtggggacgacgtcagatcatcatgccccttatgaccctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagttcggtggcctaacctttatggacgggtaccctaaggcgggacagatgatctggggtgaagtcgtaacaaggta

SEQ ID NO: 33 - DNA, 1521 bp, Lactobacillus rhamnosus
  misc_feature: L. rhamnosus partial 16S sequence NCBI Reference Sequence NR_043408.1
  misc_feature (407)..(407): n is a, c, g, or t
  misc_feature (655)..(655): n is a, c, g, or t
  misc_feature (1476)..(1476): n is a, c, g, or t
grtsaacgctsgcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactctacgggaggcagcagtagggaatcttccacaatggacgcaagtctgatggagcaacgccgcgtgagtnaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtncagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggncaaatgattagggtgaagtcgtaacaaggtagccgtaggagaacc

SEQ ID NO: 34 - DNA, 1561 bp, Lactobacillus sake
  misc_feature: L. sakei partial 16S sequence NCBI Reference Sequence NR_042443.1
aagtttgattattgctcaggacgaacgctggcggcgtgcctaatacatgcaagtcgaacgcactctcgtttagattgaaggagcttgctcctgattgataaacatttgagtgagtggcggacgggtgagtaacacgtgggtaacctgccctaaagtgggggataacatttggaaacagatgctaataccgcataaaacctaacaccgcatggtgtagggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaatgtatctgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggatgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagccggaatcgctagtaatcgcggatcagcatgccgcggtgaatacgttcccgggccttgtacacaccgcccgtcacaccatgagagtttgtaacacccaaagccggtgaggtaacccttcggggagccagccgtctaaggtgggacagatgattagggtgaagtcgtaacaaggtagccgtagagaacctgcggctggatcacctc

SEQ ID NO: 35 - DNA, 1542 bp, Lactobacillus salivarius
  misc_feature: L. salivarius partial 16S sequence NCBI Reference Sequence NR_028725.2
agagtttgatcctggctcaggacgaacgctggcggcgtgcctaatacatgcaagtcgaacgaaactttcttacaccgaatgcttgcrttcatcgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggtccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaaggtagccgtaggagaacctgcggctg

SEQ ID NO: 36 - DNA, 1522 bp, Lactobacillus paracasei
  misc_feature: L. paracasei  partial 16S sequence NCBI Reference Sequence NR_025880.1
gatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacc

SEQ ID NO: 37 - DNA, 1565 bp, L. kisonensis
  misc_feature: L. kisonensis partial 16S sequence NCBI Reference Sequence NR_041658.1
agagtttgatcctggctcaggacgaacgctggcggcgtgcctaatacatgcaagtcgaacgcgtcttggttatcgaagggaagtgcttgcatttccttgacttaacattgagacgagtggcgaactggtgagtaacacgtgggtaacctgcccttgaagtaggggataacacttggaaacaggtgctaataccgtataacaaccaaagccccatgactttggtttaaaagatggcttcggctatcactttaggatggacccgcggcgtattagcttgttggtgaggtaacggctcaccaaggcaatgatacgtagccgacctgagagggtaatcggccacattgggactgagacacggcccaaactcctacgggaggcagcagtagggaatcttccacaatggacgaaagtctgatggagcaacgccgcgtgagtgatgaagggtttcggctcgtaaaactctgttgttggagaagaacgggtgtgagagtaactgttcacatcgtgacggtatccaaccagaaagccacggctaactacgtgccagcagccgcggtaatacgtaggtggcaagcgttgtccggatttattgggcgtaaagcgagcgcaggcggttttttaggtctgatgtgaaagccttcggcttaaccggagaagtgcatcggaaaccgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgatgctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattgttagttgccagcatttagttgggcactctagcaagactgccggtgacaaaccggaggaaggtggggatgacgtcaaatcatcatgccccttatgacctgggctacacacgtgctacaatggacggtacaacgagtcgcgaaaccgcgaggtcaagctaatctcttaaagccgttctcagttcggattgtaggctgcaactcgcctacatgaagttggaatcgctagtaatcgtggatcagcatgccacggtgaatacgttcccgggccttgtacacaccgcccgtcacaccatgagagtttgtaacacccaaagccggtgaggtaaccttcggggaccagccgtctaaggtgggacagatgattagggtgaagtcgtaacaaggtagccgtaggagaacctgcggctggatcacctcc

SEQ ID NO: 38 - DNA, 1522 bp, L. paralimentarius
  misc_feature: L. para-limentarius partial 16S sequence NCBI Reference Sequence NR_025045.1
gacgaacgctggcggcatgcctaatacatgcaagtcgaacgaaccatcctgaagattgaagcttgcttcatgattcagaccttggtgagtggcggacgggtgagtaacacgtgggtaacctgcccagaagtgggggataacatttggaaacaagtgctaataccgcataacaacttagatcacatgatctttgtttaaaagatggttttgctatctcttctggatggacccgcggcgtattagctagttggtgaggtaatagctcaccaaggcgatgatacgtagccgacctgagagggtaatcggccacattgggactgagacacggcccaaactcctacgggaggcagcagtagggaatcttccacaatggacgaaagtctgatggagcaatgccgcgtgagtgaagaaggttttcggatcgtaaaactctgttgttgaagaagaacatatgtgagagtaactgttcacgtactgacggtattcaaccagaaagccacggctaactacgtgccagcagccgcggtaatacgtaggtggcaagcgttgtccggatttattgggcgtaaagagaatgtaggcggtttattaagtttgaagtgaaagccctcggctcaaccgaggaagtgcttcgaaaactggtaaacttgagtgcagaagaggaaagtggaactccatgtgtagcggtggaatgcgtagatatatggaagaacaccagtggcgaaggcggctttctggtctgtaactgacgctgagattcgaaagcatgggtagcaaacaggattagataccctggtagtccatgccgtaaacgatgagtgctaagtgttggagggtttccgcccttcagtgctgcagctaacgcattaagcactccgcctggggagtacgatcgcaagattgaaactcaaaggaattgacgggggcccgcacaagcggtggagcatgtggtttaattcgaagcaacgcgaagaaccttaccaggtcttgacataccatgaaaagcttagagataagtctttcccttcggggacatggatacaggtggtgcatggttgtcgtcagctcgtgtcgtgagatgttgggttaagtcccgcaacgagcgcaacccttattatcagttgccagcattcagttgggcactctggtgagactgccggtgataaaccggaggaaggtggggacgacgtcaaatcatcatgccccttatgacctgggctacacacgtgctacaatggtcggtacaacgtgctgcgaactcgcgagggcaagcaaatcacttaaaaccgatctcagttcggattgtaggctgcaactcgcctacatgaagctggaatcgctagtaatcgcggatcagcatgccgcggtgaatacgttcccgggccttgtacacaccgcccgtcacaccatgagagtttgtaacacccaaagtcggtggggtaacccttcggggaactagccgcctaaggtgggacaaatgattagggtgaagtcgtaacaaggtagccgtaggagaacctgc

SEQ ID NO: 39 - DNA, 1554 bp, L. perolens
  misc_feature: L. perolens partial 16S sequence NCBI Reference Sequence NR_029360.1
tggctcaggacgaacgctggcggcgtgcctaatacatgcaagtcgaacgaggtttgatcagtttgcggtggtgcttgcatcaccaattaccgattaaaccgagtggcggacgggtgagtaacacgtgggtaacctgcccttcagcaggggataacatttggaaacagatgctaataccgtataaccacggagaccgcatggtctccgggtaaaagatggcgaaagctatcactgagggatggacccgcggcgtattagccagttggtggggtaatggcctaccaaagcgatgatacgtagccgacctgagagggtaatcggccacattgggactgagacacggcccaaactcctacgggaggcagcagtagggaatcttccacaatggacgcaagtctgatggagcaacgccgcgtgagtgaagaaggctttcgggtcgtaaaactctgttattgaagaagaacgtgtgtgagagtaactgctcatgcagtgacggtattcaaccagaaagtcacggctaactacgtgccagcagccgcggtaatacgtaggtggcaagcgttgtccggatttattgggcgtaaagcgagtgcaggcggttttttaagtctgatgtgaaagccttcggcttaaccgaagaagtgcatcggaaagtgggaaacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacaggattagataccctggtagtccacgccgtaaacgatgaatactaagtgttggggggtttccgcccctcagtgctgcagctaacgcattaagtattccgcctggggagtacgaccgcaaggttgaaactcaaaggaattgacgggggcccgcacaagcggtggagcatgtggtttaattcgaagcaacgcgaagaaccttaccaggtcttgacatcttctgccaagctgagagatcagccgttccttcggggacagaatgacaggtggtgcatggttgtcgtcagctcgtgtcgtgagatgttgggttaagtcccgcaacgagcgcaacccttatgattagttgccagcattcagttgggcactctagtcagactgccggtgacaaaccggaggaaggcggggatgacgtcaaatcatcatgccccttatgacctgggctacacacgtgctacaatgggtggtacaacgagcagcgagaccgcgaggtcaagcgaatctctaaaaaccatcctcagttcggattgtaggctgcaactcgcctacatgaagctggaatcgctagtaatcgcggatcagcacgccgcggtgaatacgttcccgggccttgtacacaccgcccgtcacaccatgagagtttgtaacacccaaagccggtaggacaaccgcaaggagtcagccgtctaaggtgggacaaatgattagggtgaagtcgtaacaaggtagccgtaggagaacctgcggctggatcacctcctttct

SEQ ID NO: 40 - DNA, 1428 bp, L. apis
  misc_feature: L. apis partial 16S sequence NCBI Reference Sequence NR_125702.1
tgcagtcgagcgagcaagttaaggaatacttcggtaggaatttaatagcgcgagcggcggatgggtgagtaacacgtgggcaacctgccctttagcttgggataccacttggaaacaggtgctaataccaaataagaagtaagagcgcatgctcaagctatgaaaggcggctttcgagctgtcactaaaggatgggcccgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggacatgggtagtaactgatctatgtttgacggtaatcaaccagaaagtcacggctaactacgtgccagcagccgcggtaatacgtaggtggcaagcgttgtccggatttattgggcgtaaagcgaacgcaggcgggagaacaagtcagctgtgaaagccctcggcttaaccgaggaacggcaactgaaactgtttttcttgagtgcagaagaggagagtggaactccatgtgtagcggtgaaatgcgtagatatatggaagaacaccagtggcgaaggcggctctctggtctgtaactgacgctgaggttcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttaaaactcaaaggaattgacgggggcccgcacaagcggtggagcatgtggtttaattcgaagcaacgcgaagaaccttaccaggtcttgacatctagtgccaaccctagagatagggtgtttccttcgggaacactaagacaggtggtgcatggctgtcgtcagctcgtgtcgtgagatgttgggttaagtcccgcaacgagcgcaacccttattattagttgccagcattaagttgggcactctaatgagactgccggtgacaaaccggaggaaggtggggacgacgtcaagtcatcatgccccttatgacctgggctacacacgtgctacaatggttagtacaacgaggagcgaacctgtgaaggcaagcgaatctcttaaagctaatctcagttcggattgcactctgcaactcgagtgcatgaagctggaatcgctagtaatcgcggatcagcatgccgcggtgaatacgttcccgggccttgtacacaccgcccgtcacaccatgagagtttgtaatacccaaagccggtgagataacctgtaaaggagtcagc

SEQ ID NO: 41 - DNA, 1460 bp, L. ghanensis
  misc_feature: L. ghanensis partial 16S sequence NCBI Reference Sequence NR_043896.1
gcggcgtgcctaatacatgcaagtcgaacgaagttgttttaactggtgcttgcaccgacttaaacaactgagtggcgaacgggtgagtaacacgtgggtaacctgccccaaagcgggggataacatttggaaacagatgctaataccgcataaccacagaaacctcatggtttctgtgtcaaagatggtttcggctatcactttgggatggacccgcggcgtattagctagttggtaaggtaacggcttaccaaggcaatgatacgtagccgaactgagaggttgatcggccacattgggactgagacacggcccaaactcctacgggaggcagcagtagggaatcttccacaatggacgaaagtctgatggagcaacgccgcgtgagtgaagaaggttttcggatcgtaaaactctgttgtcagagaagaacgtgtgcgagagtaactgttcgtgcagtgacggtatctgaccagaaagccacggctaactacgtgccagcagccgcggtaatacgtaggtggcaagcgttgtccggatttattgggcgtaaagggaacgcaggcggttttttaagtctgatgtgaaagccttcggcttaaccgaagtcgtgcattggaaactggagaacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgctgtaaacgatgaatgctaagtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttctgcaagcctgagagatcaggtgttcccttcggggacagaatgacaggtggtgcatggttgtcgtcagctcgtgtcgtgagatgttgggttaagtcccgcaacgagcgcaacccttattgccagttgccagcattcagttgggcactctgacaagactgccggtgacaaaccggaggaaggtggggatgacgtcaaatcatcatgccccttatgacctgggctacacacgtgctacaatggacgatacaacgagttgctagaccgcgaggttaagctaatctcttaaagtcgttctcagttcggattgcaggctgcaactcgcctgcatgaagtcggaatcgctagtaatcgcggatcagcatgccgcggtgaatacgttcccgggccttgtacacaccgcccgtcacaccatgagagtttgtaacacccaaagccggtggggtaaccagtgggaaccagccgtctaaggtgggacaga

SEQ ID NO: 42 - DNA, 1464 bp, L. dextrinicus
  misc_feature: L. dextrinicus partial 16S sequence NCBI Reference Sequence NR_036861.1
  misc_feature (394)..(394): n is a, c, g, or t
  misc_feature (397)..(397): n is a, c, g, or t
  misc_feature (781)..(781): n is a, c, g, or t
ctggcggcgtgcctaatacatgcaagtcgaacgagtttgcttttaatgaaggcggtgcttgcaccaactgatttaaaaataaacgagtggcggacgggtgagtaacacgtgggtaacctaccctaaagtgggggataacatttggaaacagatgctaataccgcataatatcaaaaaccacatggtttttaattgaaagacggcgtaagctgtcacttttggatggacccgcggcgtattagctagttggtgaggtaacggctcaccaaggcgatgatacgtagccgacctgagagggtgatcggccacattgggactgagacacggcccaaactcctacgggaggcagcagtagggaatcttccacaatggacgaaagtctgatggagcaacnccncgtgagtgaagaaggttttcggatcgtaaaactctgttattggagaagaacgtatttggtagtaactggccagatagtgacggtatccaatcagaaagccacggctaactacgtgccagcagccgcggtaatacgtaggtggcaagcgttgtccggatttattgggcgtaaagcgagtgcaggcggttttttaagtctgatgtgaaagccttcggcttaaccgaagaaatgcattggaaactgggaaacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacagnattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagcgccaatcctagagataggacgttcccttcggggacgctaagacaggtggtgcatggttgtcgtcagctcgtgtcgtgagatgttgggttaagtcccgcaacgagcgcaacccttattatcagttgccagcatttagttgggcactctggtgagactgccggtgacaaaccggaggaaggtggggatgacgtcaaatcatcatgccccttatgacctgggctacacacgtgctacaatggctagtacaacgagttgcgagaccgcgaggtcaagctaatctctaaaagctagtctcagttcggattgtaggctgcaactcgcctacatgaagttggaatcgctagtaatcgcggatcagcacgccgcggtgaatacgttcccgggccttgtacacaccgcccgtcacaccatgagagtttgtaacacccgaagccggtggagtaacccgtaagggagctagccgtct

SEQ ID NO: 43 - DNA, 1499 bp, Lactococcus lactis
  misc_feature: Lactococcus lactis Partial 16S Sequence NCBI Reference Sequence NR_040954.1
gacgaacgctggcggcgtgcctaatacatgcaagttgagcgatgaagattggtgcttgcaccaatttgaagagcagcgaacgggtgagtaacgcgtggggaatctgcctttgagcgggggacaacatttggaaacgaatgctaataccgcataacaactttaaacataagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaacaaggtagccgtatcggaagg

SEQ ID NO: 44 - DNA, 1510 bp, Bacteroides vulgatus
  misc_feature: Bacteroides vulgatus partial 16S sequence NCBI Reference Sequence NR_074515.1
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 45 - DNA, 1529 bp, Bacteroides fragilis
  misc_feature: Bacteroides fragilis partial 16S sequence NCBI Reference Sequence  NR_074784.2
acaatgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctggaacacctccttt

SEQ ID NO: 46 - DNA, 1539 bp, S. thermophilus
  misc_feature: S. thermophilus partial 16S sequence NCBI Reference Sequence NR_042778.1
atgggagagtttgatcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaatggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgggtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcgagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggacagatgattggggtgaagtcgtaacaaggtagccgtatcggaaggtgcggctggatcac

SEQ ID NO: 47 - DNA, 1436 bp, P. parvulus
  misc_feature: P. parvulus partial 16S sequence NCBI Reference Sequence NR_029136.1
  misc_feature (1345)..(1345): n is a, c, g, or t
acatgcaagtcgaacgcactttcgttgaatgaattagaggtgcttgcaccgaagatgattttagctataaagtgagtggcgaacgggtgagtaacacgtgggtaacctgcccagaagtgggggataacacctggaaacagatgctaataccgcataacaaagtaaaccgcatggtttacttttaaaagatggcttcggctatcacttctggatggacccgcggcgtattagctagttggtgagataaaggctcaccaaggcagtgatacgtagccgacctgagagggtaatcggccacattgggactgagacacggcccagactcctacgggaggcagcagtagggaatcttccacaatggacgaaagtctgatggagcaacgccgcgtgagtgatgaaggctttagggtcgtaaaactctgttgttggagaagaacgtgtgtgagagtaactgctcatgcagtgacggtatccaaccagaaagccacggctaactacgtgccagcagccgcggtaatacgtaggtggcaagcgttatccggatttattgggcgtaaagcgagcgcaggcggtcttttaagtctaatgtgaaagccttcggcttaaccgaagaagtgcattggaaactggaagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaagtgttggagggtttccgcccttcagtgctgcagctaacgcattaagcattccgcctggggagtacgaccgcaaggttgaaactcaaaagaattgacgggggcccgcacaagcggtggagcatgtggtttaattcgaagctacgcgaagaaccttaccaggtcttgacatcttctgccaatctaagagattagacgttcccttcggggacagaatgacaggtggtgcatggttgtcgtcagctcgtgtcgtgagatgttgggttaagtcccgcaacgagcgcaacccttattattagttgccagcattaagttgggcactctagtgagactgccggtgacaaaccggaggaaggtggggacgacgtcaaatcatcatgccccttatgacctgggctacacacgtgctacaatggacggtacaacgagttgcgagaccgcgaggtttagctaatctcttaaaaccgttctcagttcggactgcaggctgcaactcgcctgcacgaagttggaatcgctagtaatcgcggatcancatgccgcggtgaatacgttcccgggccttgtacacaccgcccgtcacaccatgagagtttgtaacacccaaagccggtggagtaaccttc

SEQ ID NO: 48 - DNA, 1540 bp, P. lolii
  misc_feature: P. lolii partial 16S sequence NCBI Reference Sequence NR_041640.1
caagtcgaacgaacttccgttaattgattatgacgtgcttgcactgaatgagattttaacacgaagtgagtggcggacgggtgagtaacacgtgggtaacctgcccagaagcaggggataacacctggaaacagatgctaataccgtataacagagaaaaccgcctggttttcttttaaaagatggctctgctatcacttctggatggacccgcggcgcattagctagttggtgaggtaacggctcaccaaggcgatgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggtaatagggtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctgttagtaccctgctgtcaacgataagagtgattactaagtgttggagggtttccgcccttcagtgctgcagctaacgcattaagttatccgcctggggagtacgaccgcaaggttgaaactcaaagaattgacgggggccgcacaagcgtggagcatgtggttaattcgaagtacgcgaagaacttaccaggtcttgacatctttgccaacctaagagattaggcgttccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagtcgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctttctaaggaataatacggaacc

SEQ ID NO: 49 - DNA, 1540 bp, P. acidilactici; identical to SEQ ID NO: 48
  misc_feature: P. acidilactici partial 16S sequence NCBI Reference Sequence NR_041640.1

SEQ ID NO: 50 - DNA, 1492 bp, P. argentinicus
  misc_feature: P. claussenii partial 16S sequence NCBI Reference Sequence NR_042232.1
gatgaacgctggcggcgtgcctaatacatgcaagtcgaacgcacttccgtcgaatgatttcaaggtgcttgcaccgcgaatgaaaatgacatgaagtgagtggcgaacgggtgagtaacacgtgggtaacctgcccagaagtaggggataacacctggaaacagatgctaataccgtataatagagaaaaccgcatggttttcttttgaaagatggctctgctatcacttctggatggacccgcggcgtattagctagttggtgaggtaackgctcaccaaggcagtgatacgtagccgacctgagagggtaatcggccacattgggactgagacacggcccagactcctacgggaggcagcagtagggaatcttccacaatggacgaaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtttcttaagtctaatgtgaaagccttcggctcaaccgaagaagtgcattggaaactgggaaacttgagtgcagaagaggatagtggaactccatgtgtagcggtgaaatgcgtagatatatggaagaacaccagtggcgaaggcggctatctggtctgcaactgacgctgaggctcgaaagcatgggtagcgaacaggattagataccctggtagtccatgccgtaaacgatgaatgctaagtgttggagggtttccgcccttcagtgctgcagctaacgcattaagcattccgcctggggagtacgaccgcaaggttgaaactcaaaagaattgacgggggcccgcacaagcggtggagcatgtggtttaattcgaagctacgcgaagaaccttaccaggtcttgacatcttctgccaatctaagagattagacgttcccttcggggacagaatgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgat

SEQ ID NO: 51 - DNA, 1472 bp, P. claussenii
  misc_feature: NCBI Reference Sequence NR_042232.1
tgcaagtcgaacgcatttccgttaatagaatcagaagtgcttgcacggatagatgattttaacaatgaaatgagtggcgaacgggtgagtaacacgtgggtaacctgcccagaagagggggataacacttggaaacaggtgctaataccgcataataaagaaaaccgcatggttttcctttaaaagatggtttcggctatcacttctggatggacccgcggcgtattagctagttggtaaggtaaaggcttaccaaggcagtgatacgtacccgacctgagagggtaatcggccacattgggactgacacacggcccatactcctacgggaggcagcaatagggaatcttccacagtggacgaaagtctgatggagcaacgccgcgtgagtgaagaagggtttcggctcgtaaaactctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatactaagtgttggagggtttccgcccttcagtgctgcagctaacgcattaagtattccgcctggggagtacgaccgcaaggttgaaactcaaaagaattgacgggggcccgcacaagcggtggagcatgtggtttaattcgaagctacgcgaagaaccttaccaggtcttgacatcttctgacattctaagagattagaagttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgaaaccgcgaggtttagctaatctcttaaagccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattaggg

SEQ ID NO: 52 - DNA, 1569 bp, P. pentosaceus
  misc_feature: P. pentosaceus partial 16S sequence NCBI Reference Sequence NR_042058.1
  misc_feature (120)..(120): n is a, c, g, or t
agagtttgatcatggctcaggatgaacgctggcggcgtgcctaatacatgcaagtcgaacgaacttccgttaattgattatgacgtacttgtactgattgagattttaacacgaagtagntggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggtaaaggcccaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctt

SEQ ID NO: 53 - DNA, 1529 bp, P. stilesii
  misc_feature: P. stilesii partial 16S sequence NCBI Reference Sequence NR_042401.1
gatgaacgctggcggcgtgcctaatacatgcaagtcgaacgaacttccgttaattgattaagcggtacttgtaccaattgagattttaacacgaagtgagtggcgaacgggtgagtaacacgtgggtaayctgcccagaagtaggggataacacctggaaacagatgctaataccgtataatagagaaaaccgcatggttttcttttgaaagatggctctgctatcacttctggatggacccgcggcgcattagctagttggtaaggtaaaggcttaccaaggcagtgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgaatgctaagtgttggagggtttccgcccttcagtgctgcagctaacgcattaagcattccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatgatacaacgagtcgcgaaaccgcgaggtttagctaatctcttaaaatcattctcagttcggactgtaggctgcaactcgccyacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaagaaaacc

SEQ ID NO: 54 - DNA, 20 bp, Bifidobacterium infantis
  misc_feature: forward primer sequence
atacagcagaaccttggcct

SEQ ID NO: 55 - DNA, 20 bp, Bifidobacterium infantis
  misc_feature: reverse primer sequence
gcgatcacatggacgagaac

SEQ ID NO: 56 - DNA, 24 bp, Artificial Sequence
  Probe sequence
  misc_feature (1)..(1): FAM dye
  misc_feature (9)..(10): ZEN quencher
  misc_feature (24)..(24): IABkFQ quencher
tttcacggatcaccggaccatacg

SEQ ID NO: 57 - DNA, 1113 bp, Bifidobacterium infantis
  misc_feature: pksS;Polyketide_biosynthesis_cytochrome_P450_PksS
gtgataattggaaaacgtctcggtgaagatcggttcaaattttacaaaaccgtccttgccggaaatacagatcagctgctattcgacgaggaatttaatgcatggtttgtgttcaatgtgcgtaaccttcatactttgataagagatgagcggctcaaagccaacactaaaactgtcatagataaactccctctgaagatagaaactatacggacattaaagaatttttattccaactgggttatgttcaccgacggagagtctcataaacagcttcgtcgcctagtgggaacaatcattaatactcgttatcacgccataaattacacatggccgcaaatcaataaaaattgtgacttcactacagagtatgcaaggccttatgtgtacggtatccttgctcaactggtgggtgtctctgtagaagacatttcaagaatggtctcggcatcggaaaccattaactcgttcttactgcgcgaacggcttacgctagacgatatcgaacaagtcgcgcattcaatcgaatacgcgtatcaggttgtcaaagaaatcgaagataagcatgtaggggagccgctgtacataggcaacgaattgcttgacttgcctcaggagacacgatatcccctcatcattaatctagtaaccgatggttttgccccattcgtagctgcgcttgatttcctggcgttcaatctgttgactcacccatatttagaggaagagctcaatgcaaaagcagggcaaatatcccttgaaagtcttcggttgttccctccattcacgacaataagcaggacatgcgtgcatgaaataccttttaaggaaaaaattattagaccaaggcagttggtaatttttgatttatacagcattaatcgcgacccggaagtttttcccgatccggaaaaatttaaccttgagaacactgcaagagcatactcctttggggctgcgcagcatctatgctccggaaacccgctggtgagaaaagccctggaacaagtaacgcgccaaagcgaatcactatataaatacaagatacagtcttattgcttcaaaaattcatacggttttaccgacatgaatttgtctattgaattaaaataa

SEQ ID NO: 58 - DNA, 333 bp, Bifidobacterium infantis
  misc_feature: higB2;Putative_toxin_HigB2
atggaacccatcaaagactggctgaccggcctcgaccgagaatccaaactgcagatcctcgccgccgtcgaagtcctgaaagaacaaggacccaacctcagacgaccactggtaggcaaaatcgaaggctccaaaatcaaatcgatgaaggaactccgacccggctccgcaggcaaaagcgaaatacgaatactgttcgtgttcgacccgaaccggcaggcgataatgctcgtcggcggagacaagcaaaacaaatggtcgaaatggtacaaaacggccataccggaagcggaagaccggtatgaggcgtggcttaaacagaacaacaaatga

SEQ ID NO: 59 - DNA, 1101 bp, Bifidobacterium infantis
  misc_feature: putative Riboflavin biosynthesis gene
atggacgatgcgcagtacatgggactcgcgatcgagctggcgaagcgaggggccggatacgtgaaccccaatcccatggtcggcgcggtgatcgtcaaggacgatcggatcatcgggcaggggtatcacgagatgttcggcggattgcacgccgagaggaacgcgttgcggcattgcacgcagtcacccgccggggcgacgttgtacgtcacgcttgagccgtgctgccattacggcaaaacgccgccatgcacggaggcgatcgtggaaagcggcatcgcaagggtcgtcgtcggaaccttggactgcaatcccgtggtgtccggcaagggcgtgcgcatgctcgaagaccacggcatacgggtcgatgtgggggtgctggccgacgagtgccggcatctgatacgggtgttcagcaaatacatcaccacgcatacgccgtatgtcatcatgaaatacgccatgacgatggacgggaagatagcgacccataccaatcaatcacggtggatcagcggcgaggagtcgaggcgtcgggtccatcagctccgtcgatccgtcgcggcggtcatggtgggggtgaacacggtgatcgaggacgacccgttgctgacatgccgcatggcacacggaaggaacccggttcgcgtcgtctgcgatacgcgattgaggacgcccctcacctcgcggatcgtgcagacggcgaatgacgtcaggacctatatcgccaccgcctgcgatgacgaacgcaaggcggaggactaccggcggcacggctgcgaaatactcgccgtcggaaggaagggcgaccacgtcgatctggcggacgtggtgcggcgcctgggggatatgcagatggacagcgtgctgctggaaggcgggagcgcgatgaattggagcgccctcgaacagcggatcgtcgatgaggcgcacgtgtacatcgcgccgaagatattcggcggcaccgcgaaaagcccggtgggcggccaaggggtcgccctgccttccgacgccgtcatgctccggccccgcgcctgctcccgagtgggagaggattatctggtggaaagcgaggtggtgtattcatgttcacgggaatag

SEQ ID NO: 60 - DNA, 1638 bp, Bifidobacterium infantis
  misc_feature: Endo-B-N-acetylglucosaminadase
atgactttcatcaaacagatgatgccgcgttatgtggcgtctatgaccgcaggcatcgtagcggcagcgatggccgcaacatgcgcgtttgcaccggtagcgaatgcggacgccgtttctccgacacaggaaaccatacaatcgaccggtcgccatttcatggtgtactaccgcgcgtggcgtgatgtcacgatgaagggtgtcaacaccgaccttccagacgataactggatttcgatgtacgatattccgtacggcgtcgacgtggtcaatatattcagctatgttccatcgggacaggaagaacaggcgcaaccgttctacgataagctcaaatctgattatgcaccgtacttgcattcgcgtggcatcaagctggttcggggcattgattacaccggcgtggcggtcaatggttttcgtactttcatgaaagagcagaacaaaaccgagagcgaggcgaccgaagctgattatgatgcctatgctaagcaagtaatcgataaatacatgatctccgttggcttggatggtctggatatcgacatggaggcgcacccgaatgatgccgatgtgaagatctccgacaatgtgattcgtgcactgtccaaacacatcggtcccaaatccgccaagccggataccacgatgttcctttatgacacgaatggatcttatctcaatccgttcaagaatgtggcggaatgctttgactacgtggcataccagcagtacggttcttcctccgatcgcaccgctagggccgccgccgattatcagccttatatcggcaacgagtttgtgccgggcctgacgttccccgaagaaggggacatgaacaatcgctggtatgatgccaccgagccgtatgaagaaagccatttctatcaagtggcgtcatatgtgcgtgagcataatcttggcggcatgttcgtttacgcgctcgaccgagacggtcgcaactatgatgaagacctgcgtcggattgtaccgtccaacttgctgtggaccaagaccgccattgcggagagcgagggcatggcgttggatacggcaaagactgcagcgaaccattaccttgatcgcatgtcgttgcgtcaggtgattgacgacaacgccgcatccgcggataaggcgcgggatatggtggggaaggccgctaacctatacgagacaaataaagcagtccttggtggtgactacggagaaggtttctccaacacatatgatccgacattggaagctggattattagggattgacatctccgtgttgcaacagcagattgataaatccagtgagatcattggtgccgatacggcggaatcggatgcaaaaactgcactccgtatggcacgtgatgccgccatcgatggtttgaccggcaagatctacacagcggaccaagtatcagcgtggtcgcaggcattgaaggcggcgcttgatgccaccgtgccggttccgacgccagattccaccgatcagaatggcaaccgtgacaaggttaccaatcacaaagtgcaaggccaaccgaagcaactgagtgcgaccggcatctcgacggatattatcgttgccgtaggcgtgactcttgccatcgcaggggtggctctgtctctatcacgtaagctctcctga

SEQ ID NO: 61 - DNA, 876 bp, Bifidobacterium infantis
  misc_feature: HTH-type transcriptional activator RhaR
atgcagtacgttaagggcaccaatcaggaactattgaccacatcaccgcagttccctgccctggtcatcgcacatcagggattcgacaaagccaggttgcattggcatgaaggcttcgaggtggtatacgtccgacgctgtcgggccacagtgatgaaaggcacccaacgttcgatgcatcatgccggtgatgtagtactgattcctccacgctgcctgcatagcatcgaacttattcaggatcttcggcaaccatcagctgtaccgcaggcgctttccgtaacgatctcgcccacggagttgctaccgtcatatccgtatattacccaaatccaacaacagctggattacgactacattggcgaaaaagatcatcaacaattgctgaaatgctgcgaacaaatgtttgcggcactgatcagtggcaagcagacacggttccttgaagcgaattcctggttctacaccatgcttaccaatgtttttgactacgccaaaccattggctgcagcgaaccaagaagacaccgaggatgaaaccatactccaggatggacgagtcattagacgaatacgccactacgtgcaacgtcattatcgcgagcccctatccaccgcacaggtgtcgcagaaattcggatattcacgagagcatttttcaaggatattcagcaaatattctggagtgacatttaaagactacgtaacgcgtgtacgtctgctggcggcctgcgatttgctcaccaacacggatatgccaatcaatcaaatcgtccgggaatccggattccccagttctcaaaccatgcgaggatcgtttgaccgcgaattcgcatgcacgcccagcgaatatcgctctcgttcgatggaagaataa

SEQ ID NO: 62 - DNA, 696 bp, Bifidobacterium infantis
  misc_feature: Putative N-acetylmannosamine-6-phosphate 2-epimerase
atgaatactaacgatacttattcgaaacgtcttattgattcgctccacggcacactgatagtcagctgtcaagcatatcccggcgagccaatgcggcatcccgaaaccatggcccaagtcgctcaatcagtagtaatcggcggcgctgccgccattcgatgccaaggtcttgccgatatcagcgccatcaaaggccaagtaaaagttccagtcatcggcatatggaaggaaggcgataacggcgtatacatcacgccaacgttacgccacgccagagcctgcattatggcgggagccgacattgtggcactcgatgctacagatcggcctcggccagacggtctaactctgcagcagaccgcgcggcaactcaaaagcgagggcgcaatactgatggccgactgcggctgcatcgaagactccgatgcagccgtggatgcaggattcgacatcatctccaccacgctggctggatacaccgattcccgcgccaaaacagaaggaccggattacgagctcctcgctcaaatgctggagcggcacccccacgtaccggtgatatgcgagggtaggatacatactccgtccgatgcggccaaagccatagaaatgggtgcctgggcagccgtagtcggtaccgccatcacccaccccatgacgataacgtcatggttcgccgatgcagtaaggtcctga

SEQ ID NO: 63 - DNA, 1020 bp, Bifidobacterium infantis
  misc_feature: glucokinase
atgagttttgcaagcataatccatcagcatacggtgatatccgtggacatcggcggcaccaaaatcgccgctggtctcatgtgcgtagacctcgacgaacaaggaagaccgcgggacatgccagcgttgattcgacggtacgtcataccgacgaaagcacgtctcggcggcacctccgtattgcaacgcgtcgttcaagccattcgccagtgtctggacgatggtgagattcctttcccactattggggatcggtgtagcgagcgcaggagtcattgacggcaacggttcggtggtatcagccacttcgttgattcccggttgggccggtattcaactgcaagccgaattatcggcaacgttcaacgtgcctgccatcgtcatcggagatgttcaggcacacgcccttggtgaagcgcactggggatgcggacgacaataccaatcactactggtcgccgccatcggcaccggtatcggaggcgcgataatcatcgacgggaaactggtacgcggcgtccacggagcctgtggccatatcggccatctgccgcatcccgacgccattggtatatcctgctcatgtggatgtgagggccatgtggagtcaatcgcctcaggtaccggcattgccgataattatcgacgtgctctgcaacagaacggacacaatactctccccaccgacatcaatggacgcgctatcgccgaactggccagtcaaggtagtacggaagccgtaaccagtatcactctggccggcacgagtctcggcgatgttcttggcggactaatcaatgcccttgatcccgatgccgtgatcctttccggctccgtagtccattctggagagctatggatgaaatctgtgaaacaaggcatccaaagacagaccttgggcattctttcccgaacaccggttttgacaggaacactcggcggcagagcccctctcatcggagcaacatcagctctgtgcgccgacctcagatcaaataaggagcagaaatatgaatactaa

SEQ ID NO: 64 - DNA, 948 bp, Bifidobacterium infantis
  misc_feature: Lactose Transport Protein
atggaagagaggcgcgagaagtcgattgcggtgactgccctgaatgagggcgggggagggaaatctcatcgctcaggtcgatattccgccgagcggcagccccatgcgtggctgttgctgttcccggccttggcgctgatcaccgtgttcaatctgcttcccctggtgcgtatcttcatcatgtcgctgcagggcgggacgttgacccgtcaacggtttgtgggaatgcgtaattttgttttcgtgttcgcggatcccgaattccgcaaggccatcgccaatacggcgctgttcgcattcgtcgtggtgtcggtcgggctggtgctgtcgatggcggtggccgtggccatcaatggaaagcttcgcggaggcagggtcttcgaaatcctgttcttcatcccctatctgacctcggtgatcgccatcggcatggtgttccggtatctgttcaacggcgattacggtcttgtcaattacgtgttgggtctgtgcggccttggaccctatgattttctcaacgatccgaagttcaatatgcccacgctcatcatcttcggcatatggtcgtccctggcgttcaacatcatcattctgttgtcgggcctgcgcggcatagacaaggaatattacaaggtggccgacatgttcggcgccacggcctgggagaagttccgccggatcacgttgccgcagatggtgccgattctgacgttcctgtccatcgtggatttcatcaattccttcaaggtgtatacggaggtgtacgccttgttcaacgggaaggccggtatcggcgatagcgccacgacggcggtgttctacgtgttcaacaagttctacgtggacaacaagtatgggcagggcatggccgcggcggtggtgctgttcctggtgattctggctttcaccatcatccaaggcatcgtattgaggaggctggcgaagtga

SEQ ID NO: 65 - DNA, 1185 bp, Bifidobacterium infantis
  misc_feature: Sialidase gene
atgacggagaacgggatgatgaatacgaacaatactgtgtgcggcgcgaaccatgacggagcgatgagtctggcggccccaggcgattacggtgtggcctgctaccggattccggcccttgccgaggcgcccaacggctggatcctcgcggcgttcgacgcgcggccccataactgccaggatgccccgcaagccaattcgatcgtgcagcgtatctccaaagacggcggccggtcattcgagccgcagcatgttgtggccgccgggcatgatggcgtcgacaaatacgggtattccgatccgtcctatgtggtggaccggcagacgggagaggtgttcctgttcttcgtcaaatcctatgacgccggtttcggaacctcccaggcgggcgtcgatccctctgcgcgtgaggtgcttcaagccgccgtcaccagttccatcgacaatggcgtgacgtggagcgagccgcgcatcatcaccgccgacatcacgaacagcgaatcatggatttcacggttcgcttcttccggtgccggcatccagctcacgtatggcgagcatgcggggcgcctgatccagcagtacaccatcaaggagctcgacggccgctaccgtgcggtatcggtcttctccgacgatcacggtgcaacctggcatgcaggcacccccgtcggcgatcacatggacgagaacaaagtcgtggaactttccgatggccgtgtgatgctgaactcgcgttcctccgatggaaacggttgccgctatgtcgccatctcccgggacggtggcgccacgtatggtccggtgatccgtgaaacgcagctgcccgatcccgagaacaacgcgcagattgcccgtgcgttccccgatgcccccgaggggtcggcgcaggccaaggttctgctgtattcctcctcgtcgccttcggacaggatcgatggtctggtgcgcgtctcgatcgatgacggcaagacctggagtgccggccgacggttcacgacagggccgatggcgtattcggtgatcgccgcattgagccacaaggccggcggcggctatggcctgctgtatgaaggtgataataataacattatgtacacccgtatctcgctcgactggctcaacggccagctgaacgtcgacggaatcggcggttttccgctgtctggtgagggagggtgctga

SEQ ID NO: 66 - DNA, 438 bp, Bifidobacterium infantis
  misc_feature: L-fucose mutarotase
atgctgaagggaataccgccgatcatacagcccgatctgctgaagatactcagcgagatgggccacggcgatgccatagtccttgcggacgcccactttcccgccgaatcggtgggcgtccgatcccacgtgatcaggtatgacggccagcccatcgagccgctgctcgacgcggtgctgcagctgataccgctggaccaatacacggaacacccggtgctgctgatggacaaggttcccggagacaccgtggacaccccgatatgggaccggtaccgtcaggtcatcgacaggcacgagcccggcaagcaagcgggcatcgggatgctggaacggttcgccttctacgaggaggccggcaggtcctattgcatcgtcgccaccggcgaacaatcgcagtatgcgaacatcatcatcagaaaaggcgtcattcgctaa

SEQ ID NO: 67 - DNA, 705 bp, Bifidobacterium infantis
  misc_feature: Hypothetical gene
atgtccgtacgaaaaacgtctatcatgtgcactatggggaacccaaatgaaatcagactggcgatagtcgataacgacgacttcgtgctgatgggtttggcagcgttcttgtcgcgtcatctgccgaatgttcggttagcttggaaggcgaataccggaaccgatgctctggaatatgcgacggatcccgcaaatgaagcggacattctgctggttgacatgagtctggaggacatgcccggagacatggtgtgccgggaaatcagaagtcgtaacaggatgttgccgttgctggcggtgacatcgttcagtttaactcgctatgcgcgacgtgctgctgagggtggtgctcaaggcattgtgtcaaaagctgattttccagtactgtgtaaagcggtcaagctcgtcagcgatggtcatactctctgtgttcgagtaggaggggagactattggattcgaggatgtagatgctgcatatcatcgtctggttcgacttcccgtgaatagaatcgaaagattgtcggaacgggaaaaatatgccatggaactatattcacagtcgtataagcccactcagattgcccggatgatggatgtttcggcagggacggtgaaaacctatcttgaccgtgctcagaacaaactccatcttacttccagagccgaactgattgcctattggtggaggcgggaacgatgctga

SEQ ID NO: 68 - DNA, 1722 bp, Bifidobacterium infantis
  misc_feature: ureC Urease subunit alpha
atgagaaccatcaccagaagcgactatgcgggcatgttcgggccgaccaccggcgatcgggtgcgcttgggggatacgggtctgctgatcgaggtcgagaaggattacgcgcgttatggcgatgaattgaaattcggcggcggaaaatccttccgggacggcatggggcagtcatccgtccagaaggactccgaatcgccggacacggtcatcaccaacgcgctcgtcgtcgattacaccggcatctacaaagcggatatcggcatcaaggatgggaagatcagcgcgatcggcaaggccggcaacccgcagaccatggacggggtgaccccgggtcttgcagtcggcgcgtgcacggaggcaatcgccggtgaagggctcatcctcaccgccggcggcatcgacacccatattcatttcatcgcgccgcagcaagtccgcacggctcttgccggtggcgtcaccaccatggtcggcggcggcaccgggccggctgacggcaccaacgccaccacatgctcgccgggcgcgttccacatcgcccgcatgatcgaagccgccgaagccatgccggtgaacatcgcgtatctgggcaaaggcaacggatcatcgccggaaccattgcgcgagcagattcgtgcgggagcggccggattgaagatccacgaggattggggcgcgaccccggccgtcatcgacacgtgcctcggtgtcgccgacgacatggacgtgcaggtggccatccatacggacaccctgaatgagggaggatgcgtcgaagacaccatcgccgcattcaagggccggaccatccacacctatcacaccgaaggggcgggcggcggccacgcgcccgacatcatccgcgcggccagtttccccaacgtgctcccgagctccacgaacccgaccatgccgttcacccgtaacaccatcgatgaacatctcgacatgatgatggtcacgcatcatcttgaccgcaatgtgccggaagacatcgccttcgccgattcgcgtattcgccccgagaccatcggggccgaggatgtgcttcatgaccttgggctgatctccatgatgagttcggactcgcaggccatgggacgggtcggagaggtcatcacccgtacctggcagacggccgacaagatgaaaaagcaacgcggcccgctccccgaggatgcgcacgacgacaaccgcaacgacaatttccgtgtgaagcggtacgtgtcgaaatacaccatcaatccggccatcacgcacggcatctccgactacgtcggttccgtcgaagtcgggaaaatggccgatctggtgctttggcagccggcgctgttcggcgccaaaccggagatgatcatcaagggaggatcgatcctccatgcgcgtatgggcgacgcgaatgcgtccatacctactcccgagccggtgctgtaccgcgacatgttcggcgcgatgggcaaagccttgggctcctcatgcgccacattcgtctcgcaggccgcccatgacgacgacatcgccggccggctcggtctcgaacggcaggtgcttccggtgcgccactgccgtggcatcggtaagaaggatctgaagttcaatgacaccatcgcggatattcaggtcaaccccgagacgttccaagtgagcgtcgacggcgagccgattcacagcgaaccggtggcggaactgccgttggcgcagcggtacttcctgttctag

SEQ ID NO: 69 - DNA, 963 bp, Bifidobacterium infantis
  misc_feature: LacG Lactose Transport System
atgacaagcacatccatcaatccggcgccggtccgcggcgcggggtcggacgacatcccgttcaacccgaagctgaggcagaggaggagcgtgtccgactgggtcgtcgacatcgtgatctggctgttgatcgcgttggtcgtggtggcgatcgtctatccgatctggttcatcgtgatcgcctcggtctcggaccagacgatggtcagccagggcaaggtgttcctgttgcccgcgaagatgaacttcggcggttacgccaaggtgttcaccgattcgaggatctgggtcggctaccgcaacaccatcttctattcggtggcgggcacggccctgaacatgctggtcacgatcccggcggcgttcgcgttgtctcgccgcgagttcaagccgcgccgcgtgatcctgttcctgatgacgttcacgatgttcttctcgggcggcatgatcccgagcttcctgctgtacaagcagctgggtctgctgaactcggtgtgggtgttcatccttccgggcgcggtgagcgtgtggaacctgatcgtggcgcgttcgttcttcgagagctcgatcccggagagcctgcatgacgccgcgcagatcgacggcctggggtatttcgggtatttcctgcggatcgtgctgccgttgagctccgcgatcctggcggtgatgacgttgtattacttcgtgggtcattggaacgacttcttcactggcttggtgtacatccgcgacgcggacaagctgccgttgcagaacgtgttgcgttccatcctgttgtcgaaccagacgaacatcaccggtcagagttcgggtggcatggacgtggtgcagcagcgtgatttcgcgaaccagatcaagtacggcgtgatcatcgtgtcgacgttgccgttgctggtgttgtatccgttcctgcagaagtatttcaacaagggcgtgatgatcggtgccgtgaagggctga

SEQ ID NO: 70 - DNA, 954 bp, Bifidobacterium infantis
  misc_feature: putative multiple-sugar transport system permease YteP
atgtctgtggtgaagcagggtgaagtggtggataacgcgttggcggctcgttccgcgccgttgccggtgcggttggggcgtcatttccgtcggtacggcgcgttgtggctgatgacgttgccggcgttggtgttcgtcggtctgttcgcgtacgtgccgatgtacggtcttcgtctcgcgttctatgatttcgatccggtcgagggtctgatgggcggcaggttcgcggggctgcggtatttcgagcagttcttccggtcgggcatgttcgtgaacatcatggtcaacacgttgcggatcagcctgtggacgctggtcatggggttcatcgccccgatcgtgctggcgttgctgatcaaccagatctcgtcgtcgaagatcaaggggttcgtgcagacggtgacgtacatgccgcacttcatctcgacggtcgtgatcgtgtcgatgatcaacatcttcctgtcgccgagcacgggcatgatcgggcgcctgttcccgggcaccgacctgttgggcgagccggggctgttcacgccgatctactggatcagcgaggtgtggcagcacatgggttggaactgcatcatctatctggcggcgttgagttcggtggacctgtccctgtacgaggccgcgaagatcgacggggccggtcgtctgcagctcatccgctacgtcgacatcccggcgatcatgccgacggtgggcatcatgctgatcatgaacatgggttccgtgctcaacgtgggcttcgagaaggtgctgttgatgcagaacccgatgaacctgtccgtgtcggaggtcatcgcgacgtacacgtaccgcatgggtattctgggcaaccagttcagctacacgacggccataggcctgttcaacaccgtggtcaacttcttcttcctggtgctcgccaacttcatttcgaagagggcgtcggacaccagcatcttctag

SEQ ID NO: 71 - DNA, 1395 bp, Bifidobacterium infantis
  misc_feature: por;PolyolNADP_oxidoreductase
atgactgatacacatgatttcacgtcattgcccagattgaaccgcgcagagcacggccgcgccgcagcgccggtgcgcatcatccatcttggtatcggcaatttcaccagggcgcatcaagcctggtacacggaacatgcgccggacaccgcgcagtggggcatcgccggtttcaccggacgttctcgccgaatggtcgacaagctggcccctcaggacaacgtctacacgctgatcacctcggcaccggaaggcgattcgttcgaagtcatctcttcgatttcatcgatgcatgagggcggcgacatggcggcactgcacaagtacttcgcggatccgaacgtgtccatcgtcacctccacggttaccgaagctggatacaaaaggaacgcccacggtgatctagatgtcaacgacaccgacgtgtcggaggacctcgccaaactgcgtaacgatgtgaacgtgtccgaacttgccaccgtgcctgcacgcattgtggccgggctggaggcgcgtcgcgcggcaaacgccggagcgatcactatccttccctgcgacaacctcgcaggcaacggagccgcgttccgtcgcgtcgtcgagcaggccattgaggccgtggaccccaccttgctggaatggactcgcgacaacgtggcatgggccacttcgatggtcgaccgcatcacgccggccaccaccgacgccgaactgcagaccgtggcacaggacgagggatggtacgacgtggcgccggtgcgcacggaaccgttcctcgaatgggtcatccgaggcgacttcccgaagggacgtcctgcatgggacaaggccggtgccgttatcacggatgatgtggaaccctacgagcagcgcaaactgtggatgctcaatggttcgcattccactctcgcctatgtcggcccgctgttcggtcatgagtcggtggccgaggccatcgctgacccgcagctgcgggcatgggtgaatgaatggtgggatctggccggatcatacctgtccgtctccagtgacgactaccgtgccaagctgttggagcgattctccaatccgcgcattcaccatcaccttttccaaatcgccaccgatggttcgcaaaagctgccggtacgaatcgtgcctgtcgccaagcacgcgcttgcggacggtcgcgacatcacggctccggcacgtgctgtcgccgcatggatcgtgttcctgcgtcgatttggaaacacggaggccaaggacgtgaatctcgacgaggtgaggaaacttgccgtcaacgatgacgtcaccccagccgtgtcctacctggacaaggatcttggcgccaacgccactttcaccaacaaggtgagcgaactcgtcaacgagctgtcttcggcgacagccgcgtga

SEQ ID NO: 72 - DNA, 1431 bp, Bifidobacterium infantis
  misc_feature: UxaC uronate
atggtatccgcgctcaatgaagatcgcctattcccaacggatccggaacaacgtttaatcgcacgtcgactgtacgaggcaatcaaggatcgtcccatcatctcccctcacggacacgtccctatcgactggttcgcggaagacaagcatttcaagaaccccaccgatctattcatcactcctgatcactacgtgacccgtatcatgcacggacatagtgttcctttcagcgagctgggtgtgggccagaagaacttcaccgaagaacaatctcgcaacgccttccgcctccttggaaagtactggtatgcctatgctggcacgccgatgcgttactggatggaggattcgctgtccaatgtgttcggcatcaacaagccactcaatgaagacaccgcagacagcatctacgatgagctcaacgagctccttgcctccgacgatttcaccacccgcaagcttgtgaagcgtttcaacatcggcttcatctccaccaccgatgaccccaccgatgacctggtcctgcacgataaggtgcgcgccgacgccaatttcccggcgcgtcttgcaccgtgtttccgtccggatcgctaccttgcggtcgatcgtgtggattgggctcagctctgcgaccagctgggtgaatcggctggcgtaaacaccgccacttatgaaggattcgtagaggccatgcgccgtcgtcgtctgttcttcaagcagcatggcgcagtcgccgccgattacggtgtggatccttcgcttgacgaagtcaattggtccggcgacaccacacgcctgtcggatgatgtggcgattcgcttgtacaccaaggctcgtagtggcagcctgacttccgatgaggcacgcaagctgcacgcccatctgctcaacgaccaggcaaagctcgcgcaggacgacggtctggtaatgaccttgcaccccggcgtgatgcgcaaccactaccgcaagcagctcgtcaactacggtccggactgtggagcggattgcccgatgccggctgattgggctcactggctgcgccccatgctcaacgaatacggcgagaatcctgatttccaccttgtggcattcaccatggatgagaccgcatactcccgcgagctcgctccaatggcggcatactacccggcgctgtacatcggagccccgtggtggttcctggatgcgcctgagccgattctgcgctactacgaggatgtcgttccgtacgctggtttcgccaagctgtcgggattcatcgatgacacccgtgcgctgtgctccatccccgcccgccacgacatgaaccgtcgtctgaccgcacgttacatctccggtctggtcgccgaccaccggttgagttatgaggaaggcgagcagatcgccattcgttccgtggacggtcagccttctgacgtgttcaagctctga

SEQ ID NO: 73 - DNA, 1536 bp, Bifidobacterium infantis
  misc_feature: epsK;putative_membrane_protein_EpsK
atgggagataagaaacaactgcttgtgaacatggtcgcaagtttggtgaatttcacggtttccgttggaatcggtctggtgcttacgccatatatcgtgcgtagcatcggcgctgaagcctatgggttcgtcggattggcgaacacgttcgtgagctatgcgcagctgcttaccatcgcattgaattcggtggccgggcgcttcatcacggtcgcataccatgaaggcgatgattccaaggcgaacgggtattattcgtctacgcttgcggcaaacggcgtcatggtcgccatactggtggtcgtcgcggttccggtggtgacgttccttgacaagctggtgaacatctctccacatcttgtgggagacgtgaaggctctgttcgtcttcatattcctgaatttcatgctttcgaccatcgccacggtgtattccgtcgccacattcgtgaagaacaagctgtatctgagcagtatcgcgaatctcgcgttttccttggtacgtgtggtggccatggtcgcgcttttcgggattctgccgccaaaggtctactacgtgggcttggccgtatgcttggcgacggcggtgatgacgctgatgaatcgctcgtatacgcgcagattacttcccgacatttccttcgacaggaaatcggtatcgtggacgagcattcgtgagatgctttccgccggcgtgtggaacgtggtcacgaaactccagcagattatgatgttcggattgcagttgctggtggcgaacctgatgatcagcccgtatcttatgggtatgctctccatcgcccagaccgttccgaaccagatcagcggactaatgtggactgtgtcgagcctgttttatcctgaacagacgaaatactatgcgcagggcaagcataaggaactcatcgaagacctcaaatcgggcatgaaggtgaccggtttcttcacgaatattatttttgtggcgatgcttgttgcaggatatgatttcatgagtttgtggcagcatggtcaggatacggagctgttgtatcagttgcttacccttaccatgctgggcttgctgatttccggtgtcgctacgacgttgcagaatctgccgctgatcgtcaaccgtttgaagaagtattctatcggatggctcgtctacagtgcgatatccatggtcgtgctgattgcctttatcgaggttcttccgaagtggggcgttttcctcgtggcggcgattccgcctctgtttgagattttggcaaatgtgacattcgtgcccgtttatgcctccagatgcttgggcatcgggaaattcgagttctatcccatctatattcggtacttcgcctccacagcggtggctgccgccgtctgctggggaatcaggcatgtgttcgcgctggtcgccaatggctgggtctcgttgattctgacgtgttgcctgtatgcattggtcacaatgcttttggatgtagtgttgttgctcggaaagaaagaacgttccatgttggtaggtatgctgaagaagaagctacatatcaactag

SEQ ID NO: 74 - DNA, 897 bp, Bifidobacterium infantis
  misc_feature: putative 2-dehydro-3-deoxy-D-pentonate aldolase YjhH
atgaccagcaacgccatgttcgaaggcgtgttctgcccctccatcaccatcatgaacgcggacggaaccatagattacgacaactggggcaagcacctcgaccacctcgtcgacgcaggcgtcgacggcgtgctgctgttcggcagcatcggcgaattctacgccatcgacgtcaagacgaaggcggaagcggctcgcttcgccgtctcgaaggtcgccggacgcatgaaggtgctcgtcggcgtgggagacaccaacctggacaacgtgaaggcgttggcggcggaaagcgaagcggccggagtcgacgcgctgctcgccgtgtccccgtactatttcggcccgtccccggattgcgcgaaacggtacttctcggcggtggccaaggcgacgaccctgcccgtcatcctgtacaacttcccggcccgaacgggcaacgacctcacgcccgagctggtggccgaactcgccggcgagaacccgaacatcgtcggcatcaaagacaccgtcgacaccatcagtcacaccaggaaggtcatcgcggccgtccgcaaggtgaacccgtcgttcagcgtgctgtccggattcgacgaatactacatcgtcaaccggatcagcggcggcaacggcgtgctgagtggtctgaccaacgtggaacccgagacgttcgtcaaactgcaccgcgcatgggaggccggcgaccacgccgcggtcgtcgaagcggccgagcgcgtctcctacctgatgcgcctgtacgacaccgccgacctgttcatcagcgccatcaagggcgcggtcaaggccaagggactacccatcgacacgtccgtccacgagcccgccgtgcagctgaccgacgagcagtatcgcaccatccgcgccatcctggacaagtga

SEQ ID NO: 75 - DNA, 1266 bp, Bifidobacterium infantis
  misc_feature: Fucosidase
atgacatttcccgaaggattccagtttggtacggccacggcctcgtatcagatcgaaggcggcgccaccgagggcggccgcgcgccatcgatttgggacacgttctcccacacgcccggcaaaaccgtgaacggcgataccggtgatgtggcctgtgattcctaccatttgtggcagaaggacatcgatttgctcgccgatctgggcgtggactcctaccgtctgtctgtggccatgccgcgtgtgatgccgaccgaggacggcccggtcaacgaggagggccttgactactacgagcgcgtggtcgacgcgttgctcgacaagggcatcaagcccacggtcacgctctaccactgggatctgccgcagtacctcggcgatgggaacggttggctcaaccgcgacaccgcctacaaactggccgactatgcgcgcatcgtggctcaccgtctgggcgaccgcgtcgaaacctacaccacgttgaacgagccgtggtgctcctcctacctgagctacggcgccaccgagcacgcacccggtctgggccttggtcccggtgccttcccggccgtgcatcatctgaatctggctcatggtctgatggcccaggcggtgcgtagcgaggtcggcgacaggtcgcagctgtcggtcacgctgaacctgcagttcaaccgtggcgacgccgacgccgtgcaccgtctcgacctgatcggcaaccgcgtgtggctcgacccgatgctgcgcggctactacccgaacgagctgttcgccatcaccaagggcatctgcgattgggagttcgtcaaggacggcgacctcgaacagatccatcagccgctggacgtgctcggcatcaactactattcctccggcttggtcacgatgagcggacgcccgcagttcccgcagtccacgggcccgagcaccgcgcctggtgccagcgacgtcgactggctgccgacgcccggcgagcacaccgacatgggctggaacatcgatccgaagggcctatatgacctgctgatgcgcgtgcataacgattatccagagattccgctgatggtcaccgaaaacggcatcgcggtcgagggcggcgaccgggtcgtcaccgaggcggatggcaccaaggccgtgcacgaccccaagcgcatcgactacctgaagcggcacttcgaggccgcactcaaggctatcgaggacggcgtggatttgcgcggctacttcgtgtggtccatcgcaccagctgccaagcagtccggctactga

SEQ ID NO: 76 - DNA, 165 bp, Bifidobacterium infantis
  misc_feature: Hypothetical gene
atgacattgccgccgcaatcgcgaaaagacgatgactgcggcggcatcctcaacatcactccaacagaaagcatgacccacggcaccacgcgtcaggcgctcagtctcatcaccaatcccatcatcaccatcgtgctttttgcgctcgccgtgttcgcctgctga

SEQ ID NO: 77 - DNA, 1462 bp, Faecalibacterium prausnitzii
  misc_feature: Faecalibacterium prausnitzii partial 16S sequence NCBI Reference Sequence NR_121697.2
gatcctggctcaggcgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtggccgatgtttactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtac

SEQ ID NO: 78 - DNA, 1521 bp, Agathobacter rectalis
  misc_feature: E. rectale (Agathobacter rectalis partial 16S sequence NCBI Reference Sequence NR_074634.1
agagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacct

SEQ ID NO: 79 - DNA, 1479 bp, Anaerobutyricum hallii
  misc_feature: Anaerobutyricum hallii 16S gene NCBI Reference Sequence NR_118673.1
  misc_feature (617)..(617): n is a, c, g, or t
  misc_feature (955)..(955): n is a, c, g, or t
  misc_feature (1087)..(1087): n is a, c, g, or t
  misc_feature (1098)..(1098): n is a, c, g, or t
  misc_feature (1184)..(1185): n is a, c, g, or t
  misc_feature (1227)..(1227): n is a, c, g, or t
  misc_feature (1259)..(1259): n is a, c, g, or t
  misc_feature (1269)..(1269): n is a, c, g, or t
  misc_feature (1374)..(1375): n is a, c, g, or t
  misc_feature (1391)..(1391): n is a, c, g, or t
tttatttgagagtttgatcctggctcaggatgaacgctggcggcgtgcctaacacatgcaagtcgaacgaagcaccttaccwgattcttcggatgaaagwytggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggngctgcatttgaaactgcwyrgctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggctycggtgccgccgctaacgcagtaagtattccacctggggagtacgttcgcaagaatgaaactcaaaggaattgacggggacccgcacaagcggtggagcatgtggtttaattcgaagnaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccncaacgagcgcnacccctatcttcagtagccagcaggtaaggctgggcactctggagagactgccagggataacctggaggaaggtggggacgacgtnnaatcatcatgccccttatgatctgggcgacacacgtgctacnatggcggtcacagagtgaggcgaaccygcganggggagcaanccacaaaaaggccgtcccagttcggactgtagtctgcaacccgactacacgaagctggaatcgctagtaatcgcgaatcagaatgtcgcggtgaatacgttcccnngtcttgtacacaccgnccgtcacaccatgggagtcggaaatgcccgaagccagtgacccaacctttatggagggagctgtcgaaggtggagccggtaactgggg

SEQ ID NO: 80 - DNA, 1482 bp, Anaerobutyricum soehngenii
  misc_feature: Anaerobutyricum soehngenii 16S GenBank AJ270490.2
tgatcctggctcaggatgaacgctggcggcgtgcctaacacatgcaagtcgaacgaagcaccttttaagattcttcggatgattgatcggtgactgagtggcggacgggtgagtaacgcgtgggtaacctgccctgtacagggggataacagttggaaacggctgctaataccgcataagcgcacgagaggacatcctcttgtgtgaaaaactccggtggtacaggatgggcccgcgtctgattagctggttggcagggtaacggcctaccaaggcgacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatcctaggtgtcggggccgtataggcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccactccgtaatgggagtcttccttcgggacagaagagacaggtggtgcatggttgtccgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacaaagtgaggcaaacctgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccttttggagggarctgtcgaaggtggagccggtaactggggtgaagtcgtaacaaggg

SEQ ID NO: 81 - DNA, 1456 bp, Anaerostipes caccae
  misc_feature: Anaerostipes caccae 16S gene NCBI Reference Sequence NR_028915.1
gcgcttaatacatgtcaagtcgaacgaagcatttaggattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggtaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatcccaatgaccgaaccttaaccggttttttctttcgagacattggagacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaagg

SEQ ID NO: 82 - DNA, 1493 bp, Blautia producta
  misc_feature: Blautia producta 16S NCBI Reference Sequence NR_113270.1
agagtttgatcctggctcaggatgaacgctggcggcgtgcttaacacatgcaagtcgagcgaagcactaagacggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacggggacttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtaacc

SEQ ID NO: 83 - DNA, 1505 bp, Clostridium leptum
  misc_feature: Clostridium leptum 16S NCBI Reference Sequence NR_114789.1
cgacgctggcggcgtgcctaacacatgcaagtcgaacggagttaaattcgacacccgagtatccggccgggaggcggggtgctgggggttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatggactcgcgtccgattagccagttggcggggtaacggcccaccaaagcgacgatcggtagccggactgagaggttgaacggccacattgggactgagacacggcccagactcctacgggaggcagcagtgggggatattgcacaatgggggaaaccctgatgcagcaacgccgcgtgagggaagaaggttttcggattgtaaacctctgttcttagtgacgataatgacggtagctaaggagaaagctccggctaactacgtgccagcagccgcggtaatacgtagggagcgagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccggtgtagcggtgaaatgcgtagagatcgggaggaacaccagtggcgaaggcggcctactgggctttaactgacgctgaagcacgaaagcatgggtagcaaacaggattagataccctggtagtccatgccgtaaacgatgattactaggtgtggggggtctgaccccctccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgtctaacgaagcagagatgcattaggtgcccttcggggaaaggcgagacaggtggtgcatggttgtcgtcagctcgtgtcgtgagatgttgggttaagtcccgcaacgagcgcaacccttgtttctagttgctacgcaagagcactctagagagactgccgttgacaaaacggaggaaggtggggacgacgtcaaatcatcatgccccttatgacctgggccacacacgtactacaatggctgtaaacagagggaagcaaagccgcgaggtggagcaaaaccctaaaagcagtcccagttcggatcgcaggctgcaacccgcctgcgtgaagtcggaattgctagtaatcgcggatcagcatgccgcggtgaatacgttcccgggccttgtacacaccgcccgtcacaccatgggagccggtaatacccgaagccagtagttcaaccgcaaggagagcgctgtcgaaggtaggattggcgactggggtgaagtcgtaacaaggtagccgtatcggaaggtgcggc

SEQ ID NO: 84 - DNA, 1485 bp, Roseburia hominis
  misc_feature: Roseburia hominis 16S NCBI Reference Sequence NR_042008.1
gatcctggctcaggatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgcggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacagagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggactgataactggggtgaagtcgtaacaagggtacg

SEQ ID NO: 85 - DNA, 1482 bp, Roseburia intestinalis
  misc_feature: Roseburia intestinalis 16S NCBI Reference Sequence NR_027557.1
ttgatcctggctcaggatgaacgctggcggcgtgcttaacacatgcaagtcgaacgaagcactttatttgatttcttcggaatgaagattttgtgactgagtggcggacgggtgagtaacgcgtgggtaacctgcctcatacagggggataacagttggaaacgactgctaataccgcataagcgcacagggtcgcatgacctggtgtgaaaaactccggtggtatgagatggacccgcgtctgattagccagttggtggggtaacggcctaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggtacggcaagtctgatgtgaaagcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgatgacagaacatgtaatgtgttttctcttcggagcatcggtgacaggtggtgcatggttgtcgtcagctcgtgtcgtgagatgttgggttaagtcccgcaacgagcgcaacccctattcttagtagccagcgggtmagccgggcactctagggagactgccagggataacctggaggaaggtggggatgacgtcaaatcatcatgccccttatgacttgggctacacacgtgctacaatggcgtaaacaaagggaagcgagcctgcgagggggagcaaatctcaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaagcaggatcgataactggggtgaagtcgtaacaag

SEQ ID NO: 86 - DNA, 1454 bp, Clostridium aminovalericum
  misc_feature: Anaerocolumna aminovalerica (Clostridium aminovalericum) 16S NCBI Reference Sequence NR_029245.1
  misc_feature (465)..(466): n is a, c, g, or t
  misc_feature (572)..(573): n is a, c, g, or t
  misc_feature (774)..(774): n is a, c, g, or t
ttaacacatgcaagtcaacggagttaatcaagcgcgaagtcttcggatggaagcttgttaacttagtggcggacgggtgagtaacgcgtgggtaacctgccttatacagggggataacagccggaaacggttgctaataccgcataagcatacagtgccgcatggtacagtgtgaaaaagattaagttcaacttcgttcaactctggtataagatggacccgcgtctgattagctagttggtgaggtaatggctcaccaatggcgacgatcagtagccggcttgagagagtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggacgataatgacggtacctgactaagaagccnnggctaactacgtgccagcagccgcggtaatacgtagggggcaagcgttatccggatttactgggtgtaaagggagcgtagacggtctggtaagtcaggtgtgaaanncccggggctcaaccccggattgcacttgaaactacccgactagagtgcaggagaggtaagtggaattcctagtgtagcggtgaaatgcgtagatattaggaggaacaccagtggcgaaggcggcttactggactgtaactgacgttgaggctcgaaagcgtggggagcgaaacaggattagataccctggtagtccacgcngtaaacgatgaatactaggtgttggggtccataaggacttcggtgccgtcgcaaacgcaataagtattccacctggggagtacgttcgcaagaatgaaactcaaaggaattgacggggacccgcacaagcgtgggagcatgtggtttaattcgaagcaacgcgaagaaccttaccaggtcttgacatccctctgaccggtccgtaatgggacctttccttcgggacagaggaacaggtggtgcatggttgtcgtcagctcgtgtcgtgagatgttgggttaagtcccgcaacgagcgcaacccctatctttagtagccagcagttcggctgggcactctagagagactgccagggataacctggaggaaggcggggatgacgtcaaatcatcatgccccttatgatctgggctacacacgtgctacaatggtgactacaaagagaagcaaagctgtgaagtggagccaatctcaaaaaggtcatctcagttcggattgtagtctgcaactcgactacatgaagctggaatcgctagtaatcgcagatcagaatgctgtggtgaatacgttcccgggtcttgtacacaccgcccgtcacaccatgggagttggatatgcccgaagtcagtgacccaaccgtaaggagggagctgccgaaggtggagccaatgactggggtg

SEQ ID NO: 87 - DNA, 1495 bp, Anaerostipes butyraticus
  misc_feature: Anaerostipes butyraticus 16S NCBI Reference Sequence NR_113319.1
agagtttgatcctggctcaggatgaacgctggcggcgtgcttaacacatgcaagtcgaacgaagcttgatcccggatcccttcggggtgacgggtgatatgactgagtggcggacgggtgagtaacgcgtgggcaacctgcctcatacagggggataacagttagaaatgactgctaagaccgcataagaccacagcaccgcatggtggagtggtaaaagctccggtggtatgagatgggcccgcgtctgattagctggttggtgaggtaacggctctaccaaggcaacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggcagggcaagtcagaagtgaaaacccggggctcaactccgggactgcttttgaaactgtccagctggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagcaaacgcattaagtattccacctggggagtacgttcgcaagaatgaaactcaaaggaattgacggggacccgcacaagcggtggagcatgtggtttaattcgaagcaacgcgaagaaccttacctggtcttgacatccccttgaccgctccttaatcggagttttccttcgggacaagggtgacaggtggtgcatggttgtcgtcagctcgtgtcgtgagatgttgggttaagtcccgcaacgagcgcaacccctatctttagtagccagcacggaaaggtgggcactctagagagactgccagggagaacctggaggaaggtggggacgacgtcaaatcatcatgccccttatgaccagggctacacacgtgctacaatggcgtaaacaaagggaagcgaccctgtgaaggcaagcaaatcccaaaaataacgtcacagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtaacc

SEQ ID NO: 88 - DNA, 1515 bp, Anaerostipes hadrus
  misc_feature: Anaerostipes hadrus 16S NCBI Reference Sequence NR_104799.1
tggctcaggatgaacgctggcggcgtgcttaacacatgcaagtcgaacgaagctgcttaactgatcttcttcggaattgacgttttgtagactgagtggcggacgggtgagtaacgcgtgggcaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatttcaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctttc

SEQ ID NO: 89 - DNA, 1382 bp, Anaerostipes rhamnosivorans
  misc_feature: Anaerostipes rhamnosivorans 16S GenBank JX273468.1
  misc_feature (727)..(727): n is a, c, g, or t
  misc_feature (933)..(935): n is a, c, g, or t
tattttggattgaagttttcggatggatctccttaatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggcggggtaacggcccaccaaggcgacgatcagtagccggcttgagagagtggacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaataagacggtacctgactaagaagccccggctaactacgtgccagcagccgcggtaatacgtagggggcaagcgttatccggaattactgggtgtaaagggtgcgtaggtggcatgataagtcagaagtgaaagcccggggcttaaccccgggactgcttttgaaactgtaatgctagagtgcaggagaggtaagcggaattcctagtgtagcggtgaaatgcgtagatattaggaggaacaccagtggcgaaggcggcttactggactgtcactgacactgaggcacgaaagcgtggggagcaaacaggattagataccctggtagtcnacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggttnnnaaccgaacctttccttcgggacagaagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatggccagggctacacacgtgctacaatggcgtaaacaaagggaagcgaccccgcgagggcaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaaggaggaga

SEQ ID NO: 90 - DNA, 1365 bp, Anaerotruncus colihominis
  misc_feature: Anaerotruncus colihominis 16S
aacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggcgtaatagcctagagagtaggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccagaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtccgggtaacacccgaagccagtag

SEQ ID NO: 91 - DNA, 1530 bp, Blautia argi
  misc_feature: Blautia argi 16S NCBI Reference Sequence NR_171388.1
ttatcagagagtttgatcctggctcaggatgaacgctggcggcgtgcttaacacatgcaagtcgagcgaagcacttgccattgactcttcggaagatctggcaaatgactgagcggcggacgggtgagtaacgcgtgggtaacctgcctcatacaggggaataacagttagaaatggctgctaatgccgcataagcgcacagggccgcatggtctggtgtgaaaaactccggtggtatgagatggacccgcgtctgattaggtagttggcggggtaacggcccaccaagccgacgatcagtagccgacctgagagggtgaccggccacattgggactgagacacggcccagactcctacgggaggcagcagtggggaatattgcacaatggaggaaactctgatgcagcgacgccgcgtgaaggaagaagtatctcggtatgtaaacttctatcagcagggaagaaaatgacggtacctgactaagaagccccggctaactacgtgccagcagccgcggtaatacgtaggggcaagcgttatccggatttactgggtgtaaagggagcgtagacggacgggcaagtctgatgtgaaagcccggggcttaaccccgggactgcattggaaactgtccatcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggttgcaaagcaattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatctgcctgaccgcaccttaaccggtgttttccttcgggacaggcaagacaggtggtgcatggttgtcgtcagctcgtgtcgtgagatgttgggttaagtcccgcaacgagcgcaacccctgtcctcagtagccagcagtccggctgggcactctggggagactgccggggataacccggaggaaggcggggacgacgtcaaatcatcatgccccttatgatttgggctacacacgtgctacaatggcgtaaacaaagggaagcaagagtgcgagcttaagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttagggagggagctgccgaaggcgggaccgataactggggtgaagtcgtaacaaggtagccgtatcggaaggtgcggctggatcacctccttt

SEQ ID NO: 92 - DNA, 1378 bp, Blautia caecimuris
  misc_feature: Blautia caecimuris 16S NCBI Reference Sequence NR_144607.1
attttgacagagacttcggttgaagtcgttataatcctagtggcggacgggtgagtaacgcgtgggtaacctgcctcacactgggggataacagtcagaaatgactgctaataccgcataagcgcacgggattgcatgatccagtgtgaaaaactccggtggtgtgagatggacccgcgttggattagccagttggcagggtaacggcctaccaaagcgacgatccatagccggcctgagagggtggacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaaaaagccccggctaactacgtgccagcagccgcggtaatacgtagggggcaagcgttatccggatttactgggtgtaaagggagcgtagacggagcagcaagtctgatgtgaaaggcgggggctcaacccccggactgcattggaaactgttgatcttgagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgcagcaaacgcagtaagtattccacctgggggagtacgttcgcaagaatgaaactcaaaggaattgacggggacccgcacaagcggtggagcatgtggtttaattcgaagcaacgcgaagaaccttaccaagtcttgacatccctatgaccggcgcgtaacgtcgccttttcttcggagcatcggagacaggtggtgcatggttgtcgtcagctcgtgtcgtgagatgttgggttaagtcccgcaacgagcgcaacccctatccttagtagccagcggtttggccgggcactctgaggagactgccagggataacctggaggaaggcggggatgacgtcaaatcatcatgccccttatgatttgggctacacacgtgctacaatggcgtaaacaaagggaagcgagagtgtgagcttaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcaggaccgaaccgaaaggacggagc

SEQ ID NO: 93 - DNA, 1493 bp, Blautia coccoides
  misc_feature: Blautia coccoides 16S
agagtttgatcctggctcaggatgaacgctggcggcgtgcttaacacatgcaagtcgagcgaagcgctaagacagatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtaacc

SEQ ID NO: 94 - DNA, 1534 bp, Blautia faecicola
  misc_feature: Blautia faecicola 16S NCBI Reference Sequence NR_170495.1
tatttagagagtttgatcctggctcaggatgaacgctggcggcgtgcttaacacatgcaagtcgaacgaagcacttgaaacggattcttcggatgaagtttcctgtgactgagtggcggacgggtgagtaacgcgtgggtaacctgcctcatacagggggataacagttagaaatggctgctaataccgcataagcgcacagcatcgcatgatgcagtgtgaaaaactccggtggtatgagatggacccgcgttggattagctagttggcagggtaacggcctaccaaggcgacgatccatagccggcctgagagggtggacggccacattgggactgagacacggcccagactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggaatggcaagtctgatgtgaaaggccggggctcaaccccgggactgcattggaaactgccaatctagagtaccggaggggtaagtggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttgggtagcaaagctattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatcgatccgaccggaccgtaatgggtcctttcccttcggggacggagaagacaggtggtgcatggttgtcgtcagctcgtgtcgtgagatgttgggttaagtcccgcaacgagcgcaacccttatcctcagtagccagcacataaaggtgggcactctggggagactgccagggataacctggaggaaggtggggatgacgtcaaatcatcatgccccttatgatttgggctacacacgtgctacaatggcgtaaacaaagggaagcgatcacgcgagtgtgagcaaatctcaaaaataacgtctcagttcggattgtagtctgcaactcgactacatgaagctggaatcgctagtaatcgcaggtcagcatactgcggtgaatacgttcccgggtcttgtacacaccgcccgtcacaccatgggagtcagtaacacccgaagccggtgacctaaccgaaaggaaggagccgtcgaaggtgggaccgataactggggtgaagtcgtaacaaggtagccgtatcggaaggtgcggctggatcacctccttt

SEQ ID NO: 95 - DNA, 1262 bp, Blautia faecis
  misc_feature: Blautia faecis   16S NCBI Reference Sequence NR_109014.1
ataacagccagaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgcartggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccg

SEQ ID NO: 96 - DNA, 1468 bp, Blautia glucerasea
  misc_feature: Blautia glucerasea 16S NCBI Reference Sequence NR_112789.1
  misc_feature (990)..(990): n is a, c, g, or t
ctcaggatgaacgctggcggcgtgcttaacacatgcaagtcgaacgagaaacattttaatgatagcttcggcagatttagtttgtttctagtggcggacgggtgagtaacgcgtgggtaacctgcctcacactgggggataacagtcagaaatgactgctaataccgcataagcgcacggaaccgcatggttttgtgtgaaaaactccggtggtgtgagatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggagcagcaagtctgatgtgaaaggcaggggctcaacccctggactgcattggaaactgttgatcttgagtaccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccagagtcttgacatccctctgaccggtccttaaccgganctttccttcgggacagaggagacaggtggtgcatggttgtcgtcagctcgtgtcgtgagatgttgggttaagtcccgcaacgagcgcaacccctatccccagtagccagcatttaaggtgggcactctgaggagactgccagggataacctggaggaaggcggggatgacgtcaaatcatcatgccccttatgatttgggctacacacgtgctacaatggcgtaaacaaagggaagcagagcggtgacgccgagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaagggaggagctgccgaaggcgggacggatgactggggtgaagtcgaac

SEQ ID NO: 97 - DNA, 1492 bp, Streptococcus hansenii
  misc_feature: Blautia hansenii (Streptococcus hansenii)  16S NCBI Reference Sequence NR_104687.1
agagtttgatcctggctcaggatgaacgctggcggcgtgcttaacacatgcaagtcgagcgaagcacttatcattgactcttcggaagatttgatatttgactgagcggcggacgggtgagtaacgcgtgggtaacctgcctcatacaggggaataacagttagaaatggctgctaatgccgcataagcgcacaggaccgcatggtctggtgtgaaaaactgaggtggtatgagatggacccgcgtctgattaggtagttggtggggtaacggcctaccaagccgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggtgcaaagcagttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctgcctgaccgttccttaaccggagctttccttcgggacaggcaagacaggtggtgcatggttgtcgtcagctcgtgtcgtgagatgttgggttaagtcccgcaacgagcgcaacccctatccttagtagccagcagtccggctgggcactctagggagactgccggggataacccggaggaaggcggggacgacgtcaaatcatcatgccccttatgatttgggctacacacgtgctacaatggcgtaaacaaagggaagcgaagcggtgacgcttagcaaatctcaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttatggagggagctgccgaaggcgggaccgataactggggtgaagtcgtaacaaggtaacc

SEQ ID NO: 98 - DNA, 1454 bp, Blautia hominis
  misc_feature: Blautia hominis 16S NCBI Reference Sequence NR_163634.1
gatgaacgctggcggcgtgcttaacacatgcaagtcgagcgaagcactaagatggatttcttcggattgaagccttagtgactgagcggcggacgggtgagtaacgcgtgggtaacctgcctcatacagggggataacagttagaaatgactgctaataccgcataagcgcacagggctgcatggcctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgacgtgcccgtaacggggcattcccttcggggcagaggagacaggtggtgcatggttgtcgtcagctcgtgtcgtgagatgttgggttaagtcccgcaacgagcgcaacccctatccttagtagccagcaggtcaagctgggcactctagggagactgccggggataacccggaggaaggcggggacgacgtcaaatcatcatgccccttatgatttgggctacacacgtgctacaatggcgtaaacaaagggaagcgagacagcgatgtttagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaacctaacggagggagctgccgaaggcgggaccgataactggggtg

SEQ ID NO: 99 - DNA, 1458 bp, Ruminococcus hydrogenotrophicus
  misc_feature: Blautia hydrogenotrophica (Ruminococcus hydrogenotrophicus) 16S
  misc_feature (1416)..(1416): n is a, c, g, or t
gatgaacgctggcggcgtgcttaacacatgcaagtcgaacgaagcgatagagaacggagatttcggttgaagttttctattgactgagtggcggacgggtgagtaacgcgtgggtaacctgccctatacagggggataacagttagaaatgactgctaataccgcataagcgcacagcttcgcatgaagcggtgtgaaaaactgaggtggtataggatggacccgcgttggattagctagttggtgaggtaacggcccaccaaggcgacgatccatagccggcctgagagggtgaacggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagaaagtgacggtacctgactaagaagccccggctaattacgtgccagcagccgcggtaatacgtaaggggcaagcgttatccggatttactgggtgtaaagggagcgtagacggtttggcaagtctgatgtgaaaggcatgggctcaacctgtggactgcattggaaactgtcagacttgagtgccggagaggcaagcggaattcctagtgtagcggtgaaatgcgtagatattaggaggaacaccagtggcgaaggcggcctgctggacggtaactgacgttgaggctcgaaagcgtggggagcaaacaggattagataccctggtagtccacgctgtaaacgatgaatactaggtgtcgggtggcaaagccattcggtgccgcagcaaacgcaataagtattcccacctggggagtacgttcgcaagaatgaaactcaaaggaattgacggggacccgcacaagcggtggagcatgtggtttaattcgaagcaacgcgaagaaccttaccaaatcttgacatccctctgaccgggaagtaatgttcccttttcttcggaacagaggagacaggtggtgcatggttgtcgtcagctcgtgtcgtgagatgttgggttaagtcccgcaacgagcgcaacccttattcttagtagccagcaggtagagctgggcactctagggagactgccagggataacctggaggaaggtggggatgacgtcaaatcatcatgccccttatgatttgggctacacacgtgctacaatggcgtaaacaaagggaagcgaaggggtgacctggagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccnaaaggagggagctgccgaaggtgggactgataactggggtga

SEQ ID NO: 100 - DNA, 1308 bp, Blautia luti
  misc_feature: Blautia luti (Ruminococcus luti) 16S NCBI Reference Sequence NR_041960.1
  misc_feature (702)..(705): n is a, c, g, or t
gtgggtaacctgccttatacagggggataacagtcagaaatgactgctaataccgcataagcgcacagagctgcatggctccggtgtgaaaaactccggtggtataagatggacccgcgttggattagctagttggtgaggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatcctaggtgtcggggagcaaannnnttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccctctgaccgagtatgtatggtacttttccttcgggagagagaggagacaggtggtgcatggttgtcgtcagctcgtgtcgtgagatgttgggttaagtcccgcaacgagcgcaacccctatccccagtagccagcggttcggccgggcactctgaggagactgccagggataacctggaggaaggcggggatgacgtcaaatcatcatgccccttatgatttgggctacacacgtgctacaatggcgtaaacaaagggaagcaagcctgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaact

SEQ ID NO: 101 - DNA, 1398 bp, Blautia obeum
  misc_feature: Blautia obeum (Ruminococcus obeum) 16S NCBI Reference Sequence NR_118692.1
  misc_feature (1083)..(1083): n is a, c, g, or t
  misc_feature (1207)..(1207): n is a, c, g, or t
  misc_feature (1233)..(1233): n is a, c, g, or t
ggcgtgcttaacacatgcaagtcgaacgggaaacttttcattgaagcttcggcagatttggtctgtttctagtggcggacgggtgagtaacgcgtgggtaacctgccttatacagggggataacaaccagaaatggttgctaataccgcataagcgcacaggaccgcatggtctggtgtgaaaaactccggtggtataagatggacccgcgttggattagctagttggcagggtaacggcctaccaaggcgacgatccatagccggcctgagagggtgaacggccacattgggactgagacacgccccagactcctcgggaggcagcagtggggaatattgcacaatgggggaaaccctgatgcagcgacgccgcgtgaaggaagaagtatctcggtatgtaaacttctatcagcagggaagatagtgacggtacctgactaagaagccccgkctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgcaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccttaaccggatctttccttcgggacaggggagacaggtggtgcatggttgtcgtcagctcgtgtcgtgagatgttgggttaagtcccgcaacgagcgcaacccctatccccagtagccagcagtncggctgggcactctgaggagactgccagggataacctggaggaaggcggggatgacgtcaaatcatcatgccccttatgatttgggctacacacgtgctacaatggcgtaaacaaagggaagcnagcctkcgraggtaagcaaatcccanaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgc

SEQ ID NO: 102 - DNA, 1482 bp, Blautia schinkii
  misc_feature: Blautia schinkii (Ruminococcus schinkii) 16S NCBI Reference Sequence NR_026312.1
  misc_feature (37)..(37): n is a, c, g, or t
  misc_feature (173)..(173): n is a, c, g, or t
  misc_feature (184)..(184): n is a, c, g, or t
  misc_feature (188)..(188): n is a, c, g, or t
  misc_feature (367)..(367): n is a, c, g, or t
  misc_feature (1342)..(1342): n is a, c, g, or t
tgaacgctggcggcgtgcttaacacatgcaagtcgancggagattgatttaacagagacttcggttgaagttgttctgatcttagtggcggacgggtgagtaacgcgtgggtaacctgccttgtactgggggataacagccagaaatgactgctaataccgcataagcgcacnggaccgcatgntctngtgtgaaaaactccggtggtacaagatggacccgcgttggattagctagttggcagggcagaggcctaccaaggcgacgatccatagccggcctgagagggtgaacggccacattgggactgagacacggcccagactcctacgggaggcagcagtggggaatattgcacaatgggggnaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggtatagcaagtctgatgtgaaaggcaggggctcaacccctggactgcattggaaactgttaaacttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccccctgaccggtccgtaacgggacctttccttcgggacaggggagacaggtggtgcatggttgtcgtcagctcgtgtcgtgagatgttgggttaagtcccgcaacgagcgcaacccctatccttagtagccagcggtttggccgggcactctagggagactgccagggataacctggaggaaggtggggatgacgtcaaatcatcatgccccttatgatttgggctacacacgtgctacaatggcgtaaacaaagggaagcgaccgggtgaccgtgagcaaatcccaaaaataacgtcccagttcggactgtagtctgcaacccgactacacgaagctggaatcgctagtaatcgcggatcagaatgccgcggtgaatacgtncccgggtcttgtacacaccgcccgtcacaccatgggagtcagtaacgcccgaagtcagtgacctaaccgaaagggaggagctgccgaaggcgggaccgatgactggggtgaagtcgtaacaaggtagccgtatcggaagg

SEQ ID NO: 103 - DNA, 1372 bp, Blautia stercoris
  misc_feature: Blautia stercoris 16S NCBI Reference Sequence NR_117867.1
tgcaagtcgagcgaagcgcttacgacagaaccttcgggggaagatgtaagggactgagcggcggacgggtgagtaacgcgtgggtaacctgcctcatacagggggataacagttggaaacggctgctaataccgcataagcgcacggtatcgcatgatacagtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcgatctgaccggttcgtaatggaacctttccttcgggacagagaagacaggtggtgcatggttgtcgtcagctcgtgtcgtgagatgttgggttaagtcccgcaacgagcgcaacccctatcctcagtagccagcaggtgaagctgggcactctgtggagactgccagggataacctggaggaaggcggggacgacgtcaaatcatcatgccccttatgatttgggctacacacgtgctacaatggcgtaaacaaagggaagcgagcccgcgagggggagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtc

SEQ ID NO: 104 - DNA, 1438 bp, Blautia wexlerae
  misc_feature: Blautia wexlerae 16S NCBI Reference Sequence NR_044054.1
  misc_feature (19)..(19): n is a, c, g, or t
caagtcgaacgggaattantttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaataactaggtgtcgggtggcaaagccattcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggt

SEQ ID NO: 105 - DNA, 1511 bp, Butyrivibrio crossotus
  misc_feature: Butyrivibrio crossotus 16S NCBI Reference Sequence NR_104735.1
tttgatcctggctcaggatgaacgctggcggcgtgcttaacacatgcaagtcgaacgaagcacttcataaagcttgctttaagaagtgacttagtggcggacgggtgagtaacgcgtgggtaacctgccttacacagggggataacagttagaaatgactgctaataccgcataaaacagcagagtcgcatgactcaactgtcaaagatttatcggtgtaagatggacccgcgtctgattagctagttggtggggtaacggcctaccaaggcgacgatcagtagccggcctgagagggtgaacggccacattgggactgagacacggcccaaactcctacgggaggcagcagtggggaatattggacaatgggcgaaagcctgatccagcgacgccgcgtgagcgatgaagtatttcggtatgtaaagctctatcagcagggaagaaaacgacggtacctgactaagaagccccggctaactacgtgccagcagccgcggtaatacgtagggggcaagcgttatccggatttactgggtgtaaagggagcgtagacggcatcacaagtcagaagtgaaaatccggggctcaaccccggaactgcttttgaaactgtggagctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttgggtttcataagaagctcggtgccggcgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccgttgacctgttatgtaatgtaacatctcttcggagcaacggagacaggtggtgcatggttgtcgtcagctcgtgtcgtgagatgttgggttaagtcccgcaacgagcgcaacccttgtccttagtagccagcagtaagatgggcactctagggagactgccggggataactcggaggaaggtggggacgacgtcaaatcatcatgccccttatgacttgggctacacacgtgctacaatggcgtaaacaaagggaagcgaacctgtgaaggcaagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgtaaatgcccgaagtcggtgacctaaccgtaaggaaggagccgccgaaggcaggtatgataactggggtgaagtcgtaacaaggtagccgtatcggaaggtgcggctggatcacctcctt

SEQ ID NO: 106 - DNA, 1445 bp, Clostridium hylemonae
  misc_feature: Clostridium hylemonae 16S NCBI Reference Sequence NR_024719.1
aggatgaacgctgccgccgtgcttaacacatgcaagtcgaacgaagcaatactgtgtgaagagattagcttgctaagatcagaactttgtattgactgagtggcggacgggtgagtaacgcgtgggcaacctgccttacacagggggataacagctagaaatggctgctaataccgcataagacctcagtaccgcatggtagaggggtaaaaactccggtggtgtaagatgggcccgcgtctgattaggtagttggtagggtaacggcctaccaagccgacgatcagtagccgacctgagagggtgaccggccacattggactgagacacggcccaaactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcatggcaagtctgaagtgaaagcccggggctcaaccccgggactgctttggaaactgtcaggctagagtgtcggagaggcaagtggaattcctagtgtagcggtgaaatgcgtagatattaggaggaacaccagtggcgaagcggcttgctggacgatgactgacgttgaggctcgaaagcgtggggagcaaacaggattagataccctggtagtccacgccgtaaacgatgattactaggtgtcgggaagcaaagcttttcggtgccgcagccaacgcaataagtaatccacctggggagtacgttcgcaagaatgaaactcaaaggaattgacggggacccgcacaagcggtggagcatgtggtttaattcgaagcaacgcgaagaaccttacctgatcttgacatcccggtgacaaagtatgtaacgtactctttcttcggaacaccggtgacaggtggtgcatggttgtcgtcagctcgtgtcgtgagatgttgggttaagtcccgcaacggcgcaacccttatctttagtagccagcatttgaggtgggcactctagagagactgccagggataacctggaggaaggtggggatgacgtcaaatcatcatgccccttatgaccagggctacacacgtgctacaatggcgtaaacaaagggaagcgaccctgtgaaggcaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatggggtcagtaacgcccgaagccggtgacctaaccgcaaaggaggagccgtcgaaggtg

SEQ ID NO: 107 - DNA, 1445 bp, Clostridium nexile; identical to SEQ ID NO: 106
  misc_feature: Clostridium nexile 16S NCBI Reference Sequence NR_024719.1

SEQ ID NO: 108 - DNA, 1529 bp, Clostridium scindens
gagagtttgatcctggctcaggatgaacgctggcggcgtgcctaacacatgcaagtcgaacgaagcgcctggccccgacttcttcggaacgaggagccttgcgactgagtggcggacgggtgagtaacgcgtgggcaacctgccttgcactgggggataacagccagaaatggctgctaataccgcataagaccgaagcgccgcatggcgcggcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattttggatgggcactctggagagactgccagggagaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagttggaatcgctagtaatcgcgaatcagaatgtcgcggtgaatacgttcccgggtcttgtacacaccgcccgtcacaccatgggagtcagtaacgcccgaagccggtgacccaacccgtaagggagggagccgtcgaaggtgggaccgataactggggtgaagtcgtaacaaggtagccgtatcggaaggtgcggctggatcacctccttc

SEQ ID NO: 109 - DNA, 1398 bp, Clostridium sporosphaeroides
  misc_feature: Clostridium sporosphaeroides 16S NCBI Reference Sequence NR_044835.2
  misc_feature (830)..(830): n is a, c, g, or t
  misc_feature (834)..(834): n is a, c, g, or t
  misc_feature (860)..(860): n is a, c, g, or t
  misc_feature (920)..(920): n is a, c, g, or t
  misc_feature (924)..(924): n is a, c, g, or t
  misc_feature (1139)..(1139): n is a, c, g, or t
  misc_feature (1167)..(1167): n is a, c, g, or t
  misc_feature (1270)..(1272): n is a, c, g, or t
  misc_feature (1276)..(1276): n is a, c, g, or t
  misc_feature (1291)..(1291): n is a, c, g, or t
  misc_feature (1373)..(1373): n is a, c, g, or t
  misc_feature (1378)..(1379): n is a, c, g, or t
tgaacttagcttagtggcggacgggtgagtaacgcgtgagtawcctgcctttcrgagggggatamcgttcctgaaaacaacgctaataccgcataatatattggtgtcgcatggcaccggtatcaaaggagcaatccgctgcaagatggactcgcgtccgatyagccagtkgatkgggtaaaggcccaccaaagcgacgatcggtagccgggttgagagactgaacggccacattgggactgagacacggcccagactcctacgggaggcagcagtgggggatattgcacaatggaggaaactctgatgcagcaatgccgcgtgagggaagacggtcttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggctctttaagtcgggcgtgaaagctgtgggcttaacccacaaattgcgttcgaaactggagggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacnagcngtggagtatgtggtttaattcgaagnaacgcgaagaaccttaccaggtgttgacatccaactaacgaggcagagatgcattaggtngctncggggaaagttgagacaggtggtccatggttgtcgtcagctcgtgtcgtgagatgttgggttaagtcccgcaacgagcgcaacccttgtgattagttstacgcaagagcactctaatcagactgccgttgacaaaacggaggaaggtggggacgacgtcaaatcatcatgccccttatgacctgggctacacacgtactacaatggtcgccaacngagggaagccaagccgcgaggtggagcnaacccccaaaagcgatctcagttcggattgtaggctgcaacccgcctacatgaagttggaattgctagtaatcgcggatcagcatgccgcggcgaatacgttnnngggncttgtacacaccgcncgtcacaccatgggagccggtaatacccgaagccaatagtctaaccgcaaggaggacgttgtcgaaggtaggattggcgacnggggnnaagtcgtaacaaggtaacc

SEQ ID NO: 110 - DNA, 1478 bp, Clostridium symbiosum
  misc_feature: Clostridium symbiosum 16S NCBI Reference Sequence NR_118730.1
  misc_feature (349)..(350): n is a, c, g, or t
  misc_feature (534)..(535): n is a, c, g, or t
  misc_feature (606)..(606): n is a, c, g, or t
  misc_feature (618)..(619): n is a, c, g, or t
  misc_feature (709)..(709): n is a, c, g, or t
  misc_feature (921)..(921): n is a, c, g, or t
  misc_feature (955)..(956): n is a, c, g, or t
  misc_feature (1012)..(1013): n is a, c, g, or t
  misc_feature (1015)..(1015): n is a, c, g, or t
  misc_feature (1081)..(1081): n is a, c, g, or t
  misc_feature (1185)..(1185): n is a, c, g, or t
  misc_feature (1240)..(1240): n is a, c, g, or t
  misc_feature (1391)..(1392): n is a, c, g, or t
  misc_feature (1470)..(1470): n is a, c, g, or t
  misc_feature (1474)..(1475): n is a, c, g, or t
aaacatgagagtttgatcctggctcaggatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtattgcatgatacagtgtgaaaaactccggtggtacaagatggacccgcgtctgattagctagttggtaaggtaacggcttaccaaggcgacgatcagtagccgacctgagagggtgaccggccacattgggactgagacacggccnnaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggnnagcgttatccggatttactgggtgtaaagggagcgtagacggtaaagcaagtctgaagtgaaagcccgcgnctcaactgcggnnctgctttggaaactgtttaactggagtgtcggagaggtaagtggaattcctagtgtagcggtgaaatgcgtagatattaggaggaacacnagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggaccngcacaagcggtggagcatgtggtttaattcgaannaacgcgaagaaccttaccaggtcttgacatcgactcgacgggggagtaacgtcccnntnccttcggggcggagaagacaggtggtgcatggttgtcgtcagctcgtgtcgtgagatgttgggttnagtcccgcaacgagcgcaacccttattctaagtagccagcggttcggccgggaactcttgggagactgccagggataacctggaggaaggtggggatgacgtcnaatcatcatgccccttatgatctgggctacacacgtgctacaatggcgtaaacanagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgnncgtcacaccatgggagtcagtaacgcccgaagtcagtgacccaaccgcaaggagggagctgccgaaggcgggaccganaacnnggg

SEQ ID NO: 111 - DNA, 1482 bp, Clostridium viride
  misc_feature: Clostridium viride 16S NCBI Reference Sequence NR_026204.1
  misc_feature (746)..(746): n is a, c, g, or t
gatgaacgctggcggcgtgcttaacacatgcaagtcgaacggagccatctggaacaagttttcggacaagggaaagatgtgcttagtggcggacgggtgagtaacgcgtgagtaacctgccttggagtggggaataacacatcgaaaggtgtgctaataccgcatgatgcaacgggatcgcatggttctgttgccaaagatttatcgctctgagatggactcgcgtctgattagctagttggtgaggtaatggctcaccaaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggccctcgggttgtaaacttcttttattcgagacgaaacaaatgacggtaccgaatgaataagccacggctaactacgtgccagcagccgcggtaatacgtaggtggcaagcgttatccggatttactgggtgtaaagggcgtgtaggcgggactgcaagtcagatgtgaaattccagggctcaactctggacctgcatttgaaactgtagttcttgagtgatggagaggcaggcggaattccgagtgtagcggtgaaatgcgtagatattcggaggaacaccagtggcgaaggcggcctgctggacattaactgacgctgaggcgcgaaagcgtggggagcanacaggattagataccctggtagtccacgctgtaaacgatggatactaggtgtggggggactgaccccttccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatccctctgaccggtctagagataggccctccctcggggcagaggtgacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtactacaatggcgcttaacagagggaggcaataccgcgaggtggagcaaacccctaaaaggcgtcccagttcggattgcaggctgaaacccgcctgtatgaagttggaatcgctagtaatcgcggatcagcatgccgcggtgaatacgttcccgggtcttgtacacaccgcccgtcacaccatgagagtcgggaacacccgaagtccgtagcctaacagcaatgagggcgcggccgaaggtgggttcgataattggggtgaagtcgtaacaaggtagccgtatcggaaggtgc

SEQ ID NO: 112 - DNA, 1315 bp, Coprococcus catus
  misc_feature: Coprococcus catus 16S NCBI Reference Sequence NR_024750.1
  misc_feature (142)..(142): n is a, c, g, or t
  misc_feature (544)..(545): n is a, c, g, or t
ggcgcgcctaacacatgcaagtcgaacggacgatgaagagcttgcttttcaaagttagtggcggacgggtgagtaacgcgtgggtaacctgcctcatacaggggaatagcagctggaaacggctgataaaaccgcataagcncacagtgccgcatggcacagtgtgaaaaactccggtggtatgagatggacccgcgtctgattagcttgttggcggggtaacggcccaccaaggcgacgatcagtagccggcctgagagggtgatcggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtaggcggcggagcaagtcagaagtgaaanncccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggccttgacatcccggtgaccgacccgtaatggggtcttctctacggagcactggagacaggtggtgcatggttgtcgtcagctcgtgtcgtgagatgttgggttaagtcccgcaacgagcgcaacccctatgttcagtagccagcatttaggatgggcactctggacagactgccggggataacccggaggaaggcggggatgacgtcaaatcatcatgccccttacggcctgggctacacacgtgctacaatggcgtaaacaaagggaagcaagagggtgacctggagcgaatcccataaataacgtcccagttcggactgtagtctgcaacccgactacacgaagctggaatcgctagtaatcgcgaatcagcatgtcgcggtgaatacg

SEQ ID NO: 113 - DNA, 1453 bp, Coprococcus comes
  misc_feature: Coprococcus comes 16S NCBI Reference Sequence NR_044048.1
gatgaacgctggcggcgtgcttaacacatgcaagtcgaacgaagcacttatmcttgattcttcggatgaagrggtttgtgactgagtggcggacgggtgagtaacgcgtgggtaacctgcctcatacagggggataacagttagaaatgactgctaataccgcataagaccacrgagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaaggtgggaccgataactggggtg

SEQ ID NO: 114 - DNA, 1440 bp, Coprococcus eutactus
  misc_feature: Coprococcus eutactus 16S NCBI Reference Sequence NR_044049.1
gatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacrgtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcraacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtg

SEQ ID NO: 115 - DNA, 714 bp, Coprococcus phoceensis
  misc_feature: Coprococcus phoceensis 16S NCBI Reference Sequence NZ_FNWC01000006.1
atgagtcagatatttgagaataagttaaaagagcttggcattgtattaaatgatgagcagaaacgtcaatttgattctttttatgaactcttggtggaatggaataaggtcatgaatctgactggtattaccgaatatgaagaggtaaatgaaaaacattttgtggatagcttgtcgataacaaaggcgattgacatgaattgtgtggataacttaattgatattggaacgggagctggttttccgggaattccgttaaagattgcatttcctcatctgaaagtagtattattggactcattaaataaaagaattaaatttttgaatacagtaattgaggaacttgaattgattgacatagaaacaattcacggaagagcagaagattttgcaaaacagggagagtatcgggaacaatttgatctttgtgtatcaagagcagtggcaaacttatccacactttctgagtattgcatgccatatatccacactgatggaatgtttatcccctacaagtctggagaaattgaggaagaggtgactggggcaaagaaagcagtgcatgtgcttggtggaaaaattgaggatgtggtgaagtttcaacttccgggaactgaaattggtcgttcttttgtaaagataaaaaaatatcaaaacacaccaaaaaaatatccgagaaaagcaggattacctgcaaaggagccaatagcataa

SEQ ID NO: 116 - DNA, 1479 bp, Dorea formicigenerans
  misc_feature: Dorea formicigenerans (Eubacterium formicigenerans) 16S NCBI Reference Sequence NR_044645.2
  misc_feature (123)..(125): n is a, c, g, or t
  misc_feature (220)..(222): n is a, c, g, or t
  misc_feature (287)..(287): n is a, c, g, or t
  misc_feature (349)..(350): n is a, c, g, or t
  misc_feature (517)..(517): n is a, c, g, or t
  misc_feature (534)..(535): n is a, c, g, or t
  misc_feature (725)..(725): n is a, c, g, or t
  misc_feature (918)..(918): n is a, c, g, or t
  misc_feature (921)..(921): n is a, c, g, or t
  misc_feature (955)..(956): n is a, c, g, or t
  misc_feature (1184)..(1185): n is a, c, g, or t
ttaaacgagagtttgatcctggctcaggatgaacgctggcggcgtgcttaacacatgcaagtcgagcgaagcacataagtttgattcttcggatgaagacttttgtgactgagcggcggacgnnngagtaacgcgtgggtaacctgcctcatacagggggataacagytagaaatggctgctaataccgcataagaccacagtactgcatggtacagtgnnnaaaactccggtggtatgagatggacccgcgtctgattaggtagttggtgaggtaacggcccaccnagccgacgatcagtagccgacctgagagggtgaccggccacattgggactgagacacggccnngactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgnggtaatacgtagggggnnagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcntactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggnccngcacaagcggtggagcatgtggtttaattcgaannaacgcgaagaaccttacctgatcttgacatcccgatgaccgcttcgtaatggaagyttttcttcggaacatcggtgacaggtggtgcatggttgtcgtcagctcgtgtcgtgagatgttgggttaagtcccgcaacgagcgcaacccttatcttcagtagccagcatttaggatgggcactctggagagactgccagggataacctggaggaaggtggggatgacgtnnaatcatcatgccccttatgaccagggctacacacgtgctacaatggcgtaaacagagggaggcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgaaaggagggagctgccgaaggtgggaccgataactggggt

SEQ ID NO: 117 - DNA, 1316 bp, Dorea longicatena
  misc_feature: Dorea longicatena 16S NCBI Reference Sequence NR_028883.1
taacgcgtgggtaacctgcctcatacagggggataacagttagaaatgactgctaataccgcataagaccacgtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcataacgcccgaagtcagtgacccaaccgtaagg

SEQ ID NO: 118 - DNA, 1390 bp, Eisenbergiella tayi
  misc_feature: Eisenbergiella tayi 16S NCBI Reference Sequence NR_118643.1
ggtataacttagtggcggacgggtgagtaacgcgtgggaaacctgccctgtaccgggggataacacttagaaataggtgctaataccgcataagcgcacggaaccgcatggttccgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgcacctgtaaagaggtgttcccttcggggcattggagacaggtggtgcatggttgtcgtcagctcgtgtcgtgagatgttgggttaagtcccgcaacgagcgcaacccttattcttagtagccagcaggtaaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcycagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgtaa

SEQ ID NO: 119 - DNA, 1395 bp, Enterocloster aldenensis
  misc_feature: Enterocloster aldenensis (Clostridium aldenense) 16S NCBI Reference Sequence NR_043680.1
tcctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgcaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgttgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggcacgtaacggtgcccttccttcgggacaggggagacaggtggtgcatggttgtcgtcagctcgtgtcgtgagatgttgggttaagtcccgcaacgagcgcaacccttatccttagtagccagcacgtcgtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccttaacaggagggagctgccgaaggcggggcaggtaactggggtgaagtcgtaac

SEQ ID NO: 120 - DNA, 1468 bp, Enterocloster asparagiformis
  misc_feature: Enterocloster asparagiformis (Clostridium asparagiforme) 16S NCBI Reference Sequence NR_042200.1
  misc_feature (831)..(840): n is a, c, g, or t
tggcggcgtgcctaacacatgcaagtcgaacgaagcattttagatgaagttttcggatggattctagatgactgagtggcggacgggtgagtaacacgtggataacctgcctcacactgggggacaacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcattnnnnnnnnnnagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaaggcagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtaaccgta

SEQ ID NO: 121 - DNA, 1498 bp, Enterocloster bolteae
  misc_feature: Enterocloster bolteae (Clostridium bolteae) 16S NCBI Reference Sequence NR_025567.1
agagtttgatcctggctcaggatgaacgctggcggcgtgcctaacacatgcaagtcgaacgaagcaattaaaatgaagttttcggatggatttttaattgactgagtggcggacgggtgagtaacgcgtggataacctgcctcacactgggggataacagttagaaatgactgctaataccgcataagcgcacagtgccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccgcctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaaggcagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtaaccgta

SEQ ID NO: 122 - DNA, 1420 bp, Enterocloster citroniae
  misc_feature: Enterocloster citroniae (Clostridium citroniae) 16S NCBI Reference Sequence NR_043681.1
cgtgcctaacacatgcaagtcgaacgaagcaatcagaatgaaattttcggatggatttctgggtgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaactgacggtacctgactaagaagccccggctaactacgtgccagcagccgcggtaatacgtagggggcaagcgttatccggatttactgggtgtaaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggc

SEQ ID NO: 123 - DNA, 1382 bp, Enterocloster clostridioformis
  misc_feature: Enterocloster clostridioformis (Clostridium clostridioforme) 16S NCBI Reference Sequence NR_171498.1
tgcaagtcgagcgagagaaatcccttcggggatggatctagcggcggacgggtgagtaacacgtgggcaacctgcctcaaagagggggatagcctcccgaaagggagattaataccgcataatgttgttttactgcatagtaaagcaaccaaaggagtaatccactttgagatgggcccgcgccgcattagttagttggtggggtaacggctcaccaaggcgacgatgcgtagccgacctgagagggtgatcggccacattggaactgagagacggtccagactcctacgggaggcagcagtggggaatattgcacaatgggcggaagcctgatgcagcaacgccgcgtgggtgatgaaggttttcggattgtaaagccctgtcttttgggacgataatgacggtaccaaaggaggaagccacggctaactacgtgccagcagccgcggtaatacgtaggtggcgagcgttgtccggatttactgggcgtaaagggtgcgtaggcggatatttaagtgggatgtgaaaaccccgggcttaacccggggactgcatttcaaactggatatctggagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatgagtactaggtgtaggaggtatcgaccccttctgtgccgcagtaaacgcaataagtactccgcctgggaagtacgatcgcaagattaaaactcaaaggaattgacgggggcccgcacaagcagcggagcatgtggtttaattcgaagcaacgcgaagaaccttacctagacttgacatcccctgaattacctgtaattagggaagccctccggggcagggagacaggtggtgcatggttgtcgtcagctcgtgtcgtgagatgttaggttaagtcctgcaacgagcgcaacccctatcattagttgctaccattaagttgagcactctagtgagactgcccgggtcaaccgggaggaaggtggggatgacgtcaaatcatcatgccccttatgtctagggctacacacgtgctacaatggtgaatacagagagatgcaaacccgcaagggtgagccaaactttaaaattcacctcagttcggattgcaggctgaaacccgcctgcatgaagctggagttgctagtaatcgcgaatcagaatgtcgcggtgaatgcgttcccgggccttgtacacaccgcccgtcacaccatgagagttggcaacacccgaagtccgtggggtaaccgtaaggaaccagcggcc

SEQ ID NO: 124 - DNA, 1454 bp, Enterocloster lavalensis
  misc_feature: Enterocloster lavalensis (Clostridium lavalense) 16S NCBI Reference Sequence NR_044289.1
gatgaacgctggcggcgtgcctaacacatgcaagtcgaacgaagcattttraatgaagttttcggatggattytgagatgactgagtggcggacgggtgagtaacacgtggataacctgcctcacactgggggacaacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagrgagacaggtggtgcatggttgtcgtcagctcgtgtcgtgagatgttgggttaagtcccgcaacgagcgcaacccttatccttagtagccagcartragatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcargagagggakctgccgaasgcggcgcakgtaactggggtg

SEQ ID NO: 125 - DNA, 1453 bp, Eubacterium siraeum
  misc_feature: Eubacterium siraeum 16S NCBI Reference Sequence NR_118675.1
  misc_feature (519)..(519): n is a, c, g, or t
  misc_feature (562)..(563): n is a, c, g, or t
  misc_feature (716)..(716): n is a, c, g, or t
  misc_feature (904)..(904): n is a, c, g, or t
  misc_feature (935)..(935): n is a, c, g, or t
  misc_feature (938)..(939): n is a, c, g, or t
  misc_feature (1150)..(1150): n is a, c, g, or t
  misc_feature (1440)..(1440): n is a, c, g, or t
ttaacaaagagtttgatcctggctcaggacgaacgctggcggcgcgcctaacacatgcaagtcgaacggtgaagaggagcttgctcctcggatcagtggcggacgggtgagtaacacgtgagcaacctggctctaagagggggacaacagttggaaacgactgctaataccgcatraygtatcgggatggcatcttcctgataccaaagattttatcgcttagagatgggctcgcgtctgattagatagttggcggggtaacggcccaccaagtcgacgatcagtagccggactgagaggttgaacggccacattgggactgagacacggcccagactcctacgggaggcagcagtgggggatattggacaatgggggcaaccctgatccagcgacgccgcgtgagggaagaaggttttcggattgtaaacctctgttgacggagaaaaaaatgatggtatccgtttagaaagccacggctaactacgtgccagcagccgcggtaatacgtaggtggcnagcgttgtccggaattactgggtgtaaagggagtgtaggcggnntatcaagtcagaagtgaaaattacgggctcaactcgtaacctgcttttgaaactgacattcttgagtgaagtagaggcaagcggaattcctagtgtagcggtgaaatgcgtagatattaggaggaacaccagtggcgaaggcggcttgctggncttttactgacgctgaggctcgaaagcgtggggagcaaacaggattagataccctggtagtccacgccgtaaacgatgattactaggtgtggggggattgaccccttcgtgccggagtaaacacaataagtaatccacctggggagtacgaccgcaaggttgaaactcaaaggaattgacgggggccngcacaagcagtggagtatgtggtttaattcnctnnaacgcgaagaaccttaccaggtcttgacatcgagtgaccgcctaagagattaggctttcccttcggggacacaaagacaggtggtgcatggttgtcgtcagctcgtgtcgtgagatgttgggttaagtcccgcaacgagcgcaacccttatcattagtygtacgcaagagcactctaatgagactgccgttgacaaaacggaggaaggtgnggatgacgtcaaatcatcatgccctttatgacctgggctacacacgtactacaatggcgtttaacaaagagaagcaaagccgcgaggcagagcaaatctccaaaaaacgtctcagttcggattgtaggctgcaactcgcctacatgaagtcggaattgctagtaatcgtaggtcagcatactacggtgaatacgttcccgggccttgtacacaccgcccgtcaaaccatgagagttggcaacacccgaagtcggtagtctaaccgcaaggaggacgccgccgaaggtggngttgatgattagg

SEQ ID NO: 126 - DNA, 1481 bp, Eubacterium ventriosum
  misc_feature: Eubacterium ventriosum 16S NCBI Reference Sequence NR_118670.1
  misc_feature (351)..(352): n is a, c, g, or t
  misc_feature (535)..(537): n is a, c, g, or t
  misc_feature (608)..(608): n is a, c, g, or t
  misc_feature (920)..(920): n is a, c, g, or t
  misc_feature (924)..(924): n is a, c, g, or t
  misc_feature (958)..(959): n is a, c, g, or t
  misc_feature (1136)..(1136): n is a, c, g, or t
  misc_feature (1243)..(1243): n is a, c, g, or t
  misc_feature (1394)..(1394): n is a, c, g, or t
tttaacgagagtttgatcctggctcaggatgaacgctggcggcgtgcttaatacatgcaagtcgaacgaagcaccttggacagaatccttcgggaggaagaccattgtgactgagtggcggacgggtgagtaacgcgtgggtaacctgccttgtacagggggataacagttggaaacgactgctaataccgcataagcgcacagtaccgcatggtacggtgtgaaaaactccggtggtacaagatggacccgcgtctgattagctggttggtgaggtaacggcccaccaaggcgacgatcagtagccgacttgagagagtgatcggccacattgggactgagacacggccnnaactcctacgggaggcagcagtagggaatattgcacaatgggggaaaccctgatgcagcgacgccgcgtgaaggaagaagtatttcggtatgtaaacttctatcagcaaggaagaaaatgacggtacttgactaagaagccccggctaaatacgtgccagcagccgcggtaatacgtatgggnnnagcgttatccggatttactgggtgtaaagggagcgtaggcggcatggcaagtcagaagtgaaagcctgggnctcaaccccggaattgcttttgaaactgtcaggctagagtgtcggaggggtaagcggaattcctagtgtagcggtgaaatgcgtagatattaggaggaacaccggtggcgaaggcggcttactggacgattactgacgctgaggctcgaaagcgtggggagcaaacaggattagataccctggtagtccacgccgtaaacgatgaatactaggtgtcgggggacaatagttcctcggtgccgaagcaaacgcattaagtattccacctggggagtacgttcgcaagaatgaaactcaaaggaattgacgggnaccngcacaagcggtggagcatgtggtttaattcgaannaacgcgaagaaccttacctgctcttgacatcccactgacaggtcagtaatgtgaccctttcttcggaacagtggagacaggtggtgcatggttgtcgtcagctcgtgtcgtgagatgttgggttaagtcccgcaacgagcgcaacccttgtctttagtagccagcagtacggctggncactctagagagactgccagggataacctggaggaaggcggggatgacgtcaaatcatcatgccccttacgagcagggctacacacgtgctacaatggcgtaaacanagggaagcgaccccgtgagggtaagcaaatctcaaaaataacgtctcagttcggattgtagtctgcaactcgactacatgaagctggaatcgctagtaatcgcgaatcagaatgtcgcggtgaatacgttcccgggtcttgtacacaccgnccgtcacaccatgggagttggatatgcccgaagtcagtgacccaaccgtaaggagggagctgccgaaggtggagccgataactgggg

SEQ ID NO: 127 - DNA, 1521 bp, Faecalicatena contorta
  misc_feature: Faecalicatena contorta (Eubacterium contortum) 16S NCBI Reference Sequence NR_104803.1
gatcctggctcaggatgaacgctggcggcgtgcttaacacatgcaagtcgagcgaagcgctttacttagatttcttcggattgaagagttttgcgactgagcggcggacgggtgagtaacgcgtgggtaacctgcctcatacagggggataacagttagaaatgactgctaataccgcataagaccacggtaccgcatggtacagtgggaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggttatgtaagtctgatgtgaaaacccggggctcaaccccgggactgcattggaaactatgtaactagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctgctcttgacatccccctgaccggcgtgtaatggtgcctttccttcgggacaggggagacaggtggtgcatggttgtcgtcagctcgtgtcgtgagatgttgggttaagtcccgcaacgagcgcaacccttatctttagtagccagcggtttggccgggcactctagagagactgccagggataacctggaggaaggtggggatgacgtcaaatcatcatgccccttatgagcagggctacacacgtgctacaatggcgtaaacaaagggaggcgaagccgtgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggtgggaccgataactggggtgaagtcgtaacaaggtagccgtatcggaaggtgcggctggatcacctcctttct

SEQ ID NO: 128 - DNA, 1522 bp, Faecalicatena fissicatena
  misc_feature: Faecalicatena fissicatena (Eubacterium fissicatena) 16S NCBI Reference Sequence NR_104800.1
tttgatcctggctcaggatgaacgctggcggcgtgcttaacacatgcaagtcgagcgaagcactttactttgatttcttcggaatgaagagtgttgtgactgagcggcggacgggtgagtaacgcgtgggtaacctgcctcatacagggggataacagttagaaatgactgctaataccgcataagaccacagtaccgcatggtacagtgggaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggttatgtaagtctgatgtgaaaacccggggctcaaccccgggactgcattggaaactatgtaactagagtgtcggagaggtaagtggaattcctagtgtagcggtgaaatgcgtagatattaggaggaacaccagtggcgaaggcggcttactggacgatcactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctgctcttgacatcccactgaccggcacgtaatggtgccttcccttcggggcagtggagacaggtggtgcatggttgtcgtcagctcgtgtcgtgagatgttgggttaagtcccgcaacgagcgcaacccttatctttagtagccagcggtttggccgggcactctagagagactgccagggataacctggaggaaggtggggatgacgtcaaatcatcatgccccttatgagcagggctacacacgtgctacaatggcgtaaacaaagggaggcaataccgcgaggttgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggcgggatcgataactggggtgaagtcgtaacaaggtagccgtatcggaaggtgcggctggatcacctccttt

SEQ ID NO: 129 - DNA, 1482 bp, Faecalicatena orotica
  misc_feature: Faecalicatena orotica (Clostridium oroticum) 16S NCBI Reference Sequence NR_118729.2
  misc_feature (538)..(539): n is a, c, g, or t
  misc_feature (604)..(609): n is a, c, g, or t
  misc_feature (832)..(832): n is a, c, g, or t
tttaacgagagtttgatcctggctcaggatgaacgctggcggcgtgcttaacacatgcaagtcgagcgaagcacatttacctttgatttcttcggattgaagagtgttgtgactgagcggcgggcgggtgagtaacgcgtgggtaacctgcctcatacagggggataacagttagaaatgactgctaataccgtataagaccacagtgccgcatggcacagtggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggnnagcgttatccggatttactgggtgtaaagggagcgtagacggtaatgcaagtctgatgtgaaacnnnnnngctcaaccccgatactgcattggaaactgtgtgactagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgaatactaggtgtcgggtngcaaagccattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctgctcttgacatcccgctgaccggccagtaatgtggccttcccttcggggcagtggagacaggtggtgcatggttgtcgtcagctcgtgtcgtgagatgttgggttaagtcccgcaacgagcgcaacccttatctttagttgccagcggcttggccgggcactctagagagactgccagggataacctggaggaaggtggggatgacgtcaaatcatcatgccccttatgagcagggctacacacgtgctacaatggcgtaaacaaagggaggcaatactgtgaagtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggtgggaccgataactgggg

SEQ ID NO: 130 - DNA, 1550 bp, Flavonifractor plautii
ggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggtaattggggtgaagtcgtaacaaggtagccgtatcggaaggtgcggctgg

SEQ ID NO: 131 - DNA, 1409 bp, Hungatella effluvia
  misc_feature: Hungatella effluvia 16S NCBI Reference Sequence NR_133762.1
atgcagtcgagcgaagcgattctctaggaagttttcggatggaataggatttgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtgtttcggcatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaaagtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccggtatccctcttcggagcaatggagacaggtggtgcatggttgtcgtcagctcgtgtcgtgagatgttgggttaagtcccgcaacgagcgcaacccttatccttagtagccagcacataatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggcg

SEQ ID NO: 132 - DNA, 1431 bp, Hungatella hathewayi
  misc_feature: Hungatella hathewayi (Clostridium hathewayi) 16S NCBI Reference Sequence NR_036928.1
  misc_feature (191)..(191): n is a, c, g, or t
  misc_feature (211)..(211): n is a, c, g, or t
  misc_feature (248)..(248): n is a, c, g, or t
  misc_feature (979)..(979): n is a, c, g, or t
ctcaggatgaacgctggcggcgtgcttaacacatgcaagtcgagcgaagcggtttcaatgaagttttcggatggatttgaaattgacttagcggcggacgggtgagtaacgcgtgggtaacctgccttacactgggggataacagttagaaatgactgctaataccgcataagcgcacagggccgcatggnctggtgtgaaaaactccggnggtgtaagatggacccgcgtctgattaggtagttggnggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaaaacacnttaaccgtgatccctcttcggagcagtggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaggcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgaaaggagggagct

SEQ ID NO: 133 - DNA, 1480 bp, Hungatella xylanolytica
  misc_feature: Hungatella xylanolytica (Bacteroides xylanolyticus) 16S NCBI Reference Sequence NR_104899.1
gatgaacgctggcggcgtgcttaacacatgcaagtcgagcgaagcgattttaaggaagttttcggatggaattaaaattgacttagcggcggacgggtgagtaacgcgtgggtaacctgcctcatacagggggataacagttagaaatgactgctaataccgcataagcgcacagtgccacatggcacagtgtgaaaaactccggtggtatgagatggacccgcgtctgattaggtagttggtgaggtaatggctcaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtgtttcggcatgtaaagctctatcagcagggaagaaaatgacggtacctgactaagaagccccggctaactacgtgccagcagccgcggtaatacgtagggggcaagcgttatccggatttactgggtgtaaagggagcgtagacggttctgcaagtctggagtgaaagcccggggctcaaccccgggactgctttggaaactgtggaactagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccgtcccgtaacgggggcttcccttcggggcattccagacaggtggtgcatggttgtcgtcagctcgtgtcgtgagatgttgggttaagtcccgcaacgagcgcaacccttatccttagtagccagcagttcggctgggcactctggggagactgccagggataacctggaggaaggtggggatgacgtcaaatcatcatgccccttatgatttgggctacacacgtgctacaatggcgtaaacaaagggaagcgaaggagtgatctggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagttggtaacgcccgaagtcagtgacccaaccgyaaggagggagctgccgaaggcgggactgataactggggtgaagtcgtaacaaggtagccgtatcgga

SEQ ID NO: 134 - DNA, 1860 bp, Lachnospira eligens
  misc_feature: Lachnospira eligens (Eubacterium eligens) 16S NCBI Reference Sequence NR_074613.1
gagagtttgatcctggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtggcctttccttcgggacaagagagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctaaggtggggatgacgtcaaatcatcatgccccttatgatttgggctacacacgtgctacaatggcgtaaacaaagggaagcgaaggagtgatctggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagttggtaacgcccgaagtcagtgacccaaccgyaaggagggagctgccgaaggcgggactgataactggggtgaagtcgtaacaaggtagccgtatcgga

SEQ ID NO: 135 - DNA, 1518 bp, Lachnospira multipara
  misc_feature: Lachnospira multipara 16S NCBI Reference Sequence NR_104758.1
tttgatcctggctcaggatgaacgctggcggcgtgcttaacacatgcaagtcgaacgaagcaacwtatcacgattccttcgggatgacgatttgttgactgagtggcggacgggtgagtaacgcgtgggtaacctgccttatacagggggatagcagctggaaacggctgataataccgcataagcgcacggcatcgcatgatgcagtgtgaaaaactccggtggtataagatggacccgcgtctgattagctagttggtgaggtaacggcccaccaaggcaacgatcagtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggagtgtaggtggcaaagcaagtcagtagtgaaaatccggggctcaacctcggaactgctattgaaactgtttagctagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactaggtgttgggtctcataagagattcggtgccgcagctaacgcaataagtattccacctggggagtacgttcgcaagaatgaaactcaaaggaattgacggggacccgcacaagcggtggagcatgtggtttaattcgaagcaacgcgaagaaccttacctagtcttgacatcccgatgaccragtatgtaatgtactctttcttcggaacatcggtgacaggtggtgcatggttgtcgtcagctcgtgtcgtgagatgttgggttaagtcccgcaacgagcgcaacccctatttctagtagccagcagttcggctgggcactctagagagactgccagggataacctggaggaaggtggggatgacgtcaaatcatcatgccccttatgactagggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcct

SEQ ID NO: 136 - DNA, 1465 bp, Lachnospira pectinoschiza
  misc_feature: Lachnospira pectinoschiza 16S NCBI Reference Sequence NR_118667.1
  misc_feature (28)..(28): n is a, c, g, or t
  misc_feature (72)..(73): n is a, c, g, or t
  misc_feature (79)..(79): n is a, c, g, or t
  misc_feature (85)..(85): n is a, c, g, or t
  misc_feature (101)..(102): n is a, c, g, or t
  misc_feature (108)..(108): n is a, c, g, or t
  misc_feature (203)..(203): n is a, c, g, or t
  misc_feature (350)..(350): n is a, c, g, or t
  misc_feature (432)..(432): n is a, c, g, or t
  misc_feature (516)..(516): n is a, c, g, or t
  misc_feature (535)..(535): n is a, c, g, or t
  misc_feature (617)..(618): n is a, c, g, or t
  misc_feature (642)..(642): n is a, c, g, or t
  misc_feature (682)..(682): n is a, c, g, or t
  misc_feature (1201)..(1201): n is a, c, g, or t
  misc_feature (1203)..(1203): n is a, c, g, or t
  misc_feature (1264)..(1264): n is a, c, g, or t
  misc_feature (1376)..(1377): n is a, c, g, or t
  misc_feature (1391)..(1391): n is a, c, g, or t
  misc_feature (1394)..(1394): n is a, c, g, or t
  misc_feature (1398)..(1398): n is a, c, g, or t
  misc_feature (1401)..(1401): n is a, c, g, or t
  misc_feature (1432)..(1432): n is a, c, g, or t
  misc_feature (1453)..(1453): n is a, c, g, or t
tatgagagtttgatcctggctcaggatnaacgctggcggcgtgcttaacacatgcaagtcgaacgaagcatnnaagacnrgattnyttcggratgaagtcnnttatgnctgagtggcggacgggtgagtaacgcgtgggtaacctgcctcacacagggggatagcagttggaaacggctgataataccgcataagcgcacagnaccgcatggtacagtgtgaaaaactccggtggtgtgagatggacccgcgtctgattagctggttggcagggtaacggcctaccaaggcaacgatcagtagccgacctgagagggtgaccggccacattgggactgagacacggcccngactcctacgggaggcagcagtggggaatattgcacaatggaggaaactctgatgcagcgacgccgcgtgagtgaagaagtnattcgttatgtaaagctctatcagcagggaagatagtgacggtacctgactaagaagctccggctaaatacgtgccagcagccncggtaatacgtatggagcnagcgttatccggatttactgggtgtaaagggagtgtaggtggcatcacaagtcagaagtgaaagcccggggctcaaccccgnnactgcttttgaaactgtggagctngagtgcaggagaggcaagtggaattcctagtgtagcggtnaaatgcgtagatattaggaggaacaccagtggcgaaggcggcttgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccrcctggggagtacgttcgcaagaatgaaactcaaaggaattgacggggacccgcacragcggtggagcatgtggtttaattcgaagcaacgcgaagaaccttaccaagtcttgacatcctccttgcccggtcagtaatgtgaccttttcttcggaacaagagtgacaggtggtgcatggttgtcgtcagctcgtgtcgtgagatgttgggttaagtcccgcaacgagcgcaacccctattcttagtagccagcatttaaggtgggcactctaggaagactgccagggataacctggaggaaggtggggatgacgtcaaatcatcatgcccntnatgacttgggctacacacgtgctacaatggcgtaaacaaagtgaagcgagagtgtgagctnaagcaaatcacaaaaataacgtctcagttcggattgtagtctgcaactcgactacatgaagctggaatcgctagtaatcgcgaatcagaatgtcgcggtgaatacgttcccnngtcttgtacacacngcncgtnacnccatgggagtcggaaatgcccgaagtcggtnacctaacgaaagaaggagccnccgaaggcaggt

SEQ ID NO: 137 - DNA, 1470 bp, Lacrimispora aerotolerans
  misc_feature: Lacrimispora aerotolerans (Clostridium aerotolerans) 16S NCBI Reference Sequence NR_119068.1
  misc_feature (1082)..(1083): n is a, c, g, or t
  misc_feature (1085)..(1089): n is a, c, g, or t
cgcgtgcttaacacatgcaagtcgagcgaagcattttaaaggaagttttcggatggaatttaagatgacttagcggcggacgggtgagtaacgcgtgggtaacctgcctcatacagggggataacagttagaaatgactgctaataccgcataagcgcacagtgctgcatagcacagtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggctctgcaagtctggagtgaaagccggggctcaaccccgggactgctttggaaactgtggagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccgtctcgtaacggagacctcccttcggggcattccagacaggtggtgcatggttgtcgtcagctcgtgtcgtgagatgttgggttaagtcccgcaacgagcgcaacccttatccttagtagccagcanntnnnnntgggcactctggggagactgccagggataacctggaggaaggtggggatgacgtcaaatcatcatgccccttatgatttgggctacacacgtgctacaatggcgtaaacaaagggaagcaaaggagcgatctggagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaagg

SEQ ID NO: 138 - DNA, 1502 bp, Lacrimispora algidixylanolytica
  misc_feature: Lacrimispora algidixylanolytica (Clostridium algidixylanolyticum) 16S NCBI Reference Sequence NR_028726.1
agagtttgatcctggctcaggatgaacgctggcggcgtgcttaacacatgcaagtcgagcgaagcgattttaaggaagccttcgggtggaattagaattgacttagcggcggacgggtgagtaacgcgtgggtaacctgcctcatacagggggataacagttagaaatgactgctaataccgcataagcgcacagtgctgcatagcacagtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgactgaagaagtatttcggtatgtaaaggtctatcagcagggaagaaaatgacggtacctgactaagaagccccggctaactacgtgccagcagccgcggtaatacgtagggggcaagcgttatccggatttactgggtgtaaagggagcgtagacggctctgcaagtctggagtgaaagcccggggctcaaccccgggactgctttggaaactgtggagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggagcaaagctcttcggtgccgtcgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgacagccttataactaaggtctccctacggggcattctagacaggtggtgcatggttgtcgtcagctcgtgtcgtgagatgttgggttaagtcccgcaacgagcgcaacccttatccttagtagccagcagttcggctgggcactctggggagactgccagggataacctggaggaaggtggggatgacgtcaaatcatcatgccccttatgatttgggctacacacgtgctacaatggcgtaaacaaagggaagcgaaggagcgatctggagcaaaccccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaga

SEQ ID NO: 139 - DNA, 1485 bp, Lacrimispora amygdalina
  misc_feature: Lacrimispora amygdalina (Clostridium amygdalinum) 16S NCBI Reference Sequence NR_115211.1
ctcaggatgaacgctggcggcgtgcttaacacatgcaagtcgagcgaagcagttttaaggaagttttcggatggaattaaaattgactgagcggcggacgggtgagtaacgcgtgggtaacctgcctcatacagggggataacagttggaaacgactgctaataccgcataagcgcacagtgctgcatggcacggtgtgaaaaactccggtggtatgagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgactgaagaagtatttcggtatgtaaaggtctatcagcagggaagaaaatgacggtacctgactaagaagccccggctaactacgtgccagcagccgcggtaatacgtagggggcaagcgttatccggatttactgggtgtaaagggagcgtagacggcgatgcaagtctggagtgaaagcccggggctcaaccccgggactgctttggaaactgtgttgctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccggtccgtaacggggccttcccttcggggcattccagacaggtggtgcatggttgtcgtcagctcgtgtcgtgagatgttgggttaagtcccgcaacgagcgcaacccttatccttagtagccagcagtacggctgggcactctggggagactgccagggataacctggaggaaggtggggatgacgtcaaatcatcatgccccttatgatttgggctacacacgtgctacaatggcgtaaacaaagggaagcgaaggagtgatccggagcaaatctcaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcgga

SEQ ID NO: 140 - DNA, 1499 bp, Lacrimispora celerecrescens
  misc_feature: Lacrimispora celerecrescens (Clostridium celerecrescens) 16S NCBI Reference Sequence NR_026100.1
  misc_feature (3)..(3): n is a, c, g, or t
  misc_feature (48)..(48): n is a, c, g, or t
  misc_feature (1118)..(1118): n is a, c, g, or t
acnctggcggcgtgcttaacacatgcaagtcgagcgaagcgatttcanggaagttttcggacggaattgaaattgactgagcggcggacgggtgagtaacgcgtgggtaacctgcctcatacagggggataacagttggaaacgactgctaataccgcataagcacacagtgccgcatggtacggtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcactgcaagtctggagtgaaagcccggggctcaaccccgggactgctttggaaactgtggtgctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccgggatgtaacgatcccttcccttcggggcattccagacaggtggtgcatggttgtcgtcagctcgtgtcgtgagatgttgggttaagtcccgcaacgagcgcaacccttatccttagtagccagcaggtcaagctgggcactctggggagactnccagggataacctggaggaaggtggggatgacgtcaaatcatcatgccccttatgatttgggctacacacgtgctacaatggcgtaaacaaagggaggcaaaggagcgatctggagcaaaccccaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagttggtaacgcccgaagtcagtgacccacccgtaaggagggagctgccgaaggcgggactgataactggggtgaagtcgtaacaaggtagccgtatcggaaggtgcggctggatcacctcctt

SEQ ID NO: 141 - DNA, 1488 bp, Lacrimispora indolis
  misc_feature: Lacrimispora indolis (Clostridium indolis) 16S NCBI Reference Sequence NR_026493.1
cctggctcaggatgaacgctggcggcgtgcttaacacatgcaagtcgagcgaagcgatttaaatgaagttttcggatggaatttaaattgactgagcggcggacgggtgagtaacgcgtgggtaacctgcctcatacagggggataacagttggaaacgactgctaataccgcataagcacacagtgccgcatggtacggtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtgtttcggcatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctggagtgaaagcccggggctcaaccccgggactgctttggaaactgtgttgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccggtccgtaacggggccttcccttcggggcattccagacaggtggtgcatggttgtcgtcagctcgtgtcgtgagatgttgggttaagtcccgcaacgagcgcaacccttatccttagtagccagcaagtcaagttgggcactctggggagactgccagggataacctggaggaaggtggggatgacgtcaaatcatcatgccccttatgatttgggctacacacgtgctacaatggcgtaaacaaagggaagcaaaggagtgatctggagcaaaccccaaaaataacgtctcacttcggattgtagtctgcaactcgactacatgaagctggaatcgctagtaatcgcggatcagaatgccgcggtgaatacgttcccgggtcttgtacacaccgcccgtcacaccatgggagttggtaacgcccgaagtcagtgacccaaccgcaaggagggagctgccgaaggcgggactgataactggggtgaagtcgtaacaaggtagccgtatc

SEQ ID NO: 142 - DNA, 1499 bp, Lacrimispora saccharolytica
  misc_feature: Lacrimispora saccharolytica (Clostridium saccharolyticum) 16S NCBI Reference Sequence NR_026494.1
tggctcaggatgaacgctggcggcgtgcttaacacatgcaagtcgagcgaagcacttttaaggaagttttcggatggaattaaaagtgactgagcggcggacgggtgagtaacgcgtgggtaacctgcctcatacagggggataacagttggaaacggctgctaataccgcataagcgcacagtgctgcatggcacagtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcactgcaagtctggagtgaaagcccggggctcaaccccgggactgctttggaaactgtggtgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccggtccgtaacggggccttcccttcggggcattccagacaggtggtgcatggttgtcgtcagctcgtgtcgtgagatgttgggttaagtcccgcaacgagcgcaacccttatccttagtagccagcagttcggctgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaaggtgcggctg

SEQ ID NO: 143 - DNA, 1495 bp, Lacrimispora sphenoides
  misc_feature: Lacrimispora sphenoides (Bacillus sphenoides) 16S NCBI Reference Sequence NR_026409.1
gatcctggctcaggatgaacgctggtggcgtgcttaacacatgcaagtcgagcgaagcgattcaaatgaagttttcggatggattttgaattgacttagcggcggacgggtgagtaacgcgtgggtaacctgcctcatacagggggataacagtcggaaacgactgctaataccgcataagcacacagtgccgcatggtacggtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcactgcaagtctggagtgaaagcccggggctcaaccccgggactgctttggaaactgtggtgctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccgggatgtaacgatcccttccctacggggcattccagacaggtggtgcatggttgtcgtcagctcgtgtcgtgagatgttgggttaagtcccgcaacgagcgcaacccttatccttagtagccagcaagtcaagttgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaa

SEQ ID NO: 144 - DNA, 1441 bp, Lacrimispora xylanolytica
  misc_feature: Lacrimispora xylanolytica (Clostridium xylanolyticum) 16S NCBI Reference Sequence NR_037068.1
  misc_feature (830)..(830): n is a, c, g, or t
  misc_feature (1001)..(1001): n is a, c, g, or t
  misc_feature (1386)..(1386): n is a, c, g, or t
gatcctggctcaggatgaacgctggcggcgtgcttaacacatgcaagtcgagcgaagcattttaaaggaagttttcggatggaatttagaatgacttagcggcggacgggtgagtaacgcgtgggtaacctgcctcatacagggggataacagttagaaatgactgctaataccgcataagcgcacagtgctgcatagtacagtgtgaaaaactccggtggtatgagatggacccgcgtctgattaggtagttggtgaggtaacggcccaccaagccgacgatcagtagccgacctgagagggtgaccggccacattgggactgagacacggcccaaactcctacgggaggcagcagtggggaatattggacaatgggggaaaccctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggctctgcaagtctggagtgaaagcccggggctcaaccccgggactgctttggaaactgtggagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgttggggagcaaagctcttcggtnccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccgtctcgtaacggagacctnccttcggggcattccagacaggtggtgcatggttgtcgtcagctcgtgtcgtgagatgttgggttaagtcccgcaacgagcgcaacccttatccttagtagccagcatgtwatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacnccatgggagttggtaacgcccgaagtcagtgacccaaccgtaaggagggagctgc

SEQ ID NO: 145 - DNA, 1438 bp, Lactonifactor longoviformis
  misc_feature: Lactonifactor longoviformis 16S NCBI Reference Sequence NR_043551.1
gagtttgattatggctcaggatgaacgctggcggcgtgcttaacacatgcaagtcgaacgaagcatatagagacgagtatttcggtatgagtaactatatgactgagtggcggacgggtgagtaacgcgtggataacctgcctcatacagggggataacagttagaaatgactgctaataccgcataagcgcacagtgctgcatggcacagtgtgaaaagctccggcggtatgagatggatccgcgtttgattagctagttggtggggtaaaggcctaccaaggcgacgatcaatagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaattacgtgccagcagccgcggtaatacgtaaggggcaagcgttatccggatttactgggtgtaaagggagcgtagacggcagtgcaagtctgatgtgaaagcccggggctcaaccccgggactgcattggaaactgtgcagctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatactaggtgtcgggcgccaaaggcgttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctgcctgaccggtccgtaacaggacccttccttcgggacaggcaagacaggtggtgcatggttgtcgtcagctcgtgtcgtgagatgttgggttaagtcccgcaacgagcgcaacccttatccttagtagccagcaggtagagctgggcactctagggagactgccagggacaacctggaggaaggtggggatgacgtcaaatcatcatgccccttatgatttgggctacacacgtgctacaatggcgtaaacaaagggaagcgaaggggtgacctgaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggatatgcccgaagccggtgaccgaacccgaaa

SEQ ID NO: 146 - DNA, 1456 bp, Marvinbryantia formatexigens
  misc_feature: Marvinbryantia formatexigens (Bryantella formatexigen) 16S NCBI Reference Sequence NR_114807.1
tggcggcgtgcttaacacatgcaagtcgagcgaagcattttaaatgaagttttcggacggaatttaaaatgactgagcggcggacgggtgagtaacgcgtggataacctgccttatacagggggataacagccagaaatggctgctaataccgcataagcgcacggtaccgcatggtacagtgtgaaaaactccggtggtataagatgggtccgcgttggattaggcagttggcggggtaaaggcccaccaaaccgacgatccatagccggcctgagagggtggacggccacattgggactgagacacggcccagactcctacgggaggcagcagtggggaatattgcacaatgggggaaaccctgatgcagcgacgccgcgtgggtgaagaagtatttcggtatgtaaagccctatcagcagggaagaaaatgacggtacctgaccaagaagccccggctaactacgtgccagcagccgcggtaatacgtagggggcaagcgttatccggatttactgggtgtaaagggagcgtagacggccatgcaagtctggtgtgaaaggcgggggctcaacccccggactgcattggaaactgtatggcttgagtgccggagaggtaagcggaattcctggtgtagcggtgaaatgcgtagatatcaggaggaacaccagtggcgaaggcggcttactggacggtaactgacgttgaggctcgaaagcgtggggagcaaacaggattagataccctggtagtccacgccgtaaacgatgaataccaggtgtcgggggacacggtccttcggtgccgcagcaaacgcactaagtattccacctggggagtacgttcgcaagaatgaaactcaaaggaattgacggggacccgcacaagcggtggagcatgtggtttaattcgaagcaacgcgaagaaccttaccaggtcttgacatccggacgaccggacagtaacgtgtccttcccttcggggcgtccgagacaggtggtgcatggttgtcgtcagctcgtgtcgtgagatgttgggttaagtcccgcaacgagcgcaacccctgttcccagtagccagcattcaggatgggcactctggggagactgccagggataacctggaggaaggcggggatgacgtcaaatcatcatgccccttatgatctgggctacacacgtgctacaatggcgtgaacagagggaagcgaacccgcgagggggagcaaatcccagaaataacgtcccagttcggattgtagtctgcaacccggctacatgaagctggaatcgctagtaatcgcggatcagcatgccgcggtgaatacgttcccgggtcttgtacacaccgcccgtcacaccatgggagtcggaaatgcccgaagtcagtgacccaaccggaaggagggagctgccgaaggcggggccggtaactggggtgaagtcgtaacaa

SEQ ID NO: 147 - DNA, 1493 bp, Mediterraneibacter butyricigenes
  misc_feature: Mediterraneibacter butyricigenes 16S GenBank LC423447.1
tcaggatgaacgctggcggcgtgcttaacacatgcaagtcgaacgaagcacttgatttgattttcttcggagatgaagatcttgtgactgagtggcggacgggtgagtaacgcgtgggcaacctgccttgtacagggggataacagttagaaatgactgctaataccgcataagaccacagcatcacatgatgcaggggtaaaaactgaggtggtacaagatgggcccgcgtctgattagctggttggtgaggtaacggctcaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggaacagcaagtctgatgtgaaaacccggggctcaaccccgggactgcattggaaactgttgatctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgcccttgacatccgaatgaccggtgagtaatgtcgcctttccttcgggacattcgagacaggtggtgcatggttgtcgtcagctcgtgtcgtgagatgttgggttaagtcccgcaacgagcgcaacccttatctttagtagccagcaattaggatgggcactctagagagactgccagggataacctggaggaaggtggggatgacgtcaaatcatcatgccccttatgggcagggctacacacgtgctacaatggcgtaaacaaagggaagcagagtcgtgaggccgagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggtgggaccgataactgggggtgaagtcgtaacaaggtagccgtatcggaagggcg

SEQ ID NO: 148 - DNA, 1404 bp, Mediterraneibacter faecis
  misc_feature: Mediterraneibacter faecis (Ruminococcus faecis) 16S NCBI Reference Sequence NR_116747.1
atgcaagtcgaacgaagcaccttgatttgattcttcggatgaagatcttggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggtttggccgggcactctagagagactgccagggataacctggaggaaggtggggatgacgtcaaatcatcatgccccttatgagcagggctacacacgtgctacaatggcgtaaacaaagggaggcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggaggagctgccgaag

SEQ ID NO: 149 - DNA, 1493 bp, Mediterraneibacter glycyrrhizinilyticus
  misc_feature: Mediterraneibacter glycyrrhizinilyticus (Clostridium glycyrrhizinilyticum) 16S NCBI Reference Sequence NR_112553.1
  misc_feature (207)..(207): n is a, c, g, or t
agagtttgatcctggctcaggatgaacgctggcggcgtgcttaacacatgcaagtcgaacgaagcactttaccggatttcttcggaatgaaagttttgtgactgagtggcggacgggtgagtaacgcgtgggtaacctgcctcatacagggggataacagttagaaatgactgctaataccgcataagaccacaggaccgcatggtncggtggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaattaggacggtacctgactaagaagcaccggctaaatacgtgccagcagccgcggtaatacgtatggtgcaagcgttatccggatttactgggtgtaaagggagcgtagacggagaggcaagtctgatgtgaaaacccggggctcaaccccgggactgcattggaaactgtttttctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggaggcaaagcctttcggtgccgcagcaaacgcaataagcagtccacctggggagtacgttcgcaagaatgaaactcaaaggaattgacggggacccgcacaagcggtggagcatgtggtttaattcgaagcaacgcgaagaaccttacctgcccttgacatccggctgaccggcgagtaatgtcgcctttccttcgggacagccgagacaggtggtgcatggttgtcgtcagctcgtgtcgtgagatgttgggttaagtcccgcaacgagcgcaacccttatctttagtagccagcatttcggatgggcactctagagagactgccagggataacctggaggaaggtggggatgacgtcaaatcatcatgccccttatgggcagggctacacacgtgctacaatggcgtaaacaaagggaggcaagcctgcgagggtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggtgggaccgataactggggtgaagtcgtaacaaggtaacc

SEQ ID NO: 150 - DNA, 1481 bp, Mediterraneibacter gnavus
  misc_feature: Mediterraneibacter gnavus (Ruminococcus gnavus) 16S
  misc_feature (148)..(148): n is a, c, g, or t
  misc_feature (1071)..(1071): n is a, c, g, or t
  misc_feature (1341)..(1341): n is a, c, g, or t
tgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacgnctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcancccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgtncccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaagg

SEQ ID NO: 151 - DNA, 1402 bp, Mediterraneibacter lactaris
  misc_feature: Mediterraneibacter lactaris (Ruminococcus lactaris) 16S NCBI Reference Sequence NR_027579.1
  misc_feature (11)..(11): n is a, c, g, or t
  misc_feature (330)..(330): n is a, c, g, or t
  misc_feature (333)..(333): n is a, c, g, or t
  misc_feature (357)..(357): n is a, c, g, or t
  misc_feature (927)..(927): n is a, c, g, or t
  misc_feature (1237)..(1237): n is a, c, g, or t
cggcgtgcttnacmcrtgcgagtcgagcgaagcacttaggwaagattcttcggatgatttcctatttgactgagcggcggacgggtgagtaacgcgtgggtaacctccctcatacagggggataacagttagaaatgactgctaataccgcataagaccacagcaccgcatggtgcaggggtaaaaactccggtggtatgagatggacccgcgtctgattagttagttggtggggtaacggcctaccaaggcgacgatcagtagccgacctgagagggtgaccggccacattgggactgagacacggcccaaactcctacgggaggcagnagnggggaatattgcacaatggggganaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggagcagcaagtctgatgtgaaaacccggggctcaaccccgggactgcattggaaactgttgatctggagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggtggcaaagccattcggtgccgcaggcaacgcaataagtagtccacctggggagtacgttcgcaagaatgaaactcaaaggaattgacggggacccgcacaagcggtggagcatgtggtttaattcgaagcaacgcgaagnaaccttacctgctcttgacatcccggtgacggcagagtaatgtctgcttttcttcggaacaccggtgacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcgaacccgcgagggtgggcaaatcccanaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgt

SEQ ID NO: 152 - DNA, 837 bp, Mediterraneibacter massiliensis
  misc_feature: Mediterraneibacter massiliensis 16S NCBI Reference Sequence NZ_LN913000.1
atgtcaggaacactttacttatgcgcaacgccaattggaaatttggaagatatgacgtttcgtgtgatacgtattttaaaagaagtagatttaattgctgcagaagatacgagaaacagtataaaattacttaatcattttgaaatacagactcccatgaccagttatcatgagtataataaatatgaaaaagggaagaaattgatcgaaaaactgcaggcagggcaaaatattgcattgatcacagacgcaggcacgccgggaatttcagatccgggagaagaattggtgcgtatgtgttatgaggcgggtatcacggtgacatcactgcctggcgcagccgcatgtattacggcgctgacactttcagggctttctacccggaggtttgcatttgaggcatttttgccgtcagataaaaaagaacgggagtgtatcctaaaagaacttactgccgaaacaagaacgattattttgtatgaggcgccgcacagacttttaaagacattggtactattaagagaaacacttggaaacaggaaagtcactgtatgcagagaattgacaaaaaagcatgaaaacgtgatgacatttactctggaagaggcaacggcattttatgcggaacatacgccaaaaggggaatgtgtactggcgatagagggaaaaagtgcagaagaagtaaagcaggaagaacgggctaaatgggaggaaatgagtatagaggaacacatgcagttctacatggaagcaggaaaagagaaaaaggaagccatgaagtgtgttgcaaaagacagaggagtatccaaaagagaaatatataattatctgctgtaa

SEQ ID NO: 153 - DNA, 1384 bp, Mediterraneibacter torques
  misc_feature: Mediterraneibacter torques (Ruminococcus torques) 16S GenBank MT903152.1
atgcagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctggttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccgggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaacc

SEQ ID NO: 154 - DNA, 1433 bp, Murimonas intestine
  misc_feature: Murimonas intestine 16S NCBI Reference Sequence NR_134772.1
tgcagtcgagcggagtatttaacggaagtcttcggatggaagtttggatacttagcggcggacgggtgagtaacgcgtgggcaacctgccccataccgggggataacagccagaaatggctgctaataccgcataagcgcacagcgccacatggcgcggtgtgaaaaactccggtggtatgggatgggcccgcgtctgattaggtagttggcggggtaacggcccaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggccaggcaagtctgatgtgaaaggcaggggctcaacccctggactgcattggaaactgtcaggctggagtgccggagaggtaagcggaattcctagtgtagcggtgaaatgcgtagatattaggaggaacaccagtggcgaaggcggcttactggacggtaactgacgttgatgctcgaaagcgtggggagcaaacaggattagataccctggtagtccacgccgtaaacgatgaatactaggtgtcggggggcaaagcccttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagccttgacatcccattgaagccagagtaacgtctgacggccttcgggacaatggagacaggtggtgcatggttgtcgtcagctcgtgtcgtgagatgttgggttaagtcccgcaacgagcgcaacccttatcttcagtagccagcacgcgatggtgggcactctggagagactgccagggacaacctggaggaaggtggggatgacgtcaaatcatcatgccccttatggcttgggctacacacgtgctacaatggcgtaaacagagggaagcagccctgcgaaggtgagcgaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacctaaaccgaaaggaaggagctgccgaaggcgggactggtaactggggtgaagtcgtaa

SEQ ID NO: 155 - DNA, 1409 bp, Oscillibacter massiliensis
  misc_feature: Oscillibacter massiliensis 16S NCBI Reference Sequence NR_118156.1
taccatgcagtcgaacggaacacccttgacagaggtttcggccaatgaagaggaatgtttagtggcggactggtgagtaacgcgtgaggaacctgcctttcagagggggacaacagttggaaacgactgctaataccgcatgaagcagcgaggggacatccccttgctgtcaaagatttatcgctgaaagatggcctcgcgtctgattagctagttggtggggtaacggcccaccaaggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttaacagggaagagaagaagacggtacctgttgaataagccacggctaactacgtgccagcagccgcggtaatacgtaggtggcaagcgttgtccggatttactgggtgtaaagggcgtgtagccgggaaggcaagtcagatgtgaaatctggaggctcaacctccaaactgcatttgaaactgcttttcttgagtatcggagaggtaatcggaattccttgtgtagcggtgaaatgcgtagatataaggaagaacaccagtggcgaaggcggattactggacgacaactgacggtgaggcgcgaaagcgtggggagcaaacaggattagataccctggtagtccacgctgtaaacgatcaatactaggtgtgcggggactgaccccctgcgtgccgcagttaacacaataagtattgcacctggggagtacgatcgcaaggttgaaactcaaaggaattgacgggggcccgcacaagcggtggattatgtggtttaattcgaagcaacgcgaagaaccttaccaggacttgacatcctactaacgaggtagagatacgtcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaagccgtgaggcagagcgaacccctaaaagccgtctcagttcggatcgtaggctgcaactcgcctacgtgaagtcggaatcgctagtaatcgcggatcagaatgccgcggtgaatacgttcccgggccttgtacacaccgcccgtcacaccatgagagtcgggaacacccgaagcccgtagcctaactgcaaagagggcgcgtcgaaggtt

SEQ ID NO: 156 - DNA, 1525 bp, Robinsoniella peoriensis
  misc_feature: Robinsoniella peoriensis 16S NCBI Reference Sequence NR_041882.1
gagagtttgatcctggctcaggatgaacgctggcggcgtgcttaacacatgcaagtcgagcgaagcattaaaaccgatttcttcggattgaagtttttgtgactgagcggcggacgggtgagtaacgcgtgggtgacctgcctcatacagggggataacagacagaaatgtctgctaataccgcataagcgcacagtgctgcatggcacagtgtgaaaaactccggtggtatgagatggacccgcgtctgattagctagttggtaaggtaacggcttaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcatggcaagtctggtgtgaaaggcaggggctcaacccctggactgcattggaaactgtcaggctggagtgcaggagaggtaagtggaattcctagtgtagcggtaaaatgcgtagatattaggaggaacaccagtggcgaaggcgacttactggactgtaactgacgttgaggctcgaaagcgtggggagcaaacaggattagataccctggtagtccacgccgtaaacgatgaatactaggtgtcagggagcaaagctctttggtgccgcagctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggacagtaacgtgtcctttccttcgggacaagagtgacaggtggtgcatggttgtcgtcagctcgtgtcgtgagatgttgggttaagtcccgcaacgagcgcaacccttatcttcagtagccagcaattcggttgggcactctggagagactgccagggataacctggaggaaggtggggatgacgtcaaatcatcatgccccttatgatttgggctacacacgtgctacaatggcgtaaacaaagggaagcagccctgtgaaggtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcggtgacccaaccgtaaggagggagctgccgaaggtggagccggtaactggggtgaagtcgtaacaaggtagccgtatcggaaggtgcggctggatcacctccttc

SEQ ID NO: 157 - DNA, 1456 bp, Roseburia faecis
  misc_feature: Roseburia faecis 16S NCBI Reference Sequence NR_042832.1
gatgaacgctggcggcgtgcttaacacatgcaagtcgaacgaagcactctatttgattttcttcggaaatgaagattttgtgactgagtggcggacgggtgagtaacgcgtgggtaacctgcctcatacagggggataacagttggaaacgactgctaataccgcataagcgcacaggatcgcatgatccggtgtgaaaaactccggtggtatgagatggacccgcgtctgattagccagttggcagggtaacggcctaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgatgacagagtatgtaatgtacyttctcttcggagcatcggtgacaggtggtgcatggttgtcgtcagctcgtgtcgtgagatgttgggttaagtcccgcaacgagcgcaacccctgtccttagtagccagcggttcggccgggcactctagggagactgccagggataacctggaggaaggcggggatgacgtcaaatcatcatgccccttatgacttgggctacacacgtgctacaatggcgtaaacaaagggaagcggagccgtgaggccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgccgaaggcaggttcgataactggggtg

SEQ ID NO: 158 - DNA, 1454 bp, Roseburia inulinivorans
  misc_feature: Roseburia inulinivorans 16S NCBI Reference Sequence NR_042007.1
ttgatcctggctcaggatgaacgctggcggcgtgcttaacacatgcaagtcgaacgaagcacttttacagatttcttcggaatgaagttttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggaaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggacagtaatgtgtcctttccttcgggacagaagtgacaggtggtgcatggttgtcgtcagctcgtgtcgtgagatgttgggttaagtcccgcaacgagcgcaacccttatccccagtagccagcggttcggacgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgcgaaggcaggt

SEQ ID NO: 159 - DNA, 1407 bp, Ruminococcus albus
  misc_feature: Ruminococcus albus 16S NCBI Reference Sequence NR_025929.1
ttgatcctggctcaggacgaacgctggcggcacgcttaacacatgcaagtcgaacgagcgaaagagtgcttgcactctctagctagtggcgggcgggtgagtaacacgtgagcaatctgcctttcggagagggataccaattggaaacgattgttaatacctcataacataacgaagccgcatgactttgttatcaaatgaatttcgccgaaagatgagctcgcgtctgattaggtagttggtgaggtaacggcccaccaagccgacgatcagtagccggactgagaggttgaacggccacattgggactgagacacggcccagactcctacgggaggcagcagtggggaatattgcacaatgggcgaaagcctgatgcagcgatgccgcgtgagggaagaaggttttaggattgtaaacctctgtctttggggacgataatgacggtacccaaggaggaagctccggctaactacgtgccagcagccgcggtaatacgtagggagcgagcgttgtccggaattactgggtgtaaagggagcgtaggcgggattgcaagtcaggtgtgaaatttaggggcttaacccctgaactgcacttgaaactgtagttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggctgaaactcaaaggaattgacgggggcccgcacaagcagtggagtatgtggtttaattcgaagcaacgcgaagaaccttaccaggtcttgacatcgtacgcatagcatagagatatgtgaaatcccttcggggacgtatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggctacacacgtactacaatggctgttaacagagggaagcaaaacagtgatgtggagcaaaaccctaaaagcagtcttagttcggattgtaggctgcaacccgcctacatgaagtaggaattgctagtaatcgcggatcagcatgccgcggtgaatacgttcccgggccttgtacacaccgcccgtcacgccatgggagtcggtaaccccgaagcctgtgttctaaccgc

SEQ ID NO: 160 - DNA, 1380 bp, Ruminococcus bromii
  misc_feature: Ruminococcus bromii 16S NCBI Reference Sequence NR_025930.1
  misc_feature (11)..(11): n is a, c, g, or t
  misc_feature (541)..(541): n is a, c, g, or t
  misc_feature (830)..(831): n is a, c, g, or t
  misc_feature (1063)..(1063): n is a, c, g, or t
  misc_feature (1283)..(1283): n is a, c, g, or t
tcgaacggaantgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttctgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcaccaacgccgcgtgaaggatgaaggttttcggattgtaaacttcttttattaaggacgaaaaatgacggtacttaatgaataagctccggctaactacgtgccagcagccgcggtaatacgtagggagcaagcgttgtccggatttactgggtgtaaagggtgcgtaggcggctttgcaagtcagatgtgaaantctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggagggtctgaccccttccgtgccggagttaacacaataagtaatccacctggggagtacggmcnnaargttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttggkttaagtcccgcaacgagcgcaacccttgctattagttgntacgcaagagcactctaataggactgccgttgacaaaacggaggaaggtggggacgacgtcaaatcatcatgccccttatgacctgggctacacacgtactacaatggatgttaacagagggaagcaagacagtgatgtggagcaaacccctaaaaacattctcagttcagattgcaggctgcaacccgcctgcatgaagatggaattgctagtaatcgnggatcagaatgmcgcggtgaatacgttcccgggccttgtacacaccgcccgtcacaccatgggagccggtaatacccgaagtcagtagtccaacctc

SEQ ID NO: 161 - DNA, 1380 bp, Subdoligranulum variabile; identical to SEQ ID NO: 160
  misc_feature: Subdoligranulum variabile 16S
  misc_feature (11)..(11): n is a, c, g, or t
  misc_feature (541)..(541): n is a, c, g, or t
  misc_feature (830)..(831): n is a, c, g, or t
  misc_feature (1063)..(1063): n is a, c, g, or t
  misc_feature (1283)..(1283): n is a, c, g, or t

SEQ ID NO: 162 - DNA, 1461 bp, Syntrophococcus sucromutans
  misc_feature: Syntrophococcus sucromutans 16S NCBI Reference Sequence NR_119287.1
gatcctggctcaggatgaacgctggcggcgtgcttaacacatgcaagtcgagcgaagcacttttgatttcttcggattgaaggagtgactgagcggcggacgggtgagtaacgcgtggggaacctgccccataccgggggataacagttggaaacgactgctaataccgcataagcgcacaggaaggcatcttccagtgtgaaaaactccggtggtatgggatggacccgcgttggattagccagttggtggggtaacggcctaccaaagcgacgatccatagccggcctgagagggcgaccggccacattgggactgagacacggcccaaactcctacgggaggcagcagtggggaatattgcacaatgggggaaaccctgatgcagcgacgccgcgtgagtgatgaagtatttcggtatgtaaagctctatcagcagggaagaagacgacggtacctgactaagaagccccggctaactacgtgccagcagccgcggtaatacgtagggggcaagcgttatccggatttactgggtgtaaagggagcgcatacggcagtgcaagtctgaagtgaaaacccgaggctcaaccacgggattgctttggaaactgtacagctagagtgtcggaggggtaagcggaattcctagtgtagcggtgaaatgcgtagatattaggaggaacaccggtggcgaaggcggcttactggacgataactgacgttgaggctcgaaagcgtggggagcaaacaggattagataccctggtagtccacgccgtaaacgatgaatactaggtgttggcacgcaaggcgtgtcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccggccggtaacgcggcccttccttcgggacaggcgagacaggtggtgcatggttgtcgtcagctcgtgtcgtgagatgttgggttaagtcccgcaacgagcgcaacccttatcctcagtagccagcacgaaaggtggggcactctggggagactgcccaggagaacctggaggaaggtggggatgacgtcaaatcatcatgccccttatgatttgggctacacacgtgctacaatggcgtgaacagagggaagcgaaggagcgatccggagcaaatctcaaaaaacacgtcccagttcggactgtaggctgcaacccgactgcacgaagctggaatcgctagtaatcgcagatcagaatgctgcggtgaatacgttcccgggtcttgtacacaccgcccgtcacaccatgggagtcggtaatgcccgaagcctgtgacccaaccagcaatggagggagcagtcgaaggcagggccggtgactgggg

SEQ ID NO: 163 - DNA, 1492 bp, Faecalimonas umbilicate
  misc_feature: Faecalimonas umbilicate  16S NCBI Reference Sequence NR_156907.1
agagtttgatcctggctcaggatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccgctctttaatcggagctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtaacc

SEQ ID NO: 164 - DNA, 1397 bp, Merdimonas faecis
  misc_feature: Merdimonas faecis 16S NCBI Reference Sequence NR_157642.1
tgcagtcgagcgaagcgcttaagttggatttcttcggaatgacgatttttgtgactgagcggcggacgggtgagtaacgcgtgggtaacctgcctcatacagggggataacagttagaaatgactgctaataccgcataagaccccagcttcgcatgaagcggtggtaaaaactccggtggtatgagatggacccgcgtctgattagctagttggtaaggtaacggcttaccaaggcgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaaagtgacggtacctgactaagaagccccggctaactacgtgccagcagccgcggtaatacgtagggggcaagcgttatccggatttactgggtgtaaagggagcgtagacgggatagcaagtctgaagtgaaagcccggggctcaaccccgggactgctttggaaactgttgacctggagtgctggagaggcaagtggaattcctagtgtagcggtgaaatgcgtagatattaggaggaacaccagtggcgaaggcggcttgctggacagtgactgacgttgaggctcgaaagcgtggggagcaaacaggattagataccctggtagtccacgccgtaaacgatgactactaggtgtcggtgagcaaagctcatcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgatcttgacatccccctgaccggtgagtaatgtcacctttccttcgggacaggggagacaggtggtgcatggttgtcgtcagctcgtgtcgtgagatgttgggttaagtcccgcaacgagcgcaacccttatccttagtagccagcattacggatgggcactctagggagactgccagggacaacctggaggaaggtggggatgacgtcaaatcatcatgccccttatgaccagggctacacacgtgctacaatggcgtaaacaaagggaagcgaaggagtgatccggagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagtcagtaacgcccgaagccggtgacccaacctacaaggagggag

SEQ ID NO: 165 - DNA, 1475 bp, Clostridium innocuum
  misc_feature: Clostridium innocuum 16S
  misc_feature (905)..(905): n is a, c, g, or t
  misc_feature (942)..(942): n is a, c, g, or t
atggagagtttgatcctggctcaggatgaacgctggcggcatgcctaatacatgcaagtcgaacgaagtcttcaggaagcttgcttccaaaaagacttagtggcgaacgggtgagtaacacgtaggtaacctgcccatgtgtccgggataactgctggaaacggtagctaaaaccggataggtatacggagcgcatgctctgtatattaaagcgcccttcaaggcgtgaacatggatggacctgcgacgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagyagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggagaaattcagtgctgcagttaacgcaataagttctccgcctggggagtatgcacgcaagttngaaactcaaaggaattgacgggggcccgcacaagcgntggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacttggtgacaagaagcgaatctcataaagatcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 166 - DNA, 1519 bp, Erysipelatoclostridium ramosum
  misc_feature: Erysipelatoclostridium ramosum 16S NCBI Reference Sequence NR_029247.1
gagagtttgatcctggctcaggatgaacgctggcggcgtgcctaatacatgcaagtcgaacgcgagcacttgtgctcgagtggcgaacgggtgagtaatacataagtaacctgccctagacagggggataactattggaaacggatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaagaagaacggcggctacaggaaatggtagccgagtgacggtactttattttagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaaccgcgaggtgaagcaaaacccataaaaaccattctcagttcggattgtagtctgcaactcgactacatgaagttggaatcgctagtaatcgcgaatcagcatgtcgcggtgaatacgttctcgggccttgtacacaccgcccgtcacaccacgagagttgataacacccgaagccggtggcctaaccgcaaggaaggagctgtctaaggtgggattgatgattggggtgaagtcgtaacaaggtatccctacgggaacgtgcggctggatcacctcc

SEQ ID NO: 167 - DNA, 1296 bp, Ruminococcaceae
  misc_feature: Ruminococcaceae isolate S16
ggtgtgacgggcggtgtgtacaaggcccgggaacgtattcaccgcggcatgctgatccgcgattactagcaattccgacttcatacaggcgagttgcagcctgcaatctgaactgggaccatttttaggggtttgctctgcatcgctgctttgcttccctctgttgatggccattgtagtacgtgtgtagcccaggtcataaggggcatgatgatttgacgtcgtccccaccttcctccgttttgtcaacggcagtcctattagagtgctcttgcgtagcaactaatagcaagggttgcgctcgttgcgggacttaacccaacatctcacgacacgagctgacgacaaccatgcaccacctgtctcaactttccccgaagggcacctaatgtatctctacttcgttagttggatgtcaagacctggtaaggttcttcgcgttgcttcgaattaaaccacatactccactgcttgtgcgggcccccgtcaattcctttgagtttcaaccttgcggccgtactccccaggtggattacttattgtgttaactgcggcacggagggggtcagaccccccacacctagtaatcatcgtttacggcgtggactaccagggtatctaatcctgtttgctacccacgctttcgtgcctcagcgtcagttaaagcccagcaggccgccttcgccactggtgttcctccctatctctacgcatttcaccgctacacagggaattccgcctgcctctacttcactcaagcaaaacagtttcaaatgcagttcgtgggttaagcccacggatttcacatctgacttgctctgccgcctacgcaccctttacacccagtaaatccggacaacgcttgctccctacgtattaccgcggctgctggcacgtagttagccggagcttattcattaagtaccgtcacttcttcgtccttaataaaagaactttacaatccgaagaccttcatcgttcacgcggcgttgctgcgtcagggttgcccccattgcgcaatatcccccactgctgcctcccgtaggagtctgggccgtgtctcagtcccaatgtggccgttcaacctctcagtccggctaccgatcgtcgctttggtgggccgttaccccgccaactggctaatcggacgcgagtccatcttcaagcgataaaatctttgatacggttgccatgcgacatccgtatgttatgcggtattagcgttcttttcagaacgttattcccctcttgaaggcaggttactcacgcgttactcacccgtccgccactaaactt

SEQ ID NO: 168 - DNA, 498 bp, Eubacterium rectale
  misc_feature: Eubacterium rectale butyryl-CoAacetate CoA-transferase GenBank GQ231300.1
ggcggttatgagagaaagcttatcaagagaggctgttctttctactcaccaatccgttactcagagcttcctcgttattatagagattcaacaacacctgacgatgtcgctatgttccaggttgcaccgatggattcacacggttacttcaactttggtcctaacgcatctcacctcggtgctgtatgcgagacttcaaagaaaatcatcgttgaggtcaatgaaaacatgcctcgctgtcacggcggttcagaggccaatgtacatatttcacaggtttcttacattgttgagggtgacaaccctgcaatcggagagcttggtgccggtggtcctgcaacagatgtagacaagaaggttgcagagcttatcgttgaccagattccaaacggtgcctgtctccagctcggtatcggcggaatgccaaacgcagtaggctcactcatcgctgagtcagacctcaaggatctcggtgtacacacagagatgtacgttgac

SEQ ID NO: 169 - DNA, 498 bp, Eubacterium hallii
  misc_feature: Eubacterium hallii strain butyryl-CoAacetate CoA-transferase
ggtggtttggagagaaaggcaatcagtaaaggatttgcatactattctccattaaaatattcagaacttccacgttattacagagagaatatcaagcatttgaatgttgcgatgttccaggttgctccaatggataaacatggattctttaactttggaccaaatgcatcccatatgatggcagtttgtgaagctgcagatgtaattatcgtagaggttaatgagaatatgcctcgctgtttgggtggatttgaagaaggcattcatgtttctcgcgtagattacatcgtagaaggcgagaatccagctattggtgagttaggagctggcgcaccaccaacagaagtagacaaagccgtagcacagcttatcgtagaagagattccagatggagcatgtttacagcttggtatcggtggaatgcctaatacagtaggttccatgattgctgaatctgatttaaaggacttaggtgtacatacagagatgtacgttgat

SEQ ID NO: 170 - DNA, 1341 bp, Anaerobutyricum soehngenii
  misc_feature: Anaerobutyricum soehngenii butyryl-CoAacetate CoA-transferase GenBank JAHLPL010000009.1
ctatctcttattggaacgccgccatatctttcttttttcggcttccttaatcagttcatctctgaaatccggatgggcaatggaaataagcccctctgctctctcccatgttgaacgcccttccatgttgatcactccatattccgttgcaagaaaatatacctgactccgcggagatgtaataatatctccattaaactgcggcaatactcttgaaaaaagctgtccgcttttatctttatacgtagagcccatacagataaatgccttaccccctctggctgaagatgctccgattaaaaagtcaagctgtcctccggtaccactgatctgccttgcaccaaagctctcggatgatacctgtccatacagatctacagaaacacagctattgatggacaccatattgtcaatctgggcaattacatccggtctgttcacatattccaatggatatgctgctacaccatgattatcatctaaccattcatacagttctgccgaaccaatagccacaccaaatacacccttacccttatcgattgtcttctttttattggtaagcttccctgctttatataaatgcaggtacgcatctgagcaaagttcagtatgcatgccaagatccttaagatctgattccgcaaacagttcaccaactgcattcggcatacttccaattccaagctgcaaggttgatccatcacaaatatacggtagaatattctgtacaatctttcgatcagtctcagtcggtgcatagcgcctgactgtcgtaataggttcatgtcttccttctacgatataatccacttcagacacatgaatacattccccatatccaccatggatcttcggcatatgctcattaacctctacaataacaatatctgcattctgcacgataggtccggctactccggtatttaccgaataattgaaatatccatgcttgtccatcggtgaaacgctgaccattactacattgacattcagaaaatacttataataggctgcattattgtgaaataccatcggaatatagtaacatagtcctctgtcacaaagctttctctcataagtagaacaatgccatgtatgatagacaaaatgttcctgactttcatcacattctgccacttcaatcggtccctccataagattccctcgaatctttacatcaaaaagttcatctcttcttttagcaagagcccgatctaaaagaaccggctttccaagtgagctggtataatcaacccagtctccactctttactacttctaccgcctcttccggtgtacaaagcttttgtctgtactcagttgtaaaatcctttaacac

SEQ ID NO: 171 - DNA, 498 bp, Faecalibacterium prausnitzii
  misc_feature: Faecalibacterium prausnitzii butyryl-CoAacetate CoA-transferase GenBank GQ231311.1
ggcggcatcgagcgcaaagccatcgcacagggcttctcgttctactcccccatccgctattcggagctgccccgctactaccgcgagagtcccgacccgctggacatcgccgtcttccaggtgactccgatggacgagcacggctacttcaacttcggccccagcacttcccatctgggtgccgtttgtgagaaggccaggaagatcatcgtggaggtcaacaccaatatgccccgctgcctcggcggtatggagaactgtgtccatgtctcccaggtcaccggcatcgtggagggcggcaatccgcccatcggccagatggccgctgccggccccgcttccgaggtcgatttgaaggtcgcaaacctcgtcgtgccgcagatccccgacggtgcctgcctgcagctgggcatcggcggtatgcccaacgccatcggcagcctcatcgcccagagcgatttgaaggacctgggcgtccacaccgagatgtatgttgac

SEQ ID NO: 172 - DNA, 498 bp, Roseburia intestinalis
  misc_feature: Roseburia intestinalis butyryl-CoAacetate CoA-transferase GenBank GQ231304.1
tccggtatcgaacgtaaaatgatcagccgcggatgcgctttctattcaccgatccgttactccgaactgccacgttactaccgcgagcttgactgcccggatgatgtagcaatgtttcaggtagctccgatggattcccatggatatttcaatttcggaccaagtgcctcccatctcggtgcaatgtgtgagagagcaaaacacatcatcgtagaggtcaatgaaaacatgccgcgctgccttggcggaacagaatgcggaattcacatttccgatgttacctatatcgtagagggttcgaatcctccgatcggcgagcttggtgccggcggtcctgcaacagatatcgacaaaacggttgcaaaactgatcgtagacgagatcccgaacggtgcatgcttacaacttggtatcggcggtatgccaaacgctgtcggttccctgattgcagagtctgacttaaaagacctcggtgtccacacagagatgtatgtagat

SEQ ID NO: 173 - DNA, 1341 bp, Roseburia hominis
  misc_feature: Roseburia hominis butyryl-CoAacetate CoA-transferase NCBI Reference Sequence NZ_WQNU01000046.1
ctagcggttgcttcttctccagatgtgcatctgctccgcctctttgatgaggtcgtcgcggaaatcaggatgcgcgatcgagatcagagcctcggctctctgccaggtagacagaccctttaagttcaccttgccgtattcggttacaacatagtgtgtattcggtcttgcgtcggtaacgatggaaccgtttgccagcgtcgggcggatacgggactgcacctgaccgtccttggtcttgaaggtagaggataagcagataaagctcttgccgcccttggacagataagctccgagcacgaagtcaagctgtccgcctgcgccgctgatctgcttgatgcctgcagactctgcatttacctgaccatagagatcaatatccacggcattgttgatggaaataaagttatccagtgcggagatcgagcggatgtcattcgtgtagtcgaccggagcgctcatcagttccgggttgtcgtccagataatcgtacattttcttggtgccggcgccgaaagcgtaggtctggcggtaacggtcgatattctttttggaaccgttgatcttacctgcctttgcaatatcgacaaatgcatccacgtacatctcagtgtgaacgccgagatccttcaagtcggactctgcaatcagggaaccgacagcgtttggcatgccgccgataccgagctgtaagcaggcaccgttcggaatctcatcgacgatcagctttgcgacagccttatccacatctgtagcaggaccgcctgcaccaagttcaccgattggcgggttggagccttctacgatgtaggtgacatcggaaatgtggatgccgcactcggtaccgccgaggcagcgtggcatattttcattgacttctacgatgatgtgctttgcgcgctcgcacattgcacccagatgtgaggcactcggaccgaagttaaagtagccgtgcgcatccatcggagcgacctggaacatggcaacgtcatccgggcagtcaagctcgcggtagtagcgcggcagctcggagtagcggatcggacagtagtaagccacgcctctgtttatcatcttgcgctcgataccggacatatgccaggagttccagcagaaatgctcgcctgcatcctcacgtgcaaaaacagccagcggcttcatcaggataccgccgcgcagcttgacgtctgtcagttcgtcggtgcgctttgcgagagcctgatccagtgcgtcaacggtgttggtgcaccagccgtaatctacccagtctccggatttgatgagctttaccgcctcatctgcggagacaagcttctgtttgtattcttcacgaaaatccat

SEQ ID NO: 174 - DNA, 1341 bp, Anaerostipes caccae
  misc_feature: Anaerostipes caccae butyryl-CoA transferase European Nucleotide Archive Coding ABA39273.1
atgtcatttaaggaagaataccaaaagaaacttaaaacagccgatgaggccgtaaaagttgtgaaatccggcgattggctggagtacggctggtgcgtcaccacaccggctgcccttgacaaggctttggcaaaaagaatgccggaattggaaaatattaatatccggggcggaatcgtcatgtggccgctggaaatcactaagatcgactcccctgccgatcactttacatggaattcctggcacatgggcggactggagagaaaatggatcaaggaaggtttttcctactacgcaccgatccgttattccgagctgccgggatactaccgcaattacatcgatcacgtggatgttgcgatgatgcaggttgccccgatggacgaacacggattcttcaacttcgggccgagtgcatcccacctggctgcaatgcttgaaaaagccgactgtgtcatcgtggaagtcaacgaaaatatgcctcgctgcttaggcggatttgaagagggtgtacatatttccaaagttgatatgatcgtcgaaggcgaaaatcctgcgatcgcagagctgggcggaggcggagcagccaccgacgtggacaaagccgttgcaaaactcatcgtagaccagatcccgaacggcgcatgtctccagcttggcatcggcggaatgccgaatgccgtaggttccatgatcgcggaatctgaccttaaagacctgggcgttcacactgaaatgtatgtggatgcgtttgtagacattgcaaaagcaggaaagatcaccggtgccagaaaaaatatcgaccgctaccgtcagacttatgcattcgcagcagggaccaaaaaattatatgactacttaaatgacaatccggaatgtatgagtgctcctgttaactatacaaatgatattgcaagggtttcttccattgacaatttcatctctatcaacaatgccgtagatgtggatttatacggacagatcagtgctgagtcttcaggaatcaaacagatcagcggtgccggcggtcagttagacttcgttatgggagcttacttatccaacggcggaaagagttttgtatgcctgtcttccactttcactgacaaggccggacagatgcattcccgtattcttcctaccctgcataacgggtctattgtcacagacacccgtgccaatgcacactatatcgtcaccgaatacggaatggcaaacatgaaaggactctctgcatggcagcgcgcggaagccctgatcaatatcgctcatccggacttcagagaccagctgatcaaagacgcagaaaaagctcaaatctggagaagatccaacaaatag

SEQ ID NO: 175 - DNA, 1068 bp, Clostridium butyricum
  misc_feature: butyrate kinase (buk) from Clostridium butyricum
atggcttacaaattattaataattaatccaggttcaacatcaacaaaaataggtgtatatgaagatgaaaaagaattatttgaagaaactttaagacatacaaatgaagaattaaagcagtttgatgcgatttttgatcaatttcaatttagaaaagatgtaattttaaaagttttaagtgaaaaaaactttgatataaaaacattaagtgctgttgttggtagaggtggaatgcttaaaccagttgaaggtggtacttatgctgtaaatgatgcaatggtagaagatttaaaggtaggagttcaaggacctcatgcttctaacttaggaggtatacttgcaaggtcaatagctgatgaaataggagtaccatctttcattgttgatccagttgttacagatgaacttgcagatgttgcaagattatctggtacaccagatattccaagaaaaagtaagttccatgcattaaatcaaaaggcagttgcaaagagatatggaaaagaaagtggaaaaggatatgaaaatttaaatcttgttgttgtacacatgggtggtggagtttctgttggagctcacaatcatggaaaagttgttgatgtaaacaatgcacttgatggagatggtccattttctccagaaagagctggatcagttccagctggagatttaattaaaatgtgtttcagtggtaagtatagcgaatcagaagtatacagcaaaatagttggtaaaggtggattcgttggatatttaaatactaatgatgtaaaaggaactattgataagatggaagctggagataaggaatgtgaaaacatatataaagctttcttatatcaaataacaaaagcaattggagaaatgtcagctgcattaaatggtaaagttgatcagatagttttaacaggtggtattgcatattctccaacacttgtaccagatttaaaagctaatgttgaatggattgcaccagtaacagtatatccaggagaagatgaattgttagctttagctcaaggtgctataagagttcttgatggagaagaaaaggctaaagtttattaa

SEQ ID NO: 176 - DNA, 1296 bp, Flavonifractor plautii
  misc_feature: 4-hydroxybutyrate CoA-transferase/4Hbt (Flavonifractor plautii)
atgaattggaaggaatattacaagcagcacctgatgagcgcggaggacgcggtcaaccacatccagtcgggcaaccgggtggtggtggcccacgcctgcggcgagccgtcctatctgctggacgtaatggtggccaacgccaaggcctacagggacgtggagatcgtccacatggtggccatgggcaaggccgagtactgcaagcccgagtatgccggtaacttccgccacaacgccttctttgtgggcggcaccagccgggacgccatcgccgagggccggggcgacttcacccccagcttcttctttgaggtgccccgccagttcagctccactatgccggtggacgtggccatggtcatggtgacgcctcccgatgagaacggcatgtgctccctgggcgtgtcggtggactacaccctggaggccgtcaagcaggccaagctggtcatcgcccaggtgaacccccagatgccctggaccggcccctacagcctggtgtccgtcaaggatctggactgcatcgtggagcatgaggcccccatcattgagctcaagccccccaagatcggcgacattgagaaggccatcggagagcactgcgcctccctggtgcccgacggggccaccctccagttgggcatcggcgccatccccgacgccgtgctcctcttcctcaagcacaagaaggacctgggcatccactccgagatgttctccgacggcgtggtggagctggccgaggccggcgtcatcaccaacgccaaaaagaccctccaccccggcaagtttgaggtggcgttcctcatgggcacccgccgcctttacgacttcgtggatcacaaccccgacgtggagctgcgccctgtggactatgtcaacaacccctttgtcattgcccagaacgagaacatggtgtccatcaactcctgcgtacaggtggatctgatgggccaggtggcctccgagagcatcggccccaagcagatctccggcgtgggcggccaggtggacttcgtccgcggcgcttccgccagcaagggcggcgtgtccatcatggccatgccctccaccgtcaagggcaaaatctccaagatcgtgccgctgctggatgagggcgccgccgtcaccaccagccgcaacgacgtggactacatcgtcaccgagtacggcgtggccgcgctgaagggccagaccctccgccagcgcgcccgcaacctcatcgaaattgcccaccccgacttccgcgacgagctcaaggcggagtacgagaagcgcttccactgcaagtattaa

SEQ ID NO: 177 - DNA, 660 bp, Butyricimonas faecalis
  misc_feature: Butyrate-acetoacetate CoA-transferase subunit B from Butyricimonas faecalis)
atggataaagatcaagtaagagaagttatcgcgaaacgggtagctctcgaattaaaagacggcgacgtggtaaacttgggaatcgggcttccgacgatggtaccgaactacttgcccaaaaacgtgcacgtgatcctacaatccgagaacggattattaggtatgggtccgaaaccggaagaaggtcaagaaaatccggagttgacggatgctggtggtggttacatcacggcaatcgaaggagcctgcagctttgacagtgcaacctcttttggacttatccgcggcggacacgtggatgtcaccattcttggtgctttacaggtagacgagaaaggaaacctggcaaactggatcatccccggcaaaaaagccccgggtatgggtggagccatggaccttttggttggtgctaaaaaagtgatccttgccatggaacacacggcaaaagggaatcacaagattttgaaagagtgcaccctgccgttgacggctgccggggaagttgacatgattatcaccgaaatgggtgttatgaacgttacccccgaaggtatcgaactggtagaattaaaccctgaatttacgttagatgacatcaaagcagctacaggctgtgaacttataatttcaaaaaatttaaaagcaatgcctcaatga

SEQ ID NO: 178 - DNA, 645 bp, Butyricimonas faecalis
  misc_feature: Butyrate-acetoacetate CoA-transferase subunit B from Butyricimonas faecalis
atgagtaaatttatttctatcgaagaggctgtatcaaaagtaaaagatggcatgaccatcatggtaggcggattcctcgcaaacggaaccccgaacaagatcgttgatgcattagcgaaatcaggcgttaagaacttgacgttgatctgtaatgacacggcttacccggacaaaggagtaggtcaattgattgccaacaaacaagtaaagaaacttttcgtgtcacacatcgggaccaacccgcacacgagcgaacaaatgaatagcggagagctagaaatcgaattctgcccgcaaggttcattggcagagcgtgtgcgtgccggaggttgcggactgggaggtatcctgactcaaaccggaatgggtactatcgtggctgaagggaaacagatcgtcaacgtggatgggaaagactatttgttggaaaaaacgttgagagccgacatcgcgttggttggcgccagccttggggataaagcagggaatctggtttaccgggggacatctcaaaatttcaacccgttaatggcttctgccgcagacctggtaattgccgaggttaacgagttggttgaagtcggtgaaattgccgcagaaaacgtgaagacaccttccatcatggtggacttcatcgttaaataa